SEQUENCE LISTING (314 sequences)

SEQ ID NO: 1 - DNA, 873 bp, Glycine max
cagctttgaacccaacacttgctttagtctctgtttgtggtgacataaatccctccctacaactcatgaattaagaattctcgaaacaacaaggtctactcgacatgacttaggagttaatgaaatctttcatttcacaggaatctttcccaaataataaactaatattatcaattaatccccaaggttcgttattattaaacgatggtatttgatttgtcaaatacatcattattgtatactcttttatatgtatggcgcaacccaattttttttttttttttttcaatttttaatctcctatccctttttttaattttggaatcgaaatatctaatctaaataatccaaaaattcactcttaatcttgacagtgatatatgttgtatatgtaaatcctagatgtaaaaataggcagacttctcccgggaaaaaaagtaggtaaaaaaatatgctaagtataaattgtctactgatgaaatacgaaagctgatgatattgaaaaaaaaagaattgtaaataaggttcaggttcgacttccatagagaatatagatgatattgtctataatgatagaaaaaaataaaaaactttcgaaagattttatccaattgaaattttaaaaatgactgggataaacttaagaattcaattattgaaattgaataagtaaacaatttaattggattggtcgaatggtatcaaaaaaatggagtactaactcccatttcttatttattattgaattaaccgatcaacttgctttgttatcgaacatttctttttgatttcaaaaattttccggaaaaaatttgaatctatttttatatgcgaatcaatcctactacttctggtcctgaggtttctgcgctt

SEQ ID NO: 2 - DNA, 183 bp, Glycine max
ccaaaaattcactcttaatcttgacagtgatatatgttgtatatgtaaatcctagatgtaaaaataggcagacttctcccgggaaaaaaagtaggtaaaaaaatatgctaagtataaattgtctactgatgaaatacgaaagctgatgatattgaaaaaaaaagaattgtaaataaggttcag

SEQ ID NO: 3 - DNA, 530 bp, Glycine max
cagctttgaacccaacacttgctttagtctctgtttgtggtgacataaatccctccctacaactcatgaattaagaattctcgaaacaacaaggtctactcgacatgacttaggagttaatgaaatctttcatttcacaggaatctttcccaaataataaactaatattatcaattaatccccaaggttcgttattattaaacgatggtatttgatttgtcaaatacatcattattgtatactcttttatatgtatggcgcaacccaattttttttttttttttttcaatttttaatctcctatccctttttttaattttggaatcgaaatatctaatctaaataatccaaaaattcactcttaatcttgacagtgatatatgttgtatatgtaaatcctagatgtaaaaataggcagacttctcccgggaaaaaaagtaggtaaaaaaatatgctaagtataaattgtctactgatgaaatacgaaagctgatgatattgaaaaaaaaagaattgtaaataaggttcag

SEQ ID NO: 4 - DNA, 526 bp, Glycine max
ccaaaaattcactcttaatcttgacagtgatatatgttgtatatgtaaatcctagatgtaaaaataggcagacttctcccgggaaaaaaagtaggtaaaaaaatatgctaagtataaattgtctactgatgaaatacgaaagctgatgatattgaaaaaaaaagaattgtaaataaggttcaggttcgacttccatagagaatatagatgatattgtctataatgatagaaaaaaataaaaaactttcgaaagattttatccaattgaaattttaaaaatgactgggataaacttaagaattcaattattgaaattgaataagtaaacaatttaattggattggtcgaatggtatcaaaaaaatggagtactaactcccatttcttatttattattgaattaaccgatcaacttgctttgttatcgaacatttctttttgatttcaaaaattttccggaaaaaatttgaatctatttttatatgcgaatcaatcctactacttctggtcctgaggtttctgcgctt

SEQ ID NO: 5 - DNA, 1274 bp, Glycine max
cgtagcgtctaccaatttcgccatatccccctctttcatctatactcgataccattatttgcttgaattagaaatgattctattatctatctattcacaattcaatatacacagatatataccacatatctatttcgattctatgcccctacttctattattttttcattaaattttgaatactcgaatggtcgattcttttttttttttcatcttagttttttatcttctctttccgactgaaaacttgggagtcttttattatgatctaggttgggcttttctctttctttcatttttttcttttttccttaaagcaaacagatacagactatctataacaaaacaaaaaaatgaaaattttcatttttttgtttttgttgaaataaacaatccatttattcatatagaattgatagaactaaaacgaatcgaatggctataagtaaccattcttttaatgtagaattagacattaatttagaaatatgaaattcctaattattattattagttactttactaaaatttacaataataatatttttgaaataatcaaatagatatctatttgataaatcgatcgaaatttattatattaatatgaattattaatccctattttgtactttatgttatgcactaaaatatcaaaaaaataatataaataagaaatattggattggatattctatattttctatgtttgtattaatttgaattatgttataaataactaacaatttataattaagatcccttttattaaattcctatgcatacgcatattttggttatgttagcccgcttagctcagaggttagagcatcgcatttgtaatgcgatggtcatcggttcgaatccgatagccggcttttctcaatttgttttcaattttttacataattgagaatatcttttttataaataaaaaaattgggagaattcgatcgttgtggaacaaatcaagaaaagaacctacttcttttctttttttttctatacaatagaataaagttcggatattgatagaattcgtctaattcttctttgtagtataatactttagtagattttgcttcatttataatagaatatttttcatttagtttagttttaattttgatttataagattttccattaaaaaaggagtctttatgtcacgttacagaggacctcgtttcaaaaaaatacgtcgtctggggtctttaccaggactaactagtaaaaggccaacagtcaaaagcgaacttagaaatcaatcgcgctcca

SEQ ID NO: 6 - DNA, 255 bp, Glycine max
atttttttcttttttccttaaagcaaacagatacagactatctataacaaaacaaaaaaatgaaaattttcatttttttgtttttgttgaaataaacaatccatttattcatatagaattgatagaactaaaacgaatcgaatggctataagtaaccattcttttaatgtagaattagacattaatttagaaatatgaaattcctaattattattattagttactttactaaaatttacaataataatatttttg

SEQ ID NO: 7 - DNA, 549 bp, Glycine max
cgtagcgtctaccaatttcgccatatccccctctttcatctatactcgataccattatttgcttgaattagaaatgattctattatctatctattcacaattcaatatacacagatatataccacatatctatttcgattctatgcccctacttctattattttttcattaaattttgaatactcgaatggtcgattcttttttttttttcatcttagttttttatcttctctttccgactgaaaacttgggagtcttttattatgatctaggttgggcttttctctttctttcatttttttcttttttccttaaagcaaacagatacagactatctataacaaaacaaaaaaatgaaaattttcatttttttgtttttgttgaaataaacaatccatttattcatatagaattgatagaactaaaacgaatcgaatggctataagtaaccattcttttaatgtagaattagacattaatttagaaatatgaaattcctaattattattattagttactttactaaaatttacaataataatatttttg

SEQ ID NO: 8 - DNA, 980 bp, Glycine max
atttttttcttttttccttaaagcaaacagatacagactatctataacaaaacaaaaaaatgaaaattttcatttttttgtttttgttgaaataaacaatccatttattcatatagaattgatagaactaaaacgaatcgaatggctataagtaaccattcttttaatgtagaattagacattaatttagaaatatgaaattcctaattattattattagttactttactaaaatttacaataataatatttttgaaataatcaaatagatatctatttgataaatcgatcgaaatttattatattaatatgaattattaatccctattttgtactttatgttatgcactaaaatatcaaaaaaataatataaataagaaatattggattggatattctatattttctatgtttgtattaatttgaattatgttataaataactaacaatttataattaagatcccttttattaaattcctatgcatacgcatattttggttatgttagcccgcttagctcagaggttagagcatcgcatttgtaatgcgatggtcatcggttcgaatccgatagccggcttttctcaatttgttttcaattttttacataattgagaatatcttttttataaataaaaaaattgggagaattcgatcgttgtggaacaaatcaagaaaagaacctacttcttttctttttttttctatacaatagaataaagttcggatattgatagaattcgtctaattcttctttgtagtataatactttagtagattttgcttcatttataatagaatatttttcatttagtttagttttaattttgatttataagattttccattaaaaaaggagtctttatgtcacgttacagaggacctcgtttcaaaaaaatacgtcgtctggggtctttaccaggactaactagtaaaaggccaacagtcaaaagcgaacttagaaatcaatcgcgctcca

SEQ ID NO: 9 - DNA, 1568 bp, Glycine max
aagttacaaaggtcgtacctgtgaaccaaccccccaaggcgaaataggcgcaagggaagagtaatagaccggaccaacctacaaaaacgaaacggtccctccgcaaccagtcatccataatatcaaataaatcattttcatctttggtaaatttaccaagagctatagtcatagcgatcctcctattcaactacttcaaccatttcccaacacctcatagcattttcagggatgggacggaccctcgaggcttttatcatttatctatctatatatagagagagataattttctcgtacactttcccttcttttttaattttaattggtttcgaataaaaaataatttatgagtctattgatatctagggcaaatcgatgaactcaatgtagttattcctttctattcttaaaaatcctccaagtcataaatcataaaatctcctatgactcaaaaatccgatagaaaatttttgtaaaagaactgttcaagaaacaaatgatcccctctttcactccttatacccagcggatcctcagacatatttctctcctttcatcagagaatcttattcttgaatcttgtttgtgaacttacttctattttgtctagggaactactctaggatcatagattttataaatttctatttgatttacacttttttctttttattaattattatttgatttttttattcattattattattattttattaattattaaaaaaaatgaaatacaatattaaaaagaaaaaactgtaagtgaaagtttctgtctcacatacattaattgctggaaaaatatcgtacgcatcgatatgaaaagaaaatatttgatacgcgaagacataattgaatgtatttttcaattatttctataagataaaatctatctatatcgattttatcttatagaaataattgaaattgaaatgaattttttcttgtattcggaaaaaaagatgtttcaaatgaataaaaacttgtttggtggaacttggaataaggcccttctgagtaaagaaagggcatagcccttttttttataggaccttcacaggaacaagaagatttaattggaaatatcgacagattcttagggagtctaaaccaaaaaagtattaaaaagaaaatgaaaaaatcattgttcgaatttcatctttatccaatttgaatatcagaatagtaaatataggtataattcaatgggttaggtccacttactttttgttaatctttccctttttaaaaattttacactttttttttgaatttctttctttactatattctaatgaaattctatgattacttgattttttattacaaatagaaacttgttccaaatatccacaatatttcgattcttccttaaaatagaaatacgactgctttatttctggaaaaaaagtaaaaagccccttatcggatttgaaccgatgacttacgccttaccatggcgttactctaccactgagttaaaggggccctatttgattcaattcctaaataaggaactagctaatatgactagtacatctaattgttactgcgtatacttat

SEQ ID NO: 10 - DNA, 549 bp, Glycine max
agaaacaaatgatcccctctttcactccttatacccagcggatcctcagacatatttctctcctttcatcagagaatcttattcttgaatcttgtttgtgaacttacttctattttgtctagggaactactctaggatcatagattttataaatttctatttgatttacacttttttctttttattaattattatttgatttttttattcattattattattattttattaattattaaaaaaaatgaaatacaatattaaaaagaaaaaactgtaagtgaaagtttctgtctcacatacattaattgctggaaaaatatcgtacgcatcgatatgaaaagaaaatatttgatacgcgaagacataattgaatgtatttttcaattatttctataagataaaatctatctatatcgattttatcttatagaaataattgaaattgaaatgaattttttcttgtattcggaaaaaaagatgtttcaaatgaataaaaacttgtttggtggaacttggaataaggcccttctgagtaaagaaagggcatag

SEQ ID NO: 11 - DNA, 1039 bp, Glycine max
aagttacaaaggtcgtacctgtgaaccaaccccccaaggcgaaataggcgcaagggaagagtaatagaccggaccaacctacaaaaacgaaacggtccctccgcaaccagtcatccataatatcaaataaatcattttcatctttggtaaatttaccaagagctatagtcatagcgatcctcctattcaactacttcaaccatttcccaacacctcatagcattttcagggatgggacggaccctcgaggcttttatcatttatctatctatatatagagagagataattttctcgtacactttcccttcttttttaattttaattggtttcgaataaaaaataatttatgagtctattgatatctagggcaaatcgatgaactcaatgtagttattcctttctattcttaaaaatcctccaagtcataaatcataaaatctcctatgactcaaaaatccgatagaaaatttttgtaaaagaactgttcaagaaacaaatgatcccctctttcactccttatacccagcggatcctcagacatatttctctcctttcatcagagaatcttattcttgaatcttgtttgtgaacttacttctattttgtctagggaactactctaggatcatagattttataaatttctatttgatttacacttttttctttttattaattattatttgatttttttattcattattattattattttattaattattaaaaaaaatgaaatacaatattaaaaagaaaaaactgtaagtgaaagtttctgtctcacatacattaattgctggaaaaatatcgtacgcatcgatatgaaaagaaaatatttgatacgcgaagacataattgaatgtatttttcaattatttctataagataaaatctatctatatcgattttatcttatagaaataattgaaattgaaatgaattttttcttgtattcggaaaaaaagatgtttcaaatgaataaaaacttgtttggtggaacttggaataaggcccttctgagtaaagaaagggcatag

SEQ ID NO: 12 - DNA, 1078 bp, Glycine max
agaaacaaatgatcccctctttcactccttatacccagcggatcctcagacatatttctctcctttcatcagagaatcttattcttgaatcttgtttgtgaacttacttctattttgtctagggaactactctaggatcatagattttataaatttctatttgatttacacttttttctttttattaattattatttgatttttttattcattattattattattttattaattattaaaaaaaatgaaatacaatattaaaaagaaaaaactgtaagtgaaagtttctgtctcacatacattaattgctggaaaaatatcgtacgcatcgatatgaaaagaaaatatttgatacgcgaagacataattgaatgtatttttcaattatttctataagataaaatctatctatatcgattttatcttatagaaataattgaaattgaaatgaattttttcttgtattcggaaaaaaagatgtttcaaatgaataaaaacttgtttggtggaacttggaataaggcccttctgagtaaagaaagggcatagcccttttttttataggaccttcacaggaacaagaagatttaattggaaatatcgacagattcttagggagtctaaaccaaaaaagtattaaaaagaaaatgaaaaaatcattgttcgaatttcatctttatccaatttgaatatcagaatagtaaatataggtataattcaatgggttaggtccacttactttttgttaatctttccctttttaaaaattttacactttttttttgaatttctttctttactatattctaatgaaattctatgattacttgattttttattacaaatagaaacttgttccaaatatccacaatatttcgattcttccttaaaatagaaatacgactgctttatttctggaaaaaaagtaaaaagccccttatcggatttgaaccgatgacttacgccttaccatggcgttactctaccactgagttaaaggggccctatttgattcaattcctaaataaggaactagctaatatgactagtacatctaattgttactgcgtatacttat

SEQ ID NO: 13 - DNA, 980 bp, Glycine max
ttccttaaaatagaaatacgactgctttatttctggaaaaaaagtaaaaagccccttatcggatttgaaccgatgacttacgccttaccatggcgttactctaccactgagttaaaggggccctatttgattcaattcctaaataaggaactagctaatatgactagtacatctaattgttactgcgtatacttattatataaatttatataaattatataaatgggattctaatatatgtacatcaatatataatttatataatttgcataatgacttatcaaagaacaaaaaatggtatttacctagtgaatagaatataataaaataataataattttttcatattggatttgaccaatatcaatggaatacattctttcaaaattgagtctggaagtcattctaatcataacaccaaattcatttcattgatacataaatgtactcaccaattcaaatatttcatcgaatcattgataaatggattccatttgtcctgtccctaaaccaaattcgtattttattttgaaaagaatttcaaaaacttgttgatctctatttacccaatttccagattgattatcgtttttttatttcccggcttaggattagatataaatatacctaatacctatcgaatcttagtaataaaggacagtgaaagaaatgaagttttaacctaccgtcctttccaacaaaccaatcattcccgtaaaggattttctctttcgaaatcaaaatgattaatcaaaaatttctatggaattctgaaagatgtaaaaattttttttatcgaatcatatcattccattatattgacaattcaaaaaaactgttcatactatgaacgtagtataatggcggtcgggcaagtatgcccccatcgtctagtggttcaggacatctctctttcaaggaggcagcggggattcgacttcccctgggggtagggtgctacgaaaggaggttgatcatga

SEQ ID NO: 14 - DNA, 392 bp, Glycine max
atggtatttacctagtgaatagaatataataaaataataataattttttcatattggatttgaccaatatcaatggaatacattctttcaaaattgagtctggaagtcattctaatcataacaccaaattcatttcattgatacataaatgtactcaccaattcaaatatttcatcgaatcattgataaatggattccatttgtcctgtccctaaaccaaattcgtattttattttgaaaagaatttcaaaaacttgttgatctctatttacccaatttccagattgattatcgtttttttatttcccggcttaggattagatataaatatacctaatacctatcgaatcttagtaataaaggacagtgaaagaaatgaagttttaacctac

SEQ ID NO: 15 - DNA, 686 bp, Glycine max
ttccttaaaatagaaatacgactgctttatttctggaaaaaaagtaaaaagccccttatcggatttgaaccgatgacttacgccttaccatggcgttactctaccactgagttaaaggggccctatttgattcaattcctaaataaggaactagctaatatgactagtacatctaattgttactgcgtatacttattatataaatttatataaattatataaatgggattctaatatatgtacatcaatatataatttatataatttgcataatgacttatcaaagaacaaaaaatggtatttacctagtgaatagaatataataaaataataataattttttcatattggatttgaccaatatcaatggaatacattctttcaaaattgagtctggaagtcattctaatcataacaccaaattcatttcattgatacataaatgtactcaccaattcaaatatttcatcgaatcattgataaatggattccatttgtcctgtccctaaaccaaattcgtattttattttgaaaagaatttcaaaaacttgttgatctctatttacccaatttccagattgattatcgtttttttatttcccggcttaggattagatataaatatacctaatacctatcgaatcttagtaataaaggacagtgaaagaaatgaagttttaacctac

SEQ ID NO: 16 - DNA, 686 bp, Glycine max
atggtatttacctagtgaatagaatataataaaataataataattttttcatattggatttgaccaatatcaatggaatacattctttcaaaattgagtctggaagtcattctaatcataacaccaaattcatttcattgatacataaatgtactcaccaattcaaatatttcatcgaatcattgataaatggattccatttgtcctgtccctaaaccaaattcgtattttattttgaaaagaatttcaaaaacttgttgatctctatttacccaatttccagattgattatcgtttttttatttcccggcttaggattagatataaatatacctaatacctatcgaatcttagtaataaaggacagtgaaagaaatgaagttttaacctaccgtcctttccaacaaaccaatcattcccgtaaaggattttctctttcgaaatcaaaatgattaatcaaaaatttctatggaattctgaaagatgtaaaaattttttttatcgaatcatatcattccattatattgacaattcaaaaaaactgttcatactatgaacgtagtataatggcggtcgggcaagtatgcccccatcgtctagtggttcaggacatctctctttcaaggaggcagcggggattcgacttcccctgggggtagggtgctacgaaaggaggttgatcatga

SEQ ID NO: 17 - DNA, 2352 bp, Glycine max
gttaagaaattgaaaaacccaagtagaaaacaaaatgggtactttgatcccttaactcaattttgagtatccctatagtccacttcttccccatactacgagggaaagagaaaatgaaaaaactaccattaaagcagcccaagcaagacttactatatccatgtaaattttgtctcctatctctaaaaatatgaaggaattttttaattattattcaaaaatttttcttctattattttcttttgtattattcactaaaaagactataataatagcggaattactgttagagagtcaaaaaaaggattcagggctaacaccattgacgaaaaacaagaatcctttctattagaacatctaaaaagttttttttatatgatttttagtaaaataggaaaatattaagcataaacaacacatccggaaacatcctcggaagttttaagtaaatgaatgttactaacaggtaggaataataagatttctgttttacccccccatttcattaattcattaagtaatttcattaagtataaaaaaaagaatcaagacagattcatttgcatacttatactctttcttatttctatttgaaaaaatttcgtaatgtctcccgattcgttctctaaaaaaactaaaataatcggaagatagcttgcctattcattttttttactagaggttagtgaaaagaaagattttcttttttctattcgattttagaataaaaaaattttgtaaaatattttaaattatatatataagaaattatatttatattatataattatatatatatatacttttaagattaagaaagcgaattagctaaattaaaaactattcaatctaactaatctaactaatattaatgaaatgtggaagcactaatagactttccatcagtctgtatacaaggtttttcttccgcccgttctctaactaaaaatggaattccctatccaacttatccttatccaatctaattcgagacccagtcagtagacggacactacaatagttgtgtagtgaaagaactatcatttctaaatttatggattccttttcactacttgaatccgagacgaaaatatttacatacagcattttttagaataaacctactgatgcttagaatttataatcaaacgcaagtctcaagactccttttccctagcttgcttctattggaaaaaagttttctataaaaaacgtaatacaatatttcatttcaaatgtcttattgcttaggcgacacccggatttgaactggggaaaaaggatttgcagtcccccgccttaccactcggccatgccgccaaatagatatagatatataaatatatgaagtatatgagaataccccttttttttctattgaaaaaaaaaaaaaatatcgagacaattcgcaaccgttaactattcatttatgaattattctttaagatttgaatctaaaatggtttttttcttaatgagataagaaaagaaaattgatacataaccaatccatattgcttttttattgaaaaaaaaactttctccattccaattataacagcaactgtcaatatatgtaaaccctagaacataaatttgaatagttacacagatattatctaatcgggtatcttatccatccatatatatataataccgatactatgcggaacggaattatcaagaaattgtcgtgcttctccctattttacaatttttctattcaataaattgaatagaaaaattgtaaaatagggagaagcacgacatgttatgtgttgtcccaaacaaatatgactgcaatttacaatccgaattcattttttctggtatccaattgaattggaatatggaatatcataataatgatagaaccgggatgcatttttttatattccttaaaatgaaaaaaatcttgaaatccgggtcgaatttttcaattcatttccattttgagattataatttagattctatctgtttgttttgttgcaaattccttccttcgaagttgagtataaaattctattttatttattcctcttttcataataggtatttcatacacgggattagttaaaatttcatgtaattcagtataaagaacagaaattccagtattgctaaattgattcatgtgatttgatgaagaatggcagcaaatcggtggaatatttttcgataaaattcacggggaaattgaaaatgcttggggatggaaatgaaggaatgtctacactacctggattgaatcagatacaatttgaagggttttgtaggttcattgatcggggcttaccagaagggctttttaagtttccaaaaattgaggatacagatcaagaaattgaat

SEQ ID NO: 18 - DNA, 392 bp, Glycine max
aggaaaatattaagcataaacaacacatccggaaacatcctcggaagttttaagtaaatgaatgttactaacaggtaggaataataagatttctgttttacccccccatttcattaattcattaagtaatttcattaagtataaaaaaaagaatcaagacagattcatttgcatacttatactctttcttatttctatttgaaaaaatttcgtaatgtctcccgattcgttctctaaaaaaactaaaataatcggaagatagcttgcctattcattttttttactagaggttagtgaaaagaaagattttcttttttctattcgattttagaataaaaaaattttgtaaaatattttaaattatatatataagaaattatatttatattata

SEQ ID NO: 19 - DNA, 784 bp, Glycine max
gttaagaaattgaaaaacccaagtagaaaacaaaatgggtactttgatcccttaactcaattttgagtatccctatagtccacttcttccccatactacgagggaaagagaaaatgaaaaaactaccattaaagcagcccaagcaagacttactatatccatgtaaattttgtctcctatctctaaaaatatgaaggaattttttaattattattcaaaaatttttcttctattattttcttttgtattattcactaaaaagactataataatagcggaattactgttagagagtcaaaaaaaggattcagggctaacaccattgacgaaaaacaagaatcctttctattagaacatctaaaaagttttttttatatgatttttagtaaaataggaaaatattaagcataaacaacacatccggaaacatcctcggaagttttaagtaaatgaatgttactaacaggtaggaataataagatttctgttttacccccccatttcattaattcattaagtaatttcattaagtataaaaaaaagaatcaagacagattcatttgcatacttatactctttcttatttctatttgaaaaaatttcgtaatgtctcccgattcgttctctaaaaaaactaaaataatcggaagatagcttgcctattcattttttttactagaggttagtgaaaagaaagattttcttttttctattcgattttagaataaaaaaattttgtaaaatattttaaattatatatataagaaattatatttatattata

SEQ ID NO: 20 - DNA, 1960 bp, Glycine max
aggaaaatattaagcataaacaacacatccggaaacatcctcggaagttttaagtaaatgaatgttactaacaggtaggaataataagatttctgttttacccccccatttcattaattcattaagtaatttcattaagtataaaaaaaagaatcaagacagattcatttgcatacttatactctttcttatttctatttgaaaaaatttcgtaatgtctcccgattcgttctctaaaaaaactaaaataatcggaagatagcttgcctattcattttttttactagaggttagtgaaaagaaagattttcttttttctattcgattttagaataaaaaaattttgtaaaatattttaaattatatatataagaaattatatttatattatataattatatatatatatacttttaagattaagaaagcgaattagctaaattaaaaactattcaatctaactaatctaactaatattaatgaaatgtggaagcactaatagactttccatcagtctgtatacaaggtttttcttccgcccgttctctaactaaaaatggaattccctatccaacttatccttatccaatctaattcgagacccagtcagtagacggacactacaatagttgtgtagtgaaagaactatcatttctaaatttatggattccttttcactacttgaatccgagacgaaaatatttacatacagcattttttagaataaacctactgatgcttagaatttataatcaaacgcaagtctcaagactccttttccctagcttgcttctattggaaaaaagttttctataaaaaacgtaatacaatatttcatttcaaatgtcttattgcttaggcgacacccggatttgaactggggaaaaaggatttgcagtcccccgccttaccactcggccatgccgccaaatagatatagatatataaatatatgaagtatatgagaataccccttttttttctattgaaaaaaaaaaaaaatatcgagacaattcgcaaccgttaactattcatttatgaattattctttaagatttgaatctaaaatggtttttttcttaatgagataagaaaagaaaattgatacataaccaatccatattgcttttttattgaaaaaaaaactttctccattccaattataacagcaactgtcaatatatgtaaaccctagaacataaatttgaatagttacacagatattatctaatcgggtatcttatccatccatatatatataataccgatactatgcggaacggaattatcaagaaattgtcgtgcttctccctattttacaatttttctattcaataaattgaatagaaaaattgtaaaatagggagaagcacgacatgttatgtgttgtcccaaacaaatatgactgcaatttacaatccgaattcattttttctggtatccaattgaattggaatatggaatatcataataatgatagaaccgggatgcatttttttatattccttaaaatgaaaaaaatcttgaaatccgggtcgaatttttcaattcatttccattttgagattataatttagattctatctgtttgttttgttgcaaattccttccttcgaagttgagtataaaattctattttatttattcctcttttcataataggtatttcatacacgggattagttaaaatttcatgtaattcagtataaagaacagaaattccagtattgctaaattgattcatgtgatttgatgaagaatggcagcaaatcggtggaatatttttcgataaaattcacggggaaattgaaaatgcttggggatggaaatgaaggaatgtctacactacctggattgaatcagatacaatttgaagggttttgtaggttcattgatcggggcttaccagaagggctttttaagtttccaaaaattgaggatacagatcaagaaattgaat

SEQ ID NO: 21 - DNA, 294 bp, Glycine max
caatatatgtaaaccctagaacataaatttgaatagttacacagatattatctaatcgggtatcttatccatccatatatatataataccgatactatgcggaacggaattatcaagaaattgtcgtgcttctccctattttacaatttttctattcaataaattgaatagaaaaattgtaaaatagggagaagcacgacatgttatgtgttgtcccaaacaaatatgactgcaatttacaatccgaattcattttttctggtatccaattgaattggaatatggaatatcata

SEQ ID NO: 22 - DNA, 1862 bp, Glycine max
gttaagaaattgaaaaacccaagtagaaaacaaaatgggtactttgatcccttaactcaattttgagtatccctatagtccacttcttccccatactacgagggaaagagaaaatgaaaaaactaccattaaagcagcccaagcaagacttactatatccatgtaaattttgtctcctatctctaaaaatatgaaggaattttttaattattattcaaaaatttttcttctattattttcttttgtattattcactaaaaagactataataatagcggaattactgttagagagtcaaaaaaaggattcagggctaacaccattgacgaaaaacaagaatcctttctattagaacatctaaaaagttttttttatatgatttttagtaaaataggaaaatattaagcataaacaacacatccggaaacatcctcggaagttttaagtaaatgaatgttactaacaggtaggaataataagatttctgttttacccccccatttcattaattcattaagtaatttcattaagtataaaaaaaagaatcaagacagattcatttgcatacttatactctttcttatttctatttgaaaaaatttcgtaatgtctcccgattcgttctctaaaaaaactaaaataatcggaagatagcttgcctattcattttttttactagaggttagtgaaaagaaagattttcttttttctattcgattttagaataaaaaaattttgtaaaatattttaaattatatatataagaaattatatttatattatataattatatatatatatacttttaagattaagaaagcgaattagctaaattaaaaactattcaatctaactaatctaactaatattaatgaaatgtggaagcactaatagactttccatcagtctgtatacaaggtttttcttccgcccgttctctaactaaaaatggaattccctatccaacttatccttatccaatctaattcgagacccagtcagtagacggacactacaatagttgtgtagtgaaagaactatcatttctaaatttatggattccttttcactacttgaatccgagacgaaaatatttacatacagcattttttagaataaacctactgatgcttagaatttataatcaaacgcaagtctcaagactccttttccctagcttgcttctattggaaaaaagttttctataaaaaacgtaatacaatatttcatttcaaatgtcttattgcttaggcgacacccggatttgaactggggaaaaaggatttgcagtcccccgccttaccactcggccatgccgccaaatagatatagatatataaatatatgaagtatatgagaataccccttttttttctattgaaaaaaaaaaaaaatatcgagacaattcgcaaccgttaactattcatttatgaattattctttaagatttgaatctaaaatggtttttttcttaatgagataagaaaagaaaattgatacataaccaatccatattgcttttttattgaaaaaaaaactttctccattccaattataacagcaactgtcaatatatgtaaaccctagaacataaatttgaatagttacacagatattatctaatcgggtatcttatccatccatatatatataataccgatactatgcggaacggaattatcaagaaattgtcgtgcttctccctattttacaatttttctattcaataaattgaatagaaaaattgtaaaatagggagaagcacgacatgttatgtgttgtcccaaacaaatatgactgcaatttacaatccgaattcattttttctggtatccaattgaattggaatatggaatatcata

SEQ ID NO: 23 - DNA, 784 bp, Glycine max
caatatatgtaaaccctagaacataaatttgaatagttacacagatattatctaatcgggtatcttatccatccatatatatataataccgatactatgcggaacggaattatcaagaaattgtcgtgcttctccctattttacaatttttctattcaataaattgaatagaaaaattgtaaaatagggagaagcacgacatgttatgtgttgtcccaaacaaatatgactgcaatttacaatccgaattcattttttctggtatccaattgaattggaatatggaatatcataataatgatagaaccgggatgcatttttttatattccttaaaatgaaaaaaatcttgaaatccgggtcgaatttttcaattcatttccattttgagattataatttagattctatctgtttgttttgttgcaaattccttccttcgaagttgagtataaaattctattttatttattcctcttttcataataggtatttcatacacgggattagttaaaatttcatgtaattcagtataaagaacagaaattccagtattgctaaattgattcatgtgatttgatgaagaatggcagcaaatcggtggaatatttttcgataaaattcacggggaaattgaaaatgcttggggatggaaatgaaggaatgtctacactacctggattgaatcagatacaatttgaagggttttgtaggttcattgatcggggcttaccagaagggctttttaagtttccaaaaattgaggatacagatcaagaaattgaat

SEQ ID NO: 24 - DNA, 588 bp, Glycine max
ctaaactatacccgctacaatacaattattgtatcttttgtcgaacaaagcaatcagacaaaatcaataaagaaatagaggggcataatattcaatattctaataattcgagtattgacatgtttaattagagtatcttctaatgaaatttgaaaaacggggtgaattacatttttggattttgtttttgctgaaggtatttttttttcgaataaagtacattcaattcaaataaatcattgttattcaatattcatgggaataaaaagagcccgactaggtactggccgggctctttggctgtagaaaaataaaaaaggaaactaaaaaatagaagaatatagaatataataatgaatcgaaatcaaatagaaaaaaaagagataagataaaaaataagataaaaagaagtctttttttttcaagctccattttttgctcttcttgtttatgtgatataacataaacaaagtaattctatttttcaattggggagagatggctgagtggactaaagcgtcggattgctaatccgttgtacaaatttttgtaccgagggttcgaatccctctctttccgttgataatt

SEQ ID NO: 25 - DNA, 98 bp, Glycine max
gtatttttttttcgaataaagtacattcaattcaaataaatcattgttattcaatattcatgggaataaaaagagcccgactaggtactggccgggct

SEQ ID NO: 26 - DNA, 294 bp, Glycine max
ctaaactatacccgctacaatacaattattgtatcttttgtcgaacaaagcaatcagacaaaatcaataaagaaatagaggggcataatattcaatattctaataattcgagtattgacatgtttaattagagtatcttctaatgaaatttgaaaaacggggtgaattacatttttggattttgtttttgctgaaggtatttttttttcgaataaagtacattcaattcaaataaatcattgttattcaatattcatgggaataaaaagagcccgactaggtactggccgggct

SEQ ID NO: 27 - DNA, 392 bp, Glycine max
gtatttttttttcgaataaagtacattcaattcaaataaatcattgttattcaatattcatgggaataaaaagagcccgactaggtactggccgggctctttggctgtagaaaaataaaaaaggaaactaaaaaatagaagaatatagaatataataatgaatcgaaatcaaatagaaaaaaaagagataagataaaaaataagataaaaagaagtctttttttttcaagctccattttttgctcttcttgtttatgtgatataacataaacaaagtaattctatttttcaattggggagagatggctgagtggactaaagcgtcggattgctaatccgttgtacaaatttttgtaccgagggttcgaatccctctctttccgttgataatt

SEQ ID NO: 28 - DNA, 1175 bp, Glycine max
attaaagatattaagcataacaaacattttgttctttaagaaaatataaattatttttgttttttttttaattagaattttcatttttattgtattttatatatatataaaatacaataaaaaggaaaagaaattaatattaatttatgaataaaactaaaaaaaaatgaataaaataagataaaaccctccaaaatccttctttgatttgaacttatctagttcaaaatctttttttattctgaaataaaaaaatcgaataaatcacacttctatacaatatcttaatagaattgtacgtaatgatcaataaattgagtcttatcctatctatatctagatagatatagatagatatgtagatacgcatataaaaatactagcaacaaatttttctatagaaatttttgaactggaattgacgaacaaccaatataaataatagtgtttattagtataaaatcgaaaatggggcgtggccaagtggtaaggcaacgggttttggtcccgctattcggaggttcgaatccttccgtcccagagcatatccattcatgatgtatatcatattggaatacagattgtatatcagaattaaaattatccccctttttgaattattcgtctctaatctaaatcgacttgctttcgaatatgtggattcaaaaacaagtcgagtttttgttactttcttttttgtataaaaattgttttaatgtaatcatttcattatatatatctattataattttataattttttcaattttttttttttctttttttctaatatttttaaattcccatttttctactttacaaattctaaaaagagagtagaaaatagattgataaaataccgagttaattttcatttacgtctttactttaaaaaaagttttcgttatatagaagaaaaaccctgacctaaaaaggatagattattatgtatcgattgaatcaatgactattcatgattccataattgaatcaattacatactggatccaagctaaaggaatgttatggtaaaacttcgtttgaaacgatgtggtaaaaagcaacgtgcgacttgaaagacatgattagattagctgtggattcttacatccgccattttttctagtatatagaaatgaacatgctcttgactcgacatcgtttgttctgttccac

SEQ ID NO: 29 - DNA, 173 bp, Glycine max
atccttctttgatttgaacttatctagttcaaaatctttttttattctgaaataaaaaaatcgaataaatcacacttctatacaatatcttaatagaattgtacgtaatgatcaataaattgagtcttatcctatctatatctagatagatatagatagatatgtagatacgc

SEQ ID NO: 30 - DNA, 369 bp, Glycine max
aattaaagatattaagcataacaaacattttgttctttaagaaaatataaattatttttgttttttttttaattagaattttcatttttattgtattttatatatatataaaatacaataaaaaggaaaagaaattaatattaatttatgaataaaactaaaaaaaaatgaataaaataagataaaaccctccaaaatccttctttgatttgaacttatctagttcaaaatctttttttattctgaaataaaaaaatcgaataaatcacacttctatacaatatcttaatagaattgtacgtaatgatcaataaattgagtcttatcctatctatatctagatagatatagatagatatgtagatacgc

SEQ ID NO: 31 - DNA, 980 bp, Glycine max
atccttctttgatttgaacttatctagttcaaaatctttttttattctgaaataaaaaaatcgaataaatcacacttctatacaatatcttaatagaattgtacgtaatgatcaataaattgagtcttatcctatctatatctagatagatatagatagatatgtagatacgcatataaaaatactagcaacaaatttttctatagaaatttttgaactggaattgacgaacaaccaatataaataatagtgtttattagtataaaatcgaaaatggggcgtggccaagtggtaaggcaacgggttttggtcccgctattcggaggttcgaatccttccgtcccagagcatatccattcatgatgtatatcatattggaatacagattgtatatcagaattaaaattatccccctttttgaattattcgtctctaatctaaatcgacttgctttcgaatatgtggattcaaaaacaagtcgagtttttgttactttcttttttgtataaaaattgttttaatgtaatcatttcattatatatatctattataattttataattttttcaattttttttttttctttttttctaatatttttaaattcccatttttctactttacaaattctaaaaagagagtagaaaatagattgataaaataccgagttaattttcatttacgtctttactttaaaaaaagttttcgttatatagaagaaaaaccctgacctaaaaaggatagattattatgtatcgattgaatcaatgactattcatgattccataattgaatcaattacatactggatccaagctaaaggaatgttatggtaaaacttcgtttgaaacgatgtggtaaaaagcaacgtgcgacttgaaagacatgattagattagctgtggattcttacatccgccattttttctagtatatagaaatgaacatgctcttgactcgacatcgtttgttctgttccac

SEQ ID NO: 32 - DNA, 196 bp, Glycine max
atcagaattaaaattatccccctttttgaattattcgtctctaatctaaatcgacttgctttcgaatatgtggattcaaaaacaagtcgagtttttgttactttcttttttgtataaaaattgttttaatgtaatcatttcattatatatatctattataattttataattttttcaattttttttttttcttttt

SEQ ID NO: 33 - DNA, 784 bp, Glycine max
aattaaagatattaagcataacaaacattttgttctttaagaaaatataaattatttttgttttttttttaattagaattttcatttttattgtattttatatatatataaaatacaataaaaaggaaaagaaattaatattaatttatgaataaaactaaaaaaaaatgaataaaataagataaaaccctccaaaatccttctttgatttgaacttatctagttcaaaatctttttttattctgaaataaaaaaatcgaataaatcacacttctatacaatatcttaatagaattgtacgtaatgatcaataaattgagtcttatcctatctatatctagatagatatagatagatatgtagatacgcatataaaaatactagcaacaaatttttctatagaaatttttgaactggaattgacgaacaaccaatataaataatagtgtttattagtataaaatcgaaaatggggcgtggccaagtggtaaggcaacgggttttggtcccgctattcggaggttcgaatccttccgtcccagagcatatccattcatgatgtatatcatattggaatacagattgtatatcagaattaaaattatccccctttttgaattattcgtctctaatctaaatcgacttgctttcgaatatgtggattcaaaaacaagtcgagtttttgttactttcttttttgtataaaaattgttttaatgtaatcatttcattatatatatctattataattttataattttttcaattttttttttttcttttt

SEQ ID NO: 34 - DNA, 588 bp, Glycine max
atcagaattaaaattatccccctttttgaattattcgtctctaatctaaatcgacttgctttcgaatatgtggattcaaaaacaagtcgagtttttgttactttcttttttgtataaaaattgttttaatgtaatcatttcattatatatatctattataattttataattttttcaattttttttttttctttttttctaatatttttaaattcccatttttctactttacaaattctaaaaagagagtagaaaatagattgataaaataccgagttaattttcatttacgtctttactttaaaaaaagttttcgttatatagaagaaaaaccctgacctaaaaaggatagattattatgtatcgattgaatcaatgactattcatgattccataattgaatcaattacatactggatccaagctaaaggaatgttatggtaaaacttcgtttgaaacgatgtggtaaaaagcaacgtgcgacttgaaagacatgattagattagctgtggattcttacatccgccattttttctagtatatagaaatgaacatgctcttgactcgacatcgtttgttctgttccac

SEQ ID NO: 35 - DNA, 1176 bp, Glycine max
tgctgacaaataaccaacctgcaatgaatagggagggtatagtaatgctatgaataatccagtatcggatactggtaataatatcagcaaaagaacgttctcctgtgcttccagacatgttgaactccacatattcttgtacagtcaaaagggggatcgattccataaaatcagtaagtagaaatttactgaaatgtaatccttgttagatcgtccattttgtaccaacggtgtcttttgagtataccgaatcagtatagctatccttcttctaacagagcaatgaaatttcacaatcgaaatctaggactagataagttctttcttctttcttcattgattgtctcgatgtattcaaaaatccgatagaaaattttttgatagtacatttttatagtaatagtataaaatgaaggttcttcacatttcacattattagtttcggactaaaaactaagtatcaattaaaatcggaatatggtaaattggtttcttagaattcataaaggagattttatatttctagaattcaattagatgccaaatctataaatatactttttgtggttgttgaagatattttttctattttcttctatattaattcaaaattaaagaaattgaaataacaattgaaataacaaagttatttttctttgtttttgttttaatattcatttttctttctaatagaaatagaattctattctagaataatatataaaataatacgaaagattttcattatcattagattagtttactcaattcatgagttgattaataagtctgttgatttggaatcacaggatgagtagatgtcacagatgatgaatttatttctaaattatgtcattctatttctctttttattcgtctgtaataatttattgatgaatctccaattttcaattgtatcttttaagtagtattttttcttatcttcttttttctctcaaaaatggaaatttagggaagtgctttataaacagatgtataaaaataacatatttcatttagcttctatatgctcactataactagttatttcggttttttactagtggttttaattataacctcatctctatttattggtctgagtaaaatacgacttatttgatttgtaattttgagaggaatagtcaattttggaataagacattctatagttcc

SEQ ID NO: 36 - DNA, 294 bp, Glycine max
tatagtaatagtataaaatgaaggttcttcacatttcacattattagtttcggactaaaaactaagtatcaattaaaatcggaatatggtaaattggtttcttagaattcataaaggagattttatatttctagaattcaattagatgccaaatctataaatatactttttgtggttgttgaagatattttttctattttcttctatattaattcaaaattaaagaaattgaaataacaattgaaataacaaagttatttttctttgtttttgttttaatattcatttttcttt

SEQ ID NO: 37 - DNA, 686 bp, Glycine max
tgctgacaaataaccaacctgcaatgaatagggagggtatagtaatgctatgaataatccagtatcggatactggtaataatatcagcaaaagaacgttctcctgtgcttccagacatgttgaactccacatattcttgtacagtcaaaagggggatcgattccataaaatcagtaagtagaaatttactgaaatgtaatccttgttagatcgtccattttgtaccaacggtgtcttttgagtataccgaatcagtatagctatccttcttctaacagagcaatgaaatttcacaatcgaaatctaggactagataagttctttcttctttcttcattgattgtctcgatgtattcaaaaatccgatagaaaattttttgatagtacatttttatagtaatagtataaaatgaaggttcttcacatttcacattattagtttcggactaaaaactaagtatcaattaaaatcggaatatggtaaattggtttcttagaattcataaaggagattttatatttctagaattcaattagatgccaaatctataaatatactttttgtggttgttgaagatattttttctattttcttctatattaattcaaaattaaagaaattgaaataacaattgaaataacaaagttatttttctttgtttttgttttaatattcatttttcttt

SEQ ID NO: 38 - DNA, 784 bp, Glycine max
tatagtaatagtataaaatgaaggttcttcacatttcacattattagtttcggactaaaaactaagtatcaattaaaatcggaatatggtaaattggtttcttagaattcataaaggagattttatatttctagaattcaattagatgccaaatctataaatatactttttgtggttgttgaagatattttttctattttcttctatattaattcaaaattaaagaaattgaaataacaattgaaataacaaagttatttttctttgtttttgttttaatattcatttttctttctaatagaaatagaattctattctagaataatatataaaataatacgaaagattttcattatcattagattagtttactcaattcatgagttgattaataagtctgttgatttggaatcacaggatgagtagatgtcacagatgatgaatttatttctaaattatgtcattctatttctctttttattcgtctgtaataatttattgatgaatctccaattttcaattgtatcttttaagtagtattttttcttatcttcttttttctctcaaaaatggaaatttagggaagtgctttataaacagatgtataaaaataacatatttcatttagcttctatatgctcactataactagttatttcggttttttactagtggttttaattataacctcatctctatttattggtctgagtaaaatacgacttatttgatttgtaattttgagaggaatagtcaattttggaataagacattctatagttcc

SEQ ID NO: 39 - DNA, 980 bp, Glycine max
cttttattttctgttgagtaatcgttcgagtaagtcttgaatgagctcctcgaaaacttgatgtaaataaacgaatttttgtccttcgtttccgagctatatatcctcgtttaattctggtcattgaataattgagactttgatgaataactatttgatttttcagttattcttttcttcttactagtctattaataacaaaaatggattcttccaatgtataaaaaaaaaattccaatggctcttgctactataacctccccaaccacgattttatatatatatttttttttctaaatattcaacctcaaaataataaattgtattgatactagatatcaaaaaacatagtaagtaatagtaaataaattaggacatatataaaaaatgggtgggttccatcgtttctatgattttttttagaaacgggcaggtcctttctatacaccggagcctttctttcttttcattttttattaatttaattaatgttattgttaacttgtacagttcacactctttggctctacccatccaatatttagtttagtaagtagattttttcacaatgaaatctagttatacagtaacggtatttaattatgaaaatttgctgggtagctgaccttcttattccgttcgtccaaaatccatcaaaattcattcattaaggacatactttatctttaattgaaatagtattttctccgcttaacgggtaactttatatttgttttgttaccgatgggaaagtccttctcatcttaaattgtaaattaagactattgggtttgcaccaatcgaaaccataaatttgatacaagatagaagaatgtaaagaatgtatatatataagttaagatagtctgaatgggaaaactacattcacactatcttaaccgatcaacaatactgaaaaaatgaaattggttttttcttcttagtatatgatctgaacgagtcgcacatacaccctggta

SEQ ID NO: 40 - DNA, 294 bp, Glycine max
tgggttccatcgtttctatgattttttttagaaacgggcaggtcctttctatacaccggagcctttctttcttttcattttttattaatttaattaatgttattgttaacttgtacagttcacactctttggctctacccatccaatatttagtttagtaagtagattttttcacaatgaaatctagttatacagtaacggtatttaattatgaaaatttgctgggtagctgaccttcttattccgttcgtccaaaatccatcaaaattcattcattaaggacatactttatct

SEQ ID NO: 41 - DNA, 686 bp, Glycine max
cttttattttctgttgagtaatcgttcgagtaagtcttgaatgagctcctcgaaaacttgatgtaaataaacgaatttttgtccttcgtttccgagctatatatcctcgtttaattctggtcattgaataattgagactttgatgaataactatttgatttttcagttattcttttcttcttactagtctattaataacaaaaatggattcttccaatgtataaaaaaaaaattccaatggctcttgctactataacctccccaaccacgattttatatatatatttttttttctaaatattcaacctcaaaataataaattgtattgatactagatatcaaaaaacatagtaagtaatagtaaataaattaggacatatataaaaaatgggtgggttccatcgtttctatgattttttttagaaacgggcaggtcctttctatacaccggagcctttctttcttttcattttttattaatttaattaatgttattgttaacttgtacagttcacactctttggctctacccatccaatatttagtttagtaagtagattttttcacaatgaaatctagttatacagtaacggtatttaattatgaaaatttgctgggtagctgaccttcttattccgttcgtccaaaatccatcaaaattcattcattaaggacatactttatct

SEQ ID NO: 42 - DNA, 588 bp, Glycine max
tgggttccatcgtttctatgattttttttagaaacgggcaggtcctttctatacaccggagcctttctttcttttcattttttattaatttaattaatgttattgttaacttgtacagttcacactctttggctctacccatccaatatttagtttagtaagtagattttttcacaatgaaatctagttatacagtaacggtatttaattatgaaaatttgctgggtagctgaccttcttattccgttcgtccaaaatccatcaaaattcattcattaaggacatactttatctttaattgaaatagtattttctccgcttaacgggtaactttatatttgttttgttaccgatgggaaagtccttctcatcttaaattgtaaattaagactattgggtttgcaccaatcgaaaccataaatttgatacaagatagaagaatgtaaagaatgtatatatataagttaagatagtctgaatgggaaaactacattcacactatcttaaccgatcaacaatactgaaaaaatgaaattggttttttcttcttagtatatgatctgaacgagtcgcacatacaccctggta

SEQ ID NO: 43 - DNA, 882 bp, Glycine max
cccgattcgacattaaaagtatattgatttttccccaataaccgaatacttttgtctgtaaatactgcatattttattccattcataaatctattttcttccctatgagttctagtctcaattaaaatactagtttttactgttcatatgttatgatttgaatataccacaccaattcgttatgtatggatgatgagattccattgatacagagctactcgttcttttttctctgacagatggtcagaacttcatctgagttcgaatcctactgagaggtctactagagatcagaaattgttgaagaaagaacaaaagaaacatcttgttctttccaggcgatcggaaaataaagaaatagtgaatttattcaagataattatgtacttacaaaataccgtctcaattcatcctatttcatcatatccgggatgtgatatggttccaaagggtgaactggacagttccaataagatttcattcttgaacaaaaatccgatagacttattggagggagggtcccattggcgtgcatccagtaggaattgaacctacgaattcgccaattatgagttgggcgctttaaccattcagccatggatgcttagcgggaatcctcgtacatggtgaataaccaaattccaattgaagtgaaatctttcggataaatcaatgcaattaggaggattcgatgaaaggacatcaattcaaatcctggattttcgaattgagagagatattgagagaaatcaagaattctcgctatttcttagattcatggacccaattcaattcagcgggatttttcattcatatttttttccatcaagagagttttataaaactcttggactcccgaatttggagtatcctactttcacg

SEQ ID NO: 44 - DNA, 195 bp, Glycine max
gaacaaaagaaacatcttgttctttccaggcgatcggaaaataaagaaatagtgaatttattcaagataattatgtacttacaaaataccgtctcaattcatcctatttcatcatatccgggatgtgatatggttccaaagggtgaactggacagttccaataagatttcattcttgaacaaaaatccgatagac

SEQ ID NO: 45 - DNA, 504 bp, Glycine max
cccgattcgacattaaaagtatattgatttttccccaataaccgaatacttttgtctgtaaatactgcatattttattccattcataaatctattttcttccctatgagttctagtctcaattaaaatactagtttttactgttcatatgttatgatttgaatataccacaccaattcgttatgtatggatgatgagattccattgatacagagctactcgttcttttttctctgacagatggtcagaacttcatctgagttcgaatcctactgagaggtctactagagatcagaaattgttgaagaaagaacaaaagaaacatcttgttctttccaggcgatcggaaaataaagaaatagtgaatttattcaagataattatgtacttacaaaataccgtctcaattcatcctatttcatcatatccgggatgtgatatggttccaaagggtgaactggacagttccaataagatttcattcttgaacaaaaatccgatagac

SEQ ID NO: 46 - DNA, 573 bp, Glycine max
gaacaaaagaaacatcttgttctttccaggcgatcggaaaataaagaaatagtgaatttattcaagataattatgtacttacaaaataccgtctcaattcatcctatttcatcatatccgggatgtgatatggttccaaagggtgaactggacagttccaataagatttcattcttgaacaaaaatccgatagacttattggagggagggtcccattggcgtgcatccagtaggaattgaacctacgaattcgccaattatgagttgggcgctttaaccattcagccatggatgcttagcgggaatcctcgtacatggtgaataaccaaattccaattgaagtgaaatctttcggataaatcaatgcaattaggaggattcgatgaaaggacatcaattcaaatcctggattttcgaattgagagagatattgagagaaatcaagaattctcgctatttcttagattcatggacccaattcaattcagcgggatttttcattcatatttttttccatcaagagagttttataaaactcttggactcccgaatttggagtatcctactttcacg

SEQ ID NO: 47 - DNA, 2254 bp, Glycine max
gttattgggttagccacacactttacatgtttctagcgattcacatggcattttcaaatgatacaagtcttggataagaatctacaacgcactagaacgcccttgttgacgatcctttactccgacagcatctagggttcctcgaacaatgtgatatctcacaccgggtaaatctttaaccctcccccctcttactaagactacagaatgttcttgtaaattatggccaataccgggtatataagcagtaatttcaaatccagaggttaagcgtactctggcaactttacgtaaagcagagtttggttttttgggtgtaatagtggaaaagttgacagataagtcacccttactgccactctacagaaccgtacatgagattttcacttcatatggctcctcgttcaattcttttgaagtcattggatccgtttcctagttcggttcgagaatctctccccttcttctattccgttccgaagagtaactaggaccaattctgtcacgttttcatgttccaattgaacactttccttttttattattctcaaagaagaatatttttctttctttttaccaaacatatgtggatccaatcacgatcttctaataagaacaagaaatctttttcgcgatcaatccttttgtcccattcttcaataatcagaaagatccttttcaatcaagtttgaattttttcgtttggaatcaggactcttctactgcatttttatttacttttttttatttcttttcttccatcattccttaactcccacaaggtttggtcctgtagaatctgacccatttcatcattgagcgaaaagtacgaaaaaaatcagatcgatttttcgaccaaaagtactatgtgaaatcctcggttttttcctctttctctatccctatctcgtaggtagagcgtttgaatcaatagagaaccctttcttctgtatctgtatgaatcgatattattacattccaaaattccttcccgatacctcctaaggaaccgaattggatcccaaattgacgggttagtgtgagcttatccatgcggttatgcacccttcgaataggaatccattttctgaaagatcccggctttcgtgcgttggtgggtcttcgagatcctttcgatgacctatgttgtgttgaagggatatctatatgaaaagacagttctatttctattctattagtattttcgattagtattaaattcgttttagttagtgatctcggctcagctagtcctttctttcgtgatgaactgttggcacctgtcttacattttgtctctgtggaccgaggagaaagggagctcagcggcaagaggattgtaacatgagagaagcaaggaggtcaacctttttcaaatatacaacatgggttctggcaatgcaatgtggttggactctcatgtcgatctgaatgaatcatcctttccacggaggtaaatctttgcctgctaggcaagagtatagcaaattacaaattctgtcttggtagggcatgtatttttattactattaaattgaagtagttaatggtggggttaccattatcctttttgtggtaacgaatatgtgttcctaagaaaagcaatttgtccattttttcggggtctcgaaggggcgtggaaacacataagaactcttgaattgaaatggaaaaatagatgtaactccagttacttcggaaatggtaagatctttggcgcaagaacgcaagaggaggggttgatccgtatcatcttgacttggttctgatttctctattttttaataaaatcgagtcgggttcttctcctacccgtatcgaatagaacatgcttagccaaatcttcttcatggaaaacctgctttatttagatcgggaaaatcatatggttttatgaaatcatgtgctattgctcgaatccgtggtcaatcctatttccgataggagcagttgacaatggaatccaatttttcccataattttcgtatccgtaatagtgtgaaaagaaagcctaactccaagaagttgtttaagaatagtggcgttgagtttcttgaccctttgccttaggattagtcagttctatttctcgatggaggcaagggatataactcagcggtagagtgtcaccttgacgtggtggaagttatcagttcgagcctgattatccctaaacccaatgtaagtttttctatttgtatgccgtgatcgaataataattgagaatggataagaggctcgtgggattacacga

SEQ ID NO: 48 - DNA, 1764 bp, Glycine max
agcagagtttggttttttgggtgtaatagtggaaaagttgacagataagtcacccttactgccactctacagaaccgtacatgagattttcacttcatatggctcctcgttcaattcttttgaagtcattggatccgtttcctagttcggttcgagaatctctccccttcttctattccgttccgaagagtaactaggaccaattctgtcacgttttcatgttccaattgaacactttccttttttattattctcaaagaagaatatttttctttctttttaccaaacatatgtggatccaatcacgatcttctaataagaacaagaaatctttttcgcgatcaatccttttgtcccattcttcaataatcagaaagatccttttcaatcaagtttgaattttttcgtttggaatcaggactcttctactgcatttttatttacttttttttatttcttttcttccatcattccttaactcccacaaggtttggtcctgtagaatctgacccatttcatcattgagcgaaaagtacgaaaaaaatcagatcgatttttcgaccaaaagtactatgtgaaatcctcggttttttcctctttctctatccctatctcgtaggtagagcgtttgaatcaatagagaaccctttcttctgtatctgtatgaatcgatattattacattccaaaattccttcccgatacctcctaaggaaccgaattggatcccaaattgacgggttagtgtgagcttatccatgcggttatgcacccttcgaataggaatccattttctgaaagatcccggctttcgtgcgttggtgggtcttcgagatcctttcgatgacctatgttgtgttgaagggatatctatatgaaaagacagttctatttctattctattagtattttcgattagtattaaattcgttttagttagtgatctcggctcagctagtcctttctttcgtgatgaactgttggcacctgtcttacattttgtctctgtggaccgaggagaaagggagctcagcggcaagaggattgtaacatgagagaagcaaggaggtcaacctttttcaaatatacaacatgggttctggcaatgcaatgtggttggactctcatgtcgatctgaatgaatcatcctttccacggaggtaaatctttgcctgctaggcaagagtatagcaaattacaaattctgtcttggtagggcatgtatttttattactattaaattgaagtagttaatggtggggttaccattatcctttttgtggtaacgaatatgtgttcctaagaaaagcaatttgtccattttttcggggtctcgaaggggcgtggaaacacataagaactcttgaattgaaatggaaaaatagatgtaactccagttacttcggaaatggtaagatctttggcgcaagaacgcaagaggaggggttgatccgtatcatcttgacttggttctgatttctctattttttaataaaatcgagtcgggttcttctcctacccgtatcgaatagaacatgcttagccaaatcttcttcatggaaaacctgctttatttagatcgggaaaatcatatggttttatgaaatcatgtgctattgctcgaatccgtggtcaatcctatttccgataggagcagttgacaatggaatccaatttttcccataattttcgtatccgtaatagtgtgaaaagaaagcctaactccaagaagttgtttaagaatagtggcgttgagtttcttgac

SEQ ID NO: 49 - DNA, 2058 bp, Glycine max
gttattgggttagccacacactttacatgtttctagcgattcacatggcattttcaaatgatacaagtcttggataagaatctacaacgcactagaacgcccttgttgacgatcctttactccgacagcatctagggttcctcgaacaatgtgatatctcacaccgggtaaatctttaaccctcccccctcttactaagactacagaatgttcttgtaaattatggccaataccgggtatataagcagtaatttcaaatccagaggttaagcgtactctggcaactttacgtaaagcagagtttggttttttgggtgtaatagtggaaaagttgacagataagtcacccttactgccactctacagaaccgtacatgagattttcacttcatatggctcctcgttcaattcttttgaagtcattggatccgtttcctagttcggttcgagaatctctccccttcttctattccgttccgaagagtaactaggaccaattctgtcacgttttcatgttccaattgaacactttccttttttattattctcaaagaagaatatttttctttctttttaccaaacatatgtggatccaatcacgatcttctaataagaacaagaaatctttttcgcgatcaatccttttgtcccattcttcaataatcagaaagatccttttcaatcaagtttgaattttttcgtttggaatcaggactcttctactgcatttttatttacttttttttatttcttttcttccatcattccttaactcccacaaggtttggtcctgtagaatctgacccatttcatcattgagcgaaaagtacgaaaaaaatcagatcgatttttcgaccaaaagtactatgtgaaatcctcggttttttcctctttctctatccctatctcgtaggtagagcgtttgaatcaatagagaaccctttcttctgtatctgtatgaatcgatattattacattccaaaattccttcccgatacctcctaaggaaccgaattggatcccaaattgacgggttagtgtgagcttatccatgcggttatgcacccttcgaataggaatccattttctgaaagatcccggctttcgtgcgttggtgggtcttcgagatcctttcgatgacctatgttgtgttgaagggatatctatatgaaaagacagttctatttctattctattagtattttcgattagtattaaattcgttttagttagtgatctcggctcagctagtcctttctttcgtgatgaactgttggcacctgtcttacattttgtctctgtggaccgaggagaaagggagctcagcggcaagaggattgtaacatgagagaagcaaggaggtcaacctttttcaaatatacaacatgggttctggcaatgcaatgtggttggactctcatgtcgatctgaatgaatcatcctttccacggaggtaaatctttgcctgctaggcaagagtatagcaaattacaaattctgtcttggtagggcatgtatttttattactattaaattgaagtagttaatggtggggttaccattatcctttttgtggtaacgaatatgtgttcctaagaaaagcaatttgtccattttttcggggtctcgaaggggcgtggaaacacataagaactcttgaattgaaatggaaaaatagatgtaactccagttacttcggaaatggtaagatctttggcgcaagaacgcaagaggaggggttgatccgtatcatcttgacttggttctgatttctctattttttaataaaatcgagtcgggttcttctcctacccgtatcgaatagaacatgcttagccaaatcttcttcatggaaaacctgctttatttagatcgggaaaatcatatggttttatgaaatcatgtgctattgctcgaatccgtggtcaatcctatttccgataggagcagttgacaatggaatccaatttttcccataattttcgtatccgtaatagtgtgaaaagaaagcctaactccaagaagttgtttaagaatagtggcgttgagtttcttgac

SEQ ID NO: 50 - DNA, 1960 bp, Glycine max
agcagagtttggttttttgggtgtaatagtggaaaagttgacagataagtcacccttactgccactctacagaaccgtacatgagattttcacttcatatggctcctcgttcaattcttttgaagtcattggatccgtttcctagttcggttcgagaatctctccccttcttctattccgttccgaagagtaactaggaccaattctgtcacgttttcatgttccaattgaacactttccttttttattattctcaaagaagaatatttttctttctttttaccaaacatatgtggatccaatcacgatcttctaataagaacaagaaatctttttcgcgatcaatccttttgtcccattcttcaataatcagaaagatccttttcaatcaagtttgaattttttcgtttggaatcaggactcttctactgcatttttatttacttttttttatttcttttcttccatcattccttaactcccacaaggtttggtcctgtagaatctgacccatttcatcattgagcgaaaagtacgaaaaaaatcagatcgatttttcgaccaaaagtactatgtgaaatcctcggttttttcctctttctctatccctatctcgtaggtagagcgtttgaatcaatagagaaccctttcttctgtatctgtatgaatcgatattattacattccaaaattccttcccgatacctcctaaggaaccgaattggatcccaaattgacgggttagtgtgagcttatccatgcggttatgcacccttcgaataggaatccattttctgaaagatcccggctttcgtgcgttggtgggtcttcgagatcctttcgatgacctatgttgtgttgaagggatatctatatgaaaagacagttctatttctattctattagtattttcgattagtattaaattcgttttagttagtgatctcggctcagctagtcctttctttcgtgatgaactgttggcacctgtcttacattttgtctctgtggaccgaggagaaagggagctcagcggcaagaggattgtaacatgagagaagcaaggaggtcaacctttttcaaatatacaacatgggttctggcaatgcaatgtggttggactctcatgtcgatctgaatgaatcatcctttccacggaggtaaatctttgcctgctaggcaagagtatagcaaattacaaattctgtcttggtagggcatgtatttttattactattaaattgaagtagttaatggtggggttaccattatcctttttgtggtaacgaatatgtgttcctaagaaaagcaatttgtccattttttcggggtctcgaaggggcgtggaaacacataagaactcttgaattgaaatggaaaaatagatgtaactccagttacttcggaaatggtaagatctttggcgcaagaacgcaagaggaggggttgatccgtatcatcttgacttggttctgatttctctattttttaataaaatcgagtcgggttcttctcctacccgtatcgaatagaacatgcttagccaaatcttcttcatggaaaacctgctttatttagatcgggaaaatcatatggttttatgaaatcatgtgctattgctcgaatccgtggtcaatcctatttccgataggagcagttgacaatggaatccaatttttcccataattttcgtatccgtaatagtgtgaaaagaaagcctaactccaagaagttgtttaagaatagtggcgttgagtttcttgaccctttgccttaggattagtcagttctatttctcgatggaggcaagggatataactcagcggtagagtgtcaccttgacgtggtggaagttatcagttcgagcctgattatccctaaacccaatgtaagtttttctatttgtatgccgtgatcgaataataattgagaatggataagaggctcgtgggattacacga

SEQ ID NO: 51 - DNA, 784 bp, Glycine max
tcgtatttctttttgtatttaaaaatattcgttgaacctagttaattgaaattagtctaacgtttgtatttgttacaaaaaaagctttttgaattccccgtaaaaatagattgcgctaatgaaaaagctttcaatgttgtaaaatatcccttctttttccataaagtgttccgaatccttttttttgatatagaagtgcgcttttttggaactgccatataattagtatagcttttgattgttatagaattcgatgtgaaagacatttttatgaaaatatatttatctttaattagccatatattttatctatcttaattcccattttttactatttcaagtaaaacattgaatattataatactttttctaaaattttccaaacatagatattgtttattgtaataaaaaatactaaattagttaaaaaaacgaactaatgtttttgaaaaaaaaaatattgtacaaagataaagattcagaataattaataatagatcattctattttaattaattctatgtttactttttattaaccgaaccccttctttacaaaaaagataaaataaaaaccgacgaataagaaacaaaattcattagaatcataatttttttgtttattgtatggaatatacacatcaatattcatggatcataccttttattccatttccagttcctatgttaataggagtgggacttctactttttccgacagcaaccaaatatcttcgccgtatgtgggcttttcctagtattttattgttaactatagttatgat

SEQ ID NO: 52 - DNA, 81 bp, Glycine max
acattgaatattataatactttttctaaaattttccaaacatagatattgtttattgtaataaaaaatactaaattagtta

SEQ ID NO: 53 - DNA, 426 bp, Glycine max
tcgtatttctttttgtatttaaaaatattcgttgaacctagttaattgaaattagtctaacgtttgtatttgttacaaaaaaagctttttgaattccccgtaaaaatagattgcgctaatgaaaaagctttcaatgttgtaaaatatcccttctttttccataaagtgttccgaatccttttttttgatatagaagtgcgcttttttggaactgccatataattagtatagcttttgattgttatagaattcgatgtgaaagacatttttatgaaaatatatttatctttaattagccatatattttatctatcttaattcccattttttactatttcaagtaaaacattgaatattataatactttttctaaaattttccaaacatagatattgtttattgtaataaaaaatactaaattagtta

SEQ ID NO: 54 - DNA, 439 bp, Glycine max
acattgaatattataatactttttctaaaattttccaaacatagatattgtttattgtaataaaaaatactaaattagttaaaaaaacgaactaatgtttttgaaaaaaaaaatattgtacaaagataaagattcagaataattaataatagatcattctattttaattaattctatgtttactttttattaaccgaaccccttctttacaaaaaagataaaataaaaaccgacgaataagaaacaaaattcattagaatcataatttttttgtttattgtatggaatatacacatcaatattcatggatcataccttttattccatttccagttcctatgttaataggagtgggacttctactttttccgacagcaaccaaatatcttcgccgtatgtgggcttttcctagtattttattgttaactatagttatgat

SEQ ID NO: 55 - DNA, 1176 bp, Glycine max
tgcttcatagaaatgtgtaaaaaatagaatcgctcttaaaagccgagtactctaccattgagttagcaaccccaatacaatttagaaaataacgtgggtgtatatatccaatcgcaataaaaacaataaaatgaaaagattgaattgtctaataaaatatcagacattcaaaaaaatagaaaaactcaaaatgttgaagtcgaattgattctcaacatacaaatgaacagataaaacaaaaaaaaatttctaaaaaaaaatggtataccacctctttatctactatgttatacattattatatataattattatatataattgaattacttaccttttaatcaaaaaagaatttattggttgtatcgcagaaaatccaatgaacctaccatttgatgaagtaataaagcctatttcacgtgagtaaattgatcaatttactcacgatcggattatatttatttgatacactattgtcaatattagtattgaaaaagaatacaatcgagaaaacaaacgaataaaataaaaatgataaattagaaaatttaaaatagaaatctaatagtaatgaaatattaatatttcattaagactatattttctatttcaaatattagaaattctattattatgttataagttataattgttataattatttataacaaaaattcttttaatatataaattttattagattcgaattctaaactattcgaattctaaactaaaaacttataaaaaatgatcgaaatctataaattatcgaaatctataatcgaaatctataaataaatataaaaatggaattttaatatatttaatatagaaatagaaaaaaataaagcaacttcccttagaaattggaataacttttttcgtaaaatctcgtctgcttaccacctttctattccaatctattccaatacaaaacaaaaactaatatatatatatatataaatatatatatatatatataataagtatctttagataataagtatctttgtattgtagtattgtttgtagtattgtattcggctcaatccttttaggaaaagattgggccgagtttaattgcaattaattctctgcttacaaagtatccattgcttcgaattcaaatttgatctccttccatacttcacaagcagcagctaattcaggactccat

SEQ ID NO: 56 - DNA, 588 bp, Glycine max
aagtcgaattgattctcaacatacaaatgaacagataaaacaaaaaaaaatttctaaaaaaaaatggtataccacctctttatctactatgttatacattattatatataattattatatataattgaattacttaccttttaatcaaaaaagaatttattggttgtatcgcagaaaatccaatgaacctaccatttgatgaagtaataaagcctatttcacgtgagtaaattgatcaatttactcacgatcggattatatttatttgatacactattgtcaatattagtattgaaaaagaatacaatcgagaaaacaaacgaataaaataaaaatgataaattagaaaatttaaaatagaaatctaatagtaatgaaatattaatatttcattaagactatattttctatttcaaatattagaaattctattattatgttataagttataattgttataattatttataacaaaaattcttttaatatataaattttattagattcgaattctaaactattcgaattctaaactaaaaacttataaaaaatgatcgaaatctataaattatcgaaatctataatcga

SEQ ID NO: 57 - DNA, 784 bp, Glycine max
tgcttcatagaaatgtgtaaaaaatagaatcgctcttaaaagccgagtactctaccattgagttagcaaccccaatacaatttagaaaataacgtgggtgtatatatccaatcgcaataaaaacaataaaatgaaaagattgaattgtctaataaaatatcagacattcaaaaaaatagaaaaactcaaaatgttgaagtcgaattgattctcaacatacaaatgaacagataaaacaaaaaaaaatttctaaaaaaaaatggtataccacctctttatctactatgttatacattattatatataattattatatataattgaattacttaccttttaatcaaaaaagaatttattggttgtatcgcagaaaatccaatgaacctaccatttgatgaagtaataaagcctatttcacgtgagtaaattgatcaatttactcacgatcggattatatttatttgatacactattgtcaatattagtattgaaaaagaatacaatcgagaaaacaaacgaataaaataaaaatgataaattagaaaatttaaaatagaaatctaatagtaatgaaatattaatatttcattaagactatattttctatttcaaatattagaaattctattattatgttataagttataattgttataattatttataacaaaaattcttttaatatataaattttattagattcgaattctaaactattcgaattctaaactaaaaacttataaaaaatgatcgaaatctataaattatcgaaatctataatcga

SEQ ID NO: 58 - DNA, 980 bp, Glycine max
aagtcgaattgattctcaacatacaaatgaacagataaaacaaaaaaaaatttctaaaaaaaaatggtataccacctctttatctactatgttatacattattatatataattattatatataattgaattacttaccttttaatcaaaaaagaatttattggttgtatcgcagaaaatccaatgaacctaccatttgatgaagtaataaagcctatttcacgtgagtaaattgatcaatttactcacgatcggattatatttatttgatacactattgtcaatattagtattgaaaaagaatacaatcgagaaaacaaacgaataaaataaaaatgataaattagaaaatttaaaatagaaatctaatagtaatgaaatattaatatttcattaagactatattttctatttcaaatattagaaattctattattatgttataagttataattgttataattatttataacaaaaattcttttaatatataaattttattagattcgaattctaaactattcgaattctaaactaaaaacttataaaaaatgatcgaaatctataaattatcgaaatctataatcgaaatctataaataaatataaaaatggaattttaatatatttaatatagaaatagaaaaaaataaagcaacttcccttagaaattggaataacttttttcgtaaaatctcgtctgcttaccacctttctattccaatctattccaatacaaaacaaaaactaatatatatatatatataaatatatatatatatatataataagtatctttagataataagtatctttgtattgtagtattgtttgtagtattgtattcggctcaatccttttaggaaaagattgggccgagtttaattgcaattaattctctgcttacaaagtatccattgcttcgaattcaaatttgatctccttccatacttcacaagcagcagctaattcaggactccat

SEQ ID NO: 59 - DNA, 1274 bp, Glycine max
tttttatgaaatacaagatgctcactaaatgataaaaataagaaactaatccaaacttctacaacccctgatattcaaagaatatcagtacattctcttatagatcagacaaagtaattgacattaaatcagtcagaataatggaagtatcattcacatcttattatgtatgtgagaaatcacaaaattcataaataatatagggattaaaatgggataggggttctttaagattctaaaataggaacaatacttatttattatgagccaagccgataaaataaataggatgaactacaaaaattctttatcatgaactatttaacgtgcacatcaacatcaacaattatatggccacgaaaaacaaatagtaagatcaagggagatgaagaaaacggaaaaaaacataaagaaagaaaagggctcgattctttttgtgtaatccatctaagatgaattttgaatattcccactccacccatgaactctattagagtagacttttaggtctttcaaccaactaattaaaccattcgaatattatcgaaatagaaagattttttttgtaacgcagaaaaaaggtaaacaaaatcaaataaataaagaattcattatagattcagcgaatatacctaaaaaaatgcatctattctttaatcatctaggaaaaatatatctacagatacttaaatagatattcagacaaatgaatcatgtgccaggaaccagatttgaactggtgacacgaggattttcagtcctctgctctaccaactgagctatcccgaccattcttgacgcataagactcatttttattttaaaagattactggagtatatgtcaatgaatctatatcaactaagtaaaaaaaaagacgaaaaagaaaagtttgtaagttagtttcagtagtaggaattattttgtattgttttgttaatcacgcatatgagaattccttgctcaaaaataaaatattcatttgtacgtttatcataaaaaaaattctatgtgtttcacatatatatttcaaaacttgtgacttgtaattatgataagaaatttttgaaaataaaaagacaaattccaatttagttgtgaaagaataaactgaatataaactgaataaagcacataagtaacgacttaaagatagagagggagggtatgggaaaaaattagaaagttaaacaggccttttggggatagagggacttgaaccctcacgatttcttaagtcgacggattttcctcttactataaatttcattgttg

SEQ ID NO: 60 - DNA, 457 bp, Glycine max
aagattctaaaataggaacaatacttatttattatgagccaagccgataaaataaataggatgaactacaaaaattctttatcatgaactatttaacgtgcacatcaacatcaacaattatatggccacgaaaaacaaatagtaagatcaagggagatgaagaaaacggaaaaaaacataaagaaagaaaagggctcgattctttttgtgtaatccatctaagatgaattttgaatattcccactccacccatgaactctattagagtagacttttaggtctttcaaccaactaattaaaccattcgaatattatcgaaatagaaagattttttttgtaacgcagaaaaaaggtaaacaaaatcaaataaataaagaattcattatagattcagcgaatatacctaaaaaaatgcatctattctttaatcatctaggaaaaatatatctacagatac

SEQ ID NO: 61 - DNA, 686 bp, Glycine max
tttttatgaaatacaagatgctcactaaatgataaaaataagaaactaatccaaacttctacaacccctgatattcaaagaatatcagtacattctcttatagatcagacaaagtaattgacattaaatcagtcagaataatggaagtatcattcacatcttattatgtatgtgagaaatcacaaaattcataaataatatagggattaaaatgggataggggttctttaagattctaaaataggaacaatacttatttattatgagccaagccgataaaataaataggatgaactacaaaaattctttatcatgaactatttaacgtgcacatcaacatcaacaattatatggccacgaaaaacaaatagtaagatcaagggagatgaagaaaacggaaaaaaacataaagaaagaaaagggctcgattctttttgtgtaatccatctaagatgaattttgaatattcccactccacccatgaactctattagagtagacttttaggtctttcaaccaactaattaaaccattcgaatattatcgaaatagaaagattttttttgtaacgcagaaaaaaggtaaacaaaatcaaataaataaagaattcattatagattcagcgaatatacctaaaaaaatgcatctattctttaatcatctaggaaaaatatatctacagatac

SEQ ID NO: 62 - DNA, 1045 bp, Glycine max
aagattctaaaataggaacaatacttatttattatgagccaagccgataaaataaataggatgaactacaaaaattctttatcatgaactatttaacgtgcacatcaacatcaacaattatatggccacgaaaaacaaatagtaagatcaagggagatgaagaaaacggaaaaaaacataaagaaagaaaagggctcgattctttttgtgtaatccatctaagatgaattttgaatattcccactccacccatgaactctattagagtagacttttaggtctttcaaccaactaattaaaccattcgaatattatcgaaatagaaagattttttttgtaacgcagaaaaaaggtaaacaaaatcaaataaataaagaattcattatagattcagcgaatatacctaaaaaaatgcatctattctttaatcatctaggaaaaatatatctacagatacttaaatagatattcagacaaatgaatcatgtgccaggaaccagatttgaactggtgacacgaggattttcagtcctctgctctaccaactgagctatcccgaccattcttgacgcataagactcatttttattttaaaagattactggagtatatgtcaatgaatctatatcaactaagtaaaaaaaaagacgaaaaagaaaagtttgtaagttagtttcagtagtaggaattattttgtattgttttgttaatcacgcatatgagaattccttgctcaaaaataaaatattcatttgtacgtttatcataaaaaaaattctatgtgtttcacatatatatttcaaaacttgtgacttgtaattatgataagaaatttttgaaaataaaaagacaaattccaatttagttgtgaaagaataaactgaatataaactgaataaagcacataagtaacgacttaaagatagagagggagggtatgggaaaaaattagaaagttaaacaggccttttggggatagagggacttgaaccctcacgatttcttaagtcgacggattttcctcttactataaatttcattgttg

SEQ ID NO: 63 - DNA, 129 bp, Glycine max
aagaaaagtttgtaagttagtttcagtagtaggaattattttgtattgttttgttaatcacgcatatgagaattccttgctcaaaaataaaatattcatttgtacgtttatcataaaaaaaattctatg

SEQ ID NO: 64 - DNA, 1011 bp, Glycine max
tttttatgaaatacaagatgctcactaaatgataaaaataagaaactaatccaaacttctacaacccctgatattcaaagaatatcagtacattctcttatagatcagacaaagtaattgacattaaatcagtcagaataatggaagtatcattcacatcttattatgtatgtgagaaatcacaaaattcataaataatatagggattaaaatgggataggggttctttaagattctaaaataggaacaatacttatttattatgagccaagccgataaaataaataggatgaactacaaaaattctttatcatgaactatttaacgtgcacatcaacatcaacaattatatggccacgaaaaacaaatagtaagatcaagggagatgaagaaaacggaaaaaaacataaagaaagaaaagggctcgattctttttgtgtaatccatctaagatgaattttgaatattcccactccacccatgaactctattagagtagacttttaggtctttcaaccaactaattaaaccattcgaatattatcgaaatagaaagattttttttgtaacgcagaaaaaaggtaaacaaaatcaaataaataaagaattcattatagattcagcgaatatacctaaaaaaatgcatctattctttaatcatctaggaaaaatatatctacagatacttaaatagatattcagacaaatgaatcatgtgccaggaaccagatttgaactggtgacacgaggattttcagtcctctgctctaccaactgagctatcccgaccattcttgacgcataagactcatttttattttaaaagattactggagtatatgtcaatgaatctatatcaactaagtaaaaaaaaagacgaaaaagaaaagtttgtaagttagtttcagtagtaggaattattttgtattgttttgttaatcacgcatatgagaattccttgctcaaaaataaaatattcatttgtacgtttatcataaaaaaaattctatg

SEQ ID NO: 65 - DNA, 392 bp, Glycine max
aagaaaagtttgtaagttagtttcagtagtaggaattattttgtattgttttgttaatcacgcatatgagaattccttgctcaaaaataaaatattcatttgtacgtttatcataaaaaaaattctatgtgtttcacatatatatttcaaaacttgtgacttgtaattatgataagaaatttttgaaaataaaaagacaaattccaatttagttgtgaaagaataaactgaatataaactgaataaagcacataagtaacgacttaaagatagagagggagggtatgggaaaaaattagaaagttaaacaggccttttggggatagagggacttgaaccctcacgatttcttaagtcgacggattttcctcttactataaatttcattgttg

SEQ ID NO: 66 - DNA, 686 bp, Glycine max
attatattgaagcccagaattggttgaagatcacaggacgttttgaataagaacaccttgtttttttatttttcttatattttcttcttaaattatatctatctatctataattctatctatatagatagatagatatctatagttataaatttttgtttgttgtccccatcaatcaaattaaaaactcttttctataggaacgaccaatcacagaaattaattatatccctttcaaattgttagattaagtacataataatactttttctatataaaaaaggtaaaattagttccatctaggaacttctgaataaaatatgaatacattagattaggctgcacaaaaaattatttaacttccattcaaagttcgcaaaaaaggtcttagaagattaaaaaggtccgttgagcgccccactgttatgtcataatagattcgaacacttgccccggattgacttcgagatcataattgttctagtgaataactaaagaaaatagattaaattaaagaaaatataaaatagatagctgtgagatagaagagaacaaaactcaatagaaacatctctaataagttcaaaaattctgttttatgttggcaggtctctttgtatgtgttgtctggaaagaggaggactcaatgattattcgttcaccggaaccagaagtgaaaattttggt

SEQ ID NO: 67 - DNA, 206 bp, Glycine max
atatccctttcaaattgttagattaagtacataataatactttttctatataaaaaaggtaaaattagttccatctaggaacttctgaataaaatatgaatacattagattaggctgcacaaaaaattatttaacttccattcaaagttcgcaaaaaaggtcttagaagattaaaaaggtccgttgagcgccccactgttatgtca

SEQ ID NO: 68 - DNA, 430 bp, Glycine max
attatattgaagcccagaattggttgaagatcacaggacgttttgaataagaacaccttgtttttttatttttcttatattttcttcttaaattatatctatctatctataattctatctatatagatagatagatatctatagttataaatttttgtttgttgtccccatcaatcaaattaaaaactcttttctataggaacgaccaatcacagaaattaattatatccctttcaaattgttagattaagtacataataatactttttctatataaaaaaggtaaaattagttccatctaggaacttctgaataaaatatgaatacattagattaggctgcacaaaaaattatttaacttccattcaaagttcgcaaaaaaggtcttagaagattaaaaaggtccgttgagcgccccactgttatgtca

SEQ ID NO: 69 - DNA, 462 bp, Glycine max
atatccctttcaaattgttagattaagtacataataatactttttctatataaaaaaggtaaaattagttccatctaggaacttctgaataaaatatgaatacattagattaggctgcacaaaaaattatttaacttccattcaaagttcgcaaaaaaggtcttagaagattaaaaaggtccgttgagcgccccactgttatgtcataatagattcgaacacttgccccggattgacttcgagatcataattgttctagtgaataactaaagaaaatagattaaattaaagaaaatataaaatagatagctgtgagatagaagagaacaaaactcaatagaaacatctctaataagttcaaaaattctgttttatgttggcaggtctctttgtatgtgttgtctggaaagaggaggactcaatgattattcgttcaccggaaccagaagtgaaaattttggt

SEQ ID NO: 70 - DNA, 588 bp, Glycine max
tatccgcgtttttaatgtacttgatatgttattttatttgtacagagatcggcctgatattctattaggggtaattcaaaataaatgtatataatagattatatctatatagatataatctattataatagatattctcttctaaataaaatcttttatattactattactattaattaagaattcatttttgaatgaatttctatcaataattcctacttctattattatatattctttttttctatgtatctatataatcgaattgaaattacgattctatcaaatctttttatttatttttttttttttaagaaaaaagaaagttctaattattaataaattatatctatatatatatatatacagatagatattctatatcttttatcttaattaatatttgacccctccttataatgtgttatagttttctttatttttaggaacttatccattccttttttaatgtgcccgacaatccgaaagaattctgggggaggggtcatttcgtttttgatctagaataaatagagcttcaagaaatgagagaattaagaatacccacgagaaacactaatccaatcc

SEQ ID NO: 71 - DNA, 199 bp, Glycine max
gatattctcttctaaataaaatcttttatattactattactattaattaagaattcatttttgaatgaatttctatcaataattcctacttctattattatatattctttttttctatgtatctatataatcgaattgaaattacgattctatcaaatctttttatttatttttttttttttaagaaaaaagaaagttc

SEQ ID NO: 72 - DNA, 329 bp, Glycine max
tatccgcgtttttaatgtacttgatatgttattttatttgtacagagatcggcctgatattctattaggggtaattcaaaataaatgtatataatagattatatctatatagatataatctattataatagatattctcttctaaataaaatcttttatattactattactattaattaagaattcatttttgaatgaatttctatcaataattcctacttctattattatatattctttttttctatgtatctatataatcgaattgaaattacgattctatcaaatctttttatttatttttttttttttaagaaaaaagaaagttc

SEQ ID NO: 73 - DNA, 458 bp, Glycine max
gatattctcttctaaataaaatcttttatattactattactattaattaagaattcatttttgaatgaatttctatcaataattcctacttctattattatatattctttttttctatgtatctatataatcgaattgaaattacgattctatcaaatctttttatttatttttttttttttaagaaaaaagaaagttctaattattaataaattatatctatatatatatatatacagatagatattctatatcttttatcttaattaatatttgacccctccttataatgtgttatagttttctttatttttaggaacttatccattccttttttaatgtgcccgacaatccgaaagaattctgggggaggggtcatttcgtttttgatctagaataaatagagcttcaagaaatgagagaattaagaatacccacgagaaacactaatccaatcc

SEQ ID NO: 74 - DNA, 784 bp, Glycine max
acttataatttacgtaaaaacggtaagtcaaagtgactaatttcactgaaatctgacttcttcatttttatcaatgtaatcaatgattaaaaaaaatgaaaaaggttttcaatttttggtcttagtctaaaaaaaaaagatcggactacaatcaccgtaatccagatagtagaaatcaaatatatttgatttctactatctgactgagaattgcgtccaatttttttttagtttcatctatttgatttgtattgattccaatgaatctgaaataatcaaaaaacaactcttattcttcggattctttttacaatcccggtatcatattaaaaaaagaaaaaggggggcaggaatggggattacgcggtcccccattttcgatatatataaaacctggttaagcgtcagttcatcgaaagagaaattttaagaagaattcggttggaataggaagcaatttcctattctattggattctcttgattttctcaaaatacgaatatagatataattcaaatctttgtttgattgaaagagtattttcaatttctataatatacatatacatagaatacattacactactagaatataatagaaccctgaaatgcagatctattttatatttgtttatatttgaactcaattaattagtataaatgaaatactttaattcaatagttaaagttaagaaattgaaaaacccaagtagaaaacaaaatgggtactttgatcccttaactcaattttgagtatccctatagtccacttcttccccatacta

SEQ ID NO: 75 - DNA, 237 bp, Glycine max
gattccaatgaatctgaaataatcaaaaaacaactcttattcttcggattctttttacaatcccggtatcatattaaaaaaagaaaaaggggggcaggaatggggattacgcggtcccccattttcgatatatataaaacctggttaagcgtcagttcatcgaaagagaaattttaagaagaattcggttggaataggaagcaatttcctattctattggattctcttgattttctc

SEQ ID NO: 76 - DNA, 490 bp, Glycine max
acttataatttacgtaaaaacggtaagtcaaagtgactaatttcactgaaatctgacttcttcatttttatcaatgtaatcaatgattaaaaaaaatgaaaaaggttttcaatttttggtcttagtctaaaaaaaaaagatcggactacaatcaccgtaatccagatagtagaaatcaaatatatttgatttctactatctgactgagaattgcgtccaatttttttttagtttcatctatttgatttgtattgattccaatgaatctgaaataatcaaaaaacaactcttattcttcggattctttttacaatcccggtatcatattaaaaaaagaaaaaggggggcaggaatggggattacgcggtcccccattttcgatatatataaaacctggttaagcgtcagttcatcgaaagagaaattttaagaagaattcggttggaataggaagcaatttcctattctattggattctcttgattttctc

SEQ ID NO: 77 - DNA, 531 bp, Glycine max
gattccaatgaatctgaaataatcaaaaaacaactcttattcttcggattctttttacaatcccggtatcatattaaaaaaagaaaaaggggggcaggaatggggattacgcggtcccccattttcgatatatataaaacctggttaagcgtcagttcatcgaaagagaaattttaagaagaattcggttggaataggaagcaatttcctattctattggattctcttgattttctcaaaatacgaatatagatataattcaaatctttgtttgattgaaagagtattttcaatttctataatatacatatacatagaatacattacactactagaatataatagaaccctgaaatgcagatctattttatatttgtttatatttgaactcaattaattagtataaatgaaatactttaattcaatagttaaagttaagaaattgaaaaacccaagtagaaaacaaaatgggtactttgatcccttaactcaattttgagtatccctatagtccacttcttccccatacta

SEQ ID NO: 78 - DNA, 1176 bp, Glycine max
ttcaagctctcatttttgctactttagctgcggcttatataggtgaatccatggaaggccatcattgaccagttttctacaaaatagtccttttcgtttagcttgccgcacatatattgcggctcaatataatctacttagtaaacatctatatctatctacctatatctatatagatataggtagatagatatagatagatattcgattagcatatattagcatatattagtatatattggatattcaaaaaaaaaataataaataaaaaaattaagagccataattagaattctgtatgatatttacgtttacggcgtgtgataaaaagatactatatagaactaaaggagattcccgtttcagttacattcaattcaacgattgaccaaaagagaattcaaaaaataacatttagatcacccaaattccaatatttctatgaaacaataattaaatctaacgaatttatttcgattgattgtatccatatggggtaataataaaagggggaggagagcttaaaaaaacaagataacaaataaagtataagtgcggggggtcaaactgggtgtatataatcggatcactataagacaactctttcttttgaggtttcaaagaatgattttcgaattcgattgaatctaaaacacaaaagaattggtttacaggatagaagaaacccacgtatatgtgataggatattctatatgaactagttatatatgaactaaccatatccatatatattatataaaataactaatattttatatctaaaattgccccgctactactgtaaatttcgatagggattaaaaaacaaagcctttccgtttcatctcgaattgtgaattgaatattcaatgactaaaaaaatggaataaaaaaacgaagaattcaaagaatgattccaaattgaaggtaggataagaacaaaggttctacatattcacaaaaaaaaactatgtatgtaggtagagacgaaaaaataaatctcgaagtaattcgtagagttcaataactattattattttcaattcggaattcttattaattactttaaaccgaataaatcttgataattgaaaaattaagtcataatttattggttgattatatcattaactatttctttattttatatttaggttacgaggaaattatcatgaa

SEQ ID NO: 79 - DNA, 624 bp, Glycine max
ctgtatgatatttacgtttacggcgtgtgataaaaagatactatatagaactaaaggagattcccgtttcagttacattcaattcaacgattgaccaaaagagaattcaaaaaataacatttagatcacccaaattccaatatttctatgaaacaataattaaatctaacgaatttatttcgattgattgtatccatatggggtaataataaaagggggaggagagcttaaaaaaacaagataacaaataaagtataagtgcggggggtcaaactgggtgtatataatcggatcactataagacaactctttcttttgaggtttcaaagaatgattttcgaattcgattgaatctaaaacacaaaagaattggtttacaggatagaagaaacccacgtatatgtgataggatattctatatgaactagttatatatgaactaaccatatccatatatattatataaaataactaatattttatatctaaaattgccccgctactactgtaaatttcgatagggattaaaaaacaaagcctttccgtttcatctcgaattgtgaattgaatattcaatgactaaaaaaatggaataaaaaaacgaagaattcaaagaatgattcc

SEQ ID NO: 80 - DNA, 918 bp, Glycine max
ttcaagctctcatttttgctactttagctgcggcttatataggtgaatccatggaaggccatcattgaccagttttctacaaaatagtccttttcgtttagcttgccgcacatatattgcggctcaatataatctacttagtaaacatctatatctatctacctatatctatatagatataggtagatagatatagatagatattcgattagcatatattagcatatattagtatatattggatattcaaaaaaaaaataataaataaaaaaattaagagccataattagaattctgtatgatatttacgtttacggcgtgtgataaaaagatactatatagaactaaaggagattcccgtttcagttacattcaattcaacgattgaccaaaagagaattcaaaaaataacatttagatcacccaaattccaatatttctatgaaacaataattaaatctaacgaatttatttcgattgattgtatccatatggggtaataataaaagggggaggagagcttaaaaaaacaagataacaaataaagtataagtgcggggggtcaaactgggtgtatataatcggatcactataagacaactctttcttttgaggtttcaaagaatgattttcgaattcgattgaatctaaaacacaaaagaattggtttacaggatagaagaaacccacgtatatgtgataggatattctatatgaactagttatatatgaactaaccatatccatatatattatataaaataactaatattttatatctaaaattgccccgctactactgtaaatttcgatagggattaaaaaacaaagcctttccgtttcatctcgaattgtgaattgaatattcaatgactaaaaaaatggaataaaaaaacgaagaattcaaagaatgattcc

SEQ ID NO: 81 - DNA, 882 bp, Glycine max
ctgtatgatatttacgtttacggcgtgtgataaaaagatactatatagaactaaaggagattcccgtttcagttacattcaattcaacgattgaccaaaagagaattcaaaaaataacatttagatcacccaaattccaatatttctatgaaacaataattaaatctaacgaatttatttcgattgattgtatccatatggggtaataataaaagggggaggagagcttaaaaaaacaagataacaaataaagtataagtgcggggggtcaaactgggtgtatataatcggatcactataagacaactctttcttttgaggtttcaaagaatgattttcgaattcgattgaatctaaaacacaaaagaattggtttacaggatagaagaaacccacgtatatgtgataggatattctatatgaactagttatatatgaactaaccatatccatatatattatataaaataactaatattttatatctaaaattgccccgctactactgtaaatttcgatagggattaaaaaacaaagcctttccgtttcatctcgaattgtgaattgaatattcaatgactaaaaaaatggaataaaaaaacgaagaattcaaagaatgattccaaattgaaggtaggataagaacaaaggttctacatattcacaaaaaaaaactatgtatgtaggtagagacgaaaaaataaatctcgaagtaattcgtagagttcaataactattattattttcaattcggaattcttattaattactttaaaccgaataaatcttgataattgaaaaattaagtcataatttattggttgattatatcattaactatttctttattttatatttaggttacgaggaaattatcatgaa

SEQ ID NO: 82 - DNA, 588 bp, Glycine max
gaatcaacaaacgaaattccattaatgaaataaaaaaaagggggtgtggatgacttgtatatagatttctaaaactcttttctcttttatccccccgctctcttgttctattcatacctaatttttcttatttcttgttgtttatatagaatgataggatgtagttttctatagccagaaaaagaaatgaaatttgcatcgatcttcaaaacaaaatgaaaaaatgtatagagataaaagatagaatataggaaaaagaaaaaaaatagtcactaaatgaagtaattggacttataaatagataaatagattacccctattgaggtacttttttttttaatgtttcttattatttttgattttattatcatttctttttaataccaactcgctctttattattttcagttactaatcctagttatttgatttatatactttttttatagtaaatacatgagatagaatattcaaaatggaatatttctattattttattcttcatccaatgactatacattcattatatttttgtgtaaatagaggaaaaactcgatggaaaaatggtggtttaattctatgttgttt

SEQ ID NO: 83 - DNA, 141 bp, Glycine max
ttcaaaacaaaatgaaaaaatgtatagagataaaagatagaatataggaaaaagaaaaaaaatagtcactaaatgaagtaattggacttataaatagataaatagattacccctattgaggtacttttttttttaatgttt

SEQ ID NO: 84 - DNA, 345 bp, Glycine max
gaatcaacaaacgaaattccattaatgaaataaaaaaaagggggtgtggatgacttgtatatagatttctaaaactcttttctcttttatccccccgctctcttgttctattcatacctaatttttcttatttcttgttgtttatatagaatgataggatgtagttttctatagccagaaaaagaaatgaaatttgcatcgatcttcaaaacaaaatgaaaaaatgtatagagataaaagatagaatataggaaaaagaaaaaaaatagtcactaaatgaagtaattggacttataaatagataaatagattacccctattgaggtacttttttttttaatgttt

SEQ ID NO: 85 - DNA, 384 bp, Glycine max
ttcaaaacaaaatgaaaaaatgtatagagataaaagatagaatataggaaaaagaaaaaaaatagtcactaaatgaagtaattggacttataaatagataaatagattacccctattgaggtacttttttttttaatgtttcttattatttttgattttattatcatttctttttaataccaactcgctctttattattttcagttactaatcctagttatttgatttatatactttttttatagtaaatacatgagatagaatattcaaaatggaatatttctattattttattcttcatccaatgactatacattcattatatttttgtgtaaatagaggaaaaactcgatggaaaaatggtggtttaattctatgttgttt

SEQ ID NO: 86 - DNA, 1274 bp, Glycine max
tttatttcttcatgttcaaaaaaacaagattttttagatacagaaaacaaagtaaaattctggggttttttattccccaattataatactaaattcaaaaaaaggagagtaatgagtataatctttcgctcagacgatgtactgatatattctatggcggaggttcgaagaacaagcaatttattttgggccgttttcactttgttaggttcattggggttattgttcgttgcaatttccagttatcttggtatggatttgttctttatttctgagaaaattagtgatttttcttttattccggactttatttattttccatttattccacaaggggccacgatggctttttatggaatcgcgggtctctttagtagtttttattttgggtcgattattttttgggatataggtggtggttttgatatcttcaataaaaaagggaaaaaggtacgttttgttcgttggggatttcctggaaaaaatcgtcgtattattctcgaaatacctatgaacgagcttcactccatcagaataataacagaagttaaagaagagggtatttttactcgtaccagtacttttgagagtatcgtttatctggaaaccatagaacaggggtttattcccttgactcgtattgaagataatttgaatgggacacaaattgcacataaagctggcgaattatctgtatttttgggtgtaccacttttttactgatgagaattgaacttaattagaaattaaattgcacaaaaagcttcagaattgtcctatttattttgtgtaccttttgaaatattttgaaaaaaaaaaaacgtttttttttttcaaaatatttccatttttatagatgaggcgttatttgatttcgaaatccgattattatattcctaacccgaagtgctccttggtgtattcataaaaagtaataatttttcttcgaatcttagcaattgatatcctttcgaaacaccatttttttcttcatttgaattgacagtgaattctttcgatttcttattcgatttatgtattcttttttctaaattaaacaaggcaaggactaactcccgaactgcaagtaaaaaaaatgatagaatagaatatagatttatctataagctctatgacttgtaatagaacttatgcttgatagaaaggttattatcatatagagttgacgaatgagatgaagttgagttcattaaataaagacgaaaaatgacaaaaaagaaaacattcattccccttctatatcttacatctat

SEQ ID NO: 87 - DNA, 588 bp, Glycine max
tttattccggactttatttattttccatttattccacaaggggccacgatggctttttatggaatcgcgggtctctttagtagtttttattttgggtcgattattttttgggatataggtggtggttttgatatcttcaataaaaaagggaaaaaggtacgttttgttcgttggggatttcctggaaaaaatcgtcgtattattctcgaaatacctatgaacgagcttcactccatcagaataataacagaagttaaagaagagggtatttttactcgtaccagtacttttgagagtatcgtttatctggaaaccatagaacaggggtttattcccttgactcgtattgaagataatttgaatgggacacaaattgcacataaagctggcgaattatctgtatttttgggtgtaccacttttttactgatgagaattgaacttaattagaaattaaattgcacaaaaagcttcagaattgtcctatttattttgtgtaccttttgaaatattttgaaaaaaaaaaaacgtttttttttttcaaaatatttccatttttatagatgaggcgttatttgatttcgaaatc

SEQ ID NO: 88 - DNA, 882 bp, Glycine max
tttatttcttcatgttcaaaaaaacaagattttttagatacagaaaacaaagtaaaattctggggttttttattccccaattataatactaaattcaaaaaaaggagagtaatgagtataatctttcgctcagacgatgtactgatatattctatggcggaggttcgaagaacaagcaatttattttgggccgttttcactttgttaggttcattggggttattgttcgttgcaatttccagttatcttggtatggatttgttctttatttctgagaaaattagtgatttttcttttattccggactttatttattttccatttattccacaaggggccacgatggctttttatggaatcgcgggtctctttagtagtttttattttgggtcgattattttttgggatataggtggtggttttgatatcttcaataaaaaagggaaaaaggtacgttttgttcgttggggatttcctggaaaaaatcgtcgtattattctcgaaatacctatgaacgagcttcactccatcagaataataacagaagttaaagaagagggtatttttactcgtaccagtacttttgagagtatcgtttatctggaaaccatagaacaggggtttattcccttgactcgtattgaagataatttgaatgggacacaaattgcacataaagctggcgaattatctgtatttttgggtgtaccacttttttactgatgagaattgaacttaattagaaattaaattgcacaaaaagcttcagaattgtcctatttattttgtgtaccttttgaaatattttgaaaaaaaaaaaacgtttttttttttcaaaatatttccatttttatagatgaggcgttatttgatttcgaaatc

SEQ ID NO: 89 - DNA, 980 bp, Glycine max
tttattccggactttatttattttccatttattccacaaggggccacgatggctttttatggaatcgcgggtctctttagtagtttttattttgggtcgattattttttgggatataggtggtggttttgatatcttcaataaaaaagggaaaaaggtacgttttgttcgttggggatttcctggaaaaaatcgtcgtattattctcgaaatacctatgaacgagcttcactccatcagaataataacagaagttaaagaagagggtatttttactcgtaccagtacttttgagagtatcgtttatctggaaaccatagaacaggggtttattcccttgactcgtattgaagataatttgaatgggacacaaattgcacataaagctggcgaattatctgtatttttgggtgtaccacttttttactgatgagaattgaacttaattagaaattaaattgcacaaaaagcttcagaattgtcctatttattttgtgtaccttttgaaatattttgaaaaaaaaaaaacgtttttttttttcaaaatatttccatttttatagatgaggcgttatttgatttcgaaatccgattattatattcctaacccgaagtgctccttggtgtattcataaaaagtaataatttttcttcgaatcttagcaattgatatcctttcgaaacaccatttttttcttcatttgaattgacagtgaattctttcgatttcttattcgatttatgtattcttttttctaaattaaacaaggcaaggactaactcccgaactgcaagtaaaaaaaatgatagaatagaatatagatttatctataagctctatgacttgtaatagaacttatgcttgatagaaaggttattatcatatagagttgacgaatgagatgaagttgagttcattaaataaagacgaaaaatgacaaaaaagaaaacattcattccccttctatatcttacatctat

SEQ ID NO: 90 - DNA, 980 bp, Glycine max
ggttcaattgtccgaaatgaatttctagtcccgcatatttatcaacatattaaactcataaaaataactcaatttttttgttgataatttatgtaattctattcagtataatatcagtataatagaataaaaaaattatgaaaagatctttggttctgtatagtctttttatactatatttaaagagttacttgtaacgtaatacaaataaactcgagtatggatattgtcaggaagactatttgactttgttttttcaaccccccaataaattcgaatattatggttatgacactgtttttttcagatttcatttttctataatagagagttctattatagaaatttcgattgtaaaatacaataaaattggattcgatactaaacataaaatagatatctatatagatagataatttgaaacaaaatcaattaaaatatggaaaaggtactctaggggggattttttgtcttttcctagagtccgattcctattccttcttgtttgtgtcgaaatagaaatattctaaaaatatcaataaaataatcttttttaaccccttcctgaaaaatcctatatcttagtttctattttttccaacttagaatatttctgccatataatattttattgcatataatacgtagtgaacaaataaaaagagagggggttggggaatttgattaaacaaattgaatttcttcaattaacttgtaaaaaaaaaaaagaatgaaatggagttttttgaaacatcggaaaaagtagtcgtttatatgaataaaaaaagattagaattattaataactcacctgggatccactctttctttgacgaattcgagtggatcccaggtgagttattacgctttcatttcaaaaaaaaaacaaattctaacacgattactacagagatgagcccaatccggaatatgaaccataaaagaaaataccaattaaaccgatcacagtaataccagtta

SEQ ID NO: 91 - DNA, 422 bp, Glycine max
ccaataaattcgaatattatggttatgacactgtttttttcagatttcatttttctataatagagagttctattatagaaatttcgattgtaaaatacaataaaattggattcgatactaaacataaaatagatatctatatagatagataatttgaaacaaaatcaattaaaatatggaaaaggtactctaggggggattttttgtcttttcctagagtccgattcctattccttcttgtttgtgtcgaaatagaaatattctaaaaatatcaataaaataatcttttttaaccccttcctgaaaaatcctatatcttagtttctattttttccaacttagaatatttctgccatataatattttattgcatataatacgtagtgaacaaataaaaagagagggggttggggaatttgatt

SEQ ID NO: 92 - DNA, 686 bp, Glycine max
ggttcaattgtccgaaatgaatttctagtcccgcatatttatcaacatattaaactcataaaaataactcaatttttttgttgataatttatgtaattctattcagtataatatcagtataatagaataaaaaaattatgaaaagatctttggttctgtatagtctttttatactatatttaaagagttacttgtaacgtaatacaaataaactcgagtatggatattgtcaggaagactatttgactttgttttttcaaccccccaataaattcgaatattatggttatgacactgtttttttcagatttcatttttctataatagagagttctattatagaaatttcgattgtaaaatacaataaaattggattcgatactaaacataaaatagatatctatatagatagataatttgaaacaaaatcaattaaaatatggaaaaggtactctaggggggattttttgtcttttcctagagtccgattcctattccttcttgtttgtgtcgaaatagaaatattctaaaaatatcaataaaataatcttttttaaccccttcctgaaaaatcctatatcttagtttctattttttccaacttagaatatttctgccatataatattttattgcatataatacgtagtgaacaaataaaaagagagggggttggggaatttgatt

SEQ ID NO: 93 - DNA, 716 bp, Glycine max
ccaataaattcgaatattatggttatgacactgtttttttcagatttcatttttctataatagagagttctattatagaaatttcgattgtaaaatacaataaaattggattcgatactaaacataaaatagatatctatatagatagataatttgaaacaaaatcaattaaaatatggaaaaggtactctaggggggattttttgtcttttcctagagtccgattcctattccttcttgtttgtgtcgaaatagaaatattctaaaaatatcaataaaataatcttttttaaccccttcctgaaaaatcctatatcttagtttctattttttccaacttagaatatttctgccatataatattttattgcatataatacgtagtgaacaaataaaaagagagggggttggggaatttgattaaacaaattgaatttcttcaattaacttgtaaaaaaaaaaaagaatgaaatggagttttttgaaacatcggaaaaagtagtcgtttatatgaataaaaaaagattagaattattaataactcacctgggatccactctttctttgacgaattcgagtggatcccaggtgagttattacgctttcatttcaaaaaaaaaacaaattctaacacgattactacagagatgagcccaatccggaatatgaaccataaaagaaaataccaattaaaccgatcacagtaataccagtta

SEQ ID NO: 94 - DNA, 686 bp, Glycine max
atcaatcgtttttttccagatgcattgatattcccttttttttcattctagttattgacacggaaaggggtgaagaagattatagatccaattaagtattaaatatatgtaattaatatgtaattaatctcctcttctttatttcgtcttttatttcttcttttttttacaattgtaatagaataagttaggaaagagaaagaataagggttaatttggcctaattgaaattcggagtgaaactcgggatctcgtccaggcttccatggaattgaattaataattcaattccatttctattaataaaagagtgaaagcagaaatttaaatggaatgtatagggtgggggcaacaatatcataaaaaatacttagttaaaaaaacgaaaatactgtattgcaattgtattgcaattcgaaacgaaatatagttcgagatcattgtattatttttgtactatattaatcttaatcttaattggaacgaaatctttcaaaattggatttctaattcttaatttatattttcgttttttcgttcttttctttttcttagattataatccgaaaacaaaagagttaagtgaattaaaaacccaagggaggttcatggccaaaggtaaagatatccgagtaattgttattttggaatgtaccggttgtgataaaaagagtgttaataaggaa

SEQ ID NO: 95 - DNA, 131 bp, Glycine max
ttccatggaattgaattaataattcaattccatttctattaataaaagagtgaaagcagaaatttaaatggaatgtatagggtgggggcaacaatatcataaaaaatacttagttaaaaaaacgaaaatac

SEQ ID NO: 96 - DNA, 392 bp, Glycine max
atcaatcgtttttttccagatgcattgatattcccttttttttcattctagttattgacacggaaaggggtgaagaagattatagatccaattaagtattaaatatatgtaattaatatgtaattaatctcctcttctttatttcgtcttttatttcttcttttttttacaattgtaatagaataagttaggaaagagaaagaataagggttaatttggcctaattgaaattcggagtgaaactcgggatctcgtccaggcttccatggaattgaattaataattcaattccatttctattaataaaagagtgaaagcagaaatttaaatggaatgtatagggtgggggcaacaatatcataaaaaatacttagttaaaaaaacgaaaatac

SEQ ID NO: 97 - DNA, 425 bp, Glycine max
ttccatggaattgaattaataattcaattccatttctattaataaaagagtgaaagcagaaatttaaatggaatgtatagggtgggggcaacaatatcataaaaaatacttagttaaaaaaacgaaaatactgtattgcaattgtattgcaattcgaaacgaaatatagttcgagatcattgtattatttttgtactatattaatcttaatcttaattggaacgaaatctttcaaaattggatttctaattcttaatttatattttcgttttttcgttcttttctttttcttagattataatccgaaaacaaaagagttaagtgaattaaaaacccaagggaggttcatggccaaaggtaaagatatccgagtaattgttattttggaatgtaccggttgtgataaaaagagtgttaataaggaa

SEQ ID NO: 98 - DNA, 1176 bp, Glycine max
ttggtttaactgaataactaaaaaaaaaaaaaaaatagatagacctttctttctcttcgtctcaggtcgatggatcttctcaattgaaagatcccctatatcgataatacacattccagtttgggaacgtccggtgccaaagtcattgaatgggtaagtcgccaattcctaaaacggactatttaatgtactttatccgcccgctgggttacgggtgggtattttaccagaggttttgattgtatcaaatctacccctgtgtgatttttgttgaagcatatactcctcggggagggtgcagggcggacgatttcaaaacagattacccattcattcgatagagaagatcaccaagatttcgtgattcgctgccgaacttattccaattcaataagagatctcaatattatgccttgaagaggactcgaacctccacgctctttagcacgagattttgagtctcgcgtgtctaccatttcaccaccaaggcatcttgaaagcgaatcgtattccatgaatatgatatctatctagtgtgatgtatggaatatatgacaaaggtagagtgttagagtctttttattgattggtcatgtcatataggcccgagtcggacatccaattggttcgatttgaattattcagaggatatattatctatctatatctatatagatagatattctatcaaaaaaaaaatggactaattaaacctatttctcgattcaatagaagcccaaagaggtgaatttaatagggtcctaaataatgagatatatgtaaaaaacaggtccgattacgcctattcctaatcctaaatagaatggaacaacgcagggatctatatgtaaacatagtatctatttagatacgctcgaatgaccccttcttataatgagaatgtatataaccccatttcggtcgggtccggtatggaatgaacttataatcatggaattgactcgatcatcagattttcgattataagttcataaccctagcccattcccattttgggcggagcagatctactaattctttgattccagttagtaaggtaagagttgaactaagaaatagattctagaagctaaaaaagggtatcctgagcaatttcaataatagggttcattgatattcctggtatagtagatgctatcacacatacaatcatac

SEQ ID NO: 99 - DNA, 294 bp, Glycine max
ggtcatgtcatataggcccgagtcggacatccaattggttcgatttgaattattcagaggatatattatctatctatatctatatagatagatattctatcaaaaaaaaaatggactaattaaacctatttctcgattcaatagaagcccaaagaggtgaatttaatagggtcctaaataatgagatatatgtaaaaaacaggtccgattacgcctattcctaatcctaaatagaatggaacaacgcagggatctatatgtaaacatagtatctatttagatacgctcgaatga

SEQ ID NO: 100 - DNA, 882 bp, Glycine max
ttggtttaactgaataactaaaaaaaaaaaaaaaatagatagacctttctttctcttcgtctcaggtcgatggatcttctcaattgaaagatcccctatatcgataatacacattccagtttgggaacgtccggtgccaaagtcattgaatgggtaagtcgccaattcctaaaacggactatttaatgtactttatccgcccgctgggttacgggtgggtattttaccagaggttttgattgtatcaaatctacccctgtgtgatttttgttgaagcatatactcctcggggagggtgcagggcggacgatttcaaaacagattacccattcattcgatagagaagatcaccaagatttcgtgattcgctgccgaacttattccaattcaataagagatctcaatattatgccttgaagaggactcgaacctccacgctctttagcacgagattttgagtctcgcgtgtctaccatttcaccaccaaggcatcttgaaagcgaatcgtattccatgaatatgatatctatctagtgtgatgtatggaatatatgacaaaggtagagtgttagagtctttttattgattggtcatgtcatataggcccgagtcggacatccaattggttcgatttgaattattcagaggatatattatctatctatatctatatagatagatattctatcaaaaaaaaaatggactaattaaacctatttctcgattcaatagaagcccaaagaggtgaatttaatagggtcctaaataatgagatatatgtaaaaaacaggtccgattacgcctattcctaatcctaaatagaatggaacaacgcagggatctatatgtaaacatagtatctatttagatacgctcgaatga

SEQ ID NO: 101 - DNA, 588 bp, Glycine max
ggtcatgtcatataggcccgagtcggacatccaattggttcgatttgaattattcagaggatatattatctatctatatctatatagatagatattctatcaaaaaaaaaatggactaattaaacctatttctcgattcaatagaagcccaaagaggtgaatttaatagggtcctaaataatgagatatatgtaaaaaacaggtccgattacgcctattcctaatcctaaatagaatggaacaacgcagggatctatatgtaaacatagtatctatttagatacgctcgaatgaccccttcttataatgagaatgtatataaccccatttcggtcgggtccggtatggaatgaacttataatcatggaattgactcgatcatcagattttcgattataagttcataaccctagcccattcccattttgggcggagcagatctactaattctttgattccagttagtaaggtaagagttgaactaagaaatagattctagaagctaaaaaagggtatcctgagcaatttcaataatagggttcattgatattcctggtatagtagatgctatcacacatacaatcatac

SEQ ID NO: 102 - DNA, 686 bp, Glycine max
ctcctcgcccacaaccggccaaaaaagggaaggatttttccctctgggggtaggaaaatcatgatcgggctagcggacccaaagctatggaacttgtgagtactttgtagaaatggaatggccttaccttactgttttctgtttattaaaaaaaagattcataaatcgtggatattattaaataaatccagtatgtcctaatcttttaatccccatttttttttgttttacgcttcctcagccaggtttgggcagaatagcagagcaagtaaagtattattagcatagcaaaaatgcgttcctcgtcattaatatgtttgctagtggtaattgtggcttctcgggagaattgatgaatgcatcaaagatgcacttgctattgctagtaccagtacatctaataattcttaattgtttggtttaaatagccccggactgtggaacaaaggagtatcccgaacctacaccgaggtattgacggcgattctcaaatatcgcagaacagaacagaatgggatacgatgagatagaatgcaatagaaacaaagacaagggaaaaacgagttatctactcttcttaacggtcaaagcgaacccattcattccgaattacagatttcggaatcaataaaatctccccaagtaggattcgaacctacgaccagtcagttaacagccgaccgctc

SEQ ID NO: 103 - DNA, 196 bp, Glycine max
cctaatcttttaatccccatttttttttgttttacgcttcctcagccaggtttgggcagaatagcagagcaagtaaagtattattagcatagcaaaaatgcgttcctcgtcattaatatgtttgctagtggtaattgtggcttctcgggagaattgatgaatgcatcaaagatgcacttgctattgctagtaccag

SEQ ID NO: 104 - DNA, 392 bp, Glycine max
ctcctcgcccacaaccggccaaaaaagggaaggatttttccctctgggggtaggaaaatcatgatcgggctagcggacccaaagctatggaacttgtgagtactttgtagaaatggaatggccttaccttactgttttctgtttattaaaaaaaagattcataaatcgtggatattattaaataaatccagtatgtcctaatcttttaatccccatttttttttgttttacgcttcctcagccaggtttgggcagaatagcagagcaagtaaagtattattagcatagcaaaaatgcgttcctcgtcattaatatgtttgctagtggtaattgtggcttctcgggagaattgatgaatgcatcaaagatgcacttgctattgctagtaccag

SEQ ID NO: 105 - DNA, 490 bp, Glycine max
cctaatcttttaatccccatttttttttgttttacgcttcctcagccaggtttgggcagaatagcagagcaagtaaagtattattagcatagcaaaaatgcgttcctcgtcattaatatgtttgctagtggtaattgtggcttctcgggagaattgatgaatgcatcaaagatgcacttgctattgctagtaccagtacatctaataattcttaattgtttggtttaaatagccccggactgtggaacaaaggagtatcccgaacctacaccgaggtattgacggcgattctcaaatatcgcagaacagaacagaatgggatacgatgagatagaatgcaatagaaacaaagacaagggaaaaacgagttatctactcttcttaacggtcaaagcgaacccattcattccgaattacagatttcggaatcaataaaatctccccaagtaggattcgaacctacgaccagtcagttaacagccgaccgctc

SEQ ID NO: 106 - DNA, 784 bp, Glycine max
agtagctcagtggtagagcggtcggctgttaactgactggtcgtaggttcgaatcctacttggggagattttattgattccgaaatctgtaattcggaatgaatgggttcgctttgaccgttaagaagagtagataactcgtttttcccttgtctttgtttctattgcattctatctcatcgtatcccattctgttctgttctgcgatatttgagaatcgccgtcaatacctcggtgtaggttcgggatactcctttgttccacagtccggggctatttaaaccaaacaattaagaattattagatgtactggtactagcaatagcaagtgcatctttgatgcattcatcaattctcccgagaagccacaattaccactagcaaacatattaatgacgaggaacgcatttttgctatgctaataatactttacttgctctgctattctgcccaaacctggctgaggaagcgtaaaacaaaaaaaaatggggattaaaagattaggacatactggatttatttaataatatccacgatttatgaatcttttttttaataaacagaaaacagtaaggtaaggccattccatttctacaaagtactcacaagttccatagctttgggtccgctagcccgatcatgattttcctacccccagagggaaaaatccttcccttttttggccggttgtgggcgaggagggattcgaacccccgacaccgtggttcgtagccacgtgctctaatcctctgagctacaggccccaccccgtcatctccactgg

SEQ ID NO: 107 - DNA, 294 bp, Glycine max
ctgttctgcgatatttgagaatcgccgtcaatacctcggtgtaggttcgggatactcctttgttccacagtccggggctatttaaaccaaacaattaagaattattagatgtactggtactagcaatagcaagtgcatctttgatgcattcatcaattctcccgagaagccacaattaccactagcaaacatattaatgacgaggaacgcatttttgctatgctaataatactttacttgctctgctattctgcccaaacctggctgaggaagcgtaaaacaaaaaaaaatggg

SEQ ID NO: 108 - DNA, 490 bp, Glycine max
agtagctcagtggtagagcggtcggctgttaactgactggtcgtaggttcgaatcctacttggggagattttattgattccgaaatctgtaattcggaatgaatgggttcgctttgaccgttaagaagagtagataactcgtttttcccttgtctttgtttctattgcattctatctcatcgtatcccattctgttctgttctgcgatatttgagaatcgccgtcaatacctcggtgtaggttcgggatactcctttgttccacagtccggggctatttaaaccaaacaattaagaattattagatgtactggtactagcaatagcaagtgcatctttgatgcattcatcaattctcccgagaagccacaattaccactagcaaacatattaatgacgaggaacgcatttttgctatgctaataatactttacttgctctgctattctgcccaaacctggctgaggaagcgtaaaacaaaaaaaaatggg

SEQ ID NO: 109 - DNA, 588 bp, Glycine max
ctgttctgcgatatttgagaatcgccgtcaatacctcggtgtaggttcgggatactcctttgttccacagtccggggctatttaaaccaaacaattaagaattattagatgtactggtactagcaatagcaagtgcatctttgatgcattcatcaattctcccgagaagccacaattaccactagcaaacatattaatgacgaggaacgcatttttgctatgctaataatactttacttgctctgctattctgcccaaacctggctgaggaagcgtaaaacaaaaaaaaatggggattaaaagattaggacatactggatttatttaataatatccacgatttatgaatcttttttttaataaacagaaaacagtaaggtaaggccattccatttctacaaagtactcacaagttccatagctttgggtccgctagcccgatcatgattttcctacccccagagggaaaaatccttcccttttttggccggttgtgggcgaggagggattcgaacccccgacaccgtggttcgtagccacgtgctctaatcctctgagctacaggccccaccccgtcatctccactgg

SEQ ID NO: 110 - DNA, 1177 bp, Gossypium hirsutum
agctgatcatgtccttcaagtcgcacgttgctttctaccacatcgtttcaaacgaagttttaccataacattcctctagtttggagcattgattcaattatggaatcatgaatagtcattggttcagtcgatacatagtaatctatctatactttctatatgaaatcaatttccttctatatgctatatgaaataaataaataatttaggaagaaaaagcctcaataagaattcaaactaccccatattgtatgattgtctgaatgcaatattttttgatttttttaaacttttattatacttttctattctaattaaacttttcgattctaataaaaaaaaaatcaaaataaagaatatcattaataataatattacgaaaatactaataatatcacgaatattataaataacacaaataaactaatctttttgaatctaccgtgcatcttcaaataacccgatagatcaaataactcggtagaatagatccagcaatctcacagttgttcgagtgccaatcttaagaaatcattaaaaatcaaaagcaagaatcacattttctccaatacttgactgttagaaagaaggttattaaaaacaaaaaaagagcaatttggatatggttattctgatatataaatagagtatgctctgggacggaaggattcgaacctccgcatagcgggaccaaaacccgttgccttaccacttggccacgccccatttagatttctatttgaaactactaaacactaatatggtattccttgttcgtcaattccagttccaaatagctatagctatggaatagattcttgttacgatttttgcacgtatggatagagaattaaatcgaatttattgatcattacatagaattcaattaagatattgtatgaaagtatgatttcttctattctcttgtaagaattgaaggctttttgattgggtgagttcaaagaagtattgtttagtctgccttattttcctttcttcatttttttccttatatcaaaaaacatattaataactcaatcaaaattatctccaagaacaaaattttgttatgcttaatatctttaatttgatctgtatctgttttaattctgcccttttttcgagtagttttttattcgccaaattgcccgaggcctatgcttttttgaatccaatc

SEQ ID NO: 111 - DNA, 230 bp, Gossypium hirsutum
ctattctaattaaacttttcgattctaataaaaaaaaaatcaaaataaagaatatcattaataataatattacgaaaatactaataatatcacgaatattataaataacacaaataaactaatctttttgaatctaccgtgcatcttcaaataacccgatagatcaaataactcggtagaatagatccagcaatctcacagttgttcgagtgccaatcttaagaaatcat

SEQ ID NO: 112 - DNA, 535 bp, Gossypium hirsutum
agctgatcatgtccttcaagtcgcacgttgctttctaccacatcgtttcaaacgaagttttaccataacattcctctagtttggagcattgattcaattatggaatcatgaatagtcattggttcagtcgatacatagtaatctatctatactttctatatgaaatcaatttccttctatatgctatatgaaataaataaataatttaggaagaaaaagcctcaataagaattcaaactaccccatattgtatgattgtctgaatgcaatattttttgatttttttaaacttttattatacttttctattctaattaaacttttcgattctaataaaaaaaaaatcaaaataaagaatatcattaataataatattacgaaaatactaataatatcacgaatattataaataacacaaataaactaatctttttgaatctaccgtgcatcttcaaataacccgatagatcaaataactcggtagaatagatccagcaatctcacagttgttcgagtgccaatcttaagaaatcat

SEQ ID NO: 113 - DNA, 872 bp, Gossypium hirsutum
ctattctaattaaacttttcgattctaataaaaaaaaaatcaaaataaagaatatcattaataataatattacgaaaatactaataatatcacgaatattataaataacacaaataaactaatctttttgaatctaccgtgcatcttcaaataacccgatagatcaaataactcggtagaatagatccagcaatctcacagttgttcgagtgccaatcttaagaaatcattaaaaatcaaaagcaagaatcacattttctccaatacttgactgttagaaagaaggttattaaaaacaaaaaaagagcaatttggatatggttattctgatatataaatagagtatgctctgggacggaaggattcgaacctccgcatagcgggaccaaaacccgttgccttaccacttggccacgccccatttagatttctatttgaaactactaaacactaatatggtattccttgttcgtcaattccagttccaaatagctatagctatggaatagattcttgttacgatttttgcacgtatggatagagaattaaatcgaatttattgatcattacatagaattcaattaagatattgtatgaaagtatgatttcttctattctcttgtaagaattgaaggctttttgattgggtgagttcaaagaagtattgtttagtctgccttattttcctttcttcatttttttccttatatcaaaaaacatattaataactcaatcaaaattatctccaagaacaaaattttgttatgcttaatatctttaatttgatctgtatctgttttaattctgcccttttttcgagtagttttttattcgccaaattgcccgaggcctatgcttttttgaatccaatc

SEQ ID NO: 114 - DNA, 963 bp, Gossypium hirsutum
tccactcagccatctctcctaattgagaaaagataattactatgttacagcacgcaaaaagaaaggcttgaaaaaagccctttttactttgttatatagattatgttatatagattattatttatctatatagagtattattaatccatagatatatagattctaaatttgattattattagatagattgattatatagctatatccaacttttctatagagatatagaacttttatcagtaattccatttcatttatattatattctatatcatttatatttttaaataaaacaatggctctaaagaaatatctcaaataacaaaaaaagaaagaatatcgctttgatttcgcgcaaaagggacttttaaatttccaatttccacggaccggcctgggcagtacccggccgggctcatttttttattttcaactcaaataactcattttttctatgattctgcgatctggcttgttgtaacaaaaaaatcttgttattgcattgaatcaacaatcagaagcggaagaagtcctatttccgttcctatgatatagaatcaaaatattatttctattctttttctttcttcctatttctttttatttccatttattttttattttactagaataactagaataaataataaaaaaaaaactatggatttttgcacaatccatttttatgttatttatgttatgactaagtccttttgtcgaatcctgtctatcaaaaatcctataacacaagtcttctgacaaaaggtcaaaaggtggcaaggccctaattttcaggattttttaggctcctatcttgtaatcctatcttgattacgcccaattcccctgtttgacaaagggtccctttatatttatatacaataaatatacaataaatacaataatcgcattgtagcgggtatagtttagtggtaaaagtgtgattcgttctattaatccccc

SEQ ID NO: 115 - DNA, 428 bp, Gossypium hirsutum
ctatagagatatagaacttttatcagtaattccatttcatttatattatattctatatcatttatatttttaaataaaacaatggctctaaagaaatatctcaaataacaaaaaaagaaagaatatcgctttgatttcgcgcaaaagggacttttaaatttccaatttccacggaccggcctgggcagtacccggccgggctcatttttttattttcaactcaaataactcattttttctatgattctgcgatctggcttgttgtaacaaaaaaatcttgttattgcattgaatcaacaatcagaagcggaagaagtcctatttccgttcctatgatatagaatcaaaatattatttctattctttttctttcttcctatttctttttatttccatttattttttattttactagaataactagaata

SEQ ID NO: 116 - DNA, 642 bp, Gossypium hirsutum
tccactcagccatctctcctaattgagaaaagataattactatgttacagcacgcaaaaagaaaggcttgaaaaaagccctttttactttgttatatagattatgttatatagattattatttatctatatagagtattattaatccatagatatatagattctaaatttgattattattagatagattgattatatagctatatccaacttttctatagagatatagaacttttatcagtaattccatttcatttatattatattctatatcatttatatttttaaataaaacaatggctctaaagaaatatctcaaataacaaaaaaagaaagaatatcgctttgatttcgcgcaaaagggacttttaaatttccaatttccacggaccggcctgggcagtacccggccgggctcatttttttattttcaactcaaataactcattttttctatgattctgcgatctggcttgttgtaacaaaaaaatcttgttattgcattgaatcaacaatcagaagcggaagaagtcctatttccgttcctatgatatagaatcaaaatattatttctattctttttctttcttcctatttctttttatttccatttattttttattttactagaataactagaata

SEQ ID NO: 117 - DNA, 749 bp, Gossypium hirsutum
ctatagagatatagaacttttatcagtaattccatttcatttatattatattctatatcatttatatttttaaataaaacaatggctctaaagaaatatctcaaataacaaaaaaagaaagaatatcgctttgatttcgcgcaaaagggacttttaaatttccaatttccacggaccggcctgggcagtacccggccgggctcatttttttattttcaactcaaataactcattttttctatgattctgcgatctggcttgttgtaacaaaaaaatcttgttattgcattgaatcaacaatcagaagcggaagaagtcctatttccgttcctatgatatagaatcaaaatattatttctattctttttctttcttcctatttctttttatttccatttattttttattttactagaataactagaataaataataaaaaaaaaactatggatttttgcacaatccatttttatgttatttatgttatgactaagtccttttgtcgaatcctgtctatcaaaaatcctataacacaagtcttctgacaaaaggtcaaaaggtggcaaggccctaattttcaggattttttaggctcctatcttgtaatcctatcttgattacgcccaattcccctgtttgacaaagggtccctttatatttatatacaataaatatacaataaatacaataatcgcattgtagcgggtatagtttagtggtaaaagtgtgattcgttctattaatccccc

SEQ ID NO: 118 - DNA, 2033 bp, Gossypium hirsutum
cgatccatgaacccacaaaatccttcaaattgtatctgattcaacccaggtattgtagacatttccccattttcatccccgagcattttgaatttcccatttctcaaaaaaatcccattcttttttcattcttcatcgaatcctacgaatcgatctaataatgatggaattgaatttctattctgtttactgaatcacatgaaattttatctaactccatataccatataatatatgtggaatgtatgaaatatgaaatgcgtatgaacggaagaagaaaaattatactcaaatttgaatttatatttataacaaacagatacagaaaagaattgataaatgccatttcgccgcatggagttttgtttttgtatagaatataaaaaaaaataattcaatttctaccattattatgatattacatattccaatccgattgaataccagaaaaatgaaaggaattcctgatttgatctgttcgttgagataaagacaaaataatcataaaatatatatatagggagagagttgatttttctagcgcttaattttttcatggactccattgttcaaaaaacgattcgcagagaagatagatgtttttacccactttttagttttttattttttagtagaatatttagaatatgattgaagtgcgtacgaaaagggggcttctatttattaaacatgtgcagatatagcatttctatttatatatgtctttcttttattccattataacgctctaatggagacaagacatggcgtgctctatcaaaaattcgattttttcttttattttaatctattcaatgaaaaattgaaacacgataatttcttgctgattccctatggacatcatatctagatagataaaatacctcattagataactataactgtgtaacaacttatgctctggggtttacatagactcataattgctgttatattattataatatacttgaaattaagaaagttttttttattgaaaaaaaaatcaatttagttatgtatccagttttattttcttataccattagaaaaagacaagtgaagaagaatcgtccataaatttgcagtcaatagttaatggttccaatttatttgtcctgaatttttacttttgtaactgagaatccacattttctttttcaatagaaaagaaagggaaagtttttagatattgtgtgtcttgagatactataaccaattgaaggtatggatccgatctaaaaatgaaaaagaaaagggagatactctcctttttttgtttgtaaccttttggcgacatggccgagcggtaaggcgggggactgcaaatcccttattccccagttcaaatccgggtgtcgcctgatcaacaaaaaactcgaaatcctaacgtctcctaacgtctaggattcaaggaaaaactaaagtagtggaagggaatcagtaaatcttgatatgggagccccccttactaatggagtggctactagaggagtcttgaattagattggataacgggagtgtctaattaactaatgaatggttgtagaagggttggaagatactttgtatacagacggatgaaagtctctgagtgttcaggcatccaattaatattagattggatagataattggcttttacttaatgagggacaccaaaagaaagaataagtcttattattgatacatgtgagtaacattcttttgatacttccaaaagtgttactgcgcattttgcttgtgcttaatggttctcgatttgactcgaaatcatataagagcttgttataagcggatttatcgaagtgattcatcaacggtggcgtgtcacaattcccttttctttttgactctgcgccagtaattccactattattagtgaacaataatggaaaaattgaattccttcatatttgtttcatattcatagagataggggacataatacacatggatatagtaagtcttgcttgggctgctttaatggtagtctttacattttccctttcactcgtagtatggggaagaagtg

SEQ ID NO: 119 - DNA, 749 bp, Gossypium hirsutum
acagaaaagaattgataaatgccatttcgccgcatggagttttgtttttgtatagaatataaaaaaaaataattcaatttctaccattattatgatattacatattccaatccgattgaataccagaaaaatgaaaggaattcctgatttgatctgttcgttgagataaagacaaaataatcataaaatatatatatagggagagagttgatttttctagcgcttaattttttcatggactccattgttcaaaaaacgattcgcagagaagatagatgtttttacccactttttagttttttattttttagtagaatatttagaatatgattgaagtgcgtacgaaaagggggcttctatttattaaacatgtgcagatatagcatttctatttatatatgtctttcttttattccattataacgctctaatggagacaagacatggcgtgctctatcaaaaattcgattttttcttttattttaatctattcaatgaaaaattgaaacacgataatttcttgctgattccctatggacatcatatctagatagataaaatacctcattagataactataactgtgtaacaacttatgctctggggtttacatagactcataattgctgttatattattataatatacttgaaattaagaaagttttttttattgaaaaaaaaatcaatttagttatgtatccagttttattttcttataccattagaaaaagacaagtgaagaagaat

SEQ ID NO: 120 - DNA, 1070 bp, Gossypium hirsutum
cgatccatgaacccacaaaatccttcaaattgtatctgattcaacccaggtattgtagacatttccccattttcatccccgagcattttgaatttcccatttctcaaaaaaatcccattcttttttcattcttcatcgaatcctacgaatcgatctaataatgatggaattgaatttctattctgtttactgaatcacatgaaattttatctaactccatataccatataatatatgtggaatgtatgaaatatgaaatgcgtatgaacggaagaagaaaaattatactcaaatttgaatttatatttataacaaacagatacagaaaagaattgataaatgccatttcgccgcatggagttttgtttttgtatagaatataaaaaaaaataattcaatttctaccattattatgatattacatattccaatccgattgaataccagaaaaatgaaaggaattcctgatttgatctgttcgttgagataaagacaaaataatcataaaatatatatatagggagagagttgatttttctagcgcttaattttttcatggactccattgttcaaaaaacgattcgcagagaagatagatgtttttacccactttttagttttttattttttagtagaatatttagaatatgattgaagtgcgtacgaaaagggggcttctatttattaaacatgtgcagatatagcatttctatttatatatgtctttcttttattccattataacgctctaatggagacaagacatggcgtgctctatcaaaaattcgattttttcttttattttaatctattcaatgaaaaattgaaacacgataatttcttgctgattccctatggacatcatatctagatagataaaatacctcattagataactataactgtgtaacaacttatgctctggggtttacatagactcataattgctgttatattattataatatacttgaaattaagaaagttttttttattgaaaaaaaaatcaatttagttatgtatccagttttattttcttataccattagaaaaagacaagtgaagaagaat

SEQ ID NO: 121 - DNA, 1712 bp, Gossypium hirsutum
acagaaaagaattgataaatgccatttcgccgcatggagttttgtttttgtatagaatataaaaaaaaataattcaatttctaccattattatgatattacatattccaatccgattgaataccagaaaaatgaaaggaattcctgatttgatctgttcgttgagataaagacaaaataatcataaaatatatatatagggagagagttgatttttctagcgcttaattttttcatggactccattgttcaaaaaacgattcgcagagaagatagatgtttttacccactttttagttttttattttttagtagaatatttagaatatgattgaagtgcgtacgaaaagggggcttctatttattaaacatgtgcagatatagcatttctatttatatatgtctttcttttattccattataacgctctaatggagacaagacatggcgtgctctatcaaaaattcgattttttcttttattttaatctattcaatgaaaaattgaaacacgataatttcttgctgattccctatggacatcatatctagatagataaaatacctcattagataactataactgtgtaacaacttatgctctggggtttacatagactcataattgctgttatattattataatatacttgaaattaagaaagttttttttattgaaaaaaaaatcaatttagttatgtatccagttttattttcttataccattagaaaaagacaagtgaagaagaatcgtccataaatttgcagtcaatagttaatggttccaatttatttgtcctgaatttttacttttgtaactgagaatccacattttctttttcaatagaaaagaaagggaaagtttttagatattgtgtgtcttgagatactataaccaattgaaggtatggatccgatctaaaaatgaaaaagaaaagggagatactctcctttttttgtttgtaaccttttggcgacatggccgagcggtaaggcgggggactgcaaatcccttattccccagttcaaatccgggtgtcgcctgatcaacaaaaaactcgaaatcctaacgtctcctaacgtctaggattcaaggaaaaactaaagtagtggaagggaatcagtaaatcttgatatgggagccccccttactaatggagtggctactagaggagtcttgaattagattggataacgggagtgtctaattaactaatgaatggttgtagaagggttggaagatactttgtatacagacggatgaaagtctctgagtgttcaggcatccaattaatattagattggatagataattggcttttacttaatgagggacaccaaaagaaagaataagtcttattattgatacatgtgagtaacattcttttgatacttccaaaagtgttactgcgcattttgcttgtgcttaatggttctcgatttgactcgaaatcatataagagcttgttataagcggatttatcgaagtgattcatcaacggtggcgtgtcacaattcccttttctttttgactctgcgccagtaattccactattattagtgaacaataatggaaaaattgaattccttcatatttgtttcatattcatagagataggggacataatacacatggatatagtaagtcttgcttgggctgctttaatggtagtctttacattttccctttcactcgtagtatggggaagaagtg

SEQ ID NO: 122 - DNA, 264 bp, Gossypium hirsutum
cttgatatgggagccccccttactaatggagtggctactagaggagtcttgaattagattggataacgggagtgtctaattaactaatgaatggttgtagaagggttggaagatactttgtatacagacggatgaaagtctctgagtgttcaggcatccaattaatattagattggatagataattggcttttacttaatgagggacaccaaaagaaagaataagtcttattattgatacatgtgagtaacattcttttgatac

SEQ ID NO: 123 - DNA, 1712 bp, Gossypium hirsutum
cgatccatgaacccacaaaatccttcaaattgtatctgattcaacccaggtattgtagacatttccccattttcatccccgagcattttgaatttcccatttctcaaaaaaatcccattcttttttcattcttcatcgaatcctacgaatcgatctaataatgatggaattgaatttctattctgtttactgaatcacatgaaattttatctaactccatataccatataatatatgtggaatgtatgaaatatgaaatgcgtatgaacggaagaagaaaaattatactcaaatttgaatttatatttataacaaacagatacagaaaagaattgataaatgccatttcgccgcatggagttttgtttttgtatagaatataaaaaaaaataattcaatttctaccattattatgatattacatattccaatccgattgaataccagaaaaatgaaaggaattcctgatttgatctgttcgttgagataaagacaaaataatcataaaatatatatatagggagagagttgatttttctagcgcttaattttttcatggactccattgttcaaaaaacgattcgcagagaagatagatgtttttacccactttttagttttttattttttagtagaatatttagaatatgattgaagtgcgtacgaaaagggggcttctatttattaaacatgtgcagatatagcatttctatttatatatgtctttcttttattccattataacgctctaatggagacaagacatggcgtgctctatcaaaaattcgattttttcttttattttaatctattcaatgaaaaattgaaacacgataatttcttgctgattccctatggacatcatatctagatagataaaatacctcattagataactataactgtgtaacaacttatgctctggggtttacatagactcataattgctgttatattattataatatacttgaaattaagaaagttttttttattgaaaaaaaaatcaatttagttatgtatccagttttattttcttataccattagaaaaagacaagtgaagaagaatcgtccataaatttgcagtcaatagttaatggttccaatttatttgtcctgaatttttacttttgtaactgagaatccacattttctttttcaatagaaaagaaagggaaagtttttagatattgtgtgtcttgagatactataaccaattgaaggtatggatccgatctaaaaatgaaaaagaaaagggagatactctcctttttttgtttgtaaccttttggcgacatggccgagcggtaaggcgggggactgcaaatcccttattccccagttcaaatccgggtgtcgcctgatcaacaaaaaactcgaaatcctaacgtctcctaacgtctaggattcaaggaaaaactaaagtagtggaagggaatcagtaaatcttgatatgggagccccccttactaatggagtggctactagaggagtcttgaattagattggataacgggagtgtctaattaactaatgaatggttgtagaagggttggaagatactttgtatacagacggatgaaagtctctgagtgttcaggcatccaattaatattagattggatagataattggcttttacttaatgagggacaccaaaagaaagaataagtcttattattgatacatgtgagtaacattcttttgatac

SEQ ID NO: 124 - DNA, 585 bp, Gossypium hirsutum
cttgatatgggagccccccttactaatggagtggctactagaggagtcttgaattagattggataacgggagtgtctaattaactaatgaatggttgtagaagggttggaagatactttgtatacagacggatgaaagtctctgagtgttcaggcatccaattaatattagattggatagataattggcttttacttaatgagggacaccaaaagaaagaataagtcttattattgatacatgtgagtaacattcttttgatacttccaaaagtgttactgcgcattttgcttgtgcttaatggttctcgatttgactcgaaatcatataagagcttgttataagcggatttatcgaagtgattcatcaacggtggcgtgtcacaattcccttttctttttgactctgcgccagtaattccactattattagtgaacaataatggaaaaattgaattccttcatatttgtttcatattcatagagataggggacataatacacatggatatagtaagtcttgcttgggctgctttaatggtagtctttacattttccctttcactcgtagtatggggaagaagtg

SEQ ID NO: 125 - DNA, 963 bp, Gossypium hirsutum
aaccgctagacgatgggggcatacttgcctgaccgcgaccatactatgctcatagtatgagcagttttttgaaattgtcaatataatggaatgactagagccgaaagctttttccatcttttatgattttccattttagattcacgatttatactcagtaaatcattcatcgccactctattcgatatagaaataaattagataaattcaatctattatataaatatttattagaaataaatagatattataaaaatagaaataaatagatattataaaaagaaactagattatattaataatacaaagtataagatcagtgattggtttaacataaacataaaaaagggagagttccttgcttcattttttccactttcattcattgattcactcattattacgacgagacaggcatgtttctatctcacactaagccggtaaattaacaaaaagtaaatcgggaatttttggaagaggcagcaagaagttatcgaaaattatgtgtgtgtgtttttttttctaaaaactaaaaattgggttcggtggacaaagcaaatcccttcatcacatgtttcgataaaatagattggatctatgtcgaattgctaaggtacctgtattaatcaaataaatgaatttcgttctgttctgataagccaattatgattggccttgaaacaattcattgcattgatatttatcaaacccaataccaataaaaccattttaccctctacacgttaagtaaattagaattctcattcctgagagctactagccactatgagtctactgcatatacttatatatatataaatagatcttatccgtctactaattcattcagtaattgaatcgaaccgcccttttaactcagtggtagagtaacgccatggtaaggcgtaagtcatcggttcaaatccgataaggggcttttgactttttttttcataaaactcc

SEQ ID NO: 126 - DNA, 469 bp, Gossypium hirsutum
attatataaatatttattagaaataaatagatattataaaaatagaaataaatagatattataaaaagaaactagattatattaataatacaaagtataagatcagtgattggtttaacataaacataaaaaagggagagttccttgcttcattttttccactttcattcattgattcactcattattacgacgagacaggcatgtttctatctcacactaagccggtaaattaacaaaaagtaaatcgggaatttttggaagaggcagcaagaagttatcgaaaattatgtgtgtgtgtttttttttctaaaaactaaaaattgggttcggtggacaaagcaaatcccttcatcacatgtttcgataaaatagattggatctatgtcgaattgctaaggtacctgtattaatcaaataaatgaatttcgttctgttctgataagccaattatgattggccttgaaaca

SEQ ID NO: 127 - DNA, 683 bp, Gossypium hirsutum
aaccgctagacgatgggggcatacttgcctgaccgcgaccatactatgctcatagtatgagcagttttttgaaattgtcaatataatggaatgactagagccgaaagctttttccatcttttatgattttccattttagattcacgatttatactcagtaaatcattcatcgccactctattcgatatagaaataaattagataaattcaatctattatataaatatttattagaaataaatagatattataaaaatagaaataaatagatattataaaaagaaactagattatattaataatacaaagtataagatcagtgattggtttaacataaacataaaaaagggagagttccttgcttcattttttccactttcattcattgattcactcattattacgacgagacaggcatgtttctatctcacactaagccggtaaattaacaaaaagtaaatcgggaatttttggaagaggcagcaagaagttatcgaaaattatgtgtgtgtgtttttttttctaaaaactaaaaattgggttcggtggacaaagcaaatcccttcatcacatgtttcgataaaatagattggatctatgtcgaattgctaaggtacctgtattaatcaaataaatgaatttcgttctgttctgataagccaattatgattggccttgaaaca

SEQ ID NO: 128 - DNA, 749 bp, Gossypium hirsutum
attatataaatatttattagaaataaatagatattataaaaatagaaataaatagatattataaaaagaaactagattatattaataatacaaagtataagatcagtgattggtttaacataaacataaaaaagggagagttccttgcttcattttttccactttcattcattgattcactcattattacgacgagacaggcatgtttctatctcacactaagccggtaaattaacaaaaagtaaatcgggaatttttggaagaggcagcaagaagttatcgaaaattatgtgtgtgtgtttttttttctaaaaactaaaaattgggttcggtggacaaagcaaatcccttcatcacatgtttcgataaaatagattggatctatgtcgaattgctaaggtacctgtattaatcaaataaatgaatttcgttctgttctgataagccaattatgattggccttgaaacaattcattgcattgatatttatcaaacccaataccaataaaaccattttaccctctacacgttaagtaaattagaattctcattcctgagagctactagccactatgagtctactgcatatacttatatatatataaatagatcttatccgtctactaattcattcagtaattgaatcgaaccgcccttttaactcagtggtagagtaacgccatggtaaggcgtaagtcatcggttcaaatccgataaggggcttttgactttttttttcataaaactcc

SEQ ID NO: 129 - DNA, 1605 bp, Gossypium hirsutum
aatcgaaccgcccttttaactcagtggtagagtaacgccatggtaaggcgtaagtcatcggttcaaatccgataaggggcttttgactttttttttcataaaactcctatgaaatggtagtattcatattgaatactagggatgttgtttttattggtaataaaaacccaactgtatatgtataataattgaattagaaattgaattagaaattgtataatttaactctaataagttattattctaatgagttagagtagagtaattctaaaagaaagttgaaattataatgaataatgataagccgtctcttgaagcgacaaatatatatttctattttgttttgattttgaattagattattccaatcaaatccattgtaaagaattgtaaagattagaaatcaacaaaagaaaaagtaagtggatctaacccattgaatcgtgactatatccactattctgatattaaaattcgatagagatgaaattggaacagtcgatttctttttatttcatatttatttggactccacaagaatccgtcgatatttccgattcaatcttcttgttcctaggaataaattaatattctattcctttcgagataaagagaaacatttatttcaagttacaacctaagagcctttttcaacatcttatctttctttgattccagaacacaacaagatccatttctatctatatattctatctattctataatagatagaaacctaaatcaaatcatgtcttcacatatcaaatatttccatatcgatacatatgacacttttgttctgccaatgtgatggagagtgagtgcgaaaaagaaactttcattttgagtctcctattatttaatttaatattattaaatattgtattagattgaaaaataggaacttcgaatctttttctgatatacataaagtaaatagaaaaagattcgaagtggatttctcgacccgtgaaatgaaaaaatagcctctggagtttttttgttttatattcatctgaaggaaatatgagaaagaaaacaataaataaaagaaataaaatgaaggaaatatatcagactatagtaatttaatacacatagaaattagaaaaatacataaaaatattgatttttatcaatttcacgaacaagatccaagaataagattagttgatggagggggaggggtagatctggggatcaaccagtagcgagagaagggatcgcttcttccttgaattgaagagttcgttcaaaaaattcatctatctgattgatgagccataagacaattcatggttcagacgactagtaaaaatagaaaagaataaacgaattgagttcatggatttacctaagtcaggttatggaccaatacaagaaaatatttttttatattcgaaaccaattaattattaattataaggggcagtgcacgagaaatcaaatcatacataaaaaatcgaatcccccagcgcccccataacataaggtgctcggaaatggttgaagtagttgaataggaggattgctatgactatagcccttggtaaatttaccaaagatgaaaatgatttatttgatattatggatgactggttacgt

SEQ ID NO: 130 - DNA, 1070 bp, Gossypium hirsutum
gtataatttaactctaataagttattattctaatgagttagagtagagtaattctaaaagaaagttgaaattataatgaataatgataagccgtctcttgaagcgacaaatatatatttctattttgttttgattttgaattagattattccaatcaaatccattgtaaagaattgtaaagattagaaatcaacaaaagaaaaagtaagtggatctaacccattgaatcgtgactatatccactattctgatattaaaattcgatagagatgaaattggaacagtcgatttctttttatttcatatttatttggactccacaagaatccgtcgatatttccgattcaatcttcttgttcctaggaataaattaatattctattcctttcgagataaagagaaacatttatttcaagttacaacctaagagcctttttcaacatcttatctttctttgattccagaacacaacaagatccatttctatctatatattctatctattctataatagatagaaacctaaatcaaatcatgtcttcacatatcaaatatttccatatcgatacatatgacacttttgttctgccaatgtgatggagagtgagtgcgaaaaagaaactttcattttgagtctcctattatttaatttaatattattaaatattgtattagattgaaaaataggaacttcgaatctttttctgatatacataaagtaaatagaaaaagattcgaagtggatttctcgacccgtgaaatgaaaaaatagcctctggagtttttttgttttatattcatctgaaggaaatatgagaaagaaaacaataaataaaagaaataaaatgaaggaaatatatcagactatagtaatttaatacacatagaaattagaaaaatacataaaaatattgatttttatcaatttcacgaacaagatccaagaataagattagttgatggagggggaggggtagatctggggatcaaccagtagcgagagaagggatcgcttcttccttgaattgaagagttcgttcaaaaaattcatctatctgattgatgagcca

SEQ ID NO: 131 - DNA, 1284 bp, Gossypium hirsutum
aatcgaaccgcccttttaactcagtggtagagtaacgccatggtaaggcgtaagtcatcggttcaaatccgataaggggcttttgactttttttttcataaaactcctatgaaatggtagtattcatattgaatactagggatgttgtttttattggtaataaaaacccaactgtatatgtataataattgaattagaaattgaattagaaattgtataatttaactctaataagttattattctaatgagttagagtagagtaattctaaaagaaagttgaaattataatgaataatgataagccgtctcttgaagcgacaaatatatatttctattttgttttgattttgaattagattattccaatcaaatccattgtaaagaattgtaaagattagaaatcaacaaaagaaaaagtaagtggatctaacccattgaatcgtgactatatccactattctgatattaaaattcgatagagatgaaattggaacagtcgatttctttttatttcatatttatttggactccacaagaatccgtcgatatttccgattcaatcttcttgttcctaggaataaattaatattctattcctttcgagataaagagaaacatttatttcaagttacaacctaagagcctttttcaacatcttatctttctttgattccagaacacaacaagatccatttctatctatatattctatctattctataatagatagaaacctaaatcaaatcatgtcttcacatatcaaatatttccatatcgatacatatgacacttttgttctgccaatgtgatggagagtgagtgcgaaaaagaaactttcattttgagtctcctattatttaatttaatattattaaatattgtattagattgaaaaataggaacttcgaatctttttctgatatacataaagtaaatagaaaaagattcgaagtggatttctcgacccgtgaaatgaaaaaatagcctctggagtttttttgttttatattcatctgaaggaaatatgagaaagaaaacaataaataaaagaaataaaatgaaggaaatatatcagactatagtaatttaatacacatagaaattagaaaaatacataaaaatattgatttttatcaatttcacgaacaagatccaagaataagattagttgatggagggggaggggtagatctggggatcaaccagtagcgagagaagggatcgcttcttccttgaattgaagagttcgttcaaaaaattcatctatctgattgatgagcca

SEQ ID NO: 132 - DNA, 1391 bp, Gossypium hirsutum
gtataatttaactctaataagttattattctaatgagttagagtagagtaattctaaaagaaagttgaaattataatgaataatgataagccgtctcttgaagcgacaaatatatatttctattttgttttgattttgaattagattattccaatcaaatccattgtaaagaattgtaaagattagaaatcaacaaaagaaaaagtaagtggatctaacccattgaatcgtgactatatccactattctgatattaaaattcgatagagatgaaattggaacagtcgatttctttttatttcatatttatttggactccacaagaatccgtcgatatttccgattcaatcttcttgttcctaggaataaattaatattctattcctttcgagataaagagaaacatttatttcaagttacaacctaagagcctttttcaacatcttatctttctttgattccagaacacaacaagatccatttctatctatatattctatctattctataatagatagaaacctaaatcaaatcatgtcttcacatatcaaatatttccatatcgatacatatgacacttttgttctgccaatgtgatggagagtgagtgcgaaaaagaaactttcattttgagtctcctattatttaatttaatattattaaatattgtattagattgaaaaataggaacttcgaatctttttctgatatacataaagtaaatagaaaaagattcgaagtggatttctcgacccgtgaaatgaaaaaatagcctctggagtttttttgttttatattcatctgaaggaaatatgagaaagaaaacaataaataaaagaaataaaatgaaggaaatatatcagactatagtaatttaatacacatagaaattagaaaaatacataaaaatattgatttttatcaatttcacgaacaagatccaagaataagattagttgatggagggggaggggtagatctggggatcaaccagtagcgagagaagggatcgcttcttccttgaattgaagagttcgttcaaaaaattcatctatctgattgatgagccataagacaattcatggttcagacgactagtaaaaatagaaaagaataaacgaattgagttcatggatttacctaagtcaggttatggaccaatacaagaaaatatttttttatattcgaaaccaattaattattaattataaggggcagtgcacgagaaatcaaatcatacataaaaaatcgaatcccccagcgcccccataacataaggtgctcggaaatggttgaagtagttgaataggaggattgctatgactatagcccttggtaaatttaccaaagatgaaaatgatttatttgatattatggatgactggttacgt

SEQ ID NO: 133 - DNA, 2033 bp, Gossypium hirsutum
taacgtgacataaggactccttattaatttgaattttattgaaatttcataaaccgaaattaaaactgaactaaatgaagcgaaatcgactgaaggattgtactacaaatattaatgagatgaattggatcaatatccatactttacttttttgtttttttgtattgtatataatgtatacaaaaaatagaaataaatatatatataataataatatataaatataaaaataatattaatatataaatataaaaatatataaaatatttagaatatatataataaaatagatataatatataaataaattgaatataatattaaatagataaataaattaaaccaaaagagtttccttttttttcttgatttattctgcaaagatctaactcctcaaattgatccctaattggaagtttcgacgagataataaacagattgttgacctttggaaagaattggaaagaaaaggggaaaaagctttttcactgtttgttctttgatttcaaaaaaaacattctaaattatgtaaaaaaggcaaaaagcaaacaaatagggaaagccggctatcggaatcgaaccgatgaccatcgcattacaaatgcgatgctctaacctctgagctaagcgggctcaaataatagaaatttggtatccatagggattcagcaaactattagatcttatctattaactatctattagttattcataattaatatgaattattaatatgaattatagactcgaatttagaaaaactagaattttttctaatttttaatgccatacttaatataaacttaatataaatataacatgtttaatataataatggtagattcaatctaatattcaagctaatgttgatagaatctaataattaaattaaaattttatatatatatttattatttatctatatattttatatatttaatatatttaatatctttttttatatcttagttatatttcttttatattaatatattactaatattactgactccatttttattttctagtttattttatattttagtatattttacatctaaatgatatagaaatagaaaattttttatatagatttcatttaatcgatcgatttaatagatttaatatagattaaatttattttgaatagatttaatatagattaaatttatttctatttagtttagagttctaaatagagataattagagtcaaaatctttttatgcagttatatattttatgattatataaatgaaacgttataagtctagataattagaatgaaactctttttagactcaaaaatttgaattatcttcgaattcgaaatagaaatgaatggaataattagttctagttattaaatataaataattagttctagctattatactataaaaaaaaataattaatttcattttcatttttaataatgaataaattcaattttcatttttctttttttagttattatggttatataggatagtctaataaaaggataagaataaaaatgaaattaaagaaaaaaagatgcaacccatagaaatgaaatccagatcataatgagagactcctttcttttgttttcattcataaagacagaagctaagacgaaaaaaagaatcgacccttcgagtattccaccaaattgcctgagaaaactgagagtaagagggacatatatatgttatgaaatacccatctatattgaattgcgaatacagaaatgataaaatatttttggaccaaataaaaattaatatgggtctccgatagagacgaaagacggataaacaaacaagaaaataggtaaagacccttcgatataagaatctgtatctatatctactaaattcaacggtttcgcataaaaagaaagcaaataaataaacgaaagaaaataaacaaatgaaagaagggggaaaggagggagaaagggggaaggaggggcatcctaatgagatcctaatctcaagacacaaaaggggatatggcgaaattggtagacgctacggacttaattggatt

SEQ ID NO: 134 - DNA, 214 bp, Gossypium hirsutum
tatataaatataaaaataatattaatatataaatataaaaatatataaaatatttagaatatatataataaaatagatataatatataaataaattgaatataatattaaatagataaataaattaaaccaaaagagtttccttttttttcttgatttattctgcaaagatctaactcctcaaattgatccctaattggaagtttcgacgagat

SEQ ID NO: 135 - DNA, 428 bp, Gossypium hirsutum
taacgtgacataaggactccttattaatttgaattttattgaaatttcataaaccgaaattaaaactgaactaaatgaagcgaaatcgactgaaggattgtactacaaatattaatgagatgaattggatcaatatccatactttacttttttgtttttttgtattgtatataatgtatacaaaaaatagaaataaatatatatataataataatatataaatataaaaataatattaatatataaatataaaaatatataaaatatttagaatatatataataaaatagatataatatataaataaattgaatataatattaaatagataaataaattaaaccaaaagagtttccttttttttcttgatttattctgcaaagatctaactcctcaaattgatccctaattggaagtttcgacgagat

SEQ ID NO: 136 - DNA, 1819 bp, Gossypium hirsutum
tatataaatataaaaataatattaatatataaatataaaaatatataaaatatttagaatatatataataaaatagatataatatataaataaattgaatataatattaaatagataaataaattaaaccaaaagagtttccttttttttcttgatttattctgcaaagatctaactcctcaaattgatccctaattggaagtttcgacgagataataaacagattgttgacctttggaaagaattggaaagaaaaggggaaaaagctttttcactgtttgttctttgatttcaaaaaaaacattctaaattatgtaaaaaaggcaaaaagcaaacaaatagggaaagccggctatcggaatcgaaccgatgaccatcgcattacaaatgcgatgctctaacctctgagctaagcgggctcaaataatagaaatttggtatccatagggattcagcaaactattagatcttatctattaactatctattagttattcataattaatatgaattattaatatgaattatagactcgaatttagaaaaactagaattttttctaatttttaatgccatacttaatataaacttaatataaatataacatgtttaatataataatggtagattcaatctaatattcaagctaatgttgatagaatctaataattaaattaaaattttatatatatatttattatttatctatatattttatatatttaatatatttaatatctttttttatatcttagttatatttcttttatattaatatattactaatattactgactccatttttattttctagtttattttatattttagtatattttacatctaaatgatatagaaatagaaaattttttatatagatttcatttaatcgatcgatttaatagatttaatatagattaaatttattttgaatagatttaatatagattaaatttatttctatttagtttagagttctaaatagagataattagagtcaaaatctttttatgcagttatatattttatgattatataaatgaaacgttataagtctagataattagaatgaaactctttttagactcaaaaatttgaattatcttcgaattcgaaatagaaatgaatggaataattagttctagttattaaatataaataattagttctagctattatactataaaaaaaaataattaatttcattttcatttttaataatgaataaattcaattttcatttttctttttttagttattatggttatataggatagtctaataaaaggataagaataaaaatgaaattaaagaaaaaaagatgcaacccatagaaatgaaatccagatcataatgagagactcctttcttttgttttcattcataaagacagaagctaagacgaaaaaaagaatcgacccttcgagtattccaccaaattgcctgagaaaactgagagtaagagggacatatatatgttatgaaatacccatctatattgaattgcgaatacagaaatgataaaatatttttggaccaaataaaaattaatatgggtctccgatagagacgaaagacggataaacaaacaagaaaataggtaaagacccttcgatataagaatctgtatctatatctactaaattcaacggtttcgcataaaaagaaagcaaataaataaacgaaagaaaataaacaaatgaaagaagggggaaaggagggagaaagggggaaggaggggcatcctaatgagatcctaatctcaagacacaaaaggggatatggcgaaattggtagacgctacggacttaattggatt

SEQ ID NO: 137 - DNA, 856 bp, Gossypium hirsutum
caagctaatgttgatagaatctaataattaaattaaaattttatatatatatttattatttatctatatattttatatatttaatatatttaatatctttttttatatcttagttatatttcttttatattaatatattactaatattactgactccatttttattttctagtttattttatattttagtatattttacatctaaatgatatagaaatagaaaattttttatatagatttcatttaatcgatcgatttaatagatttaatatagattaaatttattttgaatagatttaatatagattaaatttatttctatttagtttagagttctaaatagagataattagagtcaaaatctttttatgcagttatatattttatgattatataaatgaaacgttataagtctagataattagaatgaaactctttttagactcaaaaatttgaattatcttcgaattcgaaatagaaatgaatggaataattagttctagttattaaatataaataattagttctagctattatactataaaaaaaaataattaatttcattttcatttttaataatgaataaattcaattttcatttttctttttttagttattatggttatataggatagtctaataaaaggataagaataaaaatgaaattaaagaaaaaaagatgcaacccatagaaatgaaatccagatcataatgagagactcctttcttttgttttcattcataaagacagaagctaagacgaaaaaaagaatcgacccttcgagtattccaccaaattgcctgagaaaactgagagtaagagggacatatatatgttatgaaatacccatctata

SEQ ID NO: 138 - DNA, 1712 bp, Gossypium hirsutum
taacgtgacataaggactccttattaatttgaattttattgaaatttcataaaccgaaattaaaactgaactaaatgaagcgaaatcgactgaaggattgtactacaaatattaatgagatgaattggatcaatatccatactttacttttttgtttttttgtattgtatataatgtatacaaaaaatagaaataaatatatatataataataatatataaatataaaaataatattaatatataaatataaaaatatataaaatatttagaatatatataataaaatagatataatatataaataaattgaatataatattaaatagataaataaattaaaccaaaagagtttccttttttttcttgatttattctgcaaagatctaactcctcaaattgatccctaattggaagtttcgacgagataataaacagattgttgacctttggaaagaattggaaagaaaaggggaaaaagctttttcactgtttgttctttgatttcaaaaaaaacattctaaattatgtaaaaaaggcaaaaagcaaacaaatagggaaagccggctatcggaatcgaaccgatgaccatcgcattacaaatgcgatgctctaacctctgagctaagcgggctcaaataatagaaatttggtatccatagggattcagcaaactattagatcttatctattaactatctattagttattcataattaatatgaattattaatatgaattatagactcgaatttagaaaaactagaattttttctaatttttaatgccatacttaatataaacttaatataaatataacatgtttaatataataatggtagattcaatctaatattcaagctaatgttgatagaatctaataattaaattaaaattttatatatatatttattatttatctatatattttatatatttaatatatttaatatctttttttatatcttagttatatttcttttatattaatatattactaatattactgactccatttttattttctagtttattttatattttagtatattttacatctaaatgatatagaaatagaaaattttttatatagatttcatttaatcgatcgatttaatagatttaatatagattaaatttattttgaatagatttaatatagattaaatttatttctatttagtttagagttctaaatagagataattagagtcaaaatctttttatgcagttatatattttatgattatataaatgaaacgttataagtctagataattagaatgaaactctttttagactcaaaaatttgaattatcttcgaattcgaaatagaaatgaatggaataattagttctagttattaaatataaataattagttctagctattatactataaaaaaaaataattaatttcattttcatttttaataatgaataaattcaattttcatttttctttttttagttattatggttatataggatagtctaataaaaggataagaataaaaatgaaattaaagaaaaaaagatgcaacccatagaaatgaaatccagatcataatgagagactcctttcttttgttttcattcataaagacagaagctaagacgaaaaaaagaatcgacccttcgagtattccaccaaattgcctgagaaaactgagagtaagagggacatatatatgttatgaaatacccatctata

SEQ ID NO: 139 - DNA, 1177 bp, Gossypium hirsutum
caagctaatgttgatagaatctaataattaaattaaaattttatatatatatttattatttatctatatattttatatatttaatatatttaatatctttttttatatcttagttatatttcttttatattaatatattactaatattactgactccatttttattttctagtttattttatattttagtatattttacatctaaatgatatagaaatagaaaattttttatatagatttcatttaatcgatcgatttaatagatttaatatagattaaatttattttgaatagatttaatatagattaaatttatttctatttagtttagagttctaaatagagataattagagtcaaaatctttttatgcagttatatattttatgattatataaatgaaacgttataagtctagataattagaatgaaactctttttagactcaaaaatttgaattatcttcgaattcgaaatagaaatgaatggaataattagttctagttattaaatataaataattagttctagctattatactataaaaaaaaataattaatttcattttcatttttaataatgaataaattcaattttcatttttctttttttagttattatggttatataggatagtctaataaaaggataagaataaaaatgaaattaaagaaaaaaagatgcaacccatagaaatgaaatccagatcataatgagagactcctttcttttgttttcattcataaagacagaagctaagacgaaaaaaagaatcgacccttcgagtattccaccaaattgcctgagaaaactgagagtaagagggacatatatatgttatgaaatacccatctatattgaattgcgaatacagaaatgataaaatatttttggaccaaataaaaattaatatgggtctccgatagagacgaaagacggataaacaaacaagaaaataggtaaagacccttcgatataagaatctgtatctatatctactaaattcaacggtttcgcataaaaagaaagcaaataaataaacgaaagaaaataaacaaatgaaagaagggggaaaggagggagaaagggggaaggaggggcatcctaatgagatcctaatctcaagacacaaaaggggatatggcgaaattggtagacgctacggacttaattggatt

SEQ ID NO: 140 - DNA, 1070 bp, Gossypium hirsutum
aaggctacatccagtactggaccgatgatttgagaaatacgccccagattttctttttcaagcgtggaaactccaggaacagaagtagtaggatttattttcataataaagtgaaatatgtcaatttttttttccaaaattatctaatacaaaataaatcttcgacaggaggttgatcggttaattcaataagaaatgggggttagcgctcgatttcgttggtcccatccaattcaagtgcatgcaattggattgtttccttattcaatgaaggaatttttaagttcaaccaacctattttcaaaataggaagtggataaataaaaattttgggagagtctttcatttgtccatcattataggcaacaccatccatattatctatggaattcgaacccgaactctatttatgattccttatttctatctcattagccttgtttcaatttcatttcatattttattttagcatatcgatttttttttactttacgtccaacctctcccccctttttccatctatattcccttttttttcatttcagggccgaattacgcctgttttcacatctaggatttacatatacaacatatattactgtcaagggtgaatttcttattttattgagcttataatatattatataatttatattagatgttagaatattaattctattatatatttatctattctattatatatatattctaatatatattagtattatttttatattatttatctatttcttatctatttctatttcatttatttcatttgcatttagaattttgattcaaaaaaaagtaaggaattataaacttgaaaaacacggatcaggttgcgccatacatatgaaagaatatagaataatgatgtatttggcgaatcaaataccatggtctaataaggaacctttcggattcgttgataattttttgaaagatttcttcgaaaaggtttcattaactcctcatttatatcgagtagaccttgttgttgcgagaattcttaattcatgagttgtagggagggacttatgtcaccacaaacagagactaaagca

SEQ ID NO: 141 - DNA, 427 bp, Gossypium hirsutum
aaaaattttgggagagtctttcatttgtccatcattataggcaacaccatccatattatctatggaattcgaacccgaactctatttatgattccttatttctatctcattagccttgtttcaatttcatttcatattttattttagcatatcgatttttttttactttacgtccaacctctcccccctttttccatctatattcccttttttttcatttcagggccgaattacgcctgttttcacatctaggatttacatatacaacatatattactgtcaagggtgaatttcttattttattgagcttataatatattatataatttatattagatgttagaatattaattctattatatatttatctattctattatatatatattctaatatatattagtattatttttatattatttatcta

SEQ ID NO: 142 - DNA, 749 bp, Gossypium hirsutum
aaggctacatccagtactggaccgatgatttgagaaatacgccccagattttctttttcaagcgtggaaactccaggaacagaagtagtaggatttattttcataataaagtgaaatatgtcaatttttttttccaaaattatctaatacaaaataaatcttcgacaggaggttgatcggttaattcaataagaaatgggggttagcgctcgatttcgttggtcccatccaattcaagtgcatgcaattggattgtttccttattcaatgaaggaatttttaagttcaaccaacctattttcaaaataggaagtggataaataaaaattttgggagagtctttcatttgtccatcattataggcaacaccatccatattatctatggaattcgaacccgaactctatttatgattccttatttctatctcattagccttgtttcaatttcatttcatattttattttagcatatcgatttttttttactttacgtccaacctctcccccctttttccatctatattcccttttttttcatttcagggccgaattacgcctgttttcacatctaggatttacatatacaacatatattactgtcaagggtgaatttcttattttattgagcttataatatattatataatttatattagatgttagaatattaattctattatatatttatctattctattatatatatattctaatatatattagtattatttttatattatttatcta

SEQ ID NO: 143 - DNA, 748 bp, Gossypium hirsutum
aaaaattttgggagagtctttcatttgtccatcattataggcaacaccatccatattatctatggaattcgaacccgaactctatttatgattccttatttctatctcattagccttgtttcaatttcatttcatattttattttagcatatcgatttttttttactttacgtccaacctctcccccctttttccatctatattcccttttttttcatttcagggccgaattacgcctgttttcacatctaggatttacatatacaacatatattactgtcaagggtgaatttcttattttattgagcttataatatattatataatttatattagatgttagaatattaattctattatatatttatctattctattatatatatattctaatatatattagtattatttttatattatttatctatttcttatctatttctatttcatttatttcatttgcatttagaattttgattcaaaaaaaagtaaggaattataaacttgaaaaacacggatcaggttgcgccatacatatgaaagaatatagaataatgatgtatttggcgaatcaaataccatggtctaataaggaacctttcggattcgttgataattttttgaaagatttcttcgaaaaggtttcattaactcctcatttatatcgagtagaccttgttgttgcgagaattcttaattcatgagttgtagggagggacttatgtcaccacaaacagagactaaagca

SEQ ID NO: 144 - DNA, 963 bp, Gossypium hirsutum
gatgaatagggaaggtatagtaatactatgaatgacccagtatcgaatactggtaataatatcagcaaaagaacgttctcctgtgcttccagacatgccgagctccacatattcttgtaggggatcgattccgtaaaagatgggatcagtaaatgggaattcaccgaaactcaatctttgtgagatcgtcaatattgtaccaaaggtatttttagagtataccgaatcagtatagctatccttcttctgacacagcaacgcaatttcaaacagtatatcgacgtgaagtgctagataatttctttcttcttcttgcttgtcgatgtataaccaggtatcatgcaatagaaaattcctcaaattcctggaggtattggcttgggcctagaaacggaagatcaggtaacccgtgaatccaacgagttggtttataataattcatgaaggaaattatcatatttccgcaagtcaattagatgcgaaatataaaaattgactttttttcgtagttgcacaagaggttttgttttttgttggaagccctcttttttttttatctatatctaacgactactcttacttgttactagacgactaagcaattccttaaaataaaaataaaaaaaaaagaggaaggaataaaaaaaaattctactaaatttgtgaggcggcctttcttgtattcttgtattagatccaaaggttttttcttgacctaactacaaagatgatgaatcgatttctttttcttttctactcttgtcctgccgctctatttttgtgaataccgtttataatgattgatgaagcaaaaaatttcaattgaacttattatttcaattggtatttttgcttatccccctctcgtttaaaaattgaaacttaggtaagtgctttataaacatatgtataaaaagaacatatttcatttagctccttcatgcctactataactagttattt

SEQ ID NO: 145 - DNA, 215 bp, Gossypium hirsutum
cggaagatcaggtaacccgtgaatccaacgagttggtttataataattcatgaaggaaattatcatatttccgcaagtcaattagatgcgaaatataaaaattgactttttttcgtagttgcacaagaggttttgttttttgttggaagccctcttttttttttatctatatctaacgactactcttacttgttactagacgactaagcaattcc

SEQ ID NO: 146 - DNA, 606 bp, Gossypium hirsutum
gatgaatagggaaggtatagtaatactatgaatgacccagtatcgaatactggtaataatatcagcaaaagaacgttctcctgtgcttccagacatgccgagctccacatattcttgtaggggatcgattccgtaaaagatgggatcagtaaatgggaattcaccgaaactcaatctttgtgagatcgtcaatattgtaccaaaggtatttttagagtataccgaatcagtatagctatccttcttctgacacagcaacgcaatttcaaacagtatatcgacgtgaagtgctagataatttctttcttcttcttgcttgtcgatgtataaccaggtatcatgcaatagaaaattcctcaaattcctggaggtattggcttgggcctagaaacggaagatcaggtaacccgtgaatccaacgagttggtttataataattcatgaaggaaattatcatatttccgcaagtcaattagatgcgaaatataaaaattgactttttttcgtagttgcacaagaggttttgttttttgttggaagccctcttttttttttatctatatctaacgactactcttacttgttactagacgactaagcaattcc

SEQ ID NO: 147 - DNA, 572 bp, Gossypium hirsutum
cggaagatcaggtaacccgtgaatccaacgagttggtttataataattcatgaaggaaattatcatatttccgcaagtcaattagatgcgaaatataaaaattgactttttttcgtagttgcacaagaggttttgttttttgttggaagccctcttttttttttatctatatctaacgactactcttacttgttactagacgactaagcaattccttaaaataaaaataaaaaaaaaagaggaaggaataaaaaaaaattctactaaatttgtgaggcggcctttcttgtattcttgtattagatccaaaggttttttcttgacctaactacaaagatgatgaatcgatttctttttcttttctactcttgtcctgccgctctatttttgtgaataccgtttataatgattgatgaagcaaaaaatttcaattgaacttattatttcaattggtatttttgcttatccccctctcgtttaaaaattgaaacttaggtaagtgctttataaacatatgtataaaaagaacatatttcatttagctccttcatgcctactataactagttattt

SEQ ID NO: 148 - DNA, 856 bp, Gossypium hirsutum
aataaactaattttttttcgacgtctacgagctatatatccccgtttaattctggtcattgaataaaagaaattttgatgaataactaattctttctttcagttattcttttctagtctattaataacaaaacggattcttccaatgtataaaataaaaattccaatggcttttgctactctaaccgtcccgaccacgatttttccttttttctaggcatttcgccttgaaataagaaattgtattgatactaggtatcaaaaaaagcatattaactaaaataaaggagtaaatagaaatggataaataaatagtgggttccatcgtttctatggttacttcttaaacggtgaggtcctctctatacaccggagctcattccttcatttaattggtaacttgtacagttcacactcttcggctctactcataaattttccagtaatagatctttcacaacgagatctatcttatacagtaacggtatgtaaggatgaggattaattgggcagctgaccctgttagtccgttctttgcaagagtcgaagcataatctttttgctttttaaataggattttccccgcttaatggataagcatttgctaccaatggggaattttttctcatcttaaattctaagattggatttgcgccaatggaaaccataaatttcatacacaatagaaggatatggtagatctagcttttttagatagtgaacggacgaaccccattctattcactggtacggatcgttgatactgaaaaagttgtttcttttttctcagcccatgatctgaacaagtcgcacatacaccctagtacatgttcctcggcgctgaggacatcccccaa

SEQ ID NO: 149 - DNA, 321 bp, Gossypium hirsutum
aggcatttcgccttgaaataagaaattgtattgatactaggtatcaaaaaaagcatattaactaaaataaaggagtaaatagaaatggataaataaatagtgggttccatcgtttctatggttacttcttaaacggtgaggtcctctctatacaccggagctcattccttcatttaattggtaacttgtacagttcacactcttcggctctactcataaattttccagtaatagatctttcacaacgagatctatcttatacagtaacggtatgtaaggatgaggattaattgggcagctgaccctgttagtccgttcttt

SEQ ID NO: 150 - DNA, 535 bp, Gossypium hirsutum
aataaactaattttttttcgacgtctacgagctatatatccccgtttaattctggtcattgaataaaagaaattttgatgaataactaattctttctttcagttattcttttctagtctattaataacaaaacggattcttccaatgtataaaataaaaattccaatggcttttgctactctaaccgtcccgaccacgatttttccttttttctaggcatttcgccttgaaataagaaattgtattgatactaggtatcaaaaaaagcatattaactaaaataaaggagtaaatagaaatggataaataaatagtgggttccatcgtttctatggttacttcttaaacggtgaggtcctctctatacaccggagctcattccttcatttaattggtaacttgtacagttcacactcttcggctctactcataaattttccagtaatagatctttcacaacgagatctatcttatacagtaacggtatgtaaggatgaggattaattgggcagctgaccctgttagtccgttcttt

SEQ ID NO: 151 - DNA, 642 bp, Gossypium hirsutum
aggcatttcgccttgaaataagaaattgtattgatactaggtatcaaaaaaagcatattaactaaaataaaggagtaaatagaaatggataaataaatagtgggttccatcgtttctatggttacttcttaaacggtgaggtcctctctatacaccggagctcattccttcatttaattggtaacttgtacagttcacactcttcggctctactcataaattttccagtaatagatctttcacaacgagatctatcttatacagtaacggtatgtaaggatgaggattaattgggcagctgaccctgttagtccgttctttgcaagagtcgaagcataatctttttgctttttaaataggattttccccgcttaatggataagcatttgctaccaatggggaattttttctcatcttaaattctaagattggatttgcgccaatggaaaccataaatttcatacacaatagaaggatatggtagatctagcttttttagatagtgaacggacgaaccccattctattcactggtacggatcgttgatactgaaaaagttgtttcttttttctcagcccatgatctgaacaagtcgcacatacaccctagtacatgttcctcggcgctgaggacatcccccaa

SEQ ID NO: 152 - DNA, 2354 bp, Gossypium hirsutum
gatcttttgtctcatggtagcctgctccagtccccttacgaaactttcgttattgggttagccatacacttcacatgtttctagcgattcacatggcatcatcaaatgatacaagtcttggataagaatctacaacgcactagaacgcccttgttgacgatcctttactccgacagcatctagggttcctcgaacaatgtgatatctcacaccgggtaaatccttaacccttccccctcttactaagactacagaatgttcttgtgaattatggccaataccaggtatataagcagtgatttcaaacccagaggttaatcgtactctggcaactttacgtaaggcagagtttggttttttgggggtgatagtggaaaagttgacagataagtcacccttactgccactctacagaaccgtacatgagattttcacctcatacggctcctcgttcaattctttcgaagtcattgggtccttttcctcgttcgagaatctcctcccttcttccactctgtcccgaagagtaactaggacaaattcagtcacgttttcatgttccaattgaacactttccatttttgattattctcaaaagagaagattttttttaccaaacatctgcggatccaatcacacgatcttataataagaacaagagatctttctcgatcaatccctttgcccctcattcttcgagaatcagaaagatccttttcaagtttgaatttgttcatttggaatctgggttcttctacttcatttttatttacttatttattatttttattatttttttcccctctttttttgatttcttttttttttttattttattcccttccatcattccctaagtcccataggtttgatcctatagaatctgacccattttctcattgagcgaggggtacgaaataaatcagattgatttttcgatcaaaagcactatgtgaaatcttcggtttttcctctttctctatccctatcccataggtacagcatttgaatcaatagagaaccttttcctcctatatgaatcaatattattacattccaattccttactgataccccccaaggaaaatcccgaattggatccaaaattgacgggttagtgtgagcttatccatgcggttatgcactcttcgaataggaatccattttctgaaagatcctggctttcgtgctttggtgagtctccgagatcctttcgacgacctatgttgtgttgaagggatatctatatgatccgatcgattgcgtaaagcctgcggtagcaacggaaccggggaaagtatacagaaaagacagttctttctattatattagtattttctattctattttttctattctattagtattagagtattagattagtattctattagtattagattagtattagttagattagattagtattagttagtgatcccggctcagtgagtcctttcttccgtgattaactgttggcgccagtcctagtcctacattccattttgtctctgtggaccgaggagaaagggggctcagcgggaagaggattgtaccatgagagaagcaaggaggtcaacctctttcaaatatacaacatggattctggcaatgcaatggagttgggctttcatgcggatccgaatgaatcatcttttccacggaggtaaatccttgcccgctaggcaagaggatagcaagttacaaattctgtttcggtaggacatgtatttctattactatgaaattcataaatgaagtaattaatcgtggggttaccattatcctgtttgtagtgacgaatcttgtatgtgttcctaagaaaagaaaagaaatttgtcaaaatttcggggtcttaaagggtcgtggaaacccataagaactcttgaatggaactggaaaagagatgtaactccagttccttcggaaatggtaagatctttggcgcaagaagaaggggctgatccgtatcatcttaacttggttctgatttctctatttttttaagaataccgcttctcctactcgtatcgaatagaacatgctgaggcaaaccttcttcatgtaaaacctgcttgatttagatcgggagaatcgtacggttttatgaaaccatgtgtatatggctcgaatccgtagtcaatcctatttccgataggagcagttgacaattgaatccaactttttccattattttcgtatccgtaatagtgcgaaaggaaagcctggctccaagttgttcaagaatagtggcgttgttgagtttctcgatcctttgacttaggattagtcagttctatttcttgatgggggcagggaagggatataactcagcggtagagtgtcaccttgac

SEQ ID NO: 153 - DNA, 1708 bp, Gossypium hirsutum
cacctcatacggctcctcgttcaattctttcgaagtcattgggtccttttcctcgttcgagaatctcctcccttcttccactctgtcccgaagagtaactaggacaaattcagtcacgttttcatgttccaattgaacactttccatttttgattattctcaaaagagaagattttttttaccaaacatctgcggatccaatcacacgatcttataataagaacaagagatctttctcgatcaatccctttgcccctcattcttcgagaatcagaaagatccttttcaagtttgaatttgttcatttggaatctgggttcttctacttcatttttatttacttatttattatttttattatttttttcccctctttttttgatttcttttttttttttattttattcccttccatcattccctaagtcccataggtttgatcctatagaatctgacccattttctcattgagcgaggggtacgaaataaatcagattgatttttcgatcaaaagcactatgtgaaatcttcggtttttcctctttctctatccctatcccataggtacagcatttgaatcaatagagaaccttttcctcctatatgaatcaatattattacattccaattccttactgataccccccaaggaaaatcccgaattggatccaaaattgacgggttagtgtgagcttatccatgcggttatgcactcttcgaataggaatccattttctgaaagatcctggctttcgtgctttggtgagtctccgagatcctttcgacgacctatgttgtgttgaagggatatctatatgatccgatcgattgcgtaaagcctgcggtagcaacggaaccggggaaagtatacagaaaagacagttctttctattatattagtattttctattctattttttctattctattagtattagagtattagattagtattctattagtattagattagtattagttagattagattagtattagttagtgatcccggctcagtgagtcctttcttccgtgattaactgttggcgccagtcctagtcctacattccattttgtctctgtggaccgaggagaaagggggctcagcgggaagaggattgtaccatgagagaagcaaggaggtcaacctctttcaaatatacaacatggattctggcaatgcaatggagttgggctttcatgcggatccgaatgaatcatcttttccacggaggtaaatccttgcccgctaggcaagaggatagcaagttacaaattctgtttcggtaggacatgtatttctattactatgaaattcataaatgaagtaattaatcgtggggttaccattatcctgtttgtagtgacgaatcttgtatgtgttcctaagaaaagaaaagaaatttgtcaaaatttcggggtcttaaagggtcgtggaaacccataagaactcttgaatggaactggaaaagagatgtaactccagttccttcggaaatggtaagatctttggcgcaagaagaaggggctgatccgtatcatcttaacttggttctgatttctctatttttttaagaataccgcttctcctactcgtatcgaatagaacatgctgaggcaaaccttcttcatgtaaaacctgcttgatttagatcgggagaatcgtacggttttatgaaaccatgtgtatatggctcgaatccgtagt

SEQ ID NO: 154 - DNA, 2140 bp, Gossypium hirsutum
gatcttttgtctcatggtagcctgctccagtccccttacgaaactttcgttattgggttagccatacacttcacatgtttctagcgattcacatggcatcatcaaatgatacaagtcttggataagaatctacaacgcactagaacgcccttgttgacgatcctttactccgacagcatctagggttcctcgaacaatgtgatatctcacaccgggtaaatccttaacccttccccctcttactaagactacagaatgttcttgtgaattatggccaataccaggtatataagcagtgatttcaaacccagaggttaatcgtactctggcaactttacgtaaggcagagtttggttttttgggggtgatagtggaaaagttgacagataagtcacccttactgccactctacagaaccgtacatgagattttcacctcatacggctcctcgttcaattctttcgaagtcattgggtccttttcctcgttcgagaatctcctcccttcttccactctgtcccgaagagtaactaggacaaattcagtcacgttttcatgttccaattgaacactttccatttttgattattctcaaaagagaagattttttttaccaaacatctgcggatccaatcacacgatcttataataagaacaagagatctttctcgatcaatccctttgcccctcattcttcgagaatcagaaagatccttttcaagtttgaatttgttcatttggaatctgggttcttctacttcatttttatttacttatttattatttttattatttttttcccctctttttttgatttcttttttttttttattttattcccttccatcattccctaagtcccataggtttgatcctatagaatctgacccattttctcattgagcgaggggtacgaaataaatcagattgatttttcgatcaaaagcactatgtgaaatcttcggtttttcctctttctctatccctatcccataggtacagcatttgaatcaatagagaaccttttcctcctatatgaatcaatattattacattccaattccttactgataccccccaaggaaaatcccgaattggatccaaaattgacgggttagtgtgagcttatccatgcggttatgcactcttcgaataggaatccattttctgaaagatcctggctttcgtgctttggtgagtctccgagatcctttcgacgacctatgttgtgttgaagggatatctatatgatccgatcgattgcgtaaagcctgcggtagcaacggaaccggggaaagtatacagaaaagacagttctttctattatattagtattttctattctattttttctattctattagtattagagtattagattagtattctattagtattagattagtattagttagattagattagtattagttagtgatcccggctcagtgagtcctttcttccgtgattaactgttggcgccagtcctagtcctacattccattttgtctctgtggaccgaggagaaagggggctcagcgggaagaggattgtaccatgagagaagcaaggaggtcaacctctttcaaatatacaacatggattctggcaatgcaatggagttgggctttcatgcggatccgaatgaatcatcttttccacggaggtaaatccttgcccgctaggcaagaggatagcaagttacaaattctgtttcggtaggacatgtatttctattactatgaaattcataaatgaagtaattaatcgtggggttaccattatcctgtttgtagtgacgaatcttgtatgtgttcctaagaaaagaaaagaaatttgtcaaaatttcggggtcttaaagggtcgtggaaacccataagaactcttgaatggaactggaaaagagatgtaactccagttccttcggaaatggtaagatctttggcgcaagaagaaggggctgatccgtatcatcttaacttggttctgatttctctatttttttaagaataccgcttctcctactcgtatcgaatagaacatgctgaggcaaaccttcttcatgtaaaacctgcttgatttagatcgggagaatcgtacggttttatgaaaccatgtgtatatggctcgaatccgtagt

SEQ ID NO: 155 - DNA, 1922 bp, Gossypium hirsutum
cacctcatacggctcctcgttcaattctttcgaagtcattgggtccttttcctcgttcgagaatctcctcccttcttccactctgtcccgaagagtaactaggacaaattcagtcacgttttcatgttccaattgaacactttccatttttgattattctcaaaagagaagattttttttaccaaacatctgcggatccaatcacacgatcttataataagaacaagagatctttctcgatcaatccctttgcccctcattcttcgagaatcagaaagatccttttcaagtttgaatttgttcatttggaatctgggttcttctacttcatttttatttacttatttattatttttattatttttttcccctctttttttgatttcttttttttttttattttattcccttccatcattccctaagtcccataggtttgatcctatagaatctgacccattttctcattgagcgaggggtacgaaataaatcagattgatttttcgatcaaaagcactatgtgaaatcttcggtttttcctctttctctatccctatcccataggtacagcatttgaatcaatagagaaccttttcctcctatatgaatcaatattattacattccaattccttactgataccccccaaggaaaatcccgaattggatccaaaattgacgggttagtgtgagcttatccatgcggttatgcactcttcgaataggaatccattttctgaaagatcctggctttcgtgctttggtgagtctccgagatcctttcgacgacctatgttgtgttgaagggatatctatatgatccgatcgattgcgtaaagcctgcggtagcaacggaaccggggaaagtatacagaaaagacagttctttctattatattagtattttctattctattttttctattctattagtattagagtattagattagtattctattagtattagattagtattagttagattagattagtattagttagtgatcccggctcagtgagtcctttcttccgtgattaactgttggcgccagtcctagtcctacattccattttgtctctgtggaccgaggagaaagggggctcagcgggaagaggattgtaccatgagagaagcaaggaggtcaacctctttcaaatatacaacatggattctggcaatgcaatggagttgggctttcatgcggatccgaatgaatcatcttttccacggaggtaaatccttgcccgctaggcaagaggatagcaagttacaaattctgtttcggtaggacatgtatttctattactatgaaattcataaatgaagtaattaatcgtggggttaccattatcctgtttgtagtgacgaatcttgtatgtgttcctaagaaaagaaaagaaatttgtcaaaatttcggggtcttaaagggtcgtggaaacccataagaactcttgaatggaactggaaaagagatgtaactccagttccttcggaaatggtaagatctttggcgcaagaagaaggggctgatccgtatcatcttaacttggttctgatttctctatttttttaagaataccgcttctcctactcgtatcgaatagaacatgctgaggcaaaccttcttcatgtaaaacctgcttgatttagatcgggagaatcgtacggttttatgaaaccatgtgtatatggctcgaatccgtagtcaatcctatttccgataggagcagttgacaattgaatccaactttttccattattttcgtatccgtaatagtgcgaaaggaaagcctggctccaagttgttcaagaatagtggcgttgttgagtttctcgatcctttgacttaggattagtcagttctatttcttgatgggggcagggaagggatataactcagcggtagagtgtcaccttgac

SEQ ID NO: 156 - DNA, 1498 bp, Gossypium hirsutum
ttggctaatgaaaaagctttcaaggccgcccaatatcctttctttttccaaacattttttcgaatacgcttttttgatgtagaagtacgtttttttggaactgccattcaaaaaaaagttattcagtgctatagttggatgtcaaagacatctatttttaaaacaaaacgatcggtcttttgatgtcattatttatctattcctatagattttataaattatattataattatatatatactacaatacaaattgcataaaaaatgaatagagatgggaaaatcgcataaaatgcttctattctagaacaaaaaaacaatataaccccttttttatttatattccatacactatccagaaaaaaaaaagaagaatttgtatttaatttattggtacctttcttttttatatctccattcaaatctataggatagaataggatctatatctattatgatatataaatcgaattgatgaaatcaaaaaaacgcaaaaaaagtctttctttttctatctacgcccagttttttgttgtcttacatttataatttatacatctccatttatacatgaaaactacatcaaaataaaaagtgtaaaacccattttatctacctatgcaaacttgaatttgattttttccataaattggtcaagctcgaagagagggtatgcctaattttcattttcaaccaaattcgaatgagactcgtagttaatctgttaaaggatacataattttgaaaaaaaaagaatattttattatattaatagtcatttttcttttaattatatgtaaatataaagaaaatatcggatagtctttcctacaagaaaaggttcatttattgtagtggaagacaatcaatcagttccgcgatgaaaatgaactagttaataagttcgaataaacaaactcaaataattcgtttttcttttaagttctaggacatcctatgatttatatcatactttttgggggggaattctttgtatttttgatcgatgtatctaacttattgtaaatcgaaattattgtaatatgtaataataaataataattgatattttcagaaaaatattactataatgaaaaaaagaattcttttgtttttcattatagtaactcggcgagatcatttgattacttctaacttcgaaatttgaatatttttgaaatagttgcgaaagattaaaaaattaagaatagaaaattccagttaactttgtaatctctcgatttttttattgctccttttcaatcattttattgctccttttcaatcaaaagagataagagccggtgaatcagaaacgattaattaagaaagaataacaaaaaagtttttatggagcatgcagatcaatattcatggatcataccttttgttccacttcccattcctattttaataggaatgggactcctactttttccgacggcaacaaaaaatcttcgtcgtatgtgggcttttcccaatattttattgttaagtat

SEQ ID NO: 157 - DNA, 535 bp, Gossypium hirsutum
ggatagaataggatctatatctattatgatatataaatcgaattgatgaaatcaaaaaaacgcaaaaaaagtctttctttttctatctacgcccagttttttgttgtcttacatttataatttatacatctccatttatacatgaaaactacatcaaaataaaaagtgtaaaacccattttatctacctatgcaaacttgaatttgattttttccataaattggtcaagctcgaagagagggtatgcctaattttcattttcaaccaaattcgaatgagactcgtagttaatctgttaaaggatacataattttgaaaaaaaaagaatattttattatattaatagtcatttttcttttaattatatgtaaatataaagaaaatatcggatagtctttcctacaagaaaaggttcatttattgtagtggaagacaatcaatcagttccgcgatgaaaatgaactagttaataagttcgaataaacaaactcaaataattcgtttttcttttaagttctaggacatcctatgattt

SEQ ID NO: 158 - DNA, 963 bp, Gossypium hirsutum
ttggctaatgaaaaagctttcaaggccgcccaatatcctttctttttccaaacattttttcgaatacgcttttttgatgtagaagtacgtttttttggaactgccattcaaaaaaaagttattcagtgctatagttggatgtcaaagacatctatttttaaaacaaaacgatcggtcttttgatgtcattatttatctattcctatagattttataaattatattataattatatatatactacaatacaaattgcataaaaaatgaatagagatgggaaaatcgcataaaatgcttctattctagaacaaaaaaacaatataaccccttttttatttatattccatacactatccagaaaaaaaaaagaagaatttgtatttaatttattggtacctttcttttttatatctccattcaaatctataggatagaataggatctatatctattatgatatataaatcgaattgatgaaatcaaaaaaacgcaaaaaaagtctttctttttctatctacgcccagttttttgttgtcttacatttataatttatacatctccatttatacatgaaaactacatcaaaataaaaagtgtaaaacccattttatctacctatgcaaacttgaatttgattttttccataaattggtcaagctcgaagagagggtatgcctaattttcattttcaaccaaattcgaatgagactcgtagttaatctgttaaaggatacataattttgaaaaaaaaagaatattttattatattaatagtcatttttcttttaattatatgtaaatataaagaaaatatcggatagtctttcctacaagaaaaggttcatttattgtagtggaagacaatcaatcagttccgcgatgaaaatgaactagttaataagttcgaataaacaaactcaaataattcgtttttcttttaagttctaggacatcctatgattt

SEQ ID NO: 159 - DNA, 1070 bp, Gossypium hirsutum
ggatagaataggatctatatctattatgatatataaatcgaattgatgaaatcaaaaaaacgcaaaaaaagtctttctttttctatctacgcccagttttttgttgtcttacatttataatttatacatctccatttatacatgaaaactacatcaaaataaaaagtgtaaaacccattttatctacctatgcaaacttgaatttgattttttccataaattggtcaagctcgaagagagggtatgcctaattttcattttcaaccaaattcgaatgagactcgtagttaatctgttaaaggatacataattttgaaaaaaaaagaatattttattatattaatagtcatttttcttttaattatatgtaaatataaagaaaatatcggatagtctttcctacaagaaaaggttcatttattgtagtggaagacaatcaatcagttccgcgatgaaaatgaactagttaataagttcgaataaacaaactcaaataattcgtttttcttttaagttctaggacatcctatgatttatatcatactttttgggggggaattctttgtatttttgatcgatgtatctaacttattgtaaatcgaaattattgtaatatgtaataataaataataattgatattttcagaaaaatattactataatgaaaaaaagaattcttttgtttttcattatagtaactcggcgagatcatttgattacttctaacttcgaaatttgaatatttttgaaatagttgcgaaagattaaaaaattaagaatagaaaattccagttaactttgtaatctctcgatttttttattgctccttttcaatcattttattgctccttttcaatcaaaagagataagagccggtgaatcagaaacgattaattaagaaagaataacaaaaaagtttttatggagcatgcagatcaatattcatggatcataccttttgttccacttcccattcctattttaataggaatgggactcctactttttccgacggcaacaaaaaatcttcgtcgtatgtgggcttttcccaatattttattgttaagtat

SEQ ID NO: 160 - DNA, 963 bp, Gossypium hirsutum
ttaaaagccgagtactctaccgttgagttagcaacccgaaaaaaattagaatatgtagatacaatcggaataaaaaaatggaattgcactaggcaatcaaaacacgaaattagcaaaaattggaataaaaaaaagacacgaaattctcagataaaaccatagaaatagaataagtgaaaatttgcggaccgcctcctgtcttattcattccattgttatccattttttttgtttcaatacaattacaataaaaaaaacttattgtatcacaacaaattcaaagaaagaacccattatttgaatgaagtgaagtgccaaacgaggataaatggatctatttatctatgatcgaattatatttgttcgatacactgttgtcaatatgattgtaaattttgaatataaatgttgagagttgaaaaaagaataaagaatataaaaaagactacaaaaaatacaatgaaaaaaatagaattcattttttttattttttttattaattattattaatattttctctttttatatgttatatgttattttatctttttttttatcttattttatgttgttttgtaaatgtttaaatgcaattacttcatttattcacttgatcttttttctctctattttctctcaataaaattggctcaataaaatcgggttttgttgttatagaacacggtattctatattttatagtagcaatattctatagtagcaaaggaataagaaatacgaataataaattttttaaaaagtctgaatagaccaaaagaggggggaggaaataataaagaaaaatttatacaaagctagatatgaatcatccacaccctcttttttggatttcattaagtgaattttgtttgattaagagtaagttctgtaaaaacccccgccttctttaaaatatcatgaacagttgctgtgggttgagctcctttttcaaggaaatagaga

SEQ ID NO: 161 - DNA, 321 bp, Gossypium hirsutum
aggataaatggatctatttatctatgatcgaattatatttgttcgatacactgttgtcaatatgattgtaaattttgaatataaatgttgagagttgaaaaaagaataaagaatataaaaaagactacaaaaaatacaatgaaaaaaatagaattcattttttttattttttttattaattattattaatattttctctttttatatgttatatgttattttatctttttttttatcttattttatgttgttttgtaaatgtttaaatgcaattacttcatttattcacttgatcttttttctctctattttctctcaata

SEQ ID NO: 162 - DNA, 642 bp, Gossypium hirsutum
ttaaaagccgagtactctaccgttgagttagcaacccgaaaaaaattagaatatgtagatacaatcggaataaaaaaatggaattgcactaggcaatcaaaacacgaaattagcaaaaattggaataaaaaaaagacacgaaattctcagataaaaccatagaaatagaataagtgaaaatttgcggaccgcctcctgtcttattcattccattgttatccattttttttgtttcaatacaattacaataaaaaaaacttattgtatcacaacaaattcaaagaaagaacccattatttgaatgaagtgaagtgccaaacgaggataaatggatctatttatctatgatcgaattatatttgttcgatacactgttgtcaatatgattgtaaattttgaatataaatgttgagagttgaaaaaagaataaagaatataaaaaagactacaaaaaatacaatgaaaaaaatagaattcattttttttattttttttattaattattattaatattttctctttttatatgttatatgttattttatctttttttttatcttattttatgttgttttgtaaatgtttaaatgcaattacttcatttattcacttgatcttttttctctctattttctctcaata

SEQ ID NO: 163 - DNA, 642 bp, Gossypium hirsutum
aggataaatggatctatttatctatgatcgaattatatttgttcgatacactgttgtcaatatgattgtaaattttgaatataaatgttgagagttgaaaaaagaataaagaatataaaaaagactacaaaaaatacaatgaaaaaaatagaattcattttttttattttttttattaattattattaatattttctctttttatatgttatatgttattttatctttttttttatcttattttatgttgttttgtaaatgtttaaatgcaattacttcatttattcacttgatcttttttctctctattttctctcaataaaattggctcaataaaatcgggttttgttgttatagaacacggtattctatattttatagtagcaatattctatagtagcaaaggaataagaaatacgaataataaattttttaaaaagtctgaatagaccaaaagaggggggaggaaataataaagaaaaatttatacaaagctagatatgaatcatccacaccctcttttttggatttcattaagtgaattttgtttgattaagagtaagttctgtaaaaacccccgccttctttaaaatatcatgaacagttgctgtgggttgagctcctttttcaaggaaatagaga

SEQ ID NO: 164 - DNA, 1070 bp, Gossypium hirsutum
agctgatcatgtccttcaagtcgcacgttgctttctaccacatcgtttcaaacgaagttttaccataacattcctctagtttggagcattgattcaattatggaatcatgaatagtcattggttcagtcgatacatagtaatctatctatactttctatatgaaatcaatttccttctatatgctatatgaaataaataaataatttaggaagaaaaagcctcaataagaattcaaactaccccatattgtatgattgtctgaatgcaatattttttgatttttttaaacttttattatacttttctattctaattaaacttttcgattctaataaaaaaaaaatcaaaataaagaatatcattaataataatattacgaaaatactaataatatcacgaatattataaataacacaaataaactaatctttttgaatctaccgtgcatcttcaaataacccgatagatcaaataactcggtagaatagatccagcaatctcacagttgttcgagtgccaatcttaagaaatcattaaaaatcaaaagcaagaatcacattttctccaatacttgactgttagaaagaaggttattaaaaacaaaaaaagagcaatttggatatggttattctgatatataaatagagtatgctctgggacggaaggattcgaacctccgcatagcgggaccaaaacccgttgccttaccacttggccacgccccatttagatttctatttgaaactactaaacactaatatggtattccttgttcgtcaattccagttccaaatagctatagctatggaatagattcttgttacgatttttgcacgtatggatagagaattaaatcgaatttattgatcattacatagaattcaattaagatattgtatgaaagtatgatttcttctattctcttgtaagaattgaaggctttttgattgggtgagttcaaagaagtattgtttagtctgccttattttcctttcttcatttttttccttatatcaaaaaacatattaataactcaatcaaaattatctccaagaacaaaattttgtta

SEQ ID NO: 165 - DNA, 107 bp, Gossypium hirsutum
tttcgattctaataaaaaaaaaatcaaaataaagaatatcattaataataatattacgaaaatactaataatatcacgaatattataaataacacaaataaactaat

SEQ ID NO: 166 - DNA, 428 bp, Gossypium hirsutum
agctgatcatgtccttcaagtcgcacgttgctttctaccacatcgtttcaaacgaagttttaccataacattcctctagtttggagcattgattcaattatggaatcatgaatagtcattggttcagtcgatacatagtaatctatctatactttctatatgaaatcaatttccttctatatgctatatgaaataaataaataatttaggaagaaaaagcctcaataagaattcaaactaccccatattgtatgattgtctgaatgcaatattttttgatttttttaaacttttattatacttttctattctaattaaacttttcgattctaataaaaaaaaaatcaaaataaagaatatcattaataataatattacgaaaatactaataatatcacgaatattataaataacacaaataaactaat

SEQ ID NO: 167 - DNA, 749 bp, Gossypium hirsutum
tttcgattctaataaaaaaaaaatcaaaataaagaatatcattaataataatattacgaaaatactaataatatcacgaatattataaataacacaaataaactaatctttttgaatctaccgtgcatcttcaaataacccgatagatcaaataactcggtagaatagatccagcaatctcacagttgttcgagtgccaatcttaagaaatcattaaaaatcaaaagcaagaatcacattttctccaatacttgactgttagaaagaaggttattaaaaacaaaaaaagagcaatttggatatggttattctgatatataaatagagtatgctctgggacggaaggattcgaacctccgcatagcgggaccaaaacccgttgccttaccacttggccacgccccatttagatttctatttgaaactactaaacactaatatggtattccttgttcgtcaattccagttccaaatagctatagctatggaatagattcttgttacgatttttgcacgtatggatagagaattaaatcgaatttattgatcattacatagaattcaattaagatattgtatgaaagtatgatttcttctattctcttgtaagaattgaaggctttttgattgggtgagttcaaagaagtattgtttagtctgccttattttcctttcttcatttttttccttatatcaaaaaacatattaataactcaatcaaaattatctccaagaacaaaattttgtta

SEQ ID NO: 168 - DNA, 642 bp, Gossypium hirsutum
aagaatcggttacatttttcatatgatctcctattttattttagatagactaaaaaaaaagaactcggttcttttttttgtattatatcgctttgactccttttccatttattctattctatcatatttatattattctaattattctaattctatgtatttcttttttgttttaataaattattaaattttttataaaattattcataaataatgaattttcatttacctatgtctaaacggagtcaaaaatatattctatttatctagttagctagaaatgtatttttttttcaattgaaaattcccaataataataattcttattagaattagggggcaggtttcatgtcaatggcaaaatacctcattcgttgcaatactcctttaaacaataaatgagaaataaggggttctcttgaactaagaaggggaaggaagaaagcgagtcggtgtgataattcctcatcctcaaattaatccttcccatggattattgtcccaacgaataattaattgtaggggtgaaatcttgatagaattcgaaaaagcgaggagtaagtcccaagccataaaataagaaaaaaaaatacaaaattctcatgtttataggattaaacaaagggattcgcaaataagagt

SEQ ID NO: 169 - DNA, 106 bp, Gossypium hirsutum
cttattagaattagggggcaggtttcatgtcaatggcaaaatacctcattcgttgcaatactcctttaaacaataaatgagaaataaggggttctcttgaactaag

SEQ ID NO: 170 - DNA, 428 bp, Gossypium hirsutum
aagaatcggttacatttttcatatgatctcctattttattttagatagactaaaaaaaaagaactcggttcttttttttgtattatatcgctttgactccttttccatttattctattctatcatatttatattattctaattattctaattctatgtatttcttttttgttttaataaattattaaattttttataaaattattcataaataatgaattttcatttacctatgtctaaacggagtcaaaaatatattctatttatctagttagctagaaatgtatttttttttcaattgaaaattcccaataataataattcttattagaattagggggcaggtttcatgtcaatggcaaaatacctcattcgttgcaatactcctttaaacaataaatgagaaataaggggttctcttgaactaag

SEQ ID NO: 171 - DNA, 320 bp, Gossypium hirsutum
cttattagaattagggggcaggtttcatgtcaatggcaaaatacctcattcgttgcaatactcctttaaacaataaatgagaaataaggggttctcttgaactaagaaggggaaggaagaaagcgagtcggtgtgataattcctcatcctcaaattaatccttcccatggattattgtcccaacgaataattaattgtaggggtgaaatcttgatagaattcgaaaaagcgaggagtaagtcccaagccataaaataagaaaaaaaaatacaaaattctcatgtttataggattaaacaaagggattcgcaaataagagt

SEQ ID NO: 172 - DNA, 1284 bp, Gossypium hirsutum
tcatgataagttcctcgcaccaaaataaagaaatggttaatgatacaatcatccaatgaattttgacttaattatattattccatcgctaagattcaacctgccgaagtaactaagaactccgaatggaagtgagaagattattgaaccgtcagaactatttcgacatcttttttttaggtcctatccactagatttttttgaatccgcacatactttgttcttccctttcttttttccaacccattctttgctttgaattcttcgtttttgtctttattttatctctttattcgttcaattcaaagcaaagacgaaacagaggaacttctattcttctattggaatacctatctaactccacagtagtaggttggattagatatattttgagttcgtatataactagtcaatatgttcgtatataactagtcaatatctaatatcccatatacatgtgtttcttacataacgtaaaccaactattcatgtcttagattcaattggattctagaattattcctcaaaggatagacaagagctgtcttatagccattagattccatatacctaattttacccccccccccccctttttttcctaatcatttttttttatatcgttttttgtaatgtaaattcgtctcttccttttattttttatttagcattatctcacatggatacaatctaaatggacgaattcattagaccaaatcattgcatacaaataaaacatcaatatcagtatttgattgagctaagttcatgcaatttattttattatttatgaaaaaaaaaaaatttcttttggtcaatcattgaattgaatgaagaaatcgactgaaatgggaatcattctatatctatataaagaacagcttttttaaaatcatagaccctaaattgataccataagaattcctattctaattaggactcataatattgaaattgaataaacaaaaacaatatcaagataaagataatattgatcggataatattgatcgaaggtaaatatgatataaatataagtaataaaaaaaatggaccaaccctgaattttaggaaaaagatcttttcaattagatctctttcctttttttattacaattctctaatatcgtaatcttttttgttattactctagatcgtcgtatttgtatatttatggtccctaagtaagtagattatcttgagttgcgcatacattggattggaccaagctaagctaaaaaagggctagactatttcaaaaacaaaaactagtcaatgatgaccct

SEQ ID NO: 173 - DNA, 642 bp, Gossypium hirsutum
aggaacttctattcttctattggaatacctatctaactccacagtagtaggttggattagatatattttgagttcgtatataactagtcaatatgttcgtatataactagtcaatatctaatatcccatatacatgtgtttcttacataacgtaaaccaactattcatgtcttagattcaattggattctagaattattcctcaaaggatagacaagagctgtcttatagccattagattccatatacctaattttacccccccccccccctttttttcctaatcatttttttttatatcgttttttgtaatgtaaattcgtctcttccttttattttttatttagcattatctcacatggatacaatctaaatggacgaattcattagaccaaatcattgcatacaaataaaacatcaatatcagtatttgattgagctaagttcatgcaatttattttattatttatgaaaaaaaaaaaatttcttttggtcaatcattgaattgaatgaagaaatcgactgaaatgggaatcattctatatctatataaagaacagcttttttaaaatcatagaccctaaattgataccataagaattcctattctaattaggactcataatattgaaattgaataaac

SEQ ID NO: 174 - DNA, 963 bp, Gossypium hirsutum
tcatgataagttcctcgcaccaaaataaagaaatggttaatgatacaatcatccaatgaattttgacttaattatattattccatcgctaagattcaacctgccgaagtaactaagaactccgaatggaagtgagaagattattgaaccgtcagaactatttcgacatcttttttttaggtcctatccactagatttttttgaatccgcacatactttgttcttccctttcttttttccaacccattctttgctttgaattcttcgtttttgtctttattttatctctttattcgttcaattcaaagcaaagacgaaacagaggaacttctattcttctattggaatacctatctaactccacagtagtaggttggattagatatattttgagttcgtatataactagtcaatatgttcgtatataactagtcaatatctaatatcccatatacatgtgtttcttacataacgtaaaccaactattcatgtcttagattcaattggattctagaattattcctcaaaggatagacaagagctgtcttatagccattagattccatatacctaattttacccccccccccccctttttttcctaatcatttttttttatatcgttttttgtaatgtaaattcgtctcttccttttattttttatttagcattatctcacatggatacaatctaaatggacgaattcattagaccaaatcattgcatacaaataaaacatcaatatcagtatttgattgagctaagttcatgcaatttattttattatttatgaaaaaaaaaaaatttcttttggtcaatcattgaattgaatgaagaaatcgactgaaatgggaatcattctatatctatataaagaacagcttttttaaaatcatagaccctaaattgataccataagaattcctattctaattaggactcataatattgaaattgaataaac

SEQ ID NO: 175 - DNA, 963 bp, Gossypium hirsutum
aggaacttctattcttctattggaatacctatctaactccacagtagtaggttggattagatatattttgagttcgtatataactagtcaatatgttcgtatataactagtcaatatctaatatcccatatacatgtgtttcttacataacgtaaaccaactattcatgtcttagattcaattggattctagaattattcctcaaaggatagacaagagctgtcttatagccattagattccatatacctaattttacccccccccccccctttttttcctaatcatttttttttatatcgttttttgtaatgtaaattcgtctcttccttttattttttatttagcattatctcacatggatacaatctaaatggacgaattcattagaccaaatcattgcatacaaataaaacatcaatatcagtatttgattgagctaagttcatgcaatttattttattatttatgaaaaaaaaaaaatttcttttggtcaatcattgaattgaatgaagaaatcgactgaaatgggaatcattctatatctatataaagaacagcttttttaaaatcatagaccctaaattgataccataagaattcctattctaattaggactcataatattgaaattgaataaacaaaaacaatatcaagataaagataatattgatcggataatattgatcgaaggtaaatatgatataaatataagtaataaaaaaaatggaccaaccctgaattttaggaaaaagatcttttcaattagatctctttcctttttttattacaattctctaatatcgtaatcttttttgttattactctagatcgtcgtatttgtatatttatggtccctaagtaagtagattatcttgagttgcgcatacattggattggaccaagctaagctaaaaaagggctagactatttcaaaaacaaaaactagtcaatgatgaccct

SEQ ID NO: 176 - DNA, 1391 bp, Gossypium hirsutum
catagagataggggacataatacacatggatatagtaagtcttgcttgggctgctttaatggtagtctttacattttccctttcactcgtagtatggggaagaagtgggctctaggggtactcctaattggggttgaggaatcaaaccgtatcaattgttttctagatcgttttgcaaagcctttttttattttaactattttattagtcttcatttcgaaattcttggattctagtggagtaatgtattctatgaataacaccttttcaatcaaaatcaaataaacatttcaataatccccatgttcgtatttcgaaagtaaaagggatccggatgataggcaataaaaaaaaagaattcttcctaaattttctattttgatgaaccagctcttaccagagttatgaacggacaatgagggacaaggaacccccctttgttttctgtatttgcttgtttttatttccctccctctttactgttcaaagagaaaaaaaagtcttttttttatttaattttaataagatcttgccttagtgtagtcacatattagacacttaccccctgttttatttgaatttcatttatgaaatgctttattgactcatttcatatcatggatcaaagaagttaaattcaaaatcggcagggtatacccttttttcgaaacgaaatacgaagaaaagttaccatagaactctttggatcatctgtcagttgctcaacgaattacttctttggaatgttcaaaaaaaagattacttggttttctttttttttattaatttaattaatatatgcctattccattccatttttccttcatttctgattattttaataggaatctcggtatccaccaaaaaaatcaaatccatgaaatgaaagaattataaaatcgtaagacttgcctttcaattatttcagattaattgaaatcaacacaaataaaatagatcaagcgaagaaataaaattgagatactatgtacagtacatatatttaattgatatcgacatgtatgaagatacatattgtggattcatctatattaagcttgtatctttctacattacaataatagatatagatagtatggtagaaagattcatattttttttctaccatactatcgagttttataggatggtagaaagattcatattttttttctaccatactatcgagttttataggatactgctgattctagtccattcattttatttaagacgtgagatttgaatcctttttttcttaactccttgataaaaagtcaagtttcattcaaattaatcactttgactgacagtttttacgtatattataagtaaaaaagcggtaggaactagaatgaacagcgctgtagcaataaatgcgagaatattta

SEQ ID NO: 177 - DNA, 428 bp, Gossypium hirsutum
tagtgtagtcacatattagacacttaccccctgttttatttgaatttcatttatgaaatgctttattgactcatttcatatcatggatcaaagaagttaaattcaaaatcggcagggtatacccttttttcgaaacgaaatacgaagaaaagttaccatagaactctttggatcatctgtcagttgctcaacgaattacttctttggaatgttcaaaaaaaagattacttggttttctttttttttattaatttaattaatatatgcctattccattccatttttccttcatttctgattattttaataggaatctcggtatccaccaaaaaaatcaaatccatgaaatgaaagaattataaaatcgtaagacttgcctttcaattatttcagattaattgaaatcaacacaaataaaatagatcaag

SEQ ID NO: 178 - DNA, 963 bp, Gossypium hirsutum
catagagataggggacataatacacatggatatagtaagtcttgcttgggctgctttaatggtagtctttacattttccctttcactcgtagtatggggaagaagtgggctctaggggtactcctaattggggttgaggaatcaaaccgtatcaattgttttctagatcgttttgcaaagcctttttttattttaactattttattagtcttcatttcgaaattcttggattctagtggagtaatgtattctatgaataacaccttttcaatcaaaatcaaataaacatttcaataatccccatgttcgtatttcgaaagtaaaagggatccggatgataggcaataaaaaaaaagaattcttcctaaattttctattttgatgaaccagctcttaccagagttatgaacggacaatgagggacaaggaacccccctttgttttctgtatttgcttgtttttatttccctccctctttactgttcaaagagaaaaaaaagtcttttttttatttaattttaataagatcttgccttagtgtagtcacatattagacacttaccccctgttttatttgaatttcatttatgaaatgctttattgactcatttcatatcatggatcaaagaagttaaattcaaaatcggcagggtatacccttttttcgaaacgaaatacgaagaaaagttaccatagaactctttggatcatctgtcagttgctcaacgaattacttctttggaatgttcaaaaaaaagattacttggttttctttttttttattaatttaattaatatatgcctattccattccatttttccttcatttctgattattttaataggaatctcggtatccaccaaaaaaatcaaatccatgaaatgaaagaattataaaatcgtaagacttgcctttcaattatttcagattaattgaaatcaacacaaataaaatagatcaag

SEQ ID NO: 179 - DNA, 856 bp, Gossypium hirsutum
tagtgtagtcacatattagacacttaccccctgttttatttgaatttcatttatgaaatgctttattgactcatttcatatcatggatcaaagaagttaaattcaaaatcggcagggtatacccttttttcgaaacgaaatacgaagaaaagttaccatagaactctttggatcatctgtcagttgctcaacgaattacttctttggaatgttcaaaaaaaagattacttggttttctttttttttattaatttaattaatatatgcctattccattccatttttccttcatttctgattattttaataggaatctcggtatccaccaaaaaaatcaaatccatgaaatgaaagaattataaaatcgtaagacttgcctttcaattatttcagattaattgaaatcaacacaaataaaatagatcaagcgaagaaataaaattgagatactatgtacagtacatatatttaattgatatcgacatgtatgaagatacatattgtggattcatctatattaagcttgtatctttctacattacaataatagatatagatagtatggtagaaagattcatattttttttctaccatactatcgagttttataggatggtagaaagattcatattttttttctaccatactatcgagttttataggatactgctgattctagtccattcattttatttaagacgtgagatttgaatcctttttttcttaactccttgataaaaagtcaagtttcattcaaattaatcactttgactgacagtttttacgtatattataagtaaaaaagcggtaggaactagaatgaacagcgctgtagcaataaatgcgagaatattta

SEQ ID NO: 180 - DNA, 1070 bp, Gossypium hirsutum
aaatttttacttctggttccggcgaacgaataatcattgagtcctcctctttccggacaacacatacaaagagacccgccaacagtcagtaattagtgaacctatgagagacgcttataattagtttctttctcttctatctctcatctatctatctattttttatctatctattttttctttagttattcactagagcaattatgatctggaagtcgatccggggcaagtgttcggatctattatgacatatccatgaggcgctcaacggacctttttaatcttataaaacctttttcggactttacttacattggtgcaaaacccatttttttgtgcaaccggtgtagtgtattcatatctcaattagaagttccgaggtggagttttttttgtcttacatagatagaacaaacaatattactctatcccaaatcacgtgcacattcattactacgggatactccagtatcgatagttagtcatttgaggggtttttattagttttagttttaatagaaatagaaggagaaaatatatatatatattatatatattttattttgctatatccgtgtattacttatccttacgaaatatcaaacgaaatagaacgatttgagaaaggatataatgaaattctttgattggtttttcccaaagagagaaatgctccgttttatttgacccatgggtccaacaaaaccaaaccattacacaattaacaattccaattaaattaatattttttactatttaatttaaatattcaaattgactagaaaatcgaatttaataatttaaatagtaccgtaatttatttaattaaattaaatattaaataaatggatttaatagaaattaaatagaaatagaattaaatagaattctctaatttctaattcgaatttcgaatcctaaaactaaaataataataaataaacagagagctcttgttcttattcgaaacgccgcgtgatctttaaccaattctgcgcttcaatataattaccaggagtaagcgctatagcctgtttccaatactcagcggcttgatcgaaccaagcctc

SEQ ID NO: 181 - DNA, 361 bp, Gossypium hirsutum
gcaaccggtgtagtgtattcatatctcaattagaagttccgaggtggagttttttttgtcttacatagatagaacaaacaatattactctatcccaaatcacgtgcacattcattactacgggatactccagtatcgatagttagtcatttgaggggtttttattagttttagttttaatagaaatagaaggagaaaatatatatatatattatatatattttattttgctatatccgtgtattacttatccttacgaaatatcaaacgaaatagaacgatttgagaaaggatataatgaaattctttgattggtttttcccaaagagagaaatgctccgttttatttgacccatgggtcc

SEQ ID NO: 182 - DNA, 698 bp, Gossypium hirsutum
aaatttttacttctggttccggcgaacgaataatcattgagtcctcctctttccggacaacacatacaaagagacccgccaacagtcagtaattagtgaacctatgagagacgcttataattagtttctttctcttctatctctcatctatctatctattttttatctatctattttttctttagttattcactagagcaattatgatctggaagtcgatccggggcaagtgttcggatctattatgacatatccatgaggcgctcaacggacctttttaatcttataaaacctttttcggactttacttacattggtgcaaaacccatttttttgtgcaaccggtgtagtgtattcatatctcaattagaagttccgaggtggagttttttttgtcttacatagatagaacaaacaatattactctatcccaaatcacgtgcacattcattactacgggatactccagtatcgatagttagtcatttgaggggtttttattagttttagttttaatagaaatagaaggagaaaatatatatatatattatatatattttattttgctatatccgtgtattacttatccttacgaaatatcaaacgaaatagaacgatttgagaaaggatataatgaaattctttgattggtttttcccaaagagagaaatgctccgttttatttgacccatgggtcc

SEQ ID NO: 183 - DNA, 733 bp, Gossypium hirsutum
gcaaccggtgtagtgtattcatatctcaattagaagttccgaggtggagttttttttgtcttacatagatagaacaaacaatattactctatcccaaatcacgtgcacattcattactacgggatactccagtatcgatagttagtcatttgaggggtttttattagttttagttttaatagaaatagaaggagaaaatatatatatatattatatatattttattttgctatatccgtgtattacttatccttacgaaatatcaaacgaaatagaacgatttgagaaaggatataatgaaattctttgattggtttttcccaaagagagaaatgctccgttttatttgacccatgggtccaacaaaaccaaaccattacacaattaacaattccaattaaattaatattttttactatttaatttaaatattcaaattgactagaaaatcgaatttaataatttaaatagtaccgtaatttatttaattaaattaaatattaaataaatggatttaatagaaattaaatagaaatagaattaaatagaattctctaatttctaattcgaatttcgaatcctaaaactaaaataataataaataaacagagagctcttgttcttattcgaaacgccgcgtgatctttaaccaattctgcgcttcaatataattaccaggagtaagcgctatagcctgtttccaatactcagcggcttgatcgaaccaagcctc

SEQ ID NO: 184 - DNA, 1070 bp, Gossypium hirsutum
ttctaatcggggctaaaactccagaaattagaaatgccaaaataggaatagcacttgatattattagaaatgcccagaaaatatcatattcgtaaagcagaaacatagacgaactcctatgaatgtgaaaaatagatcggattagtcgcttcgacctggaattcattgtcaagtcatccgcaactgtttgtttagtcaaaacaacaattcagtttgatcgaatcgtccagtttcgtttgtttgcggtgtttccatgttgcgttttaagatttattgattggaattaatcctattttatttacattccacttatttcgatttcatttcgatatagtactaatttgtataatactagtataaatatatatttatactatatatagtataatactatacaaatccaaaacaagtaatactttttcgttttcacttcatttcggatttttctaaataaaaggaattctaatatctaactaatatctaatatttattagaagaaataaaaaaacttttaattcgaaattctatttattctaaatagaaattcgaaattatagatttcgaataaatagaaattttttattttatatctatttatatagaatcaatcaaaaatcaatctacccgcgaggattaattaagaaaatggggttttgtttattttattcttatccaagaaaaaaaaagagcgattggatccgttgaaatgcgcttttgttttcagggttatactctgagtgatctccctgtgagcgagcttatgggaatgattttaaccaagcaactggaagcgaccagttccaatcaacaaagaatacaataattaggaaaaaaactctatttttatttttagttggatttggatattctttattttattgttttagtttgctttagttttaagaatattattttcatttttcttaatttaataaatttaatgaatatttaaaattaataaaatagaaaattcgaaaatatagggctatacggactcgaaccgtagaccttctcggtaaaacagatcgaactgattattatcaaaatgattcggcctatttcaaagaccc

SEQ ID NO: 185 - DNA, 214 bp, Gossypium hirsutum
acttcatttcggatttttctaaataaaaggaattctaatatctaactaatatctaatatttattagaagaaataaaaaaacttttaattcgaaattctatttattctaaatagaaattcgaaattatagatttcgaataaatagaaattttttattttatatctatttatatagaatcaatcaaaaatcaatctacccgcgaggattaattaag

SEQ ID NO: 186 - DNA, 642 bp, Gossypium hirsutum
ttctaatcggggctaaaactccagaaattagaaatgccaaaataggaatagcacttgatattattagaaatgcccagaaaatatcatattcgtaaagcagaaacatagacgaactcctatgaatgtgaaaaatagatcggattagtcgcttcgacctggaattcattgtcaagtcatccgcaactgtttgtttagtcaaaacaacaattcagtttgatcgaatcgtccagtttcgtttgtttgcggtgtttccatgttgcgttttaagatttattgattggaattaatcctattttatttacattccacttatttcgatttcatttcgatatagtactaatttgtataatactagtataaatatatatttatactatatatagtataatactatacaaatccaaaacaagtaatactttttcgttttcacttcatttcggatttttctaaataaaaggaattctaatatctaactaatatctaatatttattagaagaaataaaaaaacttttaattcgaaattctatttattctaaatagaaattcgaaattatagatttcgaataaatagaaattttttattttatatctatttatatagaatcaatcaaaaatcaatctacccgcgaggattaattaag

SEQ ID NO: 187 - DNA, 642 bp, Gossypium hirsutum
acttcatttcggatttttctaaataaaaggaattctaatatctaactaatatctaatatttattagaagaaataaaaaaacttttaattcgaaattctatttattctaaatagaaattcgaaattatagatttcgaataaatagaaattttttattttatatctatttatatagaatcaatcaaaaatcaatctacccgcgaggattaattaagaaaatggggttttgtttattttattcttatccaagaaaaaaaaagagcgattggatccgttgaaatgcgcttttgttttcagggttatactctgagtgatctccctgtgagcgagcttatgggaatgattttaaccaagcaactggaagcgaccagttccaatcaacaaagaatacaataattaggaaaaaaactctatttttatttttagttggatttggatattctttattttattgttttagtttgctttagttttaagaatattattttcatttttcttaatttaataaatttaatgaatatttaaaattaataaaatagaaaattcgaaaatatagggctatacggactcgaaccgtagaccttctcggtaaaacagatcgaactgattattatcaaaatgattcggcctatttcaaagaccc

SEQ ID NO: 188 - DNA, 856 bp, Gossypium hirsutum
taaattagataaagtggaaaaatgagataaaccagcttcataaatcaaatttgaattcgaagcaatggatactttgtaatccagtaattactgtcggtttgttgaattgcaattaaactcggcccaatcttttactaaaaggattgagccgattctattgtctatatttttgatagatatatttttatctagatatacaagatcttaaatacaaaaataaaagactaaacacaactcaaattttctcctgggtccataattaatcctatgtatggatccttaggattggtgtattcttttctatcctatataccacggtttcggatcatggatcgagccaagcatcacaacttcttctacccatcctgtatgttgtccttttggctccgtgttggaatagaaacttattgtattagtagacgagattttacgaaaaaggttcttgataggagaaaaatttcttttttcaattttttcagtgcggattttacacaacatggggaaatcactatttctaattgaaaattgatatttctaattcaaaagaaaaaagagctctatcctatcatatatctgctactctaggttctcacaaaagagaatcgtttctttcaatattcactcattgttcgttaataaccctagtgattgtatctagtatgcgtattctgataggaaataaaatattcaattgatttttcatcgaatgactattcatctactgtactttcatggaaatagaggcaagaaagctctatggaaaaatcatggttcaatttgatcttgtctaagggagaattagaatacagatgtgggctaagtaaatcaatggatagccgccttggtcctgttgaaa

SEQ ID NO: 189 - DNA, 107 bp, Gossypium hirsutum
cggatcatggatcgagccaagcatcacaacttcttctacccatcctgtatgttgtccttttggctccgtgttggaatagaaacttattgtattagtagacgagattt

SEQ ID NO: 190 - DNA, 428 bp, Gossypium hirsutum
taaattagataaagtggaaaaatgagataaaccagcttcataaatcaaatttgaattcgaagcaatggatactttgtaatccagtaattactgtcggtttgttgaattgcaattaaactcggcccaatcttttactaaaaggattgagccgattctattgtctatatttttgatagatatatttttatctagatatacaagatcttaaatacaaaaataaaagactaaacacaactcaaattttctcctgggtccataattaatcctatgtatggatccttaggattggtgtattcttttctatcctatataccacggtttcggatcatggatcgagccaagcatcacaacttcttctacccatcctgtatgttgtccttttggctccgtgttggaatagaaacttattgtattagtagacgagattt

SEQ ID NO: 191 - DNA, 535 bp, Gossypium hirsutum
cggatcatggatcgagccaagcatcacaacttcttctacccatcctgtatgttgtccttttggctccgtgttggaatagaaacttattgtattagtagacgagattttacgaaaaaggttcttgataggagaaaaatttcttttttcaattttttcagtgcggattttacacaacatggggaaatcactatttctaattgaaaattgatatttctaattcaaaagaaaaaagagctctatcctatcatatatctgctactctaggttctcacaaaagagaatcgtttctttcaatattcactcattgttcgttaataaccctagtgattgtatctagtatgcgtattctgataggaaataaaatattcaattgatttttcatcgaatgactattcatctactgtactttcatggaaatagaggcaagaaagctctatggaaaaatcatggttcaatttgatcttgtctaagggagaattagaatacagatgtgggctaagtaaatcaatggatagccgccttggtcctgttgaaa

SEQ ID NO: 192 - DNA, 856 bp, Gossypium hirsutum
tagagtcttaagtgaaatttttttatttgtagtgaaaaggtagttagttagtttatcagaatcaaagtcaaagcccgcgtaatgggctttgactttgtgacaaaaaaaggaattgtcgaaaaacaaattatgtggataattattattatcattatccgatattactaatgagtaaacctctagcaacaagataaaaagcgaatttttccttctgtgaaatgaaattcgaatttggagagacaaataaaacgaattttatcctcctctattgcgtatgtatatagaatttaaatagagaaaaaatagaatagaaatatagagttttgcatctttctatatctcccgaaaattctatttttactaataatccctgttcttggatcggattctaacgaatcgaatctttcgacaatttgtaataaactctttctttattaaattcaaaaatgcaaattagaagacaagaacaatagaataataagaaaagtaagaataaagtaagataataaagaaagtggattatcttatcatatacatctattttatttgatttaaaaagatgggcaagtccttttatttaattctacattccttgcacttattagatatctattagatatatagattcgcttagatatacttagtatttagatataattagtataatatacgaattataatataatcgttataagatatatttctaactaaacaatataacaataacaggtacaaatattaaattgaggtacccattttatgacaactttcagcagttttccctctatttttgtgcctttagtaggcctagtatttccggcaattgcaatggcttctttatctttgtatgttcaaa

SEQ ID NO: 193 - DNA, 107 bp, Gossypium hirsutum
ttttgcatctttctatatctcccgaaaattctatttttactaataatccctgttcttggatcggattctaacgaatcgaatctttcgacaatttgtaataaactctt

SEQ ID NO: 194 - DNA, 428 bp, Gossypium hirsutum
tagagtcttaagtgaaatttttttatttgtagtgaaaaggtagttagttagtttatcagaatcaaagtcaaagcccgcgtaatgggctttgactttgtgacaaaaaaaggaattgtcgaaaaacaaattatgtggataattattattatcattatccgatattactaatgagtaaacctctagcaacaagataaaaagcgaatttttccttctgtgaaatgaaattcgaatttggagagacaaataaaacgaattttatcctcctctattgcgtatgtatatagaatttaaatagagaaaaaatagaatagaaatatagagttttgcatctttctatatctcccgaaaattctatttttactaataatccctgttcttggatcggattctaacgaatcgaatctttcgacaatttgtaataaactctt

SEQ ID NO: 195 - DNA, 535 bp, Gossypium hirsutum
ttttgcatctttctatatctcccgaaaattctatttttactaataatccctgttcttggatcggattctaacgaatcgaatctttcgacaatttgtaataaactctttctttattaaattcaaaaatgcaaattagaagacaagaacaatagaataataagaaaagtaagaataaagtaagataataaagaaagtggattatcttatcatatacatctattttatttgatttaaaaagatgggcaagtccttttatttaattctacattccttgcacttattagatatctattagatatatagattcgcttagatatacttagtatttagatataattagtataatatacgaattataatataatcgttataagatatatttctaactaaacaatataacaataacaggtacaaatattaaattgaggtacccattttatgacaactttcagcagttttccctctatttttgtgcctttagtaggcctagtatttccggcaattgcaatggcttctttatctttgtatgttcaaa

SEQ ID NO: 196 - DNA, 1070 bp, Gossypium hirsutum
attgaagtcttttgaaatgaattgcagaataaaagctttctcgccgggaggaaaaagctcaatccaagaatcctcttttttctatagcagaactaaactgaagtttcttcagaatgcccattcaaaccaaagcggacgtatacggaagagtcataacataaaaaaacagttttttttgtccacaaaaatagttttttatgcgtctgtaaatgtaaaaccgctcaacttaattcagtaacaaaataagcaagaaatcgaaatactggattcatctcatatatcaaatcgaaataaagactttcgctttttatttgcaatatttggagttgatacgttagatacagatagatcatatcttgtaaattttctctacttttcttttctcaattggcccctccccttttatcctttcttttgtgctccttaagaactaaagaactaaagaactaaacaagtggatttctttcttataacataatgataaacgtaatgataacttttctgtctttttttgtcactcatttttgatcagcataatatttttcctaatttttttttcaattattgctggactctagactatatatagtttagataacccgcgaaaattttcgacatatttgattgcgatcttgatatagacagggcgctccttcgtctcaaaatctgaatagaacaatctttattatttgaagcggttctttcgggcagttatcaaaagagggcaattgcttcgaatttgatcaattgagatatatggaaacaaaaacaatatcaatataacaataatatatatatttcattcgaattcaaagtggattcttattttctatattctgtattctttttagattcaattagattcaagggctaagcactgaatcaaaaaaaaacagagaatagattcatgggcccctactttataatataatgaaaggcatgcttgatagaaagattagatcacataaagtcaacgaatgaggtgggttcattaacaactcacagatgaaaaaatggcaaaaaagaaagcattcactccccttctatatcttgcatctatagtatttttg

SEQ ID NO: 197 - DNA, 321 bp, Gossypium hirsutum
tggagttgatacgttagatacagatagatcatatcttgtaaattttctctacttttcttttctcaattggcccctccccttttatcctttcttttgtgctccttaagaactaaagaactaaagaactaaacaagtggatttctttcttataacataatgataaacgtaatgataacttttctgtctttttttgtcactcatttttgatcagcataatatttttcctaatttttttttcaattattgctggactctagactatatatagtttagataacccgcgaaaattttcgacatatttgattgcgatcttgatataga

SEQ ID NO: 198 - DNA, 642 bp, Gossypium hirsutum
attgaagtcttttgaaatgaattgcagaataaaagctttctcgccgggaggaaaaagctcaatccaagaatcctcttttttctatagcagaactaaactgaagtttcttcagaatgcccattcaaaccaaagcggacgtatacggaagagtcataacataaaaaaacagttttttttgtccacaaaaatagttttttatgcgtctgtaaatgtaaaaccgctcaacttaattcagtaacaaaataagcaagaaatcgaaatactggattcatctcatatatcaaatcgaaataaagactttcgctttttatttgcaatatttggagttgatacgttagatacagatagatcatatcttgtaaattttctctacttttcttttctcaattggcccctccccttttatcctttcttttgtgctccttaagaactaaagaactaaagaactaaacaagtggatttctttcttataacataatgataaacgtaatgataacttttctgtctttttttgtcactcatttttgatcagcataatatttttcctaatttttttttcaattattgctggactctagactatatatagtttagataacccgcgaaaattttcgacatatttgattgcgatcttgatataga

SEQ ID NO: 199 - DNA, 749 bp, Gossypium hirsutum
tggagttgatacgttagatacagatagatcatatcttgtaaattttctctacttttcttttctcaattggcccctccccttttatcctttcttttgtgctccttaagaactaaagaactaaagaactaaacaagtggatttctttcttataacataatgataaacgtaatgataacttttctgtctttttttgtcactcatttttgatcagcataatatttttcctaatttttttttcaattattgctggactctagactatatatagtttagataacccgcgaaaattttcgacatatttgattgcgatcttgatatagacagggcgctccttcgtctcaaaatctgaatagaacaatctttattatttgaagcggttctttcgggcagttatcaaaagagggcaattgcttcgaatttgatcaattgagatatatggaaacaaaaacaatatcaatataacaataatatatatatttcattcgaattcaaagtggattcttattttctatattctgtattctttttagattcaattagattcaagggctaagcactgaatcaaaaaaaaacagagaatagattcatgggcccctactttataatataatgaaaggcatgcttgatagaaagattagatcacataaagtcaacgaatgaggtgggttcattaacaactcacagatgaaaaaatggcaaaaaagaaagcattcactccccttctatatcttgcatctatagtatttttg

SEQ ID NO: 200 - DNA, 1177 bp, Gossypium hirsutum
atgaatttctagatctgtggatttatcaacatcaaatttgtaaaaaagaacaaattattcctggcaattaggttaggtataatgaaaaaagggatgaaaagtcttttgcttgtttctattctttctctaggaattgcttttaccgcattcaaaacaaaatatgtatattgtgaagaagactatttgtctttacctcttttttttttcttttttcaatctaaattggactaaattgggggggggggggtgtaattatattcctattgtcagtgtcagatttaaatgttacagaataggttttgttttctaaattcaatttgagtagatactaaacataagataagtaagtggagaatagaaaggaaggataaattaaatgaggggaacaaacagggagtattcttcgtctttctagccttcgacacaagaaaaggaattttacacacccttttcttgtgttcttgtgtcgaaataataacaattccgggacccgttcgtcaaagatttctattcttagtttcgatttttccagatttttcagaaccctttattttttcgatttacttataatagaataagtaataaggataatataataagtataaaataagtcgaaataagtataatatactaagtaatggacaaccaaaaaaaagagaaggaatccttaccgattttttttgataaaatagaatcaaggcgataaacttataaaataatttcaaactttgatgaaatactaaaataaatcgagtaaggttagactagtctagaaaaggtttgaatctggtcgacagaaaaaaaaagaaatattaaatatatattgtattgtgtcatccaattgagtgattcctttttttcttctaaccttgaaagtatcgatagatactatcgaagagcagatgctatgaatcaattaattgaaattgagttcatttttcaaaaaaatcatcggaaaaagtgatccatttgttgtcatttatacgaccaaaatattatgatcatgatcattaagaattcaacgggacccttcctcgcgaatcagacaaagaaagaggaggcgggccccgttgagttcttacactttcatgcctataacttagttcgtcccattatccattattccatattacagggacgaacccaatccagaatatgaaccataaaagaaaatacc

SEQ ID NO: 201 - DNA, 476 bp, Gossypium hirsutum
gtagatactaaacataagataagtaagtggagaatagaaaggaaggataaattaaatgaggggaacaaacagggagtattcttcgtctttctagccttcgacacaagaaaaggaattttacacacccttttcttgtgttcttgtgtcgaaataataacaattccgggacccgttcgtcaaagatttctattcttagtttcgatttttccagatttttcagaaccctttattttttcgatttacttataatagaataagtaataaggataatataataagtataaaataagtcgaaataagtataatatactaagtaatggacaaccaaaaaaaagagaaggaatccttaccgattttttttgataaaatagaatcaaggcgataaacttataaaataatttcaaactttgatgaaatactaaaataaatcgagtaaggttagactagtctagaaaaggtttgaatctggtcgacag

SEQ ID NO: 202 - DNA, 797 bp, Gossypium hirsutum
atgaatttctagatctgtggatttatcaacatcaaatttgtaaaaaagaacaaattattcctggcaattaggttaggtataatgaaaaaagggatgaaaagtcttttgcttgtttctattctttctctaggaattgcttttaccgcattcaaaacaaaatatgtatattgtgaagaagactatttgtctttacctcttttttttttcttttttcaatctaaattggactaaattgggggggggggggtgtaattatattcctattgtcagtgtcagatttaaatgttacagaataggttttgttttctaaattcaatttgagtagatactaaacataagataagtaagtggagaatagaaaggaaggataaattaaatgaggggaacaaacagggagtattcttcgtctttctagccttcgacacaagaaaaggaattttacacacccttttcttgtgttcttgtgtcgaaataataacaattccgggacccgttcgtcaaagatttctattcttagtttcgatttttccagatttttcagaaccctttattttttcgatttacttataatagaataagtaataaggataatataataagtataaaataagtcgaaataagtataatatactaagtaatggacaaccaaaaaaaagagaaggaatccttaccgattttttttgataaaatagaatcaaggcgataaacttataaaataatttcaaactttgatgaaatactaaaataaatcgagtaaggttagactagtctagaaaaggtttgaatctggtcgacag

SEQ ID NO: 203 - DNA, 856 bp, Gossypium hirsutum
gtagatactaaacataagataagtaagtggagaatagaaaggaaggataaattaaatgaggggaacaaacagggagtattcttcgtctttctagccttcgacacaagaaaaggaattttacacacccttttcttgtgttcttgtgtcgaaataataacaattccgggacccgttcgtcaaagatttctattcttagtttcgatttttccagatttttcagaaccctttattttttcgatttacttataatagaataagtaataaggataatataataagtataaaataagtcgaaataagtataatatactaagtaatggacaaccaaaaaaaagagaaggaatccttaccgattttttttgataaaatagaatcaaggcgataaacttataaaataatttcaaactttgatgaaatactaaaataaatcgagtaaggttagactagtctagaaaaggtttgaatctggtcgacagaaaaaaaaagaaatattaaatatatattgtattgtgtcatccaattgagtgattcctttttttcttctaaccttgaaagtatcgatagatactatcgaagagcagatgctatgaatcaattaattgaaattgagttcatttttcaaaaaaatcatcggaaaaagtgatccatttgttgtcatttatacgaccaaaatattatgatcatgatcattaagaattcaacgggacccttcctcgcgaatcagacaaagaaagaggaggcgggccccgttgagttcttacactttcatgcctataacttagttcgtcccattatccattattccatattacagggacgaacccaatccagaatatgaaccataaaagaaaatacc

SEQ ID NO: 204 - DNA, 1177 bp, Gossypium hirsutum
aataaactaattttttttcgacgtctacgagctatatatccccgtttaattctggtcattgaataaaagaaattttgatgaataactaattctttctttcagttattcttttctagtctattaataacaaaacggattcttccaatgtataaaataaaaattccaatggcttttgctactctaaccgtcccgaccacgatttttccttttttctaggcatttcgccttgaaataagaaattgtattgatactaggtatcaaaaaaagcatattaactaaaataaaggagtaaatagaaatggataaataaatagtgggttccatcgtttctatggttacttcttaaacggtgaggtcctctctatacaccggagctcattccttcatttaattggtaacttgtacagttcacactcttcggctctactcataaattttccagtaatagatctttcacaacgagatctatcttatacagtaacggtatgtaaggatgaggattaattgggcagctgaccctgttagtccgttctttgcaagagtcgaagcataatctttttgctttttaaataggattttccccgcttaatggataagcatttgctaccaatggggaattttttctcatcttaaattctaagattggatttgcgccaatggaaaccataaatttcatacacaatagaaggatatggtagatctagcttttttagatagtgaacggacgaaccccattctattcactggtacggatcgttgatactgaaaaagttgtttcttttttctcagcccatgatctgaacaagtcgcacatacaccctagtacatgttcctcggcgctgaggacatcccccaagagcaggggatttcgtgacatttctaattggctgtcttgcatttctaataagttgtttaatagttggcatactgaattatatacataatagactggtttagatcgatcctaaccagatgattctgaattacttctttttctatttaatagattaataaaatattaaccccttaggctttaggcttaagttaagaaggaaaggaagaagagggattaaatcaatcttaattaaattgtggattggtgttaaatcgttgctattgctatttgttatttaacttatttaaccgctacaagatccacaattccatgagcttgggc

SEQ ID NO: 205 - DNA, 463 bp, Gossypium hirsutum
ggtaacttgtacagttcacactcttcggctctactcataaattttccagtaatagatctttcacaacgagatctatcttatacagtaacggtatgtaaggatgaggattaattgggcagctgaccctgttagtccgttctttgcaagagtcgaagcataatctttttgctttttaaataggattttccccgcttaatggataagcatttgctaccaatggggaattttttctcatcttaaattctaagattggatttgcgccaatggaaaccataaatttcatacacaatagaaggatatggtagatctagcttttttagatagtgaacggacgaaccccattctattcactggtacggatcgttgatactgaaaaagttgtttcttttttctcagcccatgatctgaacaagtcgcacatacaccctagtacatgttcctcggcgctgaggacatcccccaa

SEQ ID NO: 206 - DNA, 856 bp, Gossypium hirsutum; identical to SEQ ID NO: 148

SEQ ID NO: 207 - DNA, 784 bp, Gossypium hirsutum
ggtaacttgtacagttcacactcttcggctctactcataaattttccagtaatagatctttcacaacgagatctatcttatacagtaacggtatgtaaggatgaggattaattgggcagctgaccctgttagtccgttctttgcaagagtcgaagcataatctttttgctttttaaataggattttccccgcttaatggataagcatttgctaccaatggggaattttttctcatcttaaattctaagattggatttgcgccaatggaaaccataaatttcatacacaatagaaggatatggtagatctagcttttttagatagtgaacggacgaaccccattctattcactggtacggatcgttgatactgaaaaagttgtttcttttttctcagcccatgatctgaacaagtcgcacatacaccctagtacatgttcctcggcgctgaggacatcccccaagagcaggggatttcgtgacatttctaattggctgtcttgcatttctaataagttgtttaatagttggcatactgaattatatacataatagactggtttagatcgatcctaaccagatgattctgaattacttctttttctatttaatagattaataaaatattaaccccttaggctttaggcttaagttaagaaggaaaggaagaagagggattaaatcaatcttaattaaattgtggattggtgttaaatcgttgctattgctatttgttatttaacttatttaaccgctacaagatccacaattccatgagcttgggc

SEQ ID NO: 208 - DNA, 1819 bp, Gossypium hirsutum
aatctgttcctatctaacggaagactattggatcaaatgacaaagacattgttgagaaaaagatggcttttcccggatgaaatgaaaattggattcatgtaagaggagaaaggtttcccattacttagccggaaagatatgtggtcatgaaatagggattaagtggaacggaattgactgggtggtagagttgtggaaacacctgtttcttccatattttggaccttagctccatgaaacaatatgctactgctgaaacatggaagaattgaaatcttagatcaaaacactatgtatggatggtatgaactgcctaaacaagaattcttgaacagcgaacaaccagagctattactcactacatcaaaaaatttccattaatgaaagatgtaaatccattggaaaatcaaaaatacgcatgtcggatgaaatggttgttgctatctgctccaataacgaatcattggtttaactgaataactaaataaaatagatagacctttctcttcgtctcaggtcgatggatcttctcaactggaggatcccctatatggataatatatggataatacacattccagttgaccgagcctaattctattctaattgttttgttccgaagcaaagatatccacggggcggttcgccctattcagatattcacgaccgagaagtactggattctctttcggataggtcctgaaaggagaaggaaagctggaatgccaccaggcgtctattattgaattcacccgacccgatagtacccatttttggaacgtccagtgccaaagtcacggaatgggtaagtcgccaatccctaaaacggactatgtaatgtactttatccgctgggttacggggggggcattttaccagaggtttatattgtatcaatctacccttgtgtgcgtgattcctgttgaagcatatactcgggggtgggtgcagggcgaacgatttcaaagcggactccccattcattagatagagaagatccccaagatctcgtgatccgctgccgaacttattccaattcaatgagcattctcaatattatgccttgaagaggactcgaacctccacgctctttagcacgagattttgagtctcgcgtgtctaccatttcaccaccaaggcatcttgaaagtgaatcgtattccatgaatatgatatctatctagtgtgatgtatggaatatatgacaaaggtggagtgttggagtatttctattgatcggtcatgtcatataggcccgagtcggacatccaattgcttcgatttgaagtatccggaggatgtctatattaagattatattaaatatatatagattatattaaaaagatggacaatcaaacctatttatttctcgattcaatagaagaaaaaaagaaaagaggtgaatagggtcccaaataacgagagatatgtaaaaagaaggcccgattacgcccattcctaatcctaaatggaatgtaacgacgtagggatccatatgtaaacatagtatctatttagatacgctcgaatgaccccttctcataatgagaatgtatatagccctattccggtctggtccggtatggaatgaacttataatcatggaatcgactcgatcatcagattatagattataagttcataaccccagcccattcccattttgggcggaacagatctactaattctttgattccagttagtaaagagggaccttgaactaagaaatagattctagaagctaactagaagctaaactaaaaaagggtatcctgagcaattgcaataatcgggttcattgatattcctggtatagt

SEQ ID NO: 209 - DNA, 535 bp, Gossypium hirsutum
gaattcttgaacagcgaacaaccagagctattactcactacatcaaaaaatttccattaatgaaagatgtaaatccattggaaaatcaaaaatacgcatgtcggatgaaatggttgttgctatctgctccaataacgaatcattggtttaactgaataactaaataaaatagatagacctttctcttcgtctcaggtcgatggatcttctcaactggaggatcccctatatggataatatatggataatacacattccagttgaccgagcctaattctattctaattgttttgttccgaagcaaagatatccacggggcggttcgccctattcagatattcacgaccgagaagtactggattctctttcggataggtcctgaaaggagaaggaaagctggaatgccaccaggcgtctattattgaattcacccgacccgatagtacccatttttggaacgtccagtgccaaagtcacggaatgggtaagtcgccaatccctaaaacggactatgtaatgtactttatccgctggg

SEQ ID NO: 210 - DNA, 856 bp, Gossypium hirsutum
aatctgttcctatctaacggaagactattggatcaaatgacaaagacattgttgagaaaaagatggcttttcccggatgaaatgaaaattggattcatgtaagaggagaaaggtttcccattacttagccggaaagatatgtggtcatgaaatagggattaagtggaacggaattgactgggtggtagagttgtggaaacacctgtttcttccatattttggaccttagctccatgaaacaatatgctactgctgaaacatggaagaattgaaatcttagatcaaaacactatgtatggatggtatgaactgcctaaacaagaattcttgaacagcgaacaaccagagctattactcactacatcaaaaaatttccattaatgaaagatgtaaatccattggaaaatcaaaaatacgcatgtcggatgaaatggttgttgctatctgctccaataacgaatcattggtttaactgaataactaaataaaatagatagacctttctcttcgtctcaggtcgatggatcttctcaactggaggatcccctatatggataatatatggataatacacattccagttgaccgagcctaattctattctaattgttttgttccgaagcaaagatatccacggggcggttcgccctattcagatattcacgaccgagaagtactggattctctttcggataggtcctgaaaggagaaggaaagctggaatgccaccaggcgtctattattgaattcacccgacccgatagtacccatttttggaacgtccagtgccaaagtcacggaatgggtaagtcgccaatccctaaaacggactatgtaatgtactttatccgctggg

SEQ ID NO: 211 - DNA, 1498 bp, Gossypium hirsutum
gaattcttgaacagcgaacaaccagagctattactcactacatcaaaaaatttccattaatgaaagatgtaaatccattggaaaatcaaaaatacgcatgtcggatgaaatggttgttgctatctgctccaataacgaatcattggtttaactgaataactaaataaaatagatagacctttctcttcgtctcaggtcgatggatcttctcaactggaggatcccctatatggataatatatggataatacacattccagttgaccgagcctaattctattctaattgttttgttccgaagcaaagatatccacggggcggttcgccctattcagatattcacgaccgagaagtactggattctctttcggataggtcctgaaaggagaaggaaagctggaatgccaccaggcgtctattattgaattcacccgacccgatagtacccatttttggaacgtccagtgccaaagtcacggaatgggtaagtcgccaatccctaaaacggactatgtaatgtactttatccgctgggttacggggggggcattttaccagaggtttatattgtatcaatctacccttgtgtgcgtgattcctgttgaagcatatactcgggggtgggtgcagggcgaacgatttcaaagcggactccccattcattagatagagaagatccccaagatctcgtgatccgctgccgaacttattccaattcaatgagcattctcaatattatgccttgaagaggactcgaacctccacgctctttagcacgagattttgagtctcgcgtgtctaccatttcaccaccaaggcatcttgaaagtgaatcgtattccatgaatatgatatctatctagtgtgatgtatggaatatatgacaaaggtggagtgttggagtatttctattgatcggtcatgtcatataggcccgagtcggacatccaattgcttcgatttgaagtatccggaggatgtctatattaagattatattaaatatatatagattatattaaaaagatggacaatcaaacctatttatttctcgattcaatagaagaaaaaaagaaaagaggtgaatagggtcccaaataacgagagatatgtaaaaagaaggcccgattacgcccattcctaatcctaaatggaatgtaacgacgtagggatccatatgtaaacatagtatctatttagatacgctcgaatgaccccttctcataatgagaatgtatatagccctattccggtctggtccggtatggaatgaacttataatcatggaatcgactcgatcatcagattatagattataagttcataaccccagcccattcccattttgggcggaacagatctactaattctttgattccagttagtaaagagggaccttgaactaagaaatagattctagaagctaactagaagctaaactaaaaaagggtatcctgagcaattgcaataatcgggttcattgatattcctggtatagt

SEQ ID NO: 212 - DNA, 107 bp, Gossypium hirsutum
aagaaaagaggtgaatagggtcccaaataacgagagatatgtaaaaagaaggcccgattacgcccattcctaatcctaaatggaatgtaacgacgtagggatccata

SEQ ID NO: 213 - DNA, 1498 bp, Gossypium hirsutum
aatctgttcctatctaacggaagactattggatcaaatgacaaagacattgttgagaaaaagatggcttttcccggatgaaatgaaaattggattcatgtaagaggagaaaggtttcccattacttagccggaaagatatgtggtcatgaaatagggattaagtggaacggaattgactgggtggtagagttgtggaaacacctgtttcttccatattttggaccttagctccatgaaacaatatgctactgctgaaacatggaagaattgaaatcttagatcaaaacactatgtatggatggtatgaactgcctaaacaagaattcttgaacagcgaacaaccagagctattactcactacatcaaaaaatttccattaatgaaagatgtaaatccattggaaaatcaaaaatacgcatgtcggatgaaatggttgttgctatctgctccaataacgaatcattggtttaactgaataactaaataaaatagatagacctttctcttcgtctcaggtcgatggatcttctcaactggaggatcccctatatggataatatatggataatacacattccagttgaccgagcctaattctattctaattgttttgttccgaagcaaagatatccacggggcggttcgccctattcagatattcacgaccgagaagtactggattctctttcggataggtcctgaaaggagaaggaaagctggaatgccaccaggcgtctattattgaattcacccgacccgatagtacccatttttggaacgtccagtgccaaagtcacggaatgggtaagtcgccaatccctaaaacggactatgtaatgtactttatccgctgggttacggggggggcattttaccagaggtttatattgtatcaatctacccttgtgtgcgtgattcctgttgaagcatatactcgggggtgggtgcagggcgaacgatttcaaagcggactccccattcattagatagagaagatccccaagatctcgtgatccgctgccgaacttattccaattcaatgagcattctcaatattatgccttgaagaggactcgaacctccacgctctttagcacgagattttgagtctcgcgtgtctaccatttcaccaccaaggcatcttgaaagtgaatcgtattccatgaatatgatatctatctagtgtgatgtatggaatatatgacaaaggtggagtgttggagtatttctattgatcggtcatgtcatataggcccgagtcggacatccaattgcttcgatttgaagtatccggaggatgtctatattaagattatattaaatatatatagattatattaaaaagatggacaatcaaacctatttatttctcgattcaatagaagaaaaaaagaaaagaggtgaatagggtcccaaataacgagagatatgtaaaaagaaggcccgattacgcccattcctaatcctaaatggaatgtaacgacgtagggatccata

SEQ ID NO: 214 - DNA, 428 bp, Gossypium hirsutum
aagaaaagaggtgaatagggtcccaaataacgagagatatgtaaaaagaaggcccgattacgcccattcctaatcctaaatggaatgtaacgacgtagggatccatatgtaaacatagtatctatttagatacgctcgaatgaccccttctcataatgagaatgtatatagccctattccggtctggtccggtatggaatgaacttataatcatggaatcgactcgatcatcagattatagattataagttcataaccccagcccattcccattttgggcggaacagatctactaattctttgattccagttagtaaagagggaccttgaactaagaaatagattctagaagctaactagaagctaaactaaaaaagggtatcctgagcaattgcaataatcgggttcattgatattcctggtatagt

SEQ ID NO: 215 - DNA, 749 bp, Gossypium hirsutum
tactcctgggaacagatccagtggagacgaggtggggcctgtagctcagaggattagagcacgtggctacgaaccacggtgtcgggggttcgaatccctcctcgcccacaaccggcccaaaagggaaggacctttccctctgggggtaggaaaatcatgatcgggatagcggacccaaagctatggaacttgggcgtgggtcttttgccgaaatggagtggccttttatttattatttatcgtatatttacttttcgattttatatttatatatagtattaccggccaaaacaaaataagcatattttttttgttttacgccccgtaactcttcctcagccaggcttgggcagaatagcagagcaagtacaagtattagtagcatagaaaaaatgcgctcctcgtcattaaattaatgtgtttgctcgcggtaattgtggcctctcgggagaatcgatgactgcatctttgaggcatttgctagtactagtacatctgagaattcttaattggctggttgtaaatagccccgggactatggaacaaaggattatcccggacacctacaccgaggtattgacggtgattctcaaatatcgcagaacagaatgtgatacgatgagatagaatgcaatagaaacaaagacacagggaacgggttacctgctcttaacggtcaaagcgaaccctttcattctgacattctgaattctttaattcggaatgaatcaaatctcctcaagtaggat

SEQ ID NO: 216 - DNA, 107 bp, Gossypium hirsutum
cccgtaactcttcctcagccaggcttgggcagaatagcagagcaagtacaagtattagtagcatagaaaaaatgcgctcctcgtcattaaattaatgtgtttgctcg

SEQ ID NO: 217 - DNA, 428 bp, Gossypium hirsutum
tactcctgggaacagatccagtggagacgaggtggggcctgtagctcagaggattagagcacgtggctacgaaccacggtgtcgggggttcgaatccctcctcgcccacaaccggcccaaaagggaaggacctttccctctgggggtaggaaaatcatgatcgggatagcggacccaaagctatggaacttgggcgtgggtcttttgccgaaatggagtggccttttatttattatttatcgtatatttacttttcgattttatatttatatatagtattaccggccaaaacaaaataagcatattttttttgttttacgccccgtaactcttcctcagccaggcttgggcagaatagcagagcaagtacaagtattagtagcatagaaaaaatgcgctcctcgtcattaaattaatgtgtttgctcg

SEQ ID NO: 218 - DNA, 428 bp, Gossypium hirsutum
cccgtaactcttcctcagccaggcttgggcagaatagcagagcaagtacaagtattagtagcatagaaaaaatgcgctcctcgtcattaaattaatgtgtttgctcgcggtaattgtggcctctcgggagaatcgatgactgcatctttgaggcatttgctagtactagtacatctgagaattcttaattggctggttgtaaatagccccgggactatggaacaaaggattatcccggacacctacaccgaggtattgacggtgattctcaaatatcgcagaacagaatgtgatacgatgagatagaatgcaatagaaacaaagacacagggaacgggttacctgctcttaacggtcaaagcgaaccctttcattctgacattctgaattctttaattcggaatgaatcaaatctcctcaagtaggat

SEQ ID NO: 219 - DNA, 1284 bp, Gossypium hirsutum
ttttagaagatactatgccgccactcggactcgaaccgagatgctctagcactgcttcctaagagcagcgtgtctaccgatttcaccatagcggcttgctcgaaatcataataacccattttttagtcaatcgtcgagattgaaggtgaaggggtcaattcaatgtaaaatgtcaaatttgttaggaagcatttaattttttattgataaataacaactcgttgaaatagcaatttgaaggttcaaaaggaatctttagtatttattgtatcattttatttatttaattatcctttctatttaattaggtcgtcaatagagatttttgtgttttcctaaaagagaactcctttattggaactaaactaactagttgtaacttcaattcatagataaaattcaacaaaacaaaaaagggttctttatcattttcattttttaatgattcatttctcattcttttgtatgatttttatgaatctataaaaaactataaaaaaatgcttattaattgactgaataaatgaatgaaaaaatatgatttttagagatcacaatttcagcaccacacccaacgatttcaaaagatttatgtaatttttgtattaatcgttagcattttgcgtttgttttaaggatttatcaaaccaactagatattgggttgtacccgttacgttatatgaaaagcgtttcgattcctgtcataattgtaccatacttcaaaaaagtatttcgaattctaaaaatatgtcttgatttattttattacattttcttatacaaacataggatttctttagatcacttcaaattgatacattatttgtatatccactaaattagaaaaggggttttctccattgggttgaaaacaggggaaggagatatagtaattcagagaaataatgattcataaagttacaaaattgaaacttaatgggttagtttcgacaatccagttaaagctcttatatatatgtgctccgcgatccgctatagtataaatagaaaaaaaaattcttattctattatattctattatttaattatttattattataaattgaatattataaaaataacaaattattataacactaacataacaaatacaaatgggcgatggggaaaataagaatcctccccccggaaatgaaaaaaaagatattcaaaaattcaaaaaagtaaaacctttgtatcctattcgagttaaaccaattcagattactccaaatttatttggcgtacaaaaaaactttttgaattccccgtagaaagagat

SEQ ID NO: 220 - DNA, 535 bp, Gossypium hirsutum
ttttcattttttaatgattcatttctcattcttttgtatgatttttatgaatctataaaaaactataaaaaaatgcttattaattgactgaataaatgaatgaaaaaatatgatttttagagatcacaatttcagcaccacacccaacgatttcaaaagatttatgtaatttttgtattaatcgttagcattttgcgtttgttttaaggatttatcaaaccaactagatattgggttgtacccgttacgttatatgaaaagcgtttcgattcctgtcataattgtaccatacttcaaaaaagtatttcgaattctaaaaatatgtcttgatttattttattacattttcttatacaaacataggatttctttagatcacttcaaattgatacattatttgtatatccactaaattagaaaaggggttttctccattgggttgaaaacaggggaaggagatatagtaattcagagaaataatgattcataaagttacaaaattgaaacttaatgggttagtttcgacaatccagtt

SEQ ID NO: 221 - DNA, 963 bp, Gossypium hirsutum
ttttagaagatactatgccgccactcggactcgaaccgagatgctctagcactgcttcctaagagcagcgtgtctaccgatttcaccatagcggcttgctcgaaatcataataacccattttttagtcaatcgtcgagattgaaggtgaaggggtcaattcaatgtaaaatgtcaaatttgttaggaagcatttaattttttattgataaataacaactcgttgaaatagcaatttgaaggttcaaaaggaatctttagtatttattgtatcattttatttatttaattatcctttctatttaattaggtcgtcaatagagatttttgtgttttcctaaaagagaactcctttattggaactaaactaactagttgtaacttcaattcatagataaaattcaacaaaacaaaaaagggttctttatcattttcattttttaatgattcatttctcattcttttgtatgatttttatgaatctataaaaaactataaaaaaatgcttattaattgactgaataaatgaatgaaaaaatatgatttttagagatcacaatttcagcaccacacccaacgatttcaaaagatttatgtaatttttgtattaatcgttagcattttgcgtttgttttaaggatttatcaaaccaactagatattgggttgtacccgttacgttatatgaaaagcgtttcgattcctgtcataattgtaccatacttcaaaaaagtatttcgaattctaaaaatatgtcttgatttattttattacattttcttatacaaacataggatttctttagatcacttcaaattgatacattatttgtatatccactaaattagaaaaggggttttctccattgggttgaaaacaggggaaggagatatagtaattcagagaaataatgattcataaagttacaaaattgaaacttaatgggttagtttcgacaatccagtt

SEQ ID NO: 222 - DNA, 856 bp, Gossypium hirsutum
ttttcattttttaatgattcatttctcattcttttgtatgatttttatgaatctataaaaaactataaaaaaatgcttattaattgactgaataaatgaatgaaaaaatatgatttttagagatcacaatttcagcaccacacccaacgatttcaaaagatttatgtaatttttgtattaatcgttagcattttgcgtttgttttaaggatttatcaaaccaactagatattgggttgtacccgttacgttatatgaaaagcgtttcgattcctgtcataattgtaccatacttcaaaaaagtatttcgaattctaaaaatatgtcttgatttattttattacattttcttatacaaacataggatttctttagatcacttcaaattgatacattatttgtatatccactaaattagaaaaggggttttctccattgggttgaaaacaggggaaggagatatagtaattcagagaaataatgattcataaagttacaaaattgaaacttaatgggttagtttcgacaatccagttaaagctcttatatatatgtgctccgcgatccgctatagtataaatagaaaaaaaaattcttattctattatattctattatttaattatttattattataaattgaatattataaaaataacaaattattataacactaacataacaaatacaaatgggcgatggggaaaataagaatcctccccccggaaatgaaaaaaaagatattcaaaaattcaaaaaagtaaaacctttgtatcctattcgagttaaaccaattcagattactccaaatttatttggcgtacaaaaaaactttttgaattccccgtagaaagagat

SEQ ID NO: 223 - DNA, 1635 bp, Zea mays
ctttctaccacatcgttttaaacgaagttttaccataacattcctctaatttcattgcaaaatggtatcgagaattgatccaatatggatggaatcatgaatagtcattgcttttgtatactaattcaaacctgctatctatggagaaaattggataaaagaaataagtatttatcggggaacactctgcaaagatacaatttatttaaacccatattctatcatatgaatgaaacatagttcgaaaaagaggaataaaatagtttacttaagacttatttattattattattgaatttccattttcaacagagaactcaagatgattaatcctgaaatgagaaggatccactctttcccaacaaataaactatcaacctcaaaaaaagtggaattaatttaatgaattaatctatttttctgtaacaaaaacaattaactatataaacaaataagctatgccaatgaaaagattggtcgtttttgagtagttctaaaattctcatacttcttcgactcgaataacaaaagaaagaactcaaaataaaaaataagaaaagtacgattttttcgaatccattctatccaacgaacagttcttaccttaaccttaccgaaatggatcattctggatatttaaagaatcacagatcgagatcgttttcgcttaaccaaagaaaagaaggacgcttcttttttactaataatacaagaaaacaaataatacaacaaaaatctatctctatcataaagggataggtctcattttctatacagtgttttacctcattaattttttttttttttacttactttagttctttagttttgggaaaataaatagggggtacttcttttctttcatattgggtgtcaaatggatcctgtaagaattcccacttcatagatacggggtataaagtttatccaaaaagttccaatttcaaaacacatattcaaaacacatatgagtaatgagtagtaggggcatatcctgaatgtgcctgatagacaaaaatttttcatgaaaataaagatattaaatttgactggacttgacactttattttttgttttctgagaaagaaaaaaatgcttagaaatgcatctaatctacgagttcataagagataattattctctttaataaacttttgtgtcgtgcagggcacaatacgattctatctttcgcttcatcagaaaaaatctgggacgaaaggattcgaacctccgagtaacgggaccaaaacccgctgccttaccacttggccacgccccatgtcatttcgttttatgaaacactaataaacactattatttttatatattattcgtcaatcccacttcaattacctaaaaaatgagggatattttcttgctaggattctagacatgcagataatatagaatccaaaaaatgcattgatcattacatggaattctattaagatattatatgaaagtcgaatttcttccattctcatttgagaatgcgaatacaaggaggtattttgtgtttgggaaagtccgaagaaaaaaggatttggaatctaccttttcttttgctttttcccttataaaaataactcaatcaaaatccaattatctactctacaagaatgaaatgcttgttat

SEQ ID NO: 224 - DNA, 545 bp, Zea mays
taactatataaacaaataagctatgccaatgaaaagattggtcgtttttgagtagttctaaaattctcatacttcttcgactcgaataacaaaagaaagaactcaaaataaaaaataagaaaagtacgattttttcgaatccattctatccaacgaacagttcttaccttaaccttaccgaaatggatcattctggatatttaaagaatcacagatcgagatcgttttcgcttaaccaaagaaaagaaggacgcttcttttttactaataatacaagaaaacaaataatacaacaaaaatctatctctatcataaagggataggtctcattttctatacagtgttttacctcattaattttttttttttttacttactttagttctttagttttgggaaaataaatagggggtacttcttttctttcatattgggtgtcaaatggatcctgtaagaattcccacttcatagatacggggtataaagtttatccaaaaagttccaatttcaaaacacatattcaaaacacatatgagtaatgagta

SEQ ID NO: 225 - DNA, 981 bp, Zea mays
ctttctaccacatcgttttaaacgaagttttaccataacattcctctaatttcattgcaaaatggtatcgagaattgatccaatatggatggaatcatgaatagtcattgcttttgtatactaattcaaacctgctatctatggagaaaattggataaaagaaataagtatttatcggggaacactctgcaaagatacaatttatttaaacccatattctatcatatgaatgaaacatagttcgaaaaagaggaataaaatagtttacttaagacttatttattattattattgaatttccattttcaacagagaactcaagatgattaatcctgaaatgagaaggatccactctttcccaacaaataaactatcaacctcaaaaaaagtggaattaatttaatgaattaatctatttttctgtaacaaaaacaattaactatataaacaaataagctatgccaatgaaaagattggtcgtttttgagtagttctaaaattctcatacttcttcgactcgaataacaaaagaaagaactcaaaataaaaaataagaaaagtacgattttttcgaatccattctatccaacgaacagttcttaccttaaccttaccgaaatggatcattctggatatttaaagaatcacagatcgagatcgttttcgcttaaccaaagaaaagaaggacgcttcttttttactaataatacaagaaaacaaataatacaacaaaaatctatctctatcataaagggataggtctcattttctatacagtgttttacctcattaattttttttttttttacttactttagttctttagttttgggaaaataaatagggggtacttcttttctttcatattgggtgtcaaatggatcctgtaagaattcccacttcatagatacggggtataaagtttatccaaaaagttccaatttcaaaacacatattcaaaacacatatgagtaatgagta

SEQ ID NO: 226 - DNA, 1199 bp, Zea mays
taactatataaacaaataagctatgccaatgaaaagattggtcgtttttgagtagttctaaaattctcatacttcttcgactcgaataacaaaagaaagaactcaaaataaaaaataagaaaagtacgattttttcgaatccattctatccaacgaacagttcttaccttaaccttaccgaaatggatcattctggatatttaaagaatcacagatcgagatcgttttcgcttaaccaaagaaaagaaggacgcttcttttttactaataatacaagaaaacaaataatacaacaaaaatctatctctatcataaagggataggtctcattttctatacagtgttttacctcattaattttttttttttttacttactttagttctttagttttgggaaaataaatagggggtacttcttttctttcatattgggtgtcaaatggatcctgtaagaattcccacttcatagatacggggtataaagtttatccaaaaagttccaatttcaaaacacatattcaaaacacatatgagtaatgagtagtaggggcatatcctgaatgtgcctgatagacaaaaatttttcatgaaaataaagatattaaatttgactggacttgacactttattttttgttttctgagaaagaaaaaaatgcttagaaatgcatctaatctacgagttcataagagataattattctctttaataaacttttgtgtcgtgcagggcacaatacgattctatctttcgcttcatcagaaaaaatctgggacgaaaggattcgaacctccgagtaacgggaccaaaacccgctgccttaccacttggccacgccccatgtcatttcgttttatgaaacactaataaacactattatttttatatattattcgtcaatcccacttcaattacctaaaaaatgagggatattttcttgctaggattctagacatgcagataatatagaatccaaaaaatgcattgatcattacatggaattctattaagatattatatgaaagtcgaatttcttccattctcatttgagaatgcgaatacaaggaggtattttgtgtttgggaaagtccgaagaaaaaaggatttggaatctaccttttcttttgctttttcccttataaaaataactcaatcaaaatccaattatctactctacaagaatgaaatgcttgttat

SEQ ID NO: 227 - DNA, 1090 bp, Zea mays
cgctttagtccactcagccatctctccccgttccaaatcgaaaggttttcatgatatggcagaggcaagaaataacgattgtcaaaaatccttcctttttctttcatttcaaaagtgcataaaaattatattgccaattccattttaattatattcttttttcttaatgttaataaaaaaaagaagaaaattctggttttttctttccaaaattcgatataggctgaaagacaatcagatcttttttctcttcagcgggcagttccatatagaatttgttagaataaaacaagcgggttaaaaaaaaaaactctttttttttatttatccacaaagcaaaaaaggttcttatcaaacccacaataaaatgggaaagaaagataaagtaagtagacctgaccccttgaataatgcctctatctgctattctgatatataaattcgatgtggatgaaattggtgtataagcgcatttttgtatttccttagacttagatcgcgcaaggcaagaatttttcgctatttacgatttcatattcttgttactagacgttctataggaataagaagaaatcgcaactcttttccgttacacataataaataaagggtttcaaaagtcaatttttcttttcaatatctttcttttttttttcagaattccatttttgttcttccacccatgcaatagagagcgaatgagaaaagaggggttttcccttaaaaaatgggctttgaaatagaaatcatggaataatgctgaattcaaatgtttatttctttatttcttttctatagtataagaaaaatgaattaaatgaaatttgtggatttagcacgacctgggttgtgaccccatagataaaaatgcaaaatttctatcttcgagacgactgaaaaagggcattgaacgagaaagaaatcgtccacagataatcaaactatcgtatgccctggaagtaatatgaggtgctcgaaaatggttgaagtaattgaataggaggatcactatgactatagccgttggtagagttactaaagaagaaaacgatctatttgatattatggacgactggttacgaagggaccgt

SEQ ID NO: 228 - DNA, 436 bp, Zea mays
ataggctgaaagacaatcagatcttttttctcttcagcgggcagttccatatagaatttgttagaataaaacaagcgggttaaaaaaaaaaactctttttttttatttatccacaaagcaaaaaaggttcttatcaaacccacaataaaatgggaaagaaagataaagtaagtagacctgaccccttgaataatgcctctatctgctattctgatatataaattcgatgtggatgaaattggtgtataagcgcatttttgtatttccttagacttagatcgcgcaaggcaagaatttttcgctatttacgatttcatattcttgttactagacgttctataggaataagaagaaatcgcaactcttttccgttacacataataaataaagggtttcaaaagtcaatttttcttttcaatatctttctttttttttt

SEQ ID NO: 229 - DNA, 654 bp, Zea mays
cgctttagtccactcagccatctctccccgttccaaatcgaaaggttttcatgatatggcagaggcaagaaataacgattgtcaaaaatccttcctttttctttcatttcaaaagtgcataaaaattatattgccaattccattttaattatattcttttttcttaatgttaataaaaaaaagaagaaaattctggttttttctttccaaaattcgatataggctgaaagacaatcagatcttttttctcttcagcgggcagttccatatagaatttgttagaataaaacaagcgggttaaaaaaaaaaactctttttttttatttatccacaaagcaaaaaaggttcttatcaaacccacaataaaatgggaaagaaagataaagtaagtagacctgaccccttgaataatgcctctatctgctattctgatatataaattcgatgtggatgaaattggtgtataagcgcatttttgtatttccttagacttagatcgcgcaaggcaagaatttttcgctatttacgatttcatattcttgttactagacgttctataggaataagaagaaatcgcaactcttttccgttacacataataaataaagggtttcaaaagtcaatttttcttttcaatatctttctttttttttt

SEQ ID NO: 230 - DNA, 872 bp, Zea mays
ataggctgaaagacaatcagatcttttttctcttcagcgggcagttccatatagaatttgttagaataaaacaagcgggttaaaaaaaaaaactctttttttttatttatccacaaagcaaaaaaggttcttatcaaacccacaataaaatgggaaagaaagataaagtaagtagacctgaccccttgaataatgcctctatctgctattctgatatataaattcgatgtggatgaaattggtgtataagcgcatttttgtatttccttagacttagatcgcgcaaggcaagaatttttcgctatttacgatttcatattcttgttactagacgttctataggaataagaagaaatcgcaactcttttccgttacacataataaataaagggtttcaaaagtcaatttttcttttcaatatctttcttttttttttcagaattccatttttgttcttccacccatgcaatagagagcgaatgagaaaagaggggttttcccttaaaaaatgggctttgaaatagaaatcatggaataatgctgaattcaaatgtttatttctttatttcttttctatagtataagaaaaatgaattaaatgaaatttgtggatttagcacgacctgggttgtgaccccatagataaaaatgcaaaatttctatcttcgagacgactgaaaaagggcattgaacgagaaagaaatcgtccacagataatcaaactatcgtatgccctggaagtaatatgaggtgctcgaaaatggttgaagtaattgaataggaggatcactatgactatagccgttggtagagttactaaagaagaaaacgatctatttgatattatggacgactggttacgaagggaccgt

SEQ ID NO: 231 - DNA, 872 bp, Zea mays
gagagagagggattcgaaccctcgatagttcctagaactataccggttttcaagaccggagctatcaaccactcagccatctctccacagcccaatccctattttattcctacaaatagaacatagctatatgaaatgatctactaactcatctcagatgcaagtccactttcaatatatctctgtataaagcggtaaggaaaaagttttaataaaaagaagaatcaatggattcatgattaaacccctcctacttcttgtattttcatacaattttgattaagtgagggatcaaatagaaatatgtagtcaactttatttgaaggtagtttggaggattataaatatgaatattgctttccaattagctgtttttgcattaattgcgacttcctcagttttagtaattagtgtaccccttgtatttgcttctcctgatggttggtcaaataataaaaacgttgtattttccggtacatcattatggattggattagtctttctggtagctattctgaattctcttatttcttgaatttgtttagtatttagtaacccgatacaaaataaataaaaagaaaggccctttttcgaaaaaattaggatttttatacacattaaaatgctattttcgttccgaattattacctcttctctttcattattatgaaattctattgccattagaatattgattgatagacagttaataaaaagaaaactctaatagaaaatgaaacggtcgacccagacatagacggtcgacccagacatagacggtcgacccaggtggatataccctataaaatataggacgtagcaagcgtagttcaatgtagcgagcgtagttcagtggtaaaacatctccttgc

SEQ ID NO: 232 - DNA, 327 bp, Zea mays
gaagaatcaatggattcatgattaaacccctcctacttcttgtattttcatacaattttgattaagtgagggatcaaatagaaatatgtagtcaactttatttgaaggtagtttggaggattataaatatgaatattgctttccaattagctgtttttgcattaattgcgacttcctcagttttagtaattagtgtaccccttgtatttgcttctcctgatggttggtcaaataataaaaacgttgtattttccggtacatcattatggattggattagtctttctggtagctattctgaattctcttatttcttgaatttgtttag

SEQ ID NO: 233 - DNA, 545 bp, Zea mays
gagagagagggattcgaaccctcgatagttcctagaactataccggttttcaagaccggagctatcaaccactcagccatctctccacagcccaatccctattttattcctacaaatagaacatagctatatgaaatgatctactaactcatctcagatgcaagtccactttcaatatatctctgtataaagcggtaaggaaaaagttttaataaaaagaagaatcaatggattcatgattaaacccctcctacttcttgtattttcatacaattttgattaagtgagggatcaaatagaaatatgtagtcaactttatttgaaggtagtttggaggattataaatatgaatattgctttccaattagctgtttttgcattaattgcgacttcctcagttttagtaattagtgtaccccttgtatttgcttctcctgatggttggtcaaataataaaaacgttgtattttccggtacatcattatggattggattagtctttctggtagctattctgaattctcttatttcttgaatttgtttag

SEQ ID NO: 234 - DNA, 654 bp, Zea mays
gaagaatcaatggattcatgattaaacccctcctacttcttgtattttcatacaattttgattaagtgagggatcaaatagaaatatgtagtcaactttatttgaaggtagtttggaggattataaatatgaatattgctttccaattagctgtttttgcattaattgcgacttcctcagttttagtaattagtgtaccccttgtatttgcttctcctgatggttggtcaaataataaaaacgttgtattttccggtacatcattatggattggattagtctttctggtagctattctgaattctcttatttcttgaatttgtttagtatttagtaacccgatacaaaataaataaaaagaaaggccctttttcgaaaaaattaggatttttatacacattaaaatgctattttcgttccgaattattacctcttctctttcattattatgaaattctattgccattagaatattgattgatagacagttaataaaaagaaaactctaatagaaaatgaaacggtcgacccagacatagacggtcgacccagacatagacggtcgacccaggtggatataccctataaaatataggacgtagcaagcgtagttcaatgtagcgagcgtagttcagtggtaaaacatctccttgc

SEQ ID NO: 235 - DNA, 2834 bp, Zea mays
ccgtgacctcaaggttatgagcctcgtgagctaccaaactgctctactccgctctgtagggccgaaaactggtggacgaaagaaaaaggtggaatacaagtctctaccatgtctagacaaacaaatggaatagtctttttatacagaatggagcgggtagcgggaatcgaacccgcatcgttagcttggaaggctaggggttatagtcgacgttggttgattatttttgacgtctctaattcaaaaccgaacatgaaattttgatttcattcggctcctttatggatattctcaccacttaacatctatgtcagcttttctgtctgaatggaaccaaagctctccgctttctagatgatccctatagaataggagatagaaattctcctaaatatctatctaatctacttacttcgttccctaatttcattcaagagatcctgaggaaaagagttgggtttccaccgagctgaaacaatataatatactgatggttctagtaaaccaaaaccatcgttttttagctattgggcttccatttcctacaaaacaaaagaagatttagttacgattggaaataaccttttttgtatcttcatccatagatcctttactcatatttattcaatcggaatacttatcggaatacttaatccaatgcaaaattatgcttcgcgactacgtactcataatcgaatttgtattttagatgcaaattcaattagtctttggatactaatcgggagaatgtatattcttcctcaatatgctattgagaggaaaaggattaaatcctttataagaactaaagttttcatcggaatatgaatataaaaaaacttaaggatgccttaagtatatcatttcaaattcagttattaatagaacgaatcacacttttaccactaaactatacccgctacatgtagattatgataccaatgctaccctttgtcaagggtagccattcgagaaggaggctaattccaccttatcgaatcaaaggagaaagttcatggcggtgggcggttggtacttcaatcgcggacctttactttaggatttagatagcccctctctagtctgtaaaatacatctcttcttaccataccaatagcgtatgaaccaaatgtatgcatttcgattaggatctattctacggttatgactacaaggatcattatttgtgaggacgtaaatgtgccagactgttgtaaggaatcgtttaattattcctacaatatatactaagagatataaaggcagtacagtccccataaatccctttcttccttttcttttttgttcagaattgaacaaagaaattgcgaaagatgttttcttcctccacgtatcatgaagtgcgagccatagggaaggagtgagatgactttccaaaatttttcatagacttcgtctatcgcttgagagaagcaacaaagagtaataacttaaaaagaaaagcagatacgaatcgctggttagtcgatcctctccatttcctaatttcctctcttcttttccgctcaattctagtttattagattcttgtttaaaagaatcaaagaagatgaatagaactaagaacacataaaaaagagcatagagaaccattaccaaacgttcctcctaagaatcatattgggtatctatccccttccttttaaccctaggatcgaaaatctagaatcctcccttttcctagtgttcggaaacggaaaaccctgaacctggaggagttaggtatgtaggatatgctttttattttttgttggacaggcagtagtataattttgatactctgcagtataaattcgactcccaacccttttttttagaataaaattgttgataaaactccaacatagtttgttccaaatccactataatccttgtagaatagtcaagtaccccatttccaaggggtacctgttgaaaatcgtttcaacaaatccgtccaaaactttttaagaaaaatgaaaaagttttgaactcgaaagtttttccaaaagatgcctgctaaaaattgtttcaatctctcaccaaaacagataagaagtatatcactgaaaattactatccagccataggggtatatgaagagcgcaaattcctttataccccacccaattagaattaggggaaataaaacataaatggagaaagttctcatcataagatcagccaatagaagaaaaaagtccctaattctgcagaacattctgagcacgtgaaaagtgaatagcctaaagataaaaaaaaagtttatcatttttttaacagcagttcttattgcccctttggcctttttggcccattgttgtatccgattcaacaattgatacatatttgttctcttcttaacaaaaaaaatggaacttccggactttggtttggtgagtcgtacgagatcgtgtttacatacctatgaacttagcctcttcaagtgtatccgatatatataataatttttgggtgtgtcaattctctgttcatgtattttattatacatgatatgtatatgtatttatttatataataataaatacctatactttgtgcaagtgataagagataatatgaaagattttcttatttttcttaattttctcaagattttaaacctcgccataaataaacttcgatatctcgatctcgatatatagaaaagaaaatctctacatatctctatatatacatatattatgtacattaatatatacatgtaatatgtacattatgcagtagacccataatggtaaatgaaagtggctaattattgaataaaaagcccttttaactcagtggtagagtaatgccatggtaaggcataa

SEQ ID NO: 236 - DNA, 2398 bp, Zea mays
gattatttttgacgtctctaattcaaaaccgaacatgaaattttgatttcattcggctcctttatggatattctcaccacttaacatctatgtcagcttttctgtctgaatggaaccaaagctctccgctttctagatgatccctatagaataggagatagaaattctcctaaatatctatctaatctacttacttcgttccctaatttcattcaagagatcctgaggaaaagagttgggtttccaccgagctgaaacaatataatatactgatggttctagtaaaccaaaaccatcgttttttagctattgggcttccatttcctacaaaacaaaagaagatttagttacgattggaaataaccttttttgtatcttcatccatagatcctttactcatatttattcaatcggaatacttatcggaatacttaatccaatgcaaaattatgcttcgcgactacgtactcataatcgaatttgtattttagatgcaaattcaattagtctttggatactaatcgggagaatgtatattcttcctcaatatgctattgagaggaaaaggattaaatcctttataagaactaaagttttcatcggaatatgaatataaaaaaacttaaggatgccttaagtatatcatttcaaattcagttattaatagaacgaatcacacttttaccactaaactatacccgctacatgtagattatgataccaatgctaccctttgtcaagggtagccattcgagaaggaggctaattccaccttatcgaatcaaaggagaaagttcatggcggtgggcggttggtacttcaatcgcggacctttactttaggatttagatagcccctctctagtctgtaaaatacatctcttcttaccataccaatagcgtatgaaccaaatgtatgcatttcgattaggatctattctacggttatgactacaaggatcattatttgtgaggacgtaaatgtgccagactgttgtaaggaatcgtttaattattcctacaatatatactaagagatataaaggcagtacagtccccataaatccctttcttccttttcttttttgttcagaattgaacaaagaaattgcgaaagatgttttcttcctccacgtatcatgaagtgcgagccatagggaaggagtgagatgactttccaaaatttttcatagacttcgtctatcgcttgagagaagcaacaaagagtaataacttaaaaagaaaagcagatacgaatcgctggttagtcgatcctctccatttcctaatttcctctcttcttttccgctcaattctagtttattagattcttgtttaaaagaatcaaagaagatgaatagaactaagaacacataaaaaagagcatagagaaccattaccaaacgttcctcctaagaatcatattgggtatctatccccttccttttaaccctaggatcgaaaatctagaatcctcccttttcctagtgttcggaaacggaaaaccctgaacctggaggagttaggtatgtaggatatgctttttattttttgttggacaggcagtagtataattttgatactctgcagtataaattcgactcccaacccttttttttagaataaaattgttgataaaactccaacatagtttgttccaaatccactataatccttgtagaatagtcaagtaccccatttccaaggggtacctgttgaaaatcgtttcaacaaatccgtccaaaactttttaagaaaaatgaaaaagttttgaactcgaaagtttttccaaaagatgcctgctaaaaattgtttcaatctctcaccaaaacagataagaagtatatcactgaaaattactatccagccataggggtatatgaagagcgcaaattcctttataccccacccaattagaattaggggaaataaaacataaatggagaaagttctcatcataagatcagccaatagaagaaaaaagtccctaattctgcagaacattctgagcacgtgaaaagtgaatagcctaaagataaaaaaaaagtttatcatttttttaacagcagttcttattgcccctttggcctttttggcccattgttgtatccgattcaacaattgatacatatttgttctcttcttaacaaaaaaaatggaacttccggactttggtttggtgagtcgtacgagatcgtgtttacatacctatgaacttagcctcttcaagtgtatccgatatatataataatttttgggtgtgtcaattctctgttcatgtattttattatacatgatatgtatatgtatttatttatataataataaatacctatactttgtgcaagtgataagagataatatgaaagattttcttatttttcttaattttctcaaga

SEQ ID NO: 237 - DNA, 2616 bp, Zea mays
ccgtgacctcaaggttatgagcctcgtgagctaccaaactgctctactccgctctgtagggccgaaaactggtggacgaaagaaaaaggtggaatacaagtctctaccatgtctagacaaacaaatggaatagtctttttatacagaatggagcgggtagcgggaatcgaacccgcatcgttagcttggaaggctaggggttatagtcgacgttggttgattatttttgacgtctctaattcaaaaccgaacatgaaattttgatttcattcggctcctttatggatattctcaccacttaacatctatgtcagcttttctgtctgaatggaaccaaagctctccgctttctagatgatccctatagaataggagatagaaattctcctaaatatctatctaatctacttacttcgttccctaatttcattcaagagatcctgaggaaaagagttgggtttccaccgagctgaaacaatataatatactgatggttctagtaaaccaaaaccatcgttttttagctattgggcttccatttcctacaaaacaaaagaagatttagttacgattggaaataaccttttttgtatcttcatccatagatcctttactcatatttattcaatcggaatacttatcggaatacttaatccaatgcaaaattatgcttcgcgactacgtactcataatcgaatttgtattttagatgcaaattcaattagtctttggatactaatcgggagaatgtatattcttcctcaatatgctattgagaggaaaaggattaaatcctttataagaactaaagttttcatcggaatatgaatataaaaaaacttaaggatgccttaagtatatcatttcaaattcagttattaatagaacgaatcacacttttaccactaaactatacccgctacatgtagattatgataccaatgctaccctttgtcaagggtagccattcgagaaggaggctaattccaccttatcgaatcaaaggagaaagttcatggcggtgggcggttggtacttcaatcgcggacctttactttaggatttagatagcccctctctagtctgtaaaatacatctcttcttaccataccaatagcgtatgaaccaaatgtatgcatttcgattaggatctattctacggttatgactacaaggatcattatttgtgaggacgtaaatgtgccagactgttgtaaggaatcgtttaattattcctacaatatatactaagagatataaaggcagtacagtccccataaatccctttcttccttttcttttttgttcagaattgaacaaagaaattgcgaaagatgttttcttcctccacgtatcatgaagtgcgagccatagggaaggagtgagatgactttccaaaatttttcatagacttcgtctatcgcttgagagaagcaacaaagagtaataacttaaaaagaaaagcagatacgaatcgctggttagtcgatcctctccatttcctaatttcctctcttcttttccgctcaattctagtttattagattcttgtttaaaagaatcaaagaagatgaatagaactaagaacacataaaaaagagcatagagaaccattaccaaacgttcctcctaagaatcatattgggtatctatccccttccttttaaccctaggatcgaaaatctagaatcctcccttttcctagtgttcggaaacggaaaaccctgaacctggaggagttaggtatgtaggatatgctttttattttttgttggacaggcagtagtataattttgatactctgcagtataaattcgactcccaacccttttttttagaataaaattgttgataaaactccaacatagtttgttccaaatccactataatccttgtagaatagtcaagtaccccatttccaaggggtacctgttgaaaatcgtttcaacaaatccgtccaaaactttttaagaaaaatgaaaaagttttgaactcgaaagtttttccaaaagatgcctgctaaaaattgtttcaatctctcaccaaaacagataagaagtatatcactgaaaattactatccagccataggggtatatgaagagcgcaaattcctttataccccacccaattagaattaggggaaataaaacataaatggagaaagttctcatcataagatcagccaatagaagaaaaaagtccctaattctgcagaacattctgagcacgtgaaaagtgaatagcctaaagataaaaaaaaagtttatcatttttttaacagcagttcttattgcccctttggcctttttggcccattgttgtatccgattcaacaattgatacatatttgttctcttcttaacaaaaaaaatggaacttccggactttggtttggtgagtcgtacgagatcgtgtttacatacctatgaacttagcctcttcaagtgtatccgatatatataataatttttgggtgtgtcaattctctgttcatgtattttattatacatgatatgtatatgtatttatttatataataataaatacctatactttgtgcaagtgataagagataatatgaaagattttcttatttttcttaattttctcaaga

SEQ ID NO: 238 - DNA, 2616 bp, Zea mays
gattatttttgacgtctctaattcaaaaccgaacatgaaattttgatttcattcggctcctttatggatattctcaccacttaacatctatgtcagcttttctgtctgaatggaaccaaagctctccgctttctagatgatccctatagaataggagatagaaattctcctaaatatctatctaatctacttacttcgttccctaatttcattcaagagatcctgaggaaaagagttgggtttccaccgagctgaaacaatataatatactgatggttctagtaaaccaaaaccatcgttttttagctattgggcttccatttcctacaaaacaaaagaagatttagttacgattggaaataaccttttttgtatcttcatccatagatcctttactcatatttattcaatcggaatacttatcggaatacttaatccaatgcaaaattatgcttcgcgactacgtactcataatcgaatttgtattttagatgcaaattcaattagtctttggatactaatcgggagaatgtatattcttcctcaatatgctattgagaggaaaaggattaaatcctttataagaactaaagttttcatcggaatatgaatataaaaaaacttaaggatgccttaagtatatcatttcaaattcagttattaatagaacgaatcacacttttaccactaaactatacccgctacatgtagattatgataccaatgctaccctttgtcaagggtagccattcgagaaggaggctaattccaccttatcgaatcaaaggagaaagttcatggcggtgggcggttggtacttcaatcgcggacctttactttaggatttagatagcccctctctagtctgtaaaatacatctcttcttaccataccaatagcgtatgaaccaaatgtatgcatttcgattaggatctattctacggttatgactacaaggatcattatttgtgaggacgtaaatgtgccagactgttgtaaggaatcgtttaattattcctacaatatatactaagagatataaaggcagtacagtccccataaatccctttcttccttttcttttttgttcagaattgaacaaagaaattgcgaaagatgttttcttcctccacgtatcatgaagtgcgagccatagggaaggagtgagatgactttccaaaatttttcatagacttcgtctatcgcttgagagaagcaacaaagagtaataacttaaaaagaaaagcagatacgaatcgctggttagtcgatcctctccatttcctaatttcctctcttcttttccgctcaattctagtttattagattcttgtttaaaagaatcaaagaagatgaatagaactaagaacacataaaaaagagcatagagaaccattaccaaacgttcctcctaagaatcatattgggtatctatccccttccttttaaccctaggatcgaaaatctagaatcctcccttttcctagtgttcggaaacggaaaaccctgaacctggaggagttaggtatgtaggatatgctttttattttttgttggacaggcagtagtataattttgatactctgcagtataaattcgactcccaacccttttttttagaataaaattgttgataaaactccaacatagtttgttccaaatccactataatccttgtagaatagtcaagtaccccatttccaaggggtacctgttgaaaatcgtttcaacaaatccgtccaaaactttttaagaaaaatgaaaaagttttgaactcgaaagtttttccaaaagatgcctgctaaaaattgtttcaatctctcaccaaaacagataagaagtatatcactgaaaattactatccagccataggggtatatgaagagcgcaaattcctttataccccacccaattagaattaggggaaataaaacataaatggagaaagttctcatcataagatcagccaatagaagaaaaaagtccctaattctgcagaacattctgagcacgtgaaaagtgaatagcctaaagataaaaaaaaagtttatcatttttttaacagcagttcttattgcccctttggcctttttggcccattgttgtatccgattcaacaattgatacatatttgttctcttcttaacaaaaaaaatggaacttccggactttggtttggtgagtcgtacgagatcgtgtttacatacctatgaacttagcctcttcaagtgtatccgatatatataataatttttgggtgtgtcaattctctgttcatgtattttattatacatgatatgtatatgtatttatttatataataataaatacctatactttgtgcaagtgataagagataatatgaaagattttcttatttttcttaattttctcaagattttaaacctcgccataaataaacttcgatatctcgatctcgatatatagaaaagaaaatctctacatatctctatatatacatatattatgtacattaatatatacatgtaatatgtacattatgcagtagacccataatggtaaatgaaagtggctaattattgaataaaaagcccttttaactcagtggtagagtaatgccatggtaaggcataa

SEQ ID NO: 239 - DNA, 1199 bp, Zea mays
ctatttaagaataggcatgccatacaccccgcagggattgtagttcaattggtcagagcaccgccctgtcaaggcggaagctgcgggttcgagccccgtcagtcccgaactagggttcaatgaatggacaaattcatcttttcttttttcatggaaatttttgatgaaaaaaggggggggggggacaggaggcaagatcaaatatctatggggcacccttacttcacttttatttttttgcatttctcagtaaaaagagagcagtgcagtatagaattttttttgcactacttccggttgataaggaaaggcatacatatcatacgtggattagtataatttaggatactactccatttttatgatgataccaccctcatcttaacttcctattcgactcttatgaaatcgagtaccggtattttaccggaatccatacataaaccgtattcgtttattgttagagaatgaatttcatctaattagttactttttgggggttaaaccgcaccccttccattttctagttccaactttgtttgtgtatataattggaaagaatccacctcattctcactccatttgccgactcctttttttatggatttgctctcatctttagaccccaaaaagcgtatatggatagtaacctaataaaataaaaaccaagcaaacgctctttttcctaaatttaaatatgggatcctttttatttaagatcctcttttctccatctcatttctacttctactgcttattgggatgtctatgtgacccatagaaagttggtcatataatacatacataattgtatgtataactataagaaaaaggaagtgaaattggataagataagaaagattttgggttatacatatagaaaagcaaaagtagaaaagaatgaaaaaagaatagagggggtttcgaatccaatcaaaaaacctattttggtcttagtatggataagagatcttcctatgttatattattccactatccaccatgaattgatttgatagatgcaatattcataatattgaattgattcagtattatcagaatgcaagtcctccccttgaatttacaggatacccctttttcctctccatgggattacatcccgagttattgtgaaaaaaataaataaataaagaggttatggaagtaaatattctcgcatttattgctacagcattgtt

SEQ ID NO: 240 - DNA, 545 bp, Zea mays
ttacttcacttttatttttttgcatttctcagtaaaaagagagcagtgcagtatagaattttttttgcactacttccggttgataaggaaaggcatacatatcatacgtggattagtataatttaggatactactccatttttatgatgataccaccctcatcttaacttcctattcgactcttatgaaatcgagtaccggtattttaccggaatccatacataaaccgtattcgtttattgttagagaatgaatttcatctaattagttactttttgggggttaaaccgcaccccttccattttctagttccaactttgtttgtgtatataattggaaagaatccacctcattctcactccatttgccgactcctttttttatggatttgctctcatctttagaccccaaaaagcgtatatggatagtaacctaataaaataaaaaccaagcaaacgctctttttcctaaatttaaatatgggatcctttttatttaagatcctcttttctccatctcatttctacttctactgcttattggga

SEQ ID NO: 241 - DNA, 763 bp, Zea mays
ctatttaagaataggcatgccatacaccccgcagggattgtagttcaattggtcagagcaccgccctgtcaaggcggaagctgcgggttcgagccccgtcagtcccgaactagggttcaatgaatggacaaattcatcttttcttttttcatggaaatttttgatgaaaaaaggggggggggggacaggaggcaagatcaaatatctatggggcacccttacttcacttttatttttttgcatttctcagtaaaaagagagcagtgcagtatagaattttttttgcactacttccggttgataaggaaaggcatacatatcatacgtggattagtataatttaggatactactccatttttatgatgataccaccctcatcttaacttcctattcgactcttatgaaatcgagtaccggtattttaccggaatccatacataaaccgtattcgtttattgttagagaatgaatttcatctaattagttactttttgggggttaaaccgcaccccttccattttctagttccaactttgtttgtgtatataattggaaagaatccacctcattctcactccatttgccgactcctttttttatggatttgctctcatctttagaccccaaaaagcgtatatggatagtaacctaataaaataaaaaccaagcaaacgctctttttcctaaatttaaatatgggatcctttttatttaagatcctcttttctccatctcatttctacttctactgcttattggga

SEQ ID NO: 242 - DNA, 981 bp, Zea mays
ttacttcacttttatttttttgcatttctcagtaaaaagagagcagtgcagtatagaattttttttgcactacttccggttgataaggaaaggcatacatatcatacgtggattagtataatttaggatactactccatttttatgatgataccaccctcatcttaacttcctattcgactcttatgaaatcgagtaccggtattttaccggaatccatacataaaccgtattcgtttattgttagagaatgaatttcatctaattagttactttttgggggttaaaccgcaccccttccattttctagttccaactttgtttgtgtatataattggaaagaatccacctcattctcactccatttgccgactcctttttttatggatttgctctcatctttagaccccaaaaagcgtatatggatagtaacctaataaaataaaaaccaagcaaacgctctttttcctaaatttaaatatgggatcctttttatttaagatcctcttttctccatctcatttctacttctactgcttattgggatgtctatgtgacccatagaaagttggtcatataatacatacataattgtatgtataactataagaaaaaggaagtgaaattggataagataagaaagattttgggttatacatatagaaaagcaaaagtagaaaagaatgaaaaaagaatagagggggtttcgaatccaatcaaaaaacctattttggtcttagtatggataagagatcttcctatgttatattattccactatccaccatgaattgatttgatagatgcaatattcataatattgaattgattcagtattatcagaatgcaagtcctccccttgaatttacaggatacccctttttcctctccatgggattacatcccgagttattgtgaaaaaaataaataaataaagaggttatggaagtaaatattctcgcatttattgctacagcattgtt

SEQ ID NO: 243 - DNA, 872 bp, Zea mays
cattctagttcctactgcctttttacttattatttatgtaaaaacagccagtcaaaatgattaattggaattccaattaatcattgaagaaatgaaaaagggattaaagaaaataaaaaatccaagtcttaaatgaaagggtctggttggaatcataaagtgtggtagaaagaactacatatagttttttctaccacactttagattctttctattatattatcttgaatctacatagaatcgattagtagattgaaatagtagtctaattcaacttcttttttcactgcatccacttaatttcaatcaagtcaaaatcaaaaaaatcgaagataattcttcattgaaagaatccaaggagggggagaaaaataatacgagaatagactagaatagattatagtaaaagaaaaaaagtaaaaggaaaaaacctacgaatctttcatgcttaaaaaaggtgcgagatgcttcaatcgagatccttcaaaaaaaaaaaaaaagaacatcaaaaattttctaagaataagaaaatagtataaatggaaaatgtgcgatatattgtgaatagcttcgtgtaagaaagtctaattttcttatgttctaattttcttatgtatagaactttctttttactcataatttctagtagaatagaagttatgaattactataatcgccctttctattctattatactggaatagaacgactctttcttactttcttaaaagagtttcttacaagagtgaaacaaaactaaagaaagagagaaaaaataaagtttggcaaaattattaatgcaaaacgatcaattaaagaaaagagttgatactacaattcgactactcaatcaattggtagtatccctagag

SEQ ID NO: 244 - DNA, 218 bp, Zea mays
cgaagataattcttcattgaaagaatccaaggagggggagaaaaataatacgagaatagactagaatagattatagtaaaagaaaaaaagtaaaaggaaaaaacctacgaatctttcatgcttaaaaaaggtgcgagatgcttcaatcgagatccttcaaaaaaaaaaaaaaagaacatcaaaaattttctaagaataagaaaatagtataaatggaa

SEQ ID NO: 245 - DNA, 545 bp, Zea mays
cattctagttcctactgcctttttacttattatttatgtaaaaacagccagtcaaaatgattaattggaattccaattaatcattgaagaaatgaaaaagggattaaagaaaataaaaaatccaagtcttaaatgaaagggtctggttggaatcataaagtgtggtagaaagaactacatatagttttttctaccacactttagattctttctattatattatcttgaatctacatagaatcgattagtagattgaaatagtagtctaattcaacttcttttttcactgcatccacttaatttcaatcaagtcaaaatcaaaaaaatcgaagataattcttcattgaaagaatccaaggagggggagaaaaataatacgagaatagactagaatagattatagtaaaagaaaaaaagtaaaaggaaaaaacctacgaatctttcatgcttaaaaaaggtgcgagatgcttcaatcgagatccttcaaaaaaaaaaaaaaagaacatcaaaaattttctaagaataagaaaatagtataaatggaa

SEQ ID NO: 246 - DNA, 545 bp, Zea mays
cgaagataattcttcattgaaagaatccaaggagggggagaaaaataatacgagaatagactagaatagattatagtaaaagaaaaaaagtaaaaggaaaaaacctacgaatctttcatgcttaaaaaaggtgcgagatgcttcaatcgagatccttcaaaaaaaaaaaaaaagaacatcaaaaattttctaagaataagaaaatagtataaatggaaaatgtgcgatatattgtgaatagcttcgtgtaagaaagtctaattttcttatgttctaattttcttatgtatagaactttctttttactcataatttctagtagaatagaagttatgaattactataatcgccctttctattctattatactggaatagaacgactctttcttactttcttaaaagagtttcttacaagagtgaaacaaaactaaagaaagagagaaaaaataaagtttggcaaaattattaatgcaaaacgatcaattaaagaaaagagttgatactacaattcgactactcaatcaattggtagtatccctagag

SEQ ID NO: 247 - DNA, 1090 bp, Zea mays
tccactcctcccccatactactagcgaaagagaaaatgtaaagactaccattaaagcagcccaagtgagacttactatatccatgtaaattatgtctcctatttctatgaaagaattcttctactattgatcaataatcatagtggaatcaagggtacagagtcaaaaggccattctgccctaagactatggatgaatccgttaaaggaatttactcctaacaaattcttatatgatttctggtagaattggagagcattaagtagaaatatgatacatcgctctttccctaaaaaagagtaggggatgctctgaatagaagacgcgggaatagagagattttttctttcgtttgttaccttttttataagcaaaggacgtaagaatacaccataaaattaggatagataaatatttattggatacctaacttacccatgtttatttttatttttgacctaaaccctactgctgccccgctttctatctttctatgaaagagtaaggaggtcttatccacaatccaaaaattgatttttgatcagcctattcaatctaattctcgacagaatcagtagcctttactttagtgtatgtaggtagcataagatgtacgaagtgtatgtagtaagatgtacgataaagtgtatgtagtaagatgtacgataaataaaatgtatgataattaggaagtctttctatttcttcgcaaagtcccttcttcaatcttagattgaaaaaagaatgccccttagggacctttggctacgaagatttctgacatcttagtctcgtagttttaaattcccgaatcgaatcgatttaaattaataaaagaataaggaaacgctacctcatccctattggtagccgtttgggccactgcccacaaaacaaaccctagtttgagggtagaactatggtatggattctcaaaatccagtatcgccaaacctagttactctcgtgccccaacttaggggggtacgaatttgtcgagtttgatcagtactataatcctaagtattttattgatcaggcggcacccagatttgaactgggggtaaaggatttgcagtcccctgcctta

SEQ ID NO: 248 - DNA, 327 bp, Zea mays
catgtttatttttatttttgacctaaaccctactgctgccccgctttctatctttctatgaaagagtaaggaggtcttatccacaatccaaaaattgatttttgatcagcctattcaatctaattctcgacagaatcagtagcctttactttagtgtatgtaggtagcataagatgtacgaagtgtatgtagtaagatgtacgataaagtgtatgtagtaagatgtacgataaataaaatgtatgataattaggaagtctttctatttcttcgcaaagtcccttcttcaatcttagattgaaaaaagaatgccccttagggaccttt

SEQ ID NO: 249 - DNA, 763 bp, Zea mays
tccactcctcccccatactactagcgaaagagaaaatgtaaagactaccattaaagcagcccaagtgagacttactatatccatgtaaattatgtctcctatttctatgaaagaattcttctactattgatcaataatcatagtggaatcaagggtacagagtcaaaaggccattctgccctaagactatggatgaatccgttaaaggaatttactcctaacaaattcttatatgatttctggtagaattggagagcattaagtagaaatatgatacatcgctctttccctaaaaaagagtaggggatgctctgaatagaagacgcgggaatagagagattttttctttcgtttgttaccttttttataagcaaaggacgtaagaatacaccataaaattaggatagataaatatttattggatacctaacttacccatgtttatttttatttttgacctaaaccctactgctgccccgctttctatctttctatgaaagagtaaggaggtcttatccacaatccaaaaattgatttttgatcagcctattcaatctaattctcgacagaatcagtagcctttactttagtgtatgtaggtagcataagatgtacgaagtgtatgtagtaagatgtacgataaagtgtatgtagtaagatgtacgataaataaaatgtatgataattaggaagtctttctatttcttcgcaaagtcccttcttcaatcttagattgaaaaaagaatgccccttagggaccttt

SEQ ID NO: 250 - DNA, 654 bp, Zea mays
catgtttatttttatttttgacctaaaccctactgctgccccgctttctatctttctatgaaagagtaaggaggtcttatccacaatccaaaaattgatttttgatcagcctattcaatctaattctcgacagaatcagtagcctttactttagtgtatgtaggtagcataagatgtacgaagtgtatgtagtaagatgtacgataaagtgtatgtagtaagatgtacgataaataaaatgtatgataattaggaagtctttctatttcttcgcaaagtcccttcttcaatcttagattgaaaaaagaatgccccttagggacctttggctacgaagatttctgacatcttagtctcgtagttttaaattcccgaatcgaatcgatttaaattaataaaagaataaggaaacgctacctcatccctattggtagccgtttgggccactgcccacaaaacaaaccctagtttgagggtagaactatggtatggattctcaaaatccagtatcgccaaacctagttactctcgtgccccaacttaggggggtacgaatttgtcgagtttgatcagtactataatcctaagtattttattgatcaggcggcacccagatttgaactgggggtaaaggatttgcagtcccctgcctta

SEQ ID NO: 251 - DNA, 1308 bp, Zea mays
ccacttggccatgccgccaaaaaatccgatctaaaatcgagaaaagagcaagtattcattcacatttttttactaattactaaaacctttttttcttttatcttgaatctaattctacttacttttttagaatctttttcaaaaaatagatttctgcttttttggatccagtttcgcttattctcctcgatggattctatcttaaaacacacattgctaacactagaaaacttcccgcttctttctattctattgagatgacaaagaagaaaagaagaaaaagtggatttccagtcgcaggctgcaaaattcagaacaaattagaaccattaactataatataataaaattagaatataataatataattaactataatataataaaataaaactataatataataatataataaatataatattattattattattttttgaattgcagtagtgaacgactcttaaatcggtattcccccccattattttgaatggcataataaaatagaataagaataaatgtttccctaatctgtatatagggaaactccaggtccgaacagcattattatctacagatcccccttatgtacatattcctatggggaatcgtgctttaatttttcattgtattaaatatcttgaataaaaaaaaaaagagggaatttgctctgatatagattatagccaggccttcttggcccacctaagtgcaattacgataaaagaaaggatatgtagataggtggataactagattggcaagtacttaaaaaataaagtactttatctttattttaggattctacaacgaaatcctttattttctagcaattctactactacgaaacaaaaaagacccttcaaattctttttgaaaaagaaactaagcgtgctattctaaatcgaactaaagtcaaactttctagtgcttataaattcttatggctttatccctttcatagaaaggagataaaacgagaaatctttgctatccaatctttttagaatatcataaagtttcagtggcagaatttttttctaggactgttttatcaattcatttttattccatttcaacccctgctaaattcgaactttcgtcgaaatcgtctctattcatatgtatgaaatacatatatgaaatacgtatgtggagttccctagaatttcatgtgattcagtaaacagaatatggattccatgattgctagattgatccgtagggattgatgaagagtgagctgataatggaatttttcttctataaataggaaacttaagattcaaatgctccggaatggaa

SEQ ID NO: 252 - DNA, 654 bp, Zea mays
cattaactataatataataaaattagaatataataatataattaactataatataataaaataaaactataatataataatataataaatataatattattattattattttttgaattgcagtagtgaacgactcttaaatcggtattcccccccattattttgaatggcataataaaatagaataagaataaatgtttccctaatctgtatatagggaaactccaggtccgaacagcattattatctacagatcccccttatgtacatattcctatggggaatcgtgctttaatttttcattgtattaaatatcttgaataaaaaaaaaaagagggaatttgctctgatatagattatagccaggccttcttggcccacctaagtgcaattacgataaaagaaaggatatgtagataggtggataactagattggcaagtacttaaaaaataaagtactttatctttattttaggattctacaacgaaatcctttattttctagcaattctactactacgaaacaaaaaagacccttcaaattctttttgaaaaagaaactaagcgtgctattctaaatcgaactaaagtcaaactttctagtgcttataaattcttatggctttatccctttcatagaaaggagataaaac

SEQ ID NO: 253 - DNA, 981 bp, Zea mays
ccacttggccatgccgccaaaaaatccgatctaaaatcgagaaaagagcaagtattcattcacatttttttactaattactaaaacctttttttcttttatcttgaatctaattctacttacttttttagaatctttttcaaaaaatagatttctgcttttttggatccagtttcgcttattctcctcgatggattctatcttaaaacacacattgctaacactagaaaacttcccgcttctttctattctattgagatgacaaagaagaaaagaagaaaaagtggatttccagtcgcaggctgcaaaattcagaacaaattagaaccattaactataatataataaaattagaatataataatataattaactataatataataaaataaaactataatataataatataataaatataatattattattattattttttgaattgcagtagtgaacgactcttaaatcggtattcccccccattattttgaatggcataataaaatagaataagaataaatgtttccctaatctgtatatagggaaactccaggtccgaacagcattattatctacagatcccccttatgtacatattcctatggggaatcgtgctttaatttttcattgtattaaatatcttgaataaaaaaaaaaagagggaatttgctctgatatagattatagccaggccttcttggcccacctaagtgcaattacgataaaagaaaggatatgtagataggtggataactagattggcaagtacttaaaaaataaagtactttatctttattttaggattctacaacgaaatcctttattttctagcaattctactactacgaaacaaaaaagacccttcaaattctttttgaaaaagaaactaagcgtgctattctaaatcgaactaaagtcaaactttctagtgcttataaattcttatggctttatccctttcatagaaaggagataaaac

SEQ ID NO: 254 - DNA, 981 bp, Zea mays
cattaactataatataataaaattagaatataataatataattaactataatataataaaataaaactataatataataatataataaatataatattattattattattttttgaattgcagtagtgaacgactcttaaatcggtattcccccccattattttgaatggcataataaaatagaataagaataaatgtttccctaatctgtatatagggaaactccaggtccgaacagcattattatctacagatcccccttatgtacatattcctatggggaatcgtgctttaatttttcattgtattaaatatcttgaataaaaaaaaaaagagggaatttgctctgatatagattatagccaggccttcttggcccacctaagtgcaattacgataaaagaaaggatatgtagataggtggataactagattggcaagtacttaaaaaataaagtactttatctttattttaggattctacaacgaaatcctttattttctagcaattctactactacgaaacaaaaaagacccttcaaattctttttgaaaaagaaactaagcgtgctattctaaatcgaactaaagtcaaactttctagtgcttataaattcttatggctttatccctttcatagaaaggagataaaacgagaaatctttgctatccaatctttttagaatatcataaagtttcagtggcagaatttttttctaggactgttttatcaattcatttttattccatttcaacccctgctaaattcgaactttcgtcgaaatcgtctctattcatatgtatgaaatacatatatgaaatacgtatgtggagttccctagaatttcatgtgattcagtaaacagaatatggattccatgattgctagattgatccgtagggattgatgaagagtgagctgataatggaatttttcttctataaataggaaacttaagattcaaatgctccggaatggaa

SEQ ID NO: 255 - DNA, 981 bp, Zea mays
tcttggattatttacaagcggtattcaagctcttatttttgcaacgttagccgcagcctatataggtgaatccatggaaggtcatcattgaattaactagtttttgaaatagtctttctttttttagcttagcccaattcatgcatgattccggataatcctcttagttggaaaactaaatagttcgaaaggcgtatgaatatacaacctagagttgtaggagagagaatagactatattatggaagaggtaaagtatatatgcattcaggggggccaagtcaggttagatctatatctttaatgcctataagacagtcatcttttgtgtggctttctaaagaatgattttagaatcggattcaatagaaaatgagaaaataggcaaacaaaatagaagaaacaaatgtatatgggattttttttattcctaagttagattcattatctaatccgatatatagaattcccttctatatggaactggattccatatctagttctatgcagcatattgttatcaattgtatatcttgatttgattcctattggatttggattagttcgatttcaataggggttcttcctgtatttcgtcttttattatgttagatgaaggggaaaaaatgggaactcaaagatatcgaagagtaaaaaaaaggatggaataaaagagtgattggttgaaaagaaagagaaatagaataatgagaatcatatggatttacacaaacctctaatgattagaaactaaaaacgagatctcgaagtaattcgaacgatttcgattatcatttatttgtacttattagttacttctacccgatagagcttagaagttggaagtaataatttcttggttgattgtatccttaaccatttctttttttttgacacgaggaactcatcatgaatccactaattgctgctgcttccgttattgctgctggattggccgtagggcttgcttctattgggcccg

SEQ ID NO: 256 - DNA, 255 bp, Zea mays
tgtggctttctaaagaatgattttagaatcggattcaatagaaaatgagaaaataggcaaacaaaatagaagaaacaaatgtatatgggattttttttattcctaagttagattcattatctaatccgatatatagaattcccttctatatggaactggattccatatctagttctatgcagcatattgttatcaattgtatatcttgatttgattcctattggatttggattagttcgatttcaataggggttc

SEQ ID NO: 257 - DNA, 582 bp, Zea mays
tcttggattatttacaagcggtattcaagctcttatttttgcaacgttagccgcagcctatataggtgaatccatggaaggtcatcattgaattaactagtttttgaaatagtctttctttttttagcttagcccaattcatgcatgattccggataatcctcttagttggaaaactaaatagttcgaaaggcgtatgaatatacaacctagagttgtaggagagagaatagactatattatggaagaggtaaagtatatatgcattcaggggggccaagtcaggttagatctatatctttaatgcctataagacagtcatcttttgtgtggctttctaaagaatgattttagaatcggattcaatagaaaatgagaaaataggcaaacaaaatagaagaaacaaatgtatatgggattttttttattcctaagttagattcattatctaatccgatatatagaattcccttctatatggaactggattccatatctagttctatgcagcatattgttatcaattgtatatcttgatttgattcctattggatttggattagttcgatttcaataggggttc

SEQ ID NO: 258 - DNA, 654 bp, Zea mays
tgtggctttctaaagaatgattttagaatcggattcaatagaaaatgagaaaataggcaaacaaaatagaagaaacaaatgtatatgggattttttttattcctaagttagattcattatctaatccgatatatagaattcccttctatatggaactggattccatatctagttctatgcagcatattgttatcaattgtatatcttgatttgattcctattggatttggattagttcgatttcaataggggttcttcctgtatttcgtcttttattatgttagatgaaggggaaaaaatgggaactcaaagatatcgaagagtaaaaaaaaggatggaataaaagagtgattggttgaaaagaaagagaaatagaataatgagaatcatatggatttacacaaacctctaatgattagaaactaaaaacgagatctcgaagtaattcgaacgatttcgattatcatttatttgtacttattagttacttctacccgatagagcttagaagttggaagtaataatttcttggttgattgtatccttaaccatttctttttttttgacacgaggaactcatcatgaatccactaattgctgctgcttccgttattgctgctggattggccgtagggcttgcttctattgggcccg

SEQ ID NO: 259 - DNA, 981 bp, Zea mays
actatcggatttgaaccgatgaccctcgcattacaaatgcgatgctctaacctctgagctaagtgggcttacataacagaaatagtgtaacaaataaaaatatatatagggaatctgtaaaatgtcagatcttaattattaatcttagttattaactagttctaaattggaagttccacttagatagaaaaaaaaaaatactagaatttcataaagataaagttagcttgatatgcttaactaaatgatattcttaaataggattatagaatttattgaactttctttttatttctctaattcgcaaatggatttttttaatagaatctattccaaattctatattgaatttgatttcagatattttcaatttgatatggctcggaggaataatctaatacatagaaaagaagaaaaagaagaatatatatgaataatataataaagagaaaatgccaagagattggcattttcatttgatcattatatacatttttgagatattttgtttttttatttgttaataatttaaggataaatagttcactaaggagaagatagaatcatagcaaatgaaatttctaattcagtttagaaaaaaagaatgaatatcaagcgttatagtatgatttggaatactctaaaaaaggaaggagggaggcgggagagagaaaaacttttggatatattcattccgattgaattgcaaatatatcaacgataaaatcaatgcaattctgaattgcaataagcgagcggggtgtctcaaatagagatgagttgctagactacgtcgaataatgaattcaatgattcaaaaaaaactaagagatggattaaattatacaaggaatcctggtttcaaagaaaagtaaaatggggatatggcgaaatcggtagacgctacggacttgattgtattgagccttggtatggaaacctgctaagtggtaacttccaaattcagagaaaccctggaat

SEQ ID NO: 260 - DNA, 172 bp, Zea mays
gatatggctcggaggaataatctaatacatagaaaagaagaaaaagaagaatatatatgaataatataataaagagaaaatgccaagagattggcattttcatttgatcattatatacatttttgagatattttgtttttttatttgttaataatttaaggataaatagttc

SEQ ID NO: 261 - DNA, 545 bp, Zea mays
actatcggatttgaaccgatgaccctcgcattacaaatgcgatgctctaacctctgagctaagtgggcttacataacagaaatagtgtaacaaataaaaatatatatagggaatctgtaaaatgtcagatcttaattattaatcttagttattaactagttctaaattggaagttccacttagatagaaaaaaaaaaatactagaatttcataaagataaagttagcttgatatgcttaactaaatgatattcttaaataggattatagaatttattgaactttctttttatttctctaattcgcaaatggatttttttaatagaatctattccaaattctatattgaatttgatttcagatattttcaatttgatatggctcggaggaataatctaatacatagaaaagaagaaaaagaagaatatatatgaataatataataaagagaaaatgccaagagattggcattttcatttgatcattatatacatttttgagatattttgtttttttatttgttaataatttaaggataaatagttc

SEQ ID NO: 262 - DNA, 608 bp, Zea mays
gatatggctcggaggaataatctaatacatagaaaagaagaaaaagaagaatatatatgaataatataataaagagaaaatgccaagagattggcattttcatttgatcattatatacatttttgagatattttgtttttttatttgttaataatttaaggataaatagttcactaaggagaagatagaatcatagcaaatgaaatttctaattcagtttagaaaaaaagaatgaatatcaagcgttatagtatgatttggaatactctaaaaaaggaaggagggaggcgggagagagaaaaacttttggatatattcattccgattgaattgcaaatatatcaacgataaaatcaatgcaattctgaattgcaataagcgagcggggtgtctcaaatagagatgagttgctagactacgtcgaataatgaattcaatgattcaaaaaaaactaagagatggattaaattatacaaggaatcctggtttcaaagaaaagtaaaatggggatatggcgaaatcggtagacgctacggacttgattgtattgagccttggtatggaaacctgctaagtggtaacttccaaattcagagaaaccctggaat

SEQ ID NO: 263 - DNA, 1090 bp, Zea mays
gggctaaaagtcctgaaatccaaaataccaaaataggaataaggcttgctattattagaaatgtccaaaaaatatcatattcgtgaagcagaaacataaatgtactcccattaatgtggaataggcggaactgaattagtcaattcaagttgacattgtcaatttatccagaacttctctcttttcctcggtgaaacaagaatctgttttgctcaaaccaaaggcaaggagcgcttactttagcctttatttcttttgtgatatgtcttccttaaggattcatccaatgtaatccccacttcccttctttttgatttcccttctatttagatatgatatagacctaattcttatacaaagaaaactctttcgcttcactttgtctcactttctctagaatctctagaaaaaagaattgctctaccctttatttaatgatagtcagagactattaaataaaaaagaaaatacttactactaattggattagaacctaattaaaataggatgattaatatatattaaaataggatgattaatatatacatcctaatgaggagtaatactaaaaaaaagaactctatttcagaattatgaaacagaatatcctgttttattatttactctttctttttttttttctttcgattttttctattttatttaaagtggatcgatttagataatgtatatgatagtaatatacttcaaataaaataggaattcgcaaaatggagaaaatcgttgcagtcattataggaaagtatgggaagacttagagcatatcctatttaaaggaggatgaaattcaaatcaggtaagggatccttccttctattgatttgatcaaaattcttttttcttttcctgtctctctgatttgcgtttcgatgaatgagcctatggtaatgcttttatctctattctatggcgcaatcgaccgtcgagtctataaacaagtactaaataaggaaaagaaaactatactaaaggaaacataagatatccatcctaaaatggaaaaaagacgtatatattagggctatacggattcgaaccgtagaccttctcggtaaaacagatcaaacag

SEQ ID NO: 264 - DNA, 218 bp, Zea mays
gatagtcagagactattaaataaaaaagaaaatacttactactaattggattagaacctaattaaaataggatgattaatatatattaaaataggatgattaatatatacatcctaatgaggagtaatactaaaaaaaagaactctatttcagaattatgaaacagaatatcctgttttattatttactctttctttttttttttctttcgatttttt

SEQ ID NO: 265 - DNA, 654 bp, Zea mays
gggctaaaagtcctgaaatccaaaataccaaaataggaataaggcttgctattattagaaatgtccaaaaaatatcatattcgtgaagcagaaacataaatgtactcccattaatgtggaataggcggaactgaattagtcaattcaagttgacattgtcaatttatccagaacttctctcttttcctcggtgaaacaagaatctgttttgctcaaaccaaaggcaaggagcgcttactttagcctttatttcttttgtgatatgtcttccttaaggattcatccaatgtaatccccacttcccttctttttgatttcccttctatttagatatgatatagacctaattcttatacaaagaaaactctttcgcttcactttgtctcactttctctagaatctctagaaaaaagaattgctctaccctttatttaatgatagtcagagactattaaataaaaaagaaaatacttactactaattggattagaacctaattaaaataggatgattaatatatattaaaataggatgattaatatatacatcctaatgaggagtaatactaaaaaaaagaactctatttcagaattatgaaacagaatatcctgttttattatttactctttctttttttttttctttcgatttttt

SEQ ID NO: 266 - DNA, 654 bp, Zea mays
gatagtcagagactattaaataaaaaagaaaatacttactactaattggattagaacctaattaaaataggatgattaatatatattaaaataggatgattaatatatacatcctaatgaggagtaatactaaaaaaaagaactctatttcagaattatgaaacagaatatcctgttttattatttactctttctttttttttttctttcgattttttctattttatttaaagtggatcgatttagataatgtatatgatagtaatatacttcaaataaaataggaattcgcaaaatggagaaaatcgttgcagtcattataggaaagtatgggaagacttagagcatatcctatttaaaggaggatgaaattcaaatcaggtaagggatccttccttctattgatttgatcaaaattcttttttcttttcctgtctctctgatttgcgtttcgatgaatgagcctatggtaatgcttttatctctattctatggcgcaatcgaccgtcgagtctataaacaagtactaaataaggaaaagaaaactatactaaaggaaacataagatatccatcctaaaatggaaaaaagacgtatatattagggctatacggattcgaaccgtagaccttctcggtaaaacagatcaaacag

SEQ ID NO: 267 - DNA, 872 bp, Zea mays
aatacgccctacgcttttttcttcaattgtggaaatcccgggacgcgaagtagtaggattggttctcataattatcacataattttcaaaaaaaaaggaatttgtcgaaattttttttcttgttgaataatgccaaatcaaaaaaaatatccaaaaatccaaaagtcaaaaggaaatgaattagttaattcaataaaaaagaaaaggggactcgcacttgatttcgttgcccaagcgaatcccattcaatcgtttacttatggaatgaatccgttggaaagttcaatccatttttttcatataaatttcgccttttgtgaaggatctgtgcctactctactttcctatctaggacttcgatatacaaaatatatactactgtgaagcatagattgctgtcaacagagaattttcttagtatttaggtatttagattcaaaatatcaaaggggaagaactttaaaaattgtaaaataaagattagggtttgggttgcgctatatctatcaaagagtatacaataatgatggatttggtgaatcaaatccacggtttaataacgaaccgtgttaacttaccataacaacaactcaattcctatcgaattccttcctatcgaattcctatagtaaaattcctataggatagaacgtacacagggtgtacacattatatatgaatgaaacatattcattaacttaagcatactcctttttttatttaatgagttgatattaattaaatatctttttttttagatttttgcaaaggtttcatttacgcctaatccatatcgagtagaccctgtcgttgtgagaattcttaattcatgagttgtagggagggacttatgtcaccacaaacagaaacta

SEQ ID NO: 268 - DNA, 170 bp, Zea mays
ctactgtgaagcatagattgctgtcaacagagaattttcttagtatttaggtatttagattcaaaatatcaaaggggaagaactttaaaaattgtaaaataaagattagggtttgggttgcgctatatctatcaaagagtatacaataatgatggatttggtgaatcaaa

SEQ ID NO: 269 - DNA, 545 bp, Zea mays
aatacgccctacgcttttttcttcaattgtggaaatcccgggacgcgaagtagtaggattggttctcataattatcacataattttcaaaaaaaaaggaatttgtcgaaattttttttcttgttgaataatgccaaatcaaaaaaaatatccaaaaatccaaaagtcaaaaggaaatgaattagttaattcaataaaaaagaaaaggggactcgcacttgatttcgttgcccaagcgaatcccattcaatcgtttacttatggaatgaatccgttggaaagttcaatccatttttttcatataaatttcgccttttgtgaaggatctgtgcctactctactttcctatctaggacttcgatatacaaaatatatactactgtgaagcatagattgctgtcaacagagaattttcttagtatttaggtatttagattcaaaatatcaaaggggaagaactttaaaaattgtaaaataaagattagggtttgggttgcgctatatctatcaaagagtatacaataatgatggatttggtgaatcaaa

SEQ ID NO: 270 - DNA, 497 bp, Zea mays
ctactgtgaagcatagattgctgtcaacagagaattttcttagtatttaggtatttagattcaaaatatcaaaggggaagaactttaaaaattgtaaaataaagattagggtttgggttgcgctatatctatcaaagagtatacaataatgatggatttggtgaatcaaatccacggtttaataacgaaccgtgttaacttaccataacaacaactcaattcctatcgaattccttcctatcgaattcctatagtaaaattcctataggatagaacgtacacagggtgtacacattatatatgaatgaaacatattcattaacttaagcatactcctttttttatttaatgagttgatattaattaaatatctttttttttagatttttgcaaaggtttcatttacgcctaatccatatcgagtagaccctgtcgttgtgagaattcttaattcatgagttgtagggagggacttatgtcaccacaaacagaaacta

SEQ ID NO: 271 - DNA, 1090 bp, Zea mays
ttatcaaagcagcttgcaaatggagtgctgaactagccgcagcttgtgaaatatggaaggagatcaaatttgatggtttcaaagcgatggataccatataaaataaaaaaaaaagcaaaatagaaagagaaaaaataagttaagaaatgcagtaattcttctttattcctctaattgattgcaattcaattcggctcaatcttttctaaaaaaaaaaaagactgagccgaattgaaatagatcttgatacgatcatgagacttgacaaatcgagattcttctattctatatatctagaatatagaaaggtataatacaataaacaaatataaatcaaaatatagtattatcgtacgataatggaatcaaatacgcagtatttacagaaaagagtcttcgtttattgggaaagaatcaatatacttttaatgtcgaatcgggattcactaagacagaaataaagcattgggtcgaactcttctttggtgttaaggtagtagctgtgaatagccatcgactacccggaaagggtagaagaatgggacctattctgggacatacaatgcattacagacgtatgatcattacccttcaactgggtattctattccacttctactttttaaattaaagggtattctgaaagaggtatttcttttattttcacaatagctttcttttgaatccaatttcaagttcgattagaaggatagaaaggcgatgagaatgggaaaagaaaaataaaatattttattttaatgagttccccttttgagttccccttttttgcaattttcttattatcaattccattcatttattttatagaatacttttttattctatatctataaaaaatctttattttgtaaactaaaaaatacaatagtcaatattccttataatagatatacttaattatatcataagaatcttaagatatatttcgaatagatagaaatagtaaatttgaattgagacacatattctatgacagattttaacttaccctctattttcgtacctttagtaggcttagtatttccggcaattgcaatgacttccttatttctttatgtgcagaaaaata

SEQ ID NO: 272 - DNA, 360 bp, Zea mays
tataaatcaaaatatagtattatcgtacgataatggaatcaaatacgcagtatttacagaaaagagtcttcgtttattgggaaagaatcaatatacttttaatgtcgaatcgggattcactaagacagaaataaagcattgggtcgaactcttctttggtgttaaggtagtagctgtgaatagccatcgactacccggaaagggtagaagaatgggacctattctgggacatacaatgcattacagacgtatgatcattacccttcaactgggtattctattccacttctactttttaaattaaagggtattctgaaagaggtatttcttttattttcacaatagctttcttttgaatcc

SEQ ID NO: 273 - DNA, 687 bp, Zea mays
ttatcaaagcagcttgcaaatggagtgctgaactagccgcagcttgtgaaatatggaaggagatcaaatttgatggtttcaaagcgatggataccatataaaataaaaaaaaaagcaaaatagaaagagaaaaaataagttaagaaatgcagtaattcttctttattcctctaattgattgcaattcaattcggctcaatcttttctaaaaaaaaaaaagactgagccgaattgaaatagatcttgatacgatcatgagacttgacaaatcgagattcttctattctatatatctagaatatagaaaggtataatacaataaacaaatataaatcaaaatatagtattatcgtacgataatggaatcaaatacgcagtatttacagaaaagagtcttcgtttattgggaaagaatcaatatacttttaatgtcgaatcgggattcactaagacagaaataaagcattgggtcgaactcttctttggtgttaaggtagtagctgtgaatagccatcgactacccggaaagggtagaagaatgggacctattctgggacatacaatgcattacagacgtatgatcattacccttcaactgggtattctattccacttctactttttaaattaaagggtattctgaaagaggtatttcttttattttcacaatagctttcttttgaatcc

SEQ ID NO: 274 - DNA, 763 bp, Zea mays
tataaatcaaaatatagtattatcgtacgataatggaatcaaatacgcagtatttacagaaaagagtcttcgtttattgggaaagaatcaatatacttttaatgtcgaatcgggattcactaagacagaaataaagcattgggtcgaactcttctttggtgttaaggtagtagctgtgaatagccatcgactacccggaaagggtagaagaatgggacctattctgggacatacaatgcattacagacgtatgatcattacccttcaactgggtattctattccacttctactttttaaattaaagggtattctgaaagaggtatttcttttattttcacaatagctttcttttgaatccaatttcaagttcgattagaaggatagaaaggcgatgagaatgggaaaagaaaaataaaatattttattttaatgagttccccttttgagttccccttttttgcaattttcttattatcaattccattcatttattttatagaatacttttttattctatatctataaaaaatctttattttgtaaactaaaaaatacaatagtcaatattccttataatagatatacttaattatatcataagaatcttaagatatatttcgaatagatagaaatagtaaatttgaattgagacacatattctatgacagattttaacttaccctctattttcgtacctttagtaggcttagtatttccggcaattgcaatgacttccttatttctttatgtgcagaaaaata

SEQ ID NO: 275 - DNA, 1090 bp, Zea mays
ggccttttgtttttcttcgcatctgttattttggcacaagtttttttggttctcaaaaagaaacagtttgaaaaggttcaattgtacgaaatgaatttctagatcctgggatttcttaccatcaagtgagaaaaaagcctcgatttctggaattccttatttctatctcatgcaaaagaagaggatccaaggcaaagacgagaacaataaaaggaacaagtccttttaggaggaattgtgggtcttcttaatcctctattgggcacaagaaaaaggcttttttgcctttttcttgtgtcgattcttcttgtatcgaagtaagaatcatttttcttcttatttgttttgtcaaagattactatttattctttattcgggtctgtcttttggacttcctttgcttaggttcgggcgcggtagtggacaaacagagggaaagaaggtatggcgggggacacatttcttgtgagcaaatagaattgctttactttttgaattaagttcaactttgaacttacagaaattttgtaaaaaaaaatctataccctcccatggaagggtggttttgatttttaggctagttgagtagttttgattaaggttttattagtttattactctaaactaaatcaacgatttgcaggatacttccttcatggaataaaatactggatcctctcccctctttcttgttgcttcataagagtgaatccattttatgggcgaaaggcgggggctttaaatcgaccgatggattgcttcactcactaacatcattaacaaacaaaagaataaatggagggattccaaccatcagagcaaaggttttctctttgttatttttacaaatagaaataggtaaccaatttctagattatggaacaaaaccgcgttataacaataagaattccgcgggccctttccgctctaatcagataaaggggagtaaggacccgctaagttcctactttttcatgtttacaatctggatcctccgattactatagagatgaacccaatccagaatacgaaccgtaaaagaaaacacctattaaaccaatcacaagaataccagttacagtacctatca

SEQ ID NO: 276 - DNA, 436 bp, Zea mays
tttttcttcttatttgttttgtcaaagattactatttattctttattcgggtctgtcttttggacttcctttgcttaggttcgggcgcggtagtggacaaacagagggaaagaaggtatggcgggggacacatttcttgtgagcaaatagaattgctttactttttgaattaagttcaactttgaacttacagaaattttgtaaaaaaaaatctataccctcccatggaagggtggttttgatttttaggctagttgagtagttttgattaaggttttattagtttattactctaaactaaatcaacgatttgcaggatacttccttcatggaataaaatactggatcctctcccctctttcttgttgcttcataagagtgaatccattttatgggcgaaaggcgggggctttaaatcgaccgatggattgcttca

SEQ ID NO: 277 - DNA, 763 bp, Zea mays
ggccttttgtttttcttcgcatctgttattttggcacaagtttttttggttctcaaaaagaaacagtttgaaaaggttcaattgtacgaaatgaatttctagatcctgggatttcttaccatcaagtgagaaaaaagcctcgatttctggaattccttatttctatctcatgcaaaagaagaggatccaaggcaaagacgagaacaataaaaggaacaagtccttttaggaggaattgtgggtcttcttaatcctctattgggcacaagaaaaaggcttttttgcctttttcttgtgtcgattcttcttgtatcgaagtaagaatcatttttcttcttatttgttttgtcaaagattactatttattctttattcgggtctgtcttttggacttcctttgcttaggttcgggcgcggtagtggacaaacagagggaaagaaggtatggcgggggacacatttcttgtgagcaaatagaattgctttactttttgaattaagttcaactttgaacttacagaaattttgtaaaaaaaaatctataccctcccatggaagggtggttttgatttttaggctagttgagtagttttgattaaggttttattagtttattactctaaactaaatcaacgatttgcaggatacttccttcatggaataaaatactggatcctctcccctctttcttgttgcttcataagagtgaatccattttatgggcgaaaggcgggggctttaaatcgaccgatggattgcttca

SEQ ID NO: 278 - DNA, 763 bp, Zea mays
tttttcttcttatttgttttgtcaaagattactatttattctttattcgggtctgtcttttggacttcctttgcttaggttcgggcgcggtagtggacaaacagagggaaagaaggtatggcgggggacacatttcttgtgagcaaatagaattgctttactttttgaattaagttcaactttgaacttacagaaattttgtaaaaaaaaatctataccctcccatggaagggtggttttgatttttaggctagttgagtagttttgattaaggttttattagtttattactctaaactaaatcaacgatttgcaggatacttccttcatggaataaaatactggatcctctcccctctttcttgttgcttcataagagtgaatccattttatgggcgaaaggcgggggctttaaatcgaccgatggattgcttcactcactaacatcattaacaaacaaaagaataaatggagggattccaaccatcagagcaaaggttttctctttgttatttttacaaatagaaataggtaaccaatttctagattatggaacaaaaccgcgttataacaataagaattccgcgggccctttccgctctaatcagataaaggggagtaaggacccgctaagttcctactttttcatgtttacaatctggatcctccgattactatagagatgaacccaatccagaatacgaaccgtaaaagaaaacacctattaaaccaatcacaagaataccagttacagtacctatca

SEQ ID NO: 279 - DNA, 1308 bp, Zea mays
gaacgttctcccgtgcttccagacatgctgagctcccaaaatttttattcaaaaaaggaattgattccgtaaaagatgggatccaccagtaaatagaaaattactgatatttcatccttgtgagattgtcaattttgtaccaaaggtgtattttgagtataccgaattagtatagctatccttcctatggcacagcaatccagttttgcttggtcccgaaacagaattccttttttctcttttttgttccttgtctataggaaaattacatgttattcaaggcatcaatagaaccccacaattttttgggtcctacttattttcattgtcttcggaatagtagaataatttaatttggaatagcggccaagatcttgggaaaatctaagttaatgattaataagataaagaatttaggaaggatattctcatattgacgcaatacaaagataagtatatgcaaaatcgatccctttttagttaaaggaatttaattggttagcataaaataatatctaataaatagaaaatcgaatagcggataatccgttatgaaagaaacggaatacattctttgaagaatcaagattcgtaatcaatccttgtcttgtttgttggattaggtctaattttcttgaccaaacagcaagcatggaactttacaagaagaacttaaaagaaaaggataattgaagtaactttgaatccacttttgttggggttcaaaaaacgaataaaaataaataaaaaaaagtaaattttaggaatagttccctttttgagggggcccctcgggggtcgtggaatgcttttcttctcctcttattccatatggaatacaatgagttaaaatttgctccaaaaagaggatgaaatccatcctattaaaaaagaaataatccgaatccgaaatagaaagaattttttttttttttttcaaattcaattctttatttatcttttattccaaaattctcccgaaaatccaatttcatttttcaatggggttagatgatctagttcttaatattattagtttacttaactgacagattccacaacaaatctcttgattcggaattagggactcatgttccgtctgatgaatcgattttctcttttttttacacttctgtatctcactctatcttgttttttagtattatctaaaataaccgatgaattatgaattttccataacttaaacttaggtaagtgctttaccaacatatgtagtgtagtaaaaaaaatggaattgaaccccttcatgcttactataactagttatttcggttttctactggctgc

SEQ ID NO: 280 - DNA, 592 bp, Zea mays
gtcttcggaatagtagaataatttaatttggaatagcggccaagatcttgggaaaatctaagttaatgattaataagataaagaatttaggaaggatattctcatattgacgcaatacaaagataagtatatgcaaaatcgatccctttttagttaaaggaatttaattggttagcataaaataatatctaataaatagaaaatcgaatagcggataatccgttatgaaagaaacggaatacattctttgaagaatcaagattcgtaatcaatccttgtcttgtttgttggattaggtctaattttcttgaccaaacagcaagcatggaactttacaagaagaacttaaaagaaaaggataattgaagtaactttgaatccacttttgttggggttcaaaaaacgaataaaaataaataaaaaaaagtaaattttaggaatagttccctttttgagggggcccctcgggggtcgtggaatgcttttcttctcctcttattccatatggaatacaatgagttaaaatttgctccaaaaagaggatgaaatccatcctattaaaaaagaaataatccgaatccgaaatagaaag

SEQ ID NO: 281 - DNA, 919 bp, Zea mays
gaacgttctcccgtgcttccagacatgctgagctcccaaaatttttattcaaaaaaggaattgattccgtaaaagatgggatccaccagtaaatagaaaattactgatatttcatccttgtgagattgtcaattttgtaccaaaggtgtattttgagtataccgaattagtatagctatccttcctatggcacagcaatccagttttgcttggtcccgaaacagaattccttttttctcttttttgttccttgtctataggaaaattacatgttattcaaggcatcaatagaaccccacaattttttgggtcctacttattttcattgtcttcggaatagtagaataatttaatttggaatagcggccaagatcttgggaaaatctaagttaatgattaataagataaagaatttaggaaggatattctcatattgacgcaatacaaagataagtatatgcaaaatcgatccctttttagttaaaggaatttaattggttagcataaaataatatctaataaatagaaaatcgaatagcggataatccgttatgaaagaaacggaatacattctttgaagaatcaagattcgtaatcaatccttgtcttgtttgttggattaggtctaattttcttgaccaaacagcaagcatggaactttacaagaagaacttaaaagaaaaggataattgaagtaactttgaatccacttttgttggggttcaaaaaacgaataaaaataaataaaaaaaagtaaattttaggaatagttccctttttgagggggcccctcgggggtcgtggaatgcttttcttctcctcttattccatatggaatacaatgagttaaaatttgctccaaaaagaggatgaaatccatcctattaaaaaagaaataatccgaatccgaaatagaaag

SEQ ID NO: 282 - DNA, 981 bp, Zea mays
gtcttcggaatagtagaataatttaatttggaatagcggccaagatcttgggaaaatctaagttaatgattaataagataaagaatttaggaaggatattctcatattgacgcaatacaaagataagtatatgcaaaatcgatccctttttagttaaaggaatttaattggttagcataaaataatatctaataaatagaaaatcgaatagcggataatccgttatgaaagaaacggaatacattctttgaagaatcaagattcgtaatcaatccttgtcttgtttgttggattaggtctaattttcttgaccaaacagcaagcatggaactttacaagaagaacttaaaagaaaaggataattgaagtaactttgaatccacttttgttggggttcaaaaaacgaataaaaataaataaaaaaaagtaaattttaggaatagttccctttttgagggggcccctcgggggtcgtggaatgcttttcttctcctcttattccatatggaatacaatgagttaaaatttgctccaaaaagaggatgaaatccatcctattaaaaaagaaataatccgaatccgaaatagaaagaattttttttttttttttcaaattcaattctttatttatcttttattccaaaattctcccgaaaatccaatttcatttttcaatggggttagatgatctagttcttaatattattagtttacttaactgacagattccacaacaaatctcttgattcggaattagggactcatgttccgtctgatgaatcgattttctcttttttttacacttctgtatctcactctatcttgttttttagtattatctaaaataaccgatgaattatgaattttccataacttaaacttaggtaagtgctttaccaacatatgtagtgtagtaaaaaaaatggaattgaaccccttcatgcttactataactagttatttcggttttctactggctgc

SEQ ID NO: 283 - DNA, 763 bp, Zea mays
taaagtttgaggcaaatgaacgcatttttgttcgtcgtctccgagctatatatcctcgcggaactctggtcattgaatcaaattaaccttaatgaataactaatgatttctcttcttttaaccctcttttttcccattaataactaaacggattattccgatatataaaatattaattccaatggcttttgctactataaccttcccaaccacgattttttattctatttagttatttcgcatagaaataacaaattctaacgatactaaaaaaacagtgggttccatcgtttttatggttcccttttaaacggcgaggccctctctatacaccggagccctttctttcatcaaaaggtattgtgaacttgtatagttcacattctttggctctacctatccattatagagtaaataactcttttcacaataagagttattcatacagtgacggtatttaattatgaaagttggctaagtagctgaccctttagtccgttcttttaagataaaggagcataagcctttttctttttattactatttcctccgcttaatggataaccatttgttaccaatgggggaattgcttcttccaatctagatgattggatttgcaccaaaggaaaccataaattccatataccatagaaatctaggatagagcttctctatcctattcattggtaccgatcatggatacttcaaaaattttattatttgtttgaactcatgatctgaacgagtcgcacatacaccctag

SEQ ID NO: 284 - DNA, 61 bp, Zea mays
gttcacattctttggctctacctatccattatagagtaaataactcttttcacaataagag

SEQ ID NO: 285 - DNA, 436 bp, Zea mays
taaagtttgaggcaaatgaacgcatttttgttcgtcgtctccgagctatatatcctcgcggaactctggtcattgaatcaaattaaccttaatgaataactaatgatttctcttcttttaaccctcttttttcccattaataactaaacggattattccgatatataaaatattaattccaatggcttttgctactataaccttcccaaccacgattttttattctatttagttatttcgcatagaaataacaaattctaacgatactaaaaaaacagtgggttccatcgtttttatggttcccttttaaacggcgaggccctctctatacaccggagccctttctttcatcaaaaggtattgtgaacttgtatagttcacattctttggctctacctatccattatagagtaaataactcttttcacaataagag

SEQ ID NO: 286 - DNA, 388 bp, Zea mays
gttcacattctttggctctacctatccattatagagtaaataactcttttcacaataagagttattcatacagtgacggtatttaattatgaaagttggctaagtagctgaccctttagtccgttcttttaagataaaggagcataagcctttttctttttattactatttcctccgcttaatggataaccatttgttaccaatgggggaattgcttcttccaatctagatgattggatttgcaccaaaggaaaccataaattccatataccatagaaatctaggatagagcttctctatcctattcattggtaccgatcatggatacttcaaaaattttattatttgtttgaactcatgatctgaacgagtcgcacatacaccctag

SEQ ID NO: 287 - DNA, 3706 bp, Zea mays
gcaggaattgaacccgcaaatttaccaattatgagttgggcgctttaaccattcagccatggatgcttaacagggatcatcgtacatcgtaaataaccaattttcatatagaaagacatatcatagaaaaatgaaatcgaaaatattcggagatggcaaatattcggagatgactatgaaaacacctctccggatcctcgaattgaaagagagactgagagggatcaagaatcctaattctcgctatttggaatggatccaattctattgagtctgacccatagtgatcatttctctttagcaaagaatgaccttggttatcaaaggattgaacaaccgagatccatttacttatgatacctagttgacattgctaacaaggatctaatgaattatgagtttaatagattctctttagcagaaagacgtatattccttgctcattatcagacaatcacttattcccaaacctcgtgtggggctaatcgttttcatttaccatctcatggaaaacccttttcgttccgcttagccctatcgggtattttagtgataggttctataggaactggacaatcctatttggtcaaatacctaacgaaaaattcctattttcctttcattaaggtacgagggcttcttattccacaagaacgaaagcaccttttcattctttcatatactaggggtttttacttggaaaagacaatgttccatactaaaggattcgggtccataaccacgagttccagtgcactagatcttgtagcacttagcaacgaggccctatccattagtattccacataagaaatccattatagaaactaatacaattagattagctcttcatagacaaacttggggtttgcgagccaagataagaccggctcgggatcatgggacccttttctatcagataggaggggatcttgtacaaaatagacttctaagtaataaccccatagatcctatatctatctatataaagaggcaatcgtgtcaggaagcggctttttctttggccaaacggtacttcgaacttggaacgagcatgaagagattaacgagacttctttctcttttgagtttttctggcggaccggtcgcgcaagatctttggtcttcccccggaaccgatgaaaaagtgggtcgcttcttatggactcgctcagaatgattcttctctatctatagttcatggcctattagaagtagaaggtgctctggtgcaatccttaccgacagaaaaagattgcagtcgggttgataataatcgagtgccattacttcgtcggtccgaaccaaggaatccgttagaaatgttttgaaatggatattgttctctctttgattagggatgctatatgaaaagaagtggagtttgaaaaagggaacggaatggtcaaaccggaactgctagaggaacgaattttcaatagcataacttgggctcctagaatatggggcccttgggacaatctatttgattgcagggacaggtacgctgaatatgactggggattttcctatgggtattggagcgggtccaagcagattaaagaggatgagttgtcagagactgctatttattcgaattatttctggtatatttatcataaaaagggggaggtgcaagagactgattcggacttcttgcagagtggaacaatgcagtaccagacacgagatatatctttcaaagaagaaggcttttttcgaataagccaattgatttgggaccttcggatccattctttttcctattcaaagatcaggcctttgtctctgtgttttcacgtcgagaattctttgcagatgaagatgaagagatggcaaagcggcttagtaccagtacctggagaaaaaagcctcctaaacatatggctagcaactggttcaccaggagtacgcaagaaaggcaaaagcactacgaattattgatttaggaatgggaatgggctagaaccctgggctcttccttatataattaatggtcttttcattctaatactctatccgagagttctcagtatttagcaaagctgttcctatctaacggaaggctcctggatcaaatgacaaagacattgtttgtggagaaagaggtggcttttcccggatgaaatgaaacatttcatttgtgtaacaggagaaagatttcccactccttagccgtaaagatatgtggccatgaaaaagggaggggattcagtggaacaggattggccgggcggtagagtcgtggaaacacttgttgattcctattttggaccctagctccatggaacaatatgctactgcggaaacatggaagaattgcaatcttagatcaaaacactatgtatgggatgatatgaactgcctaaataagaattcttgagcgtcgaataacctgagtactaactacatcaaacaatttgcattaatgaaactgtgtaaatccaccggataatcaaaaatacgcatgtctgatgaaatggttgttgctatctgtttccataacgaatccttggtttaactgaataagtaaagaaaatgggccctttctcttcgtctcagatcgatggatcttctcgattggaagatctcccatatggataatacacattccagttgaccgagcctaatgctaattgttttgttccgaagcaaagatatccgcggaggccggttcgttcgtcctattctgatattcaggaccaagaggtcctggattctctttcggataggccctgaaaggagaagagaagctgaaatgccaacggacctctgtctattctctaattcacccgatccgatagtacccgtttttggaacgtccagtgccaaagtcactgaatgggtaagtcaccaatccaatccctttgacaaatcgggtgtcatattagatatcatattctatatatatagaaatatcatagaatagacatatagaatttttggttgggaaattcgaatgaatcattgagtgaaaaaggagcaaagaatgacaaaagatgagactctactagtcttcactcttgtggtttcctcggtttctattttcttattcgggatcttgcttttcatggttctcatctctgcaactcgcgattttcgcgagagaaccaaatccaagttggtgaagatcatgatttgggctggcatagtagttattacctttgcaattgcggttcgaatctatccgatctttatctttttgctcaaagaacgaataaaaccccttgttgaagccctttatgataagcttccctggatctgggaagtttctctttcacggtattgggatcgtttgatcgatttccttgatcgctacttatgggcgtgcgctcaaaggatacaaacagggattcgcaaacaaaaaggggaattcgtagtcactttttcctgtcgcgtaaaaaaaaggctttacgcgagagcaatagaggttgggatacatctatctcttctgagcaacctcttttggattcttaagaccacccttgcagtaggataccgtctgctttaggttctttattatctccttcgaggggtttttaggatcattcaggctatatttagtctattttggcttttactgtctacttttctcagggagatggttaaggacctcagaagatagaggagagcgccaggcgcagatttccggaatacttctacggggaatgctcattcaatgagcattctccgtattatgccttgaa

SEQ ID NO: 288 - DNA, 3007 bp, Zea mays
gctaacaaggatctaatgaattatgagtttaatagattctctttagcagaaagacgtatattccttgctcattatcagacaatcacttattcccaaacctcgtgtggggctaatcgttttcatttaccatctcatggaaaacccttttcgttccgcttagccctatcgggtattttagtgataggttctataggaactggacaatcctatttggtcaaatacctaacgaaaaattcctattttcctttcattaaggtacgagggcttcttattccacaagaacgaaagcaccttttcattctttcatatactaggggtttttacttggaaaagacaatgttccatactaaaggattcgggtccataaccacgagttccagtgcactagatcttgtagcacttagcaacgaggccctatccattagtattccacataagaaatccattatagaaactaatacaattagattagctcttcatagacaaacttggggtttgcgagccaagataagaccggctcgggatcatgggacccttttctatcagataggaggggatcttgtacaaaatagacttctaagtaataaccccatagatcctatatctatctatataaagaggcaatcgtgtcaggaagcggctttttctttggccaaacggtacttcgaacttggaacgagcatgaagagattaacgagacttctttctcttttgagtttttctggcggaccggtcgcgcaagatctttggtcttcccccggaaccgatgaaaaagtgggtcgcttcttatggactcgctcagaatgattcttctctatctatagttcatggcctattagaagtagaaggtgctctggtgcaatccttaccgacagaaaaagattgcagtcgggttgataataatcgagtgccattacttcgtcggtccgaaccaaggaatccgttagaaatgttttgaaatggatattgttctctctttgattagggatgctatatgaaaagaagtggagtttgaaaaagggaacggaatggtcaaaccggaactgctagaggaacgaattttcaatagcataacttgggctcctagaatatggggcccttgggacaatctatttgattgcagggacaggtacgctgaatatgactggggattttcctatgggtattggagcgggtccaagcagattaaagaggatgagttgtcagagactgctatttattcgaattatttctggtatatttatcataaaaagggggaggtgcaagagactgattcggacttcttgcagagtggaacaatgcagtaccagacacgagatatatctttcaaagaagaaggcttttttcgaataagccaattgatttgggaccttcggatccattctttttcctattcaaagatcaggcctttgtctctgtgttttcacgtcgagaattctttgcagatgaagatgaagagatggcaaagcggcttagtaccagtacctggagaaaaaagcctcctaaacatatggctagcaactggttcaccaggagtacgcaagaaaggcaaaagcactacgaattattgatttaggaatgggaatgggctagaaccctgggctcttccttatataattaatggtcttttcattctaatactctatccgagagttctcagtatttagcaaagctgttcctatctaacggaaggctcctggatcaaatgacaaagacattgtttgtggagaaagaggtggcttttcccggatgaaatgaaacatttcatttgtgtaacaggagaaagatttcccactccttagccgtaaagatatgtggccatgaaaaagggaggggattcagtggaacaggattggccgggcggtagagtcgtggaaacacttgttgattcctattttggaccctagctccatggaacaatatgctactgcggaaacatggaagaattgcaatcttagatcaaaacactatgtatgggatgatatgaactgcctaaataagaattcttgagcgtcgaataacctgagtactaactacatcaaacaatttgcattaatgaaactgtgtaaatccaccggataatcaaaaatacgcatgtctgatgaaatggttgttgctatctgtttccataacgaatccttggtttaactgaataagtaaagaaaatgggccctttctcttcgtctcagatcgatggatcttctcgattggaagatctcccatatggataatacacattccagttgaccgagcctaatgctaattgttttgttccgaagcaaagatatccgcggaggccggttcgttcgtcctattctgatattcaggaccaagaggtcctggattctctttcggataggccctgaaaggagaagagaagctgaaatgccaacggacctctgtctattctctaattcacccgatccgatagtacccgtttttggaacgtccagtgccaaagtcactgaatgggtaagtcaccaatccaatccctttgacaaatcgggtgtcatattagatatcatattctatatatatagaaatatcatagaatagacatatagaatttttggttgggaaattcgaatgaatcattgagtgaaaaaggagcaaagaatgacaaaagatgagactctactagtcttcactcttgtggtttcctcggtttctattttcttattcgggatcttgcttttcatggttctcatctctgcaactcgcgattttcgcgagagaaccaaatccaagttggtgaagatcatgatttgggctggcatagtagttattacctttgcaattgcggttcgaatctatccgatctttatctttttgctcaaagaacgaataaaaccccttgttgaagccctttatgataagcttccctggatctgggaagtttctctttcacggtattgggatcgtttgatcgatttccttgatcgctacttatgggcgtgcgctcaaaggatacaaacagggattcgcaaacaaaaaggggaattcgtag

SEQ ID NO: 289 - DNA, 3379 bp, Zea mays
gcaggaattgaacccgcaaatttaccaattatgagttgggcgctttaaccattcagccatggatgcttaacagggatcatcgtacatcgtaaataaccaattttcatatagaaagacatatcatagaaaaatgaaatcgaaaatattcggagatggcaaatattcggagatgactatgaaaacacctctccggatcctcgaattgaaagagagactgagagggatcaagaatcctaattctcgctatttggaatggatccaattctattgagtctgacccatagtgatcatttctctttagcaaagaatgaccttggttatcaaaggattgaacaaccgagatccatttacttatgatacctagttgacattgctaacaaggatctaatgaattatgagtttaatagattctctttagcagaaagacgtatattccttgctcattatcagacaatcacttattcccaaacctcgtgtggggctaatcgttttcatttaccatctcatggaaaacccttttcgttccgcttagccctatcgggtattttagtgataggttctataggaactggacaatcctatttggtcaaatacctaacgaaaaattcctattttcctttcattaaggtacgagggcttcttattccacaagaacgaaagcaccttttcattctttcatatactaggggtttttacttggaaaagacaatgttccatactaaaggattcgggtccataaccacgagttccagtgcactagatcttgtagcacttagcaacgaggccctatccattagtattccacataagaaatccattatagaaactaatacaattagattagctcttcatagacaaacttggggtttgcgagccaagataagaccggctcgggatcatgggacccttttctatcagataggaggggatcttgtacaaaatagacttctaagtaataaccccatagatcctatatctatctatataaagaggcaatcgtgtcaggaagcggctttttctttggccaaacggtacttcgaacttggaacgagcatgaagagattaacgagacttctttctcttttgagtttttctggcggaccggtcgcgcaagatctttggtcttcccccggaaccgatgaaaaagtgggtcgcttcttatggactcgctcagaatgattcttctctatctatagttcatggcctattagaagtagaaggtgctctggtgcaatccttaccgacagaaaaagattgcagtcgggttgataataatcgagtgccattacttcgtcggtccgaaccaaggaatccgttagaaatgttttgaaatggatattgttctctctttgattagggatgctatatgaaaagaagtggagtttgaaaaagggaacggaatggtcaaaccggaactgctagaggaacgaattttcaatagcataacttgggctcctagaatatggggcccttgggacaatctatttgattgcagggacaggtacgctgaatatgactggggattttcctatgggtattggagcgggtccaagcagattaaagaggatgagttgtcagagactgctatttattcgaattatttctggtatatttatcataaaaagggggaggtgcaagagactgattcggacttcttgcagagtggaacaatgcagtaccagacacgagatatatctttcaaagaagaaggcttttttcgaataagccaattgatttgggaccttcggatccattctttttcctattcaaagatcaggcctttgtctctgtgttttcacgtcgagaattctttgcagatgaagatgaagagatggcaaagcggcttagtaccagtacctggagaaaaaagcctcctaaacatatggctagcaactggttcaccaggagtacgcaagaaaggcaaaagcactacgaattattgatttaggaatgggaatgggctagaaccctgggctcttccttatataattaatggtcttttcattctaatactctatccgagagttctcagtatttagcaaagctgttcctatctaacggaaggctcctggatcaaatgacaaagacattgtttgtggagaaagaggtggcttttcccggatgaaatgaaacatttcatttgtgtaacaggagaaagatttcccactccttagccgtaaagatatgtggccatgaaaaagggaggggattcagtggaacaggattggccgggcggtagagtcgtggaaacacttgttgattcctattttggaccctagctccatggaacaatatgctactgcggaaacatggaagaattgcaatcttagatcaaaacactatgtatgggatgatatgaactgcctaaataagaattcttgagcgtcgaataacctgagtactaactacatcaaacaatttgcattaatgaaactgtgtaaatccaccggataatcaaaaatacgcatgtctgatgaaatggttgttgctatctgtttccataacgaatccttggtttaactgaataagtaaagaaaatgggccctttctcttcgtctcagatcgatggatcttctcgattggaagatctcccatatggataatacacattccagttgaccgagcctaatgctaattgttttgttccgaagcaaagatatccgcggaggccggttcgttcgtcctattctgatattcaggaccaagaggtcctggattctctttcggataggccctgaaaggagaagagaagctgaaatgccaacggacctctgtctattctctaattcacccgatccgatagtacccgtttttggaacgtccagtgccaaagtcactgaatgggtaagtcaccaatccaatccctttgacaaatcgggtgtcatattagatatcatattctatatatatagaaatatcatagaatagacatatagaatttttggttgggaaattcgaatgaatcattgagtgaaaaaggagcaaagaatgacaaaagatgagactctactagtcttcactcttgtggtttcctcggtttctattttcttattcgggatcttgcttttcatggttctcatctctgcaactcgcgattttcgcgagagaaccaaatccaagttggtgaagatcatgatttgggctggcatagtagttattacctttgcaattgcggttcgaatctatccgatctttatctttttgctcaaagaacgaataaaaccccttgttgaagccctttatgataagcttccctggatctgggaagtttctctttcacggtattgggatcgtttgatcgatttccttgatcgctacttatgggcgtgcgctcaaaggatacaaacagggattcgcaaacaaaaaggggaattcgtag

SEQ ID NO: 290 - DNA, 3334 bp, Zea mays
gctaacaaggatctaatgaattatgagtttaatagattctctttagcagaaagacgtatattccttgctcattatcagacaatcacttattcccaaacctcgtgtggggctaatcgttttcatttaccatctcatggaaaacccttttcgttccgcttagccctatcgggtattttagtgataggttctataggaactggacaatcctatttggtcaaatacctaacgaaaaattcctattttcctttcattaaggtacgagggcttcttattccacaagaacgaaagcaccttttcattctttcatatactaggggtttttacttggaaaagacaatgttccatactaaaggattcgggtccataaccacgagttccagtgcactagatcttgtagcacttagcaacgaggccctatccattagtattccacataagaaatccattatagaaactaatacaattagattagctcttcatagacaaacttggggtttgcgagccaagataagaccggctcgggatcatgggacccttttctatcagataggaggggatcttgtacaaaatagacttctaagtaataaccccatagatcctatatctatctatataaagaggcaatcgtgtcaggaagcggctttttctttggccaaacggtacttcgaacttggaacgagcatgaagagattaacgagacttctttctcttttgagtttttctggcggaccggtcgcgcaagatctttggtcttcccccggaaccgatgaaaaagtgggtcgcttcttatggactcgctcagaatgattcttctctatctatagttcatggcctattagaagtagaaggtgctctggtgcaatccttaccgacagaaaaagattgcagtcgggttgataataatcgagtgccattacttcgtcggtccgaaccaaggaatccgttagaaatgttttgaaatggatattgttctctctttgattagggatgctatatgaaaagaagtggagtttgaaaaagggaacggaatggtcaaaccggaactgctagaggaacgaattttcaatagcataacttgggctcctagaatatggggcccttgggacaatctatttgattgcagggacaggtacgctgaatatgactggggattttcctatgggtattggagcgggtccaagcagattaaagaggatgagttgtcagagactgctatttattcgaattatttctggtatatttatcataaaaagggggaggtgcaagagactgattcggacttcttgcagagtggaacaatgcagtaccagacacgagatatatctttcaaagaagaaggcttttttcgaataagccaattgatttgggaccttcggatccattctttttcctattcaaagatcaggcctttgtctctgtgttttcacgtcgagaattctttgcagatgaagatgaagagatggcaaagcggcttagtaccagtacctggagaaaaaagcctcctaaacatatggctagcaactggttcaccaggagtacgcaagaaaggcaaaagcactacgaattattgatttaggaatgggaatgggctagaaccctgggctcttccttatataattaatggtcttttcattctaatactctatccgagagttctcagtatttagcaaagctgttcctatctaacggaaggctcctggatcaaatgacaaagacattgtttgtggagaaagaggtggcttttcccggatgaaatgaaacatttcatttgtgtaacaggagaaagatttcccactccttagccgtaaagatatgtggccatgaaaaagggaggggattcagtggaacaggattggccgggcggtagagtcgtggaaacacttgttgattcctattttggaccctagctccatggaacaatatgctactgcggaaacatggaagaattgcaatcttagatcaaaacactatgtatgggatgatatgaactgcctaaataagaattcttgagcgtcgaataacctgagtactaactacatcaaacaatttgcattaatgaaactgtgtaaatccaccggataatcaaaaatacgcatgtctgatgaaatggttgttgctatctgtttccataacgaatccttggtttaactgaataagtaaagaaaatgggccctttctcttcgtctcagatcgatggatcttctcgattggaagatctcccatatggataatacacattccagttgaccgagcctaatgctaattgttttgttccgaagcaaagatatccgcggaggccggttcgttcgtcctattctgatattcaggaccaagaggtcctggattctctttcggataggccctgaaaggagaagagaagctgaaatgccaacggacctctgtctattctctaattcacccgatccgatagtacccgtttttggaacgtccagtgccaaagtcactgaatgggtaagtcaccaatccaatccctttgacaaatcgggtgtcatattagatatcatattctatatatatagaaatatcatagaatagacatatagaatttttggttgggaaattcgaatgaatcattgagtgaaaaaggagcaaagaatgacaaaagatgagactctactagtcttcactcttgtggtttcctcggtttctattttcttattcgggatcttgcttttcatggttctcatctctgcaactcgcgattttcgcgagagaaccaaatccaagttggtgaagatcatgatttgggctggcatagtagttattacctttgcaattgcggttcgaatctatccgatctttatctttttgctcaaagaacgaataaaaccccttgttgaagccctttatgataagcttccctggatctgggaagtttctctttcacggtattgggatcgtttgatcgatttccttgatcgctacttatgggcgtgcgctcaaaggatacaaacagggattcgcaaacaaaaaggggaattcgtagtcactttttcctgtcgcgtaaaaaaaaggctttacgcgagagcaatagaggttgggatacatctatctcttctgagcaacctcttttggattcttaagaccacccttgcagtaggataccgtctgctttaggttctttattatctccttcgaggggtttttaggatcattcaggctatatttagtctattttggcttttactgtctacttttctcagggagatggttaaggacctcagaagatagaggagagcgccaggcgcagatttccggaatacttctacggggaatgctcattcaatgagcattctccgtattatgccttgaa

SEQ ID NO: 291 - DNA, 2725 bp, Zea mays
aaaattggatcggattttgcggttcttttttctgcagtacctcgacgtgacatgagcgtgaaagaggttcaagaatccgttttctttttataagggctaaaaacgaatcacttatttttttggctttttggccccatattgtagggtggatctcgaaagataggaaagatctccctccaagccgtacatacgactttcatcgaatacggctttccacagaattctatagggatctatgagatcgagtatggaattctgtttactcactttaaattgagtatccgtttccctccttttcccgctaggaccggaaatcctgtattttccatatccatacgatcgagtccttaggtttccgaaatagtgtaatggaaaaagaagtgcttcgaatcattgctatttgactcggacctgttctgaaaaagtcgaggtatttcgaattgtttgttgacacggacaaagtaagggaaaacctctgaaagaatttccatattgaccttggacatataagagttccgaatcgaatctctttagaaagaagatcttttgtctcatggtagcctgctccagtccccttacgaaactttcgttattgggttagccatacacttcacatgtttctagcgattcacatggcatcatcaaatgatacaagtcttggataagaatctacaacgcactagaacgcccttgttgacgattctttactgcgacagcatctagggttcctcgaataatgcgatatctcacaccgggtaaatccttaacccttcctcctcttactaatactacagaatgttcttgtaaattatggccaataccaggtatataagcagtgatttcaaatccagaggttaatcgtactctggcaactttacgtaaggcagagttgggttttttggggttgatagtggaaaagtcgacagataagtcatccttactgtccctctacagaaccgtacatgagattttcacctcatacggctcctcgttcaattctttcgaagggatccttttcctcgttcgagagtctccgcccttcttccactccgtcccgaagactaactaagaccaattgagtcacgttttcatgttctaattgaacactttccatttatgattaaaggagaagattgttcttttaccaaacatatgcggatcaaatcacgtcttataataagaagaaatctttctcggtatcaatccccttgcccctcattctttgagaatcagaaggatccttttcgagtttccatttcttcattttgaatctgggctcttctatcttcgacttatttttttggctttattctttatttatttcatttcgatttttccctcttcctctatccctatcctctaggtacagcgtttgcatcaatagagaactttttcctctgtatgaatcgatattattccaatttcttcccgaaacttcccaagaaaaatcccgaattggatccaaaattgacgggttaatgtgagcttatccatgcggttaggcactcttcaaataggaatccattttctaactggctttcgtgctttggtgagtcgtccgagatcctttcgatgacctatgttgtgttgaagggatatctatatgatccgatcgattgcataagacccgcggtagcaatagaacggggaaagtatacagaaaagacagttcttttcgatttcgattatctatatattagttcgtttctatttctagatatctatttctatatatctatatattagtattaatatctatatattagtattagttatctatatattagtattagttagtagtactattctattagttagcgatcccggctctgtgagttctttcttccgtgatgaactgtcggcaccagtcctacattttttctctgtggaccgaggagaaagggggctcagcaggaagaggattgtaccatgagagaagcacagaggtcaacccgcttcaaatatggaacatggattctggcaatgcaacggagttgggtcctcatatcgatccgaatgaatcagtctttctacagaggtcaatctttgcctattaggcaagaggatagcaagttcgaaattctgtctcggtaggacatggatttctattactatgaaattcataaattagttaatgggggggctaccattatcctttttcttgtatgtgttcctaagagaaggaatttgtccatttcatgtttcgaggtctcaaaaaaagggcgtggaaacagatagaaactcttgaatggaaattgaaaagaaatgtagccccagttccttcggaaatggtaagatctttggcgcaagaagaaggggcgatccatatcatcttgacttggttctgcttcccctctttttttaagaataccgagtcgggttcttctcctaccagtatcgaatagaacatgctgaacaagatcttcttcatggaaacccactcgatttagatcgggaaaatcgtacagattttatgaaaccatgtgctatggctcgaatccatagtcaatcctattttcgataggaccggttgacaattgaatccaatttttcccattatttgactgtccataatagtgcggaaagaaagcccggaggaagagtggccttgcgtttctcgcccctttgccttaggattcgttaattctctttctcgatgggacggggaagggatataactcagcggtagagtgtcaccttgacgtggtggaagtcatcagttcga

SEQ ID NO: 292 - DNA, 1992 bp, Zea mays
tgtaatggaaaaagaagtgcttcgaatcattgctatttgactcggacctgttctgaaaaagtcgaggtatttcgaattgtttgttgacacggacaaagtaagggaaaacctctgaaagaatttccatattgaccttggacatataagagttccgaatcgaatctctttagaaagaagatcttttgtctcatggtagcctgctccagtccccttacgaaactttcgttattgggttagccatacacttcacatgtttctagcgattcacatggcatcatcaaatgatacaagtcttggataagaatctacaacgcactagaacgcccttgttgacgattctttactgcgacagcatctagggttcctcgaataatgcgatatctcacaccgggtaaatccttaacccttcctcctcttactaatactacagaatgttcttgtaaattatggccaataccaggtatataagcagtgatttcaaatccagaggttaatcgtactctggcaactttacgtaaggcagagttgggttttttggggttgatagtggaaaagtcgacagataagtcatccttactgtccctctacagaaccgtacatgagattttcacctcatacggctcctcgttcaattctttcgaagggatccttttcctcgttcgagagtctccgcccttcttccactccgtcccgaagactaactaagaccaattgagtcacgttttcatgttctaattgaacactttccatttatgattaaaggagaagattgttcttttaccaaacatatgcggatcaaatcacgtcttataataagaagaaatctttctcggtatcaatccccttgcccctcattctttgagaatcagaaggatccttttcgagtttccatttcttcattttgaatctgggctcttctatcttcgacttatttttttggctttattctttatttatttcatttcgatttttccctcttcctctatccctatcctctaggtacagcgtttgcatcaatagagaactttttcctctgtatgaatcgatattattccaatttcttcccgaaacttcccaagaaaaatcccgaattggatccaaaattgacgggttaatgtgagcttatccatgcggttaggcactcttcaaataggaatccattttctaactggctttcgtgctttggtgagtcgtccgagatcctttcgatgacctatgttgtgttgaagggatatctatatgatccgatcgattgcataagacccgcggtagcaatagaacggggaaagtatacagaaaagacagttcttttcgatttcgattatctatatattagttcgtttctatttctagatatctatttctatatatctatatattagtattaatatctatatattagtattagttatctatatattagtattagttagtagtactattctattagttagcgatcccggctctgtgagttctttcttccgtgatgaactgtcggcaccagtcctacattttttctctgtggaccgaggagaaagggggctcagcaggaagaggattgtaccatgagagaagcacagaggtcaacccgcttcaaatatggaacatggattctggcaatgcaacggagttgggtcctcatatcgatccgaatgaatcagtctttctacagaggtcaatctttgcctattaggcaagaggatagcaagttcgaaattctgtctcggtaggacatggatttctattactatgaaattcataaattagttaatgggggggctaccattatcctttttcttgtatgtgttcctaagagaaggaatttgtccatttcatgtttcgaggtctcaaaaaaagggcgtggaaacagatagaaactcttgaatggaaattgaaaagaaatgtagccccagttccttcggaaatggtaagatctttggcgcaagaagaaggggcgatccatatcatcttgacttggttctgcttcccctc

SEQ ID NO: 293 - DNA, 2356 bp, Zea mays
aaaattggatcggattttgcggttcttttttctgcagtacctcgacgtgacatgagcgtgaaagaggttcaagaatccgttttctttttataagggctaaaaacgaatcacttatttttttggctttttggccccatattgtagggtggatctcgaaagataggaaagatctccctccaagccgtacatacgactttcatcgaatacggctttccacagaattctatagggatctatgagatcgagtatggaattctgtttactcactttaaattgagtatccgtttccctccttttcccgctaggaccggaaatcctgtattttccatatccatacgatcgagtccttaggtttccgaaatagtgtaatggaaaaagaagtgcttcgaatcattgctatttgactcggacctgttctgaaaaagtcgaggtatttcgaattgtttgttgacacggacaaagtaagggaaaacctctgaaagaatttccatattgaccttggacatataagagttccgaatcgaatctctttagaaagaagatcttttgtctcatggtagcctgctccagtccccttacgaaactttcgttattgggttagccatacacttcacatgtttctagcgattcacatggcatcatcaaatgatacaagtcttggataagaatctacaacgcactagaacgcccttgttgacgattctttactgcgacagcatctagggttcctcgaataatgcgatatctcacaccgggtaaatccttaacccttcctcctcttactaatactacagaatgttcttgtaaattatggccaataccaggtatataagcagtgatttcaaatccagaggttaatcgtactctggcaactttacgtaaggcagagttgggttttttggggttgatagtggaaaagtcgacagataagtcatccttactgtccctctacagaaccgtacatgagattttcacctcatacggctcctcgttcaattctttcgaagggatccttttcctcgttcgagagtctccgcccttcttccactccgtcccgaagactaactaagaccaattgagtcacgttttcatgttctaattgaacactttccatttatgattaaaggagaagattgttcttttaccaaacatatgcggatcaaatcacgtcttataataagaagaaatctttctcggtatcaatccccttgcccctcattctttgagaatcagaaggatccttttcgagtttccatttcttcattttgaatctgggctcttctatcttcgacttatttttttggctttattctttatttatttcatttcgatttttccctcttcctctatccctatcctctaggtacagcgtttgcatcaatagagaactttttcctctgtatgaatcgatattattccaatttcttcccgaaacttcccaagaaaaatcccgaattggatccaaaattgacgggttaatgtgagcttatccatgcggttaggcactcttcaaataggaatccattttctaactggctttcgtgctttggtgagtcgtccgagatcctttcgatgacctatgttgtgttgaagggatatctatatgatccgatcgattgcataagacccgcggtagcaatagaacggggaaagtatacagaaaagacagttcttttcgatttcgattatctatatattagttcgtttctatttctagatatctatttctatatatctatatattagtattaatatctatatattagtattagttatctatatattagtattagttagtagtactattctattagttagcgatcccggctctgtgagttctttcttccgtgatgaactgtcggcaccagtcctacattttttctctgtggaccgaggagaaagggggctcagcaggaagaggattgtaccatgagagaagcacagaggtcaacccgcttcaaatatggaacatggattctggcaatgcaacggagttgggtcctcatatcgatccgaatgaatcagtctttctacagaggtcaatctttgcctattaggcaagaggatagcaagttcgaaattctgtctcggtaggacatggatttctattactatgaaattcataaattagttaatgggggggctaccattatcctttttcttgtatgtgttcctaagagaaggaatttgtccatttcatgtttcgaggtctcaaaaaaagggcgtggaaacagatagaaactcttgaatggaaattgaaaagaaatgtagccccagttccttcggaaatggtaagatctttggcgcaagaagaaggggcgatccatatcatcttgacttggttctgcttcccctc

SEQ ID NO: 294 - DNA, 2361 bp, Zea mays
tgtaatggaaaaagaagtgcttcgaatcattgctatttgactcggacctgttctgaaaaagtcgaggtatttcgaattgtttgttgacacggacaaagtaagggaaaacctctgaaagaatttccatattgaccttggacatataagagttccgaatcgaatctctttagaaagaagatcttttgtctcatggtagcctgctccagtccccttacgaaactttcgttattgggttagccatacacttcacatgtttctagcgattcacatggcatcatcaaatgatacaagtcttggataagaatctacaacgcactagaacgcccttgttgacgattctttactgcgacagcatctagggttcctcgaataatgcgatatctcacaccgggtaaatccttaacccttcctcctcttactaatactacagaatgttcttgtaaattatggccaataccaggtatataagcagtgatttcaaatccagaggttaatcgtactctggcaactttacgtaaggcagagttgggttttttggggttgatagtggaaaagtcgacagataagtcatccttactgtccctctacagaaccgtacatgagattttcacctcatacggctcctcgttcaattctttcgaagggatccttttcctcgttcgagagtctccgcccttcttccactccgtcccgaagactaactaagaccaattgagtcacgttttcatgttctaattgaacactttccatttatgattaaaggagaagattgttcttttaccaaacatatgcggatcaaatcacgtcttataataagaagaaatctttctcggtatcaatccccttgcccctcattctttgagaatcagaaggatccttttcgagtttccatttcttcattttgaatctgggctcttctatcttcgacttatttttttggctttattctttatttatttcatttcgatttttccctcttcctctatccctatcctctaggtacagcgtttgcatcaatagagaactttttcctctgtatgaatcgatattattccaatttcttcccgaaacttcccaagaaaaatcccgaattggatccaaaattgacgggttaatgtgagcttatccatgcggttaggcactcttcaaataggaatccattttctaactggctttcgtgctttggtgagtcgtccgagatcctttcgatgacctatgttgtgttgaagggatatctatatgatccgatcgattgcataagacccgcggtagcaatagaacggggaaagtatacagaaaagacagttcttttcgatttcgattatctatatattagttcgtttctatttctagatatctatttctatatatctatatattagtattaatatctatatattagtattagttatctatatattagtattagttagtagtactattctattagttagcgatcccggctctgtgagttctttcttccgtgatgaactgtcggcaccagtcctacattttttctctgtggaccgaggagaaagggggctcagcaggaagaggattgtaccatgagagaagcacagaggtcaacccgcttcaaatatggaacatggattctggcaatgcaacggagttgggtcctcatatcgatccgaatgaatcagtctttctacagaggtcaatctttgcctattaggcaagaggatagcaagttcgaaattctgtctcggtaggacatggatttctattactatgaaattcataaattagttaatgggggggctaccattatcctttttcttgtatgtgttcctaagagaaggaatttgtccatttcatgtttcgaggtctcaaaaaaagggcgtggaaacagatagaaactcttgaatggaaattgaaaagaaatgtagccccagttccttcggaaatggtaagatctttggcgcaagaagaaggggcgatccatatcatcttgacttggttctgcttcccctctttttttaagaataccgagtcgggttcttctcctaccagtatcgaatagaacatgctgaacaagatcttcttcatggaaacccactcgatttagatcgggaaaatcgtacagattttatgaaaccatgtgctatggctcgaatccatagtcaatcctattttcgataggaccggttgacaattgaatccaatttttcccattatttgactgtccataatagtgcggaaagaaagcccggaggaagagtggccttgcgtttctcgcccctttgccttaggattcgttaattctctttctcgatgggacggggaagggatataactcagcggtagagtgtcaccttgacgtggtggaagtcatcagttcga

SEQ ID NO: 295 - DNA, 1744 bp, Zea mays
taggattcgaacctacgaccagtcagttaacagccgaccgctctaccactgagctactgaggaacaaggggagattcgacctcctagagttcaactcccgctctcaacccgtgaacaatatgagtccgaagcttctttcgtaactcccggaatttcttcgtagtggctccgttccatgcctcatttcatagggaagcccaaagtggctctatttcattctatttcacttcttagcacttcctatcatttaatatccattcctttggtcttattgacataagagatgtcatttatagtctatctctttctatatatggaaagtcaagaaattctcatcgaaacatcgagaaattgtgcatatagaaaactctaaagaaagaaaaaaaggagacccatgccatgattttcaaatcttttctacttaagtagtctaaagtagtctaagtttctcgatgaggataattaattcggtcgttgtggtcggactctattatggatttctgaccacattctccataggtccctcttagatcttctttctccaatcttgggttagggaagaaggagatattcgcgactactggcggtttcattatggggcagctcatgatcttcatatcgatctattatccacctctgcatctattctttcttagctaaacgggtggaagatccatccaatttggttatatcatggactcgaaaaacggatctgaatgtgactgaaatgcacgatcttcacaggtatcacttttcacgatacctaaacctaaaaggtggaatagcgattttcgaaccatttcctataagagaatggtttccattactttgagaaatggattctatatcaaactatagctattgcattaaagaagaaaagaaactaatagaagtcgaagacgcggaatggtagtgaatagagaaaaagattcttctgattttcttgttcctgaaaatattcgatccatctcctagacgccgtagagaattgagaattttcatgtctttcaattctcgtactcgtaattggaaagttacggaaggagatccatcattttgcaatgaaaacaacataaaaaactctggacaatttcgaaatcagaccaagcgtcttaatacatatgcaaaaaaattcattattggcccaccattgattacaagatttagcttttatgaatcgctattggtttgatacgaataatggcagtcgtttcagtatgttaaggatacagatgtatccacaattcatttagagttacttaatagcctatttcttatactatatctctatcccgtgaaattctcgagcccaaagatggatgcatatgctgtgtttcattttgctaaatgatatcaattaaatggtatatcaattctataaattggatatagcaataaataaatcagcaaaattcttttattttagatagaagaaatgtttcttctatctaaaataaaagaatgtacccttctatccaaatccaatttgcatcgataaaataaatccaaattccagattccagcagtagatgaataattgcaaatttttgtgtgtacgagattagaataacttaaaaataactgacataattttttatttttcctgatcagaaaaatacatgaaaaagaaaggaggtagaaaaatttgttgatttatggttaaagaagaaaaacaagaaaacaggggttctgttgaatttcaagtattcagtttcaccaataagatacggagacttgcttcaca

SEQ ID NO: 296 - DNA, 961 bp, Zea mays
taaagaaagaaaaaaaggagacccatgccatgattttcaaatcttttctacttaagtagtctaaagtagtctaagtttctcgatgaggataattaattcggtcgttgtggtcggactctattatggatttctgaccacattctccataggtccctcttagatcttctttctccaatcttgggttagggaagaaggagatattcgcgactactggcggtttcattatggggcagctcatgatcttcatatcgatctattatccacctctgcatctattctttcttagctaaacgggtggaagatccatccaatttggttatatcatggactcgaaaaacggatctgaatgtgactgaaatgcacgatcttcacaggtatcacttttcacgatacctaaacctaaaaggtggaatagcgattttcgaaccatttcctataagagaatggtttccattactttgagaaatggattctatatcaaactatagctattgcattaaagaagaaaagaaactaatagaagtcgaagacgcggaatggtagtgaatagagaaaaagattcttctgattttcttgttcctgaaaatattcgatccatctcctagacgccgtagagaattgagaattttcatgtctttcaattctcgtactcgtaattggaaagttacggaaggagatccatcattttgcaatgaaaacaacataaaaaactctggacaatttcgaaatcagaccaagcgtcttaatacatatgcaaaaaaattcattattggcccaccattgattacaagatttagcttttatgaatcgctattggtttgatacgaataatggcagtcgtttcagtatgttaaggatacagatgtatccacaattcatttagagttacttaatagcctatttcttatactatatctctatcccgtgaaattctcgagcccaaagatggatgcatatgctg

SEQ ID NO: 297 - DNA, 1331 bp, Zea mays
taggattcgaacctacgaccagtcagttaacagccgaccgctctaccactgagctactgaggaacaaggggagattcgacctcctagagttcaactcccgctctcaacccgtgaacaatatgagtccgaagcttctttcgtaactcccggaatttcttcgtagtggctccgttccatgcctcatttcatagggaagcccaaagtggctctatttcattctatttcacttcttagcacttcctatcatttaatatccattcctttggtcttattgacataagagatgtcatttatagtctatctctttctatatatggaaagtcaagaaattctcatcgaaacatcgagaaattgtgcatatagaaaactctaaagaaagaaaaaaaggagacccatgccatgattttcaaatcttttctacttaagtagtctaaagtagtctaagtttctcgatgaggataattaattcggtcgttgtggtcggactctattatggatttctgaccacattctccataggtccctcttagatcttctttctccaatcttgggttagggaagaaggagatattcgcgactactggcggtttcattatggggcagctcatgatcttcatatcgatctattatccacctctgcatctattctttcttagctaaacgggtggaagatccatccaatttggttatatcatggactcgaaaaacggatctgaatgtgactgaaatgcacgatcttcacaggtatcacttttcacgatacctaaacctaaaaggtggaatagcgattttcgaaccatttcctataagagaatggtttccattactttgagaaatggattctatatcaaactatagctattgcattaaagaagaaaagaaactaatagaagtcgaagacgcggaatggtagtgaatagagaaaaagattcttctgattttcttgttcctgaaaatattcgatccatctcctagacgccgtagagaattgagaattttcatgtctttcaattctcgtactcgtaattggaaagttacggaaggagatccatcattttgcaatgaaaacaacataaaaaactctggacaatttcgaaatcagaccaagcgtcttaatacatatgcaaaaaaattcattattggcccaccattgattacaagatttagcttttatgaatcgctattggtttgatacgaataatggcagtcgtttcagtatgttaaggatacagatgtatccacaattcatttagagttacttaatagcctatttcttatactatatctctatcccgtgaaattctcgagcccaaagatggatgcatatgctg

SEQ ID NO: 298 - DNA, 1374 bp, Zea mays
taaagaaagaaaaaaaggagacccatgccatgattttcaaatcttttctacttaagtagtctaaagtagtctaagtttctcgatgaggataattaattcggtcgttgtggtcggactctattatggatttctgaccacattctccataggtccctcttagatcttctttctccaatcttgggttagggaagaaggagatattcgcgactactggcggtttcattatggggcagctcatgatcttcatatcgatctattatccacctctgcatctattctttcttagctaaacgggtggaagatccatccaatttggttatatcatggactcgaaaaacggatctgaatgtgactgaaatgcacgatcttcacaggtatcacttttcacgatacctaaacctaaaaggtggaatagcgattttcgaaccatttcctataagagaatggtttccattactttgagaaatggattctatatcaaactatagctattgcattaaagaagaaaagaaactaatagaagtcgaagacgcggaatggtagtgaatagagaaaaagattcttctgattttcttgttcctgaaaatattcgatccatctcctagacgccgtagagaattgagaattttcatgtctttcaattctcgtactcgtaattggaaagttacggaaggagatccatcattttgcaatgaaaacaacataaaaaactctggacaatttcgaaatcagaccaagcgtcttaatacatatgcaaaaaaattcattattggcccaccattgattacaagatttagcttttatgaatcgctattggtttgatacgaataatggcagtcgtttcagtatgttaaggatacagatgtatccacaattcatttagagttacttaatagcctatttcttatactatatctctatcccgtgaaattctcgagcccaaagatggatgcatatgctgtgtttcattttgctaaatgatatcaattaaatggtatatcaattctataaattggatatagcaataaataaatcagcaaaattcttttattttagatagaagaaatgtttcttctatctaaaataaaagaatgtacccttctatccaaatccaatttgcatcgataaaataaatccaaattccagattccagcagtagatgaataattgcaaatttttgtgtgtacgagattagaataacttaaaaataactgacataattttttatttttcctgatcagaaaaatacatgaaaaagaaaggaggtagaaaaatttgttgatttatggttaaagaagaaaaacaagaaaacaggggttctgttgaatttcaagtattcagtttcaccaataagatacggagacttgcttcaca

SEQ ID NO: 299 - DNA, 981 bp, Zea mays
caaatcccattgacataataactggaagtgggagaagaggaattacccaggcatattgatatgtatgttccataagaaaagaaatagcaatttttagtttaaatttatagttcaatttttctataaaataaaattgtttccgattcaccaaacctattcttatctctttctaaaggaattaaaaaaacaaaataagaaaaataaaaaatactggaattatgaatttttctaatttctctcattaaaatattccaaaatttagaatgggtttagttacttaaattcaaaaagtgaatcaaagaatttcggtatctagttattagtaaaaaaaaatatttattaaaatacaaaaaaagattgaataattttactttctaatcatttttgtatttctttttgttaaaataaaagagctctcttgtttcgtaattgattgattgaattccaaagaaaacctatatttataggaaattctcgaatattgcttatttcatataaaaggaaatcctaatgactagaattctctaagttttagaatgtcttgtttaagagactaagtatttttttttattggaattcaattatggaatagccttctgaacttaattaactatttgaattgaattttaccttctttttatctccccctcttatatgagggcttatcccccataccatatatttatatatggagtatatttgatatagaaattgatttaaatagaaaatcttaaaaaactcttgtcttatccacattagacaaaatgaactaaagaagaatttagaatttaagtatctttcagtatctttcagtatctaagtataaatactaagaaaaaacagaaagaaggattgatttgtggcaatagatgtctttcacatacaactagaaaaaagtaattccctttttgaatggcagttccaaaaaaacgtacttcgatgtcaaaaaagcgtattcgtaaaaatctttggaaaaaaaag

SEQ ID NO: 300 - DNA, 292 bp, Zea mays
ggtatctagttattagtaaaaaaaaatatttattaaaatacaaaaaaagattgaataattttactttctaatcatttttgtatttctttttgttaaaataaaagagctctcttgtttcgtaattgattgattgaattccaaagaaaacctatatttataggaaattctcgaatattgcttatttcatataaaaggaaatcctaatgactagaattctctaagttttagaatgtcttgtttaagagactaagtatttttttttattggaattcaattatggaatagccttctg

SEQ ID NO: 301 - DNA, 599 bp, Zea mays
caaatcccattgacataataactggaagtgggagaagaggaattacccaggcatattgatatgtatgttccataagaaaagaaatagcaatttttagtttaaatttatagttcaatttttctataaaataaaattgtttccgattcaccaaacctattcttatctctttctaaaggaattaaaaaaacaaaataagaaaaataaaaaatactggaattatgaatttttctaatttctctcattaaaatattccaaaatttagaatgggtttagttacttaaattcaaaaagtgaatcaaagaatttcggtatctagttattagtaaaaaaaaatatttattaaaatacaaaaaaagattgaataattttactttctaatcatttttgtatttctttttgttaaaataaaagagctctcttgtttcgtaattgattgattgaattccaaagaaaacctatatttataggaaattctcgaatattgcttatttcatataaaaggaaatcctaatgactagaattctctaagttttagaatgtcttgtttaagagactaagtatttttttttattggaattcaattatggaatagccttctg

SEQ ID NO: 302 - DNA, 674 bp, Zea mays
ggtatctagttattagtaaaaaaaaatatttattaaaatacaaaaaaagattgaataattttactttctaatcatttttgtatttctttttgttaaaataaaagagctctcttgtttcgtaattgattgattgaattccaaagaaaacctatatttataggaaattctcgaatattgcttatttcatataaaaggaaatcctaatgactagaattctctaagttttagaatgtcttgtttaagagactaagtatttttttttattggaattcaattatggaatagccttctgaacttaattaactatttgaattgaattttaccttctttttatctccccctcttatatgagggcttatcccccataccatatatttatatatggagtatatttgatatagaaattgatttaaatagaaaatcttaaaaaactcttgtcttatccacattagacaaaatgaactaaagaagaatttagaatttaagtatctttcagtatctttcagtatctaagtataaatactaagaaaaaacagaaagaaggattgatttgtggcaatagatgtctttcacatacaactagaaaaaagtaattccctttttgaatggcagttccaaaaaaacgtacttcgatgtcaaaaaagcgtattcgtaaaaatctttggaaaaaaaag

SEQ ID NO: 303 - DNA, 1635 bp, Zea mays
aaccataaatcaacaaatttttctacctcctttctttttcatgtatttttctgatcaggaaaaataaaaaattatgtcagttatttttaagttattctaatctcgtacacacaaaaatttgcaattattcatctactgctggaatctggaatttggatttattttatcgatgcaaattggatttggatagaagggtacattcttttattttagatagaagaaacatttcttctatctaaaataaaagaattttgctgatttatttattgctatatccaatttatagaattgatataccatttaattgatatcatttagcaaaatgaaacacagcatatgcatccatctttgggctcgagaatttcacgggatagagatatagtataagaaataggctattaagtaactctaaatgaattgtggatacatctgtatccttaacatactgaaacgactgccattattcgtatcaaaccaatagcgattcataaaagctaaatcttgtaatcaatggtgggccaataatgaatttttttgcatatgtattaagacgcttggtctgatttcgaaattgtccagagttttttatgttgttttcattgcaaaatgatggatctccttccgtaactttccaattacgagtacgagaattgaaagacatgaaaattctcaattctctacggcgtctaggagatggatcgaatattttcaggaacaagaaaatcagaagaatctttttctctattcactaccattccgcgtcttcgacttctattagtttcttttcttctttaatgcaatagctatagtttgatatagaatccatttctcaaagtaatggaaaccattctcttataggaaatggttcgaaaatcgctattccaccttttaggtttaggtatcgtgaaaagtgatacctgtgaagatcgtgcatttcagtcacattcagatccgtttttcgagtccatgatataaccaaattggatggatcttccacccgtttagctaagaaagaatagatgcagaggtggataatagatcgatatgaagatcatgagctgccccataatgaaaccgccagtagtcgcgaatatctccttcttccctaacccaagattggagaaagaagatctaagagggacctatggagaatgtggtcagaaatccataatagagtccgaccacaacgaccgaattaattatcctcatcgagaaacttagactactttagactacttaagtagaaaagatttgaaaatcatggcatgggtctcctttttttctttctttagagttttctatatgcacaatttctcgatgtttcgatgagaatttcttgactttccatatatagaaagagatagactataaatgacatctcttatgtcaataagaccaaaggaatggatattaaatgataggaagtgctaagaagtgaaatagaatgaaatagagccactttgggcttccctatgaaatgaggcatggaacggagccactacgaagaaattccgggagttacgaaagaagcttcggactcatattgttcacgggttgagagcgggagttgaactctaggaggtcgaatctccccttgttcctcagtagctcagtggtagagcggtcggctgttaa

SEQ ID NO: 304 - DNA, 943 bp, Zea mays
acacagcatatgcatccatctttgggctcgagaatttcacgggatagagatatagtataagaaataggctattaagtaactctaaatgaattgtggatacatctgtatccttaacatactgaaacgactgccattattcgtatcaaaccaatagcgattcataaaagctaaatcttgtaatcaatggtgggccaataatgaatttttttgcatatgtattaagacgcttggtctgatttcgaaattgtccagagttttttatgttgttttcattgcaaaatgatggatctccttccgtaactttccaattacgagtacgagaattgaaagacatgaaaattctcaattctctacggcgtctaggagatggatcgaatattttcaggaacaagaaaatcagaagaatctttttctctattcactaccattccgcgtcttcgacttctattagtttcttttcttctttaatgcaatagctatagtttgatatagaatccatttctcaaagtaatggaaaccattctcttataggaaatggttcgaaaatcgctattccaccttttaggtttaggtatcgtgaaaagtgatacctgtgaagatcgtgcatttcagtcacattcagatccgtttttcgagtccatgatataaccaaattggatggatcttccacccgtttagctaagaaagaatagatgcagaggtggataatagatcgatatgaagatcatgagctgccccataatgaaaccgccagtagtcgcgaatatctccttcttccctaacccaagattggagaaagaagatctaagagggacctatggagaatgtggtcagaaatccataatagagtccgaccacaacgaccgaattaattatcctcatcgagaaacttagactactttagactacttaagtagaaaagatttgaaaatcatggcatggg

SEQ ID NO: 305 - DNA, 1270 bp, Zea mays
aaccataaatcaacaaatttttctacctcctttctttttcatgtatttttctgatcaggaaaaataaaaaattatgtcagttatttttaagttattctaatctcgtacacacaaaaatttgcaattattcatctactgctggaatctggaatttggatttattttatcgatgcaaattggatttggatagaagggtacattcttttattttagatagaagaaacatttcttctatctaaaataaaagaattttgctgatttatttattgctatatccaatttatagaattgatataccatttaattgatatcatttagcaaaatgaaacacagcatatgcatccatctttgggctcgagaatttcacgggatagagatatagtataagaaataggctattaagtaactctaaatgaattgtggatacatctgtatccttaacatactgaaacgactgccattattcgtatcaaaccaatagcgattcataaaagctaaatcttgtaatcaatggtgggccaataatgaatttttttgcatatgtattaagacgcttggtctgatttcgaaattgtccagagttttttatgttgttttcattgcaaaatgatggatctccttccgtaactttccaattacgagtacgagaattgaaagacatgaaaattctcaattctctacggcgtctaggagatggatcgaatattttcaggaacaagaaaatcagaagaatctttttctctattcactaccattccgcgtcttcgacttctattagtttcttttcttctttaatgcaatagctatagtttgatatagaatccatttctcaaagtaatggaaaccattctcttataggaaatggttcgaaaatcgctattccaccttttaggtttaggtatcgtgaaaagtgatacctgtgaagatcgtgcatttcagtcacattcagatccgtttttcgagtccatgatataaccaaattggatggatcttccacccgtttagctaagaaagaatagatgcagaggtggataatagatcgatatgaagatcatgagctgccccataatgaaaccgccagtagtcgcgaatatctccttcttccctaacccaagattggagaaagaagatctaagagggacctatggagaatgtggtcagaaatccataatagagtccgaccacaacgaccgaattaattatcctcatcgagaaacttagactactttagactacttaagtagaaaagatttgaaaatcatggcatggg

SEQ ID NO: 306 - DNA, 1308 bp, Zea mays
acacagcatatgcatccatctttgggctcgagaatttcacgggatagagatatagtataagaaataggctattaagtaactctaaatgaattgtggatacatctgtatccttaacatactgaaacgactgccattattcgtatcaaaccaatagcgattcataaaagctaaatcttgtaatcaatggtgggccaataatgaatttttttgcatatgtattaagacgcttggtctgatttcgaaattgtccagagttttttatgttgttttcattgcaaaatgatggatctccttccgtaactttccaattacgagtacgagaattgaaagacatgaaaattctcaattctctacggcgtctaggagatggatcgaatattttcaggaacaagaaaatcagaagaatctttttctctattcactaccattccgcgtcttcgacttctattagtttcttttcttctttaatgcaatagctatagtttgatatagaatccatttctcaaagtaatggaaaccattctcttataggaaatggttcgaaaatcgctattccaccttttaggtttaggtatcgtgaaaagtgatacctgtgaagatcgtgcatttcagtcacattcagatccgtttttcgagtccatgatataaccaaattggatggatcttccacccgtttagctaagaaagaatagatgcagaggtggataatagatcgatatgaagatcatgagctgccccataatgaaaccgccagtagtcgcgaatatctccttcttccctaacccaagattggagaaagaagatctaagagggacctatggagaatgtggtcagaaatccataatagagtccgaccacaacgaccgaattaattatcctcatcgagaaacttagactactttagactacttaagtagaaaagatttgaaaatcatggcatgggtctcctttttttctttctttagagttttctatatgcacaatttctcgatgtttcgatgagaatttcttgactttccatatatagaaagagatagactataaatgacatctcttatgtcaataagaccaaaggaatggatattaaatgataggaagtgctaagaagtgaaatagaatgaaatagagccactttgggcttccctatgaaatgaggcatggaacggagccactacgaagaaattccgggagttacgaaagaagcttcggactcatattgttcacgggttgagagcgggagttgaactctaggaggtcgaatctccccttgttcctcagtagctcagtggtagagcggtcggctgttaa

SEQ ID NO: 307 - DNA, 2725 bp, Zea mays
cacattaggtttagggataatcaggctcgaactgatgacttccaccacgtcaaggtgacactctaccgctgagttatatcccttccccgtcccatcgagaaagagaattaacgaatcctaaggcaaaggggcgagaaacgcaaggccactcttcctccgggctttctttccgcactattatggacagtcaaataatgggaaaaattggattcaattgtcaaccggtcctatcgaaaataggattgactatggattcgagccatagcacatggtttcataaaatctgtacgattttcccgatctaaatcgagtgggtttccatgaagaagatcttgttcagcatgttctattcgatactggtaggagaagaacccgactcggtattcttaaaaaaagaggggaagcagaaccaagtcaagatgatatggatcgccccttcttcttgcgccaaagatcttaccatttccgaaggaactggggctacatttcttttcaatttccattcaagagtttctatctgtttccacgccctttttttgagacctcgaaacatgaaatggacaaattccttctcttaggaacacatacaagaaaaaggataatggtagcccccccattaactaatttatgaatttcatagtaatagaaatccatgtcctaccgagacagaatttcgaacttgctatcctcttgcctaataggcaaagattgacctctgtagaaagactgattcattcggatcgatatgaggacccaactccgttgcattgccagaatccatgttccatatttgaagcgggttgacctctgtgcttctctcatggtacaatcctcttcctgctgagccccctttctcctcggtccacagagaaaaaatgtaggactggtgccgacagttcatcacggaagaaagaactcacagagccgggatcgctaactaatagaatagtactactaactaatactaatatatagataactaatactaatatatagatattaatactaatatatagatatatagaaatagatatctagaaatagaaacgaactaatatatagataatcgaaatcgaaaagaactgtcttttctgtatactttccccgttctattgctaccgcgggtcttatgcaatcgatcggatcatatagatatcccttcaacacaacataggtcatcgaaaggatctcggacgactcaccaaagcacgaaagccagttagaaaatggattcctatttgaagagtgcctaaccgcatggataagctcacattaacccgtcaattttggatccaattcgggatttttcttgggaagtttcgggaagaaattggaataatatcgattcatacagaggaaaaagttctctattgatgcaaacgctgtacctagaggatagggatagaggaagagggaaaaatcgaaatgaaataaataaagaataaagccaaaaaaataagtcgaagatagaagagcccagattcaaaatgaagaaatggaaactcgaaaaggatccttctgattctcaaagaatgaggggcaaggggattgataccgagaaagatttcttcttattataagacgtgatttgatccgcatatgtttggtaaaagaacaatcttctcctttaatcataaatggaaagtgttcaattagaacatgaaaacgtgactcaattggtcttagttagtcttcgggacggagtggaagaagggcggagactctcgaacgaggaaaaggatcccttcgaaagaattgaacgaggagccgtatgaggtgaaaatctcatgtacggttctgtagagggacagtaaggatgacttatctgtcgacttttccactatcaaccccaaaaaacccaactctgccttacgtaaagttgccagagtacgattaacctctggatttgaaatcactgcttatatacctggtattggccataatttacaagaacattctgtagtattagtaagaggaggaagggttaaggatttacccggtgtgagatatcgcattattcgaggaaccctagatgctgtcgcagtaaagaatcgtcaacaagggcgttctagtgcgttgtagattcttatccaagacttgtatcatttgatgatgccatgtgaatcgctagaaacatgtgaagtgtatggctaacccaataacgaaagtttcgtaaggggactggagcaggctaccatgagacaaaagatcttctttctaaagagattcgattcggaactcttatatgtccaaggtcaatatggaaattctttcagaggttttcccttactttgtccgtgtcaacaaacaattcgaaatacctcgactttttcagaacaggtccgagtcaaatagcaatgattcgaagcacttctttttccattacactatttcggaaacctaaggactcgatcgtatggatatggaaaatacaggatttccggtcctagcgggaaaaggagggaaacggatactcaatttaaagtgagtaaacagaattccatactcgatctcatagatccctatagaattctgtggaaagccgtattcgatgaaagtcgtatgtacggcttggagggagatctttcctatctttcgagatccaccctacaatatggggccaaaaagccaaaaaaataagtgattcgtttttagcccttataaaaagaaaacggattcttgaacctctttcacgctcatgtcacgtcgaggtactgcagaaaaa

SEQ ID NO: 308 - DNA, 2003 bp, Zea mays
gaggggaagcagaaccaagtcaagatgatatggatcgccccttcttcttgcgccaaagatcttaccatttccgaaggaactggggctacatttcttttcaatttccattcaagagtttctatctgtttccacgccctttttttgagacctcgaaacatgaaatggacaaattccttctcttaggaacacatacaagaaaaaggataatggtagcccccccattaactaatttatgaatttcatagtaatagaaatccatgtcctaccgagacagaatttcgaacttgctatcctcttgcctaataggcaaagattgacctctgtagaaagactgattcattcggatcgatatgaggacccaactccgttgcattgccagaatccatgttccatatttgaagcgggttgacctctgtgcttctctcatggtacaatcctcttcctgctgagccccctttctcctcggtccacagagaaaaaatgtaggactggtgccgacagttcatcacggaagaaagaactcacagagccgggatcgctaactaatagaatagtactactaactaatactaatatatagataactaatactaatatatagatattaatactaatatatagatatatagaaatagatatctagaaatagaaacgaactaatatatagataatcgaaatcgaaaagaactgtcttttctgtatactttccccgttctattgctaccgcgggtcttatgcaatcgatcggatcatatagatatcccttcaacacaacataggtcatcgaaaggatctcggacgactcaccaaagcacgaaagccagttagaaaatggattcctatttgaagagtgcctaaccgcatggataagctcacattaacccgtcaattttggatccaattcgggatttttcttgggaagtttcgggaagaaattggaataatatcgattcatacagaggaaaaagttctctattgatgcaaacgctgtacctagaggatagggatagaggaagagggaaaaatcgaaatgaaataaataaagaataaagccaaaaaaataagtcgaagatagaagagcccagattcaaaatgaagaaatggaaactcgaaaaggatccttctgattctcaaagaatgaggggcaaggggattgataccgagaaagatttcttcttattataagacgtgatttgatccgcatatgtttggtaaaagaacaatcttctcctttaatcataaatggaaagtgttcaattagaacatgaaaacgtgactcaattggtcttagttagtcttcgggacggagtggaagaagggcggagactctcgaacgaggaaaaggatcccttcgaaagaattgaacgaggagccgtatgaggtgaaaatctcatgtacggttctgtagagggacagtaaggatgacttatctgtcgacttttccactatcaaccccaaaaaacccaactctgccttacgtaaagttgccagagtacgattaacctctggatttgaaatcactgcttatatacctggtattggccataatttacaagaacattctgtagtattagtaagaggaggaagggttaaggatttacccggtgtgagatatcgcattattcgaggaaccctagatgctgtcgcagtaaagaatcgtcaacaagggcgttctagtgcgttgtagattcttatccaagacttgtatcatttgatgatgccatgtgaatcgctagaaacatgtgaagtgtatggctaacccaataacgaaagtttcgtaaggggactggagcaggctaccatgagacaaaagatcttctttctaaagagattcgattcggaactcttatatgtccaaggtcaatatggaaattctttcagaggttttcccttactttgtccgtgtcaacaaacaattcgaaatacctcgactttttcagaacaggtccgagtcaaatagcaatgattcgaagcacttctttttccattacactatttcggaa

SEQ ID NO: 309 - DNA, 2398 bp, Zea mays
cacattaggtttagggataatcaggctcgaactgatgacttccaccacgtcaaggtgacactctaccgctgagttatatcccttccccgtcccatcgagaaagagaattaacgaatcctaaggcaaaggggcgagaaacgcaaggccactcttcctccgggctttctttccgcactattatggacagtcaaataatgggaaaaattggattcaattgtcaaccggtcctatcgaaaataggattgactatggattcgagccatagcacatggtttcataaaatctgtacgattttcccgatctaaatcgagtgggtttccatgaagaagatcttgttcagcatgttctattcgatactggtaggagaagaacccgactcggtattcttaaaaaaagaggggaagcagaaccaagtcaagatgatatggatcgccccttcttcttgcgccaaagatcttaccatttccgaaggaactggggctacatttcttttcaatttccattcaagagtttctatctgtttccacgccctttttttgagacctcgaaacatgaaatggacaaattccttctcttaggaacacatacaagaaaaaggataatggtagcccccccattaactaatttatgaatttcatagtaatagaaatccatgtcctaccgagacagaatttcgaacttgctatcctcttgcctaataggcaaagattgacctctgtagaaagactgattcattcggatcgatatgaggacccaactccgttgcattgccagaatccatgttccatatttgaagcgggttgacctctgtgcttctctcatggtacaatcctcttcctgctgagccccctttctcctcggtccacagagaaaaaatgtaggactggtgccgacagttcatcacggaagaaagaactcacagagccgggatcgctaactaatagaatagtactactaactaatactaatatatagataactaatactaatatatagatattaatactaatatatagatatatagaaatagatatctagaaatagaaacgaactaatatatagataatcgaaatcgaaaagaactgtcttttctgtatactttccccgttctattgctaccgcgggtcttatgcaatcgatcggatcatatagatatcccttcaacacaacataggtcatcgaaaggatctcggacgactcaccaaagcacgaaagccagttagaaaatggattcctatttgaagagtgcctaaccgcatggataagctcacattaacccgtcaattttggatccaattcgggatttttcttgggaagtttcgggaagaaattggaataatatcgattcatacagaggaaaaagttctctattgatgcaaacgctgtacctagaggatagggatagaggaagagggaaaaatcgaaatgaaataaataaagaataaagccaaaaaaataagtcgaagatagaagagcccagattcaaaatgaagaaatggaaactcgaaaaggatccttctgattctcaaagaatgaggggcaaggggattgataccgagaaagatttcttcttattataagacgtgatttgatccgcatatgtttggtaaaagaacaatcttctcctttaatcataaatggaaagtgttcaattagaacatgaaaacgtgactcaattggtcttagttagtcttcgggacggagtggaagaagggcggagactctcgaacgaggaaaaggatcccttcgaaagaattgaacgaggagccgtatgaggtgaaaatctcatgtacggttctgtagagggacagtaaggatgacttatctgtcgacttttccactatcaaccccaaaaaacccaactctgccttacgtaaagttgccagagtacgattaacctctggatttgaaatcactgcttatatacctggtattggccataatttacaagaacattctgtagtattagtaagaggaggaagggttaaggatttacccggtgtgagatatcgcattattcgaggaaccctagatgctgtcgcagtaaagaatcgtcaacaagggcgttctagtgcgttgtagattcttatccaagacttgtatcatttgatgatgccatgtgaatcgctagaaacatgtgaagtgtatggctaacccaataacgaaagtttcgtaaggggactggagcaggctaccatgagacaaaagatcttctttctaaagagattcgattcggaactcttatatgtccaaggtcaatatggaaattctttcagaggttttcccttactttgtccgtgtcaacaaacaattcgaaatacctcgactttttcagaacaggtccgagtcaaatagcaatgattcgaagcacttctttttccattacactatttcggaa

SEQ ID NO: 310 - DNA, 2330 bp, Zea mays
gaggggaagcagaaccaagtcaagatgatatggatcgccccttcttcttgcgccaaagatcttaccatttccgaaggaactggggctacatttcttttcaatttccattcaagagtttctatctgtttccacgccctttttttgagacctcgaaacatgaaatggacaaattccttctcttaggaacacatacaagaaaaaggataatggtagcccccccattaactaatttatgaatttcatagtaatagaaatccatgtcctaccgagacagaatttcgaacttgctatcctcttgcctaataggcaaagattgacctctgtagaaagactgattcattcggatcgatatgaggacccaactccgttgcattgccagaatccatgttccatatttgaagcgggttgacctctgtgcttctctcatggtacaatcctcttcctgctgagccccctttctcctcggtccacagagaaaaaatgtaggactggtgccgacagttcatcacggaagaaagaactcacagagccgggatcgctaactaatagaatagtactactaactaatactaatatatagataactaatactaatatatagatattaatactaatatatagatatatagaaatagatatctagaaatagaaacgaactaatatatagataatcgaaatcgaaaagaactgtcttttctgtatactttccccgttctattgctaccgcgggtcttatgcaatcgatcggatcatatagatatcccttcaacacaacataggtcatcgaaaggatctcggacgactcaccaaagcacgaaagccagttagaaaatggattcctatttgaagagtgcctaaccgcatggataagctcacattaacccgtcaattttggatccaattcgggatttttcttgggaagtttcgggaagaaattggaataatatcgattcatacagaggaaaaagttctctattgatgcaaacgctgtacctagaggatagggatagaggaagagggaaaaatcgaaatgaaataaataaagaataaagccaaaaaaataagtcgaagatagaagagcccagattcaaaatgaagaaatggaaactcgaaaaggatccttctgattctcaaagaatgaggggcaaggggattgataccgagaaagatttcttcttattataagacgtgatttgatccgcatatgtttggtaaaagaacaatcttctcctttaatcataaatggaaagtgttcaattagaacatgaaaacgtgactcaattggtcttagttagtcttcgggacggagtggaagaagggcggagactctcgaacgaggaaaaggatcccttcgaaagaattgaacgaggagccgtatgaggtgaaaatctcatgtacggttctgtagagggacagtaaggatgacttatctgtcgacttttccactatcaaccccaaaaaacccaactctgccttacgtaaagttgccagagtacgattaacctctggatttgaaatcactgcttatatacctggtattggccataatttacaagaacattctgtagtattagtaagaggaggaagggttaaggatttacccggtgtgagatatcgcattattcgaggaaccctagatgctgtcgcagtaaagaatcgtcaacaagggcgttctagtgcgttgtagattcttatccaagacttgtatcatttgatgatgccatgtgaatcgctagaaacatgtgaagtgtatggctaacccaataacgaaagtttcgtaaggggactggagcaggctaccatgagacaaaagatcttctttctaaagagattcgattcggaactcttatatgtccaaggtcaatatggaaattctttcagaggttttcccttactttgtccgtgtcaacaaacaattcgaaatacctcgactttttcagaacaggtccgagtcaaatagcaatgattcgaagcacttctttttccattacactatttcggaaacctaaggactcgatcgtatggatatggaaaatacaggatttccggtcctagcgggaaaaggagggaaacggatactcaatttaaagtgagtaaacagaattccatactcgatctcatagatccctatagaattctgtggaaagccgtattcgatgaaagtcgtatgtacggcttggagggagatctttcctatctttcgagatccaccctacaatatggggccaaaaagccaaaaaaataagtgattcgtttttagcccttataaaaagaaaacggattcttgaacctctttcacgctcatgtcacgtcgaggtactgcagaaaaa

SEQ ID NO: 311 - DNA, 3706 bp, Zea mays
taaagagcgtggaggttcgagtcctcttcaaggcataatacggagaatgctcattgaatgagcattccccgtagaagtattccggaaatctgcgcctggcgctctcctctatcttctgaggtccttaaccatctccctgagaaaagtagacagtaaaagccaaaatagactaaatatagcctgaatgatcctaaaaacccctcgaaggagataataaagaacctaaagcagacggtatcctactgcaagggtggtcttaagaatccaaaagaggttgctcagaagagatagatgtatcccaacctctattgctctcgcgtaaagccttttttttacgcgacaggaaaaagtgactacgaattcccctttttgtttgcgaatccctgtttgtatcctttgagcgcacgcccataagtagcgatcaaggaaatcgatcaaacgatcccaataccgtgaaagagaaacttcccagatccagggaagcttatcataaagggcttcaacaaggggttttattcgttctttgagcaaaaagataaagatcggatagattcgaaccgcaattgcaaaggtaataactactatgccagcccaaatcatgatcttcaccaacttggatttggttctctcgcgaaaatcgcgagttgcagagatgagaaccatgaaaagcaagatcccgaataagaaaatagaaaccgaggaaaccacaagagtgaagactagtagagtctcatcttttgtcattctttgctcctttttcactcaatgattcattcgaatttcccaaccaaaaattctatatgtctattctatgatatttctatatatatagaatatgatatctaatatgacacccgatttgtcaaagggattggattggtgacttacccattcagtgactttggcactggacgttccaaaaacgggtactatcggatcgggtgaattagagaatagacagaggtccgttggcatttcagcttctcttctcctttcagggcctatccgaaagagaatccaggacctcttggtcctgaatatcagaataggacgaacgaaccggcctccgcggatatctttgcttcggaacaaaacaattagcattaggctcggtcaactggaatgtgtattatccatatgggagatcttccaatcgagaagatccatcgatctgagacgaagagaaagggcccattttctttacttattcagttaaaccaaggattcgttatggaaacagatagcaacaaccatttcatcagacatgcgtatttttgattatccggtggatttacacagtttcattaatgcaaattgtttgatgtagttagtactcaggttattcgacgctcaagaattcttatttaggcagttcatatcatcccatacatagtgttttgatctaagattgcaattcttccatgtttccgcagtagcatattgttccatggagctagggtccaaaataggaatcaacaagtgtttccacgactctaccgcccggccaatcctgttccactgaatcccctccctttttcatggccacatatctttacggctaaggagtgggaaatctttctcctgttacacaaatgaaatgtttcatttcatccgggaaaagccacctctttctccacaaacaatgtctttgtcatttgatccaggagccttccgttagataggaacagctttgctaaatactgagaactctcggatagagtattagaatgaaaagaccattaattatataaggaagagcccagggttctagcccattcccattcctaaatcaataattcgtagtgcttttgcctttcttgcgtactcctggtgaaccagttgctagccatatgtttaggaggcttttttctccaggtactggtactaagccgctttgccatctcttcatcttcatctgcaaagaattctcgacgtgaaaacacagagacaaaggcctgatctttgaataggaaaaagaatggatccgaaggtcccaaatcaattggcttattcgaaaaaagccttcttctttgaaagatatatctcgtgtctggtactgcattgttccactctgcaagaagtccgaatcagtctcttgcacctccccctttttatgataaatataccagaaataattcgaataaatagcagtctctgacaactcatcctctttaatctgcttggacccgctccaatacccataggaaaatccccagtcatattcagcgtacctgtccctgcaatcaaatagattgtcccaagggccccatattctaggagcccaagttatgctattgaaaattcgttcctctagcagttccggtttgaccattccgttccctttttcaaactccacttcttttcatatagcatccctaatcaaagagagaacaatatccatttcaaaacatttctaacggattccttggttcggaccgacgaagtaatggcactcgattattatcaacccgactgcaatctttttctgtcggtaaggattgcaccagagcaccttctacttctaataggccatgaactatagatagagaagaatcattctgagcgagtccataagaagcgacccactttttcatcggttccgggggaagaccaaagatcttgcgcgaccggtccgccagaaaaactcaaaagagaaagaagtctcgttaatctcttcatgctcgttccaagttcgaagtaccgtttggccaaagaaaaagccgcttcctgacacgattgcctctttatatagatagatataggatctatggggttattacttagaagtctattttgtacaagatcccctcctatctgatagaaaagggtcccatgatcccgagccggtcttatcttggctcgcaaaccccaagtttgtctatgaagagctaatctaattgtattagtttctataatggatttcttatgtggaatactaatggatagggcctcgttgctaagtgctacaagatctagtgcactggaactcgtggttatggacccgaatcctttagtatggaacattgtcttttccaagtaaaaacccctagtatatgaaagaatgaaaaggtgctttcgttcttgtggaataagaagccctcgtaccttaatgaaaggaaaataggaatttttcgttaggtatttgaccaaataggattgtccagttcctatagaacctatcactaaaatacccgatagggctaagcggaacgaaaagggttttccatgagatggtaaatgaaaacgattagccccacacgaggtttgggaataagtgattgtctgataatgagcaaggaatatacgtctttctgctaaagagaatctattaaactcataattcattagatccttgttagcaatgtcaactaggtatcataagtaaatggatctcggttgttcaatcctttgataaccaaggtcattctttgctaaagagaaatgatcactatgggtcagactcaatagaattggatccattccaaatagcgagaattaggattcttgatccctctcagtctctctttcaattcgaggatccggagaggtgttttcatagtcatctccgaatatttgccatctccgaatattttcgatttcatttttctatgatatgtctttctatatgaaaattggttatttacgatgtacgatgatccctgttaagcatccatggctgaatggttaaagcgcccaactcataatt

SEQ ID NO: 312 - DNA, 2988 bp, Zea mays
tttttttacgcgacaggaaaaagtgactacgaattcccctttttgtttgcgaatccctgtttgtatcctttgagcgcacgcccataagtagcgatcaaggaaatcgatcaaacgatcccaataccgtgaaagagaaacttcccagatccagggaagcttatcataaagggcttcaacaaggggttttattcgttctttgagcaaaaagataaagatcggatagattcgaaccgcaattgcaaaggtaataactactatgccagcccaaatcatgatcttcaccaacttggatttggttctctcgcgaaaatcgcgagttgcagagatgagaaccatgaaaagcaagatcccgaataagaaaatagaaaccgaggaaaccacaagagtgaagactagtagagtctcatcttttgtcattctttgctcctttttcactcaatgattcattcgaatttcccaaccaaaaattctatatgtctattctatgatatttctatatatatagaatatgatatctaatatgacacccgatttgtcaaagggattggattggtgacttacccattcagtgactttggcactggacgttccaaaaacgggtactatcggatcgggtgaattagagaatagacagaggtccgttggcatttcagcttctcttctcctttcagggcctatccgaaagagaatccaggacctcttggtcctgaatatcagaataggacgaacgaaccggcctccgcggatatctttgcttcggaacaaaacaattagcattaggctcggtcaactggaatgtgtattatccatatgggagatcttccaatcgagaagatccatcgatctgagacgaagagaaagggcccattttctttacttattcagttaaaccaaggattcgttatggaaacagatagcaacaaccatttcatcagacatgcgtatttttgattatccggtggatttacacagtttcattaatgcaaattgtttgatgtagttagtactcaggttattcgacgctcaagaattcttatttaggcagttcatatcatcccatacatagtgttttgatctaagattgcaattcttccatgtttccgcagtagcatattgttccatggagctagggtccaaaataggaatcaacaagtgtttccacgactctaccgcccggccaatcctgttccactgaatcccctccctttttcatggccacatatctttacggctaaggagtgggaaatctttctcctgttacacaaatgaaatgtttcatttcatccgggaaaagccacctctttctccacaaacaatgtctttgtcatttgatccaggagccttccgttagataggaacagctttgctaaatactgagaactctcggatagagtattagaatgaaaagaccattaattatataaggaagagcccagggttctagcccattcccattcctaaatcaataattcgtagtgcttttgcctttcttgcgtactcctggtgaaccagttgctagccatatgtttaggaggcttttttctccaggtactggtactaagccgctttgccatctcttcatcttcatctgcaaagaattctcgacgtgaaaacacagagacaaaggcctgatctttgaataggaaaaagaatggatccgaaggtcccaaatcaattggcttattcgaaaaaagccttcttctttgaaagatatatctcgtgtctggtactgcattgttccactctgcaagaagtccgaatcagtctcttgcacctccccctttttatgataaatataccagaaataattcgaataaatagcagtctctgacaactcatcctctttaatctgcttggacccgctccaatacccataggaaaatccccagtcatattcagcgtacctgtccctgcaatcaaatagattgtcccaagggccccatattctaggagcccaagttatgctattgaaaattcgttcctctagcagttccggtttgaccattccgttccctttttcaaactccacttcttttcatatagcatccctaatcaaagagagaacaatatccatttcaaaacatttctaacggattccttggttcggaccgacgaagtaatggcactcgattattatcaacccgactgcaatctttttctgtcggtaaggattgcaccagagcaccttctacttctaataggccatgaactatagatagagaagaatcattctgagcgagtccataagaagcgacccactttttcatcggttccgggggaagaccaaagatcttgcgcgaccggtccgccagaaaaactcaaaagagaaagaagtctcgttaatctcttcatgctcgttccaagttcgaagtaccgtttggccaaagaaaaagccgcttcctgacacgattgcctctttatatagatagatataggatctatggggttattacttagaagtctattttgtacaagatcccctcctatctgatagaaaagggtcccatgatcccgagccggtcttatcttggctcgcaaaccccaagtttgtctatgaagagctaatctaattgtattagtttctataatggatttcttatgtggaatactaatggatagggcctcgttgctaagtgctacaagatctagtgcactggaactcgtggttatggacccgaatcctttagtatggaacattgtcttttccaagtaaaaacccctagtatatgaaagaatgaaaaggtgctttcgttcttgtggaataagaagccctcgtaccttaatgaaaggaaaataggaatttttcgttaggtatttgaccaaataggattgtccagttcctatagaacctatcactaaaatacccgatagggctaagcggaacgaaaagggttttccatgagatggtaaatgaaaacgattagccccacacgaggtttgggaataagtgattgtctgataatgagcaaggaatatacgtctttctgc

SEQ ID NO: 313 - DNA, 3315 bp, Zea mays
taaagagcgtggaggttcgagtcctcttcaaggcataatacggagaatgctcattgaatgagcattccccgtagaagtattccggaaatctgcgcctggcgctctcctctatcttctgaggtccttaaccatctccctgagaaaagtagacagtaaaagccaaaatagactaaatatagcctgaatgatcctaaaaacccctcgaaggagataataaagaacctaaagcagacggtatcctactgcaagggtggtcttaagaatccaaaagaggttgctcagaagagatagatgtatcccaacctctattgctctcgcgtaaagccttttttttacgcgacaggaaaaagtgactacgaattcccctttttgtttgcgaatccctgtttgtatcctttgagcgcacgcccataagtagcgatcaaggaaatcgatcaaacgatcccaataccgtgaaagagaaacttcccagatccagggaagcttatcataaagggcttcaacaaggggttttattcgttctttgagcaaaaagataaagatcggatagattcgaaccgcaattgcaaaggtaataactactatgccagcccaaatcatgatcttcaccaacttggatttggttctctcgcgaaaatcgcgagttgcagagatgagaaccatgaaaagcaagatcccgaataagaaaatagaaaccgaggaaaccacaagagtgaagactagtagagtctcatcttttgtcattctttgctcctttttcactcaatgattcattcgaatttcccaaccaaaaattctatatgtctattctatgatatttctatatatatagaatatgatatctaatatgacacccgatttgtcaaagggattggattggtgacttacccattcagtgactttggcactggacgttccaaaaacgggtactatcggatcgggtgaattagagaatagacagaggtccgttggcatttcagcttctcttctcctttcagggcctatccgaaagagaatccaggacctcttggtcctgaatatcagaataggacgaacgaaccggcctccgcggatatctttgcttcggaacaaaacaattagcattaggctcggtcaactggaatgtgtattatccatatgggagatcttccaatcgagaagatccatcgatctgagacgaagagaaagggcccattttctttacttattcagttaaaccaaggattcgttatggaaacagatagcaacaaccatttcatcagacatgcgtatttttgattatccggtggatttacacagtttcattaatgcaaattgtttgatgtagttagtactcaggttattcgacgctcaagaattcttatttaggcagttcatatcatcccatacatagtgttttgatctaagattgcaattcttccatgtttccgcagtagcatattgttccatggagctagggtccaaaataggaatcaacaagtgtttccacgactctaccgcccggccaatcctgttccactgaatcccctccctttttcatggccacatatctttacggctaaggagtgggaaatctttctcctgttacacaaatgaaatgtttcatttcatccgggaaaagccacctctttctccacaaacaatgtctttgtcatttgatccaggagccttccgttagataggaacagctttgctaaatactgagaactctcggatagagtattagaatgaaaagaccattaattatataaggaagagcccagggttctagcccattcccattcctaaatcaataattcgtagtgcttttgcctttcttgcgtactcctggtgaaccagttgctagccatatgtttaggaggcttttttctccaggtactggtactaagccgctttgccatctcttcatcttcatctgcaaagaattctcgacgtgaaaacacagagacaaaggcctgatctttgaataggaaaaagaatggatccgaaggtcccaaatcaattggcttattcgaaaaaagccttcttctttgaaagatatatctcgtgtctggtactgcattgttccactctgcaagaagtccgaatcagtctcttgcacctccccctttttatgataaatataccagaaataattcgaataaatagcagtctctgacaactcatcctctttaatctgcttggacccgctccaatacccataggaaaatccccagtcatattcagcgtacctgtccctgcaatcaaatagattgtcccaagggccccatattctaggagcccaagttatgctattgaaaattcgttcctctagcagttccggtttgaccattccgttccctttttcaaactccacttcttttcatatagcatccctaatcaaagagagaacaatatccatttcaaaacatttctaacggattccttggttcggaccgacgaagtaatggcactcgattattatcaacccgactgcaatctttttctgtcggtaaggattgcaccagagcaccttctacttctaataggccatgaactatagatagagaagaatcattctgagcgagtccataagaagcgacccactttttcatcggttccgggggaagaccaaagatcttgcgcgaccggtccgccagaaaaactcaaaagagaaagaagtctcgttaatctcttcatgctcgttccaagttcgaagtaccgtttggccaaagaaaaagccgcttcctgacacgattgcctctttatatagatagatataggatctatggggttattacttagaagtctattttgtacaagatcccctcctatctgatagaaaagggtcccatgatcccgagccggtcttatcttggctcgcaaaccccaagtttgtctatgaagagctaatctaattgtattagtttctataatggatttcttatgtggaatactaatggatagggcctcgttgctaagtgctacaagatctagtgcactggaactcgtggttatggacccgaatcctttagtatggaacattgtcttttccaagtaaaaacccctagtatatgaaagaatgaaaaggtgctttcgttcttgtggaataagaagccctcgtaccttaatgaaaggaaaataggaatttttcgttaggtatttgaccaaataggattgtccagttcctatagaacctatcactaaaatacccgatagggctaagcggaacgaaaagggttttccatgagatggtaaatgaaaacgattagccccacacgaggtttgggaataagtgattgtctgataatgagcaaggaatatacgtctttctgc

SEQ ID NO: 314 - DNA, 3379 bp, Zea mays
tttttttacgcgacaggaaaaagtgactacgaattcccctttttgtttgcgaatccctgtttgtatcctttgagcgcacgcccataagtagcgatcaaggaaatcgatcaaacgatcccaataccgtgaaagagaaacttcccagatccagggaagcttatcataaagggcttcaacaaggggttttattcgttctttgagcaaaaagataaagatcggatagattcgaaccgcaattgcaaaggtaataactactatgccagcccaaatcatgatcttcaccaacttggatttggttctctcgcgaaaatcgcgagttgcagagatgagaaccatgaaaagcaagatcccgaataagaaaatagaaaccgaggaaaccacaagagtgaagactagtagagtctcatcttttgtcattctttgctcctttttcactcaatgattcattcgaatttcccaaccaaaaattctatatgtctattctatgatatttctatatatatagaatatgatatctaatatgacacccgatttgtcaaagggattggattggtgacttacccattcagtgactttggcactggacgttccaaaaacgggtactatcggatcgggtgaattagagaatagacagaggtccgttggcatttcagcttctcttctcctttcagggcctatccgaaagagaatccaggacctcttggtcctgaatatcagaataggacgaacgaaccggcctccgcggatatctttgcttcggaacaaaacaattagcattaggctcggtcaactggaatgtgtattatccatatgggagatcttccaatcgagaagatccatcgatctgagacgaagagaaagggcccattttctttacttattcagttaaaccaaggattcgttatggaaacagatagcaacaaccatttcatcagacatgcgtatttttgattatccggtggatttacacagtttcattaatgcaaattgtttgatgtagttagtactcaggttattcgacgctcaagaattcttatttaggcagttcatatcatcccatacatagtgttttgatctaagattgcaattcttccatgtttccgcagtagcatattgttccatggagctagggtccaaaataggaatcaacaagtgtttccacgactctaccgcccggccaatcctgttccactgaatcccctccctttttcatggccacatatctttacggctaaggagtgggaaatctttctcctgttacacaaatgaaatgtttcatttcatccgggaaaagccacctctttctccacaaacaatgtctttgtcatttgatccaggagccttccgttagataggaacagctttgctaaatactgagaactctcggatagagtattagaatgaaaagaccattaattatataaggaagagcccagggttctagcccattcccattcctaaatcaataattcgtagtgcttttgcctttcttgcgtactcctggtgaaccagttgctagccatatgtttaggaggcttttttctccaggtactggtactaagccgctttgccatctcttcatcttcatctgcaaagaattctcgacgtgaaaacacagagacaaaggcctgatctttgaataggaaaaagaatggatccgaaggtcccaaatcaattggcttattcgaaaaaagccttcttctttgaaagatatatctcgtgtctggtactgcattgttccactctgcaagaagtccgaatcagtctcttgcacctccccctttttatgataaatataccagaaataattcgaataaatagcagtctctgacaactcatcctctttaatctgcttggacccgctccaatacccataggaaaatccccagtcatattcagcgtacctgtccctgcaatcaaatagattgtcccaagggccccatattctaggagcccaagttatgctattgaaaattcgttcctctagcagttccggtttgaccattccgttccctttttcaaactccacttcttttcatatagcatccctaatcaaagagagaacaatatccatttcaaaacatttctaacggattccttggttcggaccgacgaagtaatggcactcgattattatcaacccgactgcaatctttttctgtcggtaaggattgcaccagagcaccttctacttctaataggccatgaactatagatagagaagaatcattctgagcgagtccataagaagcgacccactttttcatcggttccgggggaagaccaaagatcttgcgcgaccggtccgccagaaaaactcaaaagagaaagaagtctcgttaatctcttcatgctcgttccaagttcgaagtaccgtttggccaaagaaaaagccgcttcctgacacgattgcctctttatatagatagatataggatctatggggttattacttagaagtctattttgtacaagatcccctcctatctgatagaaaagggtcccatgatcccgagccggtcttatcttggctcgcaaaccccaagtttgtctatgaagagctaatctaattgtattagtttctataatggatttcttatgtggaatactaatggatagggcctcgttgctaagtgctacaagatctagtgcactggaactcgtggttatggacccgaatcctttagtatggaacattgtcttttccaagtaaaaacccctagtatatgaaagaatgaaaaggtgctttcgttcttgtggaataagaagccctcgtaccttaatgaaaggaaaataggaatttttcgttaggtatttgaccaaataggattgtccagttcctatagaacctatcactaaaatacccgatagggctaagcggaacgaaaagggttttccatgagatggtaaatgaaaacgattagccccacacgaggtttgggaataagtgattgtctgataatgagcaaggaatatacgtctttctgctaaagagaatctattaaactcataattcattagatccttgttagcaatgtcaactaggtatcataagtaaatggatctcggttgttcaatcctttgataaccaaggtcattctttgctaaagagaaatgatcactatgggtcagactcaatagaattggatccattccaaatagcgagaattaggattcttgatccctctcagtctctctttcaattcgaggatccggagaggtgttttcatagtcatctccgaatatttgccatctccgaatattttcgatttcatttttctatgatatgtctttctatatgaaaattggttatttacgatgtacgatgatccctgttaagcatccatggctgaatggttaaagcgcccaactcataatt

(19) United States
(12) Patent Application Publication
(10) Pub. No.: US 2020/0362357 A1
(43) Pub. Date: Nov. 19, 2020

(54) SITES FOR PLASTID TRANSFORMATION

(71) Applicant: MONSANTO TECHNOLOGY, LLC, St. Louis, MO (US)

(72) Inventor: Xudong Ye, Chesterfield, MO (US)

(21) Appl. No.: 16/473,999

(22) PCT Filed: Dec. 18, 2017
(86) PCT No.: PCT/US2017/067014

§ 371 Date: Jun. 26, 2019

Related U.S. Application Data
(60) Provisional application No. 62/440,855, filed on Dec. 30, 2016.

(51) Int. Cl. C12N 15/82

(52) U.S. Cl. CPC C12N 15/8214; C12N 2830/00

(57) ABSTRACT

Compositions and methods for plastid transformation and regeneration or development of transplastomic plants, plant cells, plant parts, and seeds are provided. Target sequences for plastid transformation, together with defined sequences of a first homology arm and a second homology arm are provided for the production of recombinant plastid transformation constructs that target specific sites within the plastid genome for transformation, together with methods of using such constructs.

### REFERENCE TO RELATED APPLICATION

This application claims the benefit of U.S. Provisional Application No. 62/440,855, filed Dec. 30, 2016, which is herein incorporated by reference in its entirety.

### FIELD OF THE INVENTION

The present invention relates to the field of agricultural biotechnology, and more specifically to methods and compositions for genetic transformation of plastids.

### INCORPORATION OF SEQUENCE LISTING

A sequence listing contained in the file named “MONS391WO_ST25.txt” which is 396 kilo bytes (measured in MS-Windows®) and created on Dec. 18, 2017, is filed electronically herewith and incorporated by reference in its entirety.

### BACKGROUND

Plastid transformation can provide significant advantages over conventional nuclear transformation methods for creating transgenic plants, including more abundant and reliable transgene expression, maternal inheritance, and lack of silencing mechanisms. Improved compositions and methods are needed, however, for selecting sites within the plastid genome that are suitable for transformation and unlikely to interfere with expression of plastid genes.

### SUMMARY

In one aspect, the invention provides a recombinant plastid transformation construct, said construct comprising: (i) a first homology arm comprising a sequence that is at least 95% identical to at least 100 contiguous nucleotides of any one of SEQ ID NOs: 3, 7, 11, 15, 19, 22, 26, 30, 33, 37, 41, 45, 49, 53, 57, 61, 64, 68, 72, 76, 80, 84, 88, 92, 96, 100, 104, 108, 112, 116, 120, 123, 127, 131, 135, 138, 142, 146, 150, 154, 158, 162, 166, 170, 174, 178, 182, 186, 190, 194, 198, 202, 206, 210, 213, 217, 221, 225, 229, 233, 237, 241, 245, 249, 253, 257, 261, 265, 269, 273, 277, 281, 285, 289, 293, 297, 301, 305, 309, or 313; and (ii) a second homology arm comprising a sequence that is at least 95% identical to at least 100 contiguous nucleotides of any one of SEQ ID NOs: 4, 8, 12, 16, 20, 23, 27, 31, 34, 38, 42, 46, 50, 54, 58, 62, 65, 69, 73, 77, 81, 85, 89, 93, 97, 101, 105, 109, 113, 117, 121, 124, 128, 132, 136, 139, 143, 147, 151, 155, 159, 163, 167, 171, 175, 179, 183, 187, 191, 195, 199, 203, 207, 211, 214, 218, 222, 226, 230, 234, 238, 242, 246, 250, 254, 258, 262, 266, 270, 274, 278, 282, 286, 290, 294, 298, 302, 306, 310, or 314. In certain embodiments, the first homology arm comprises at least 100 contiguous nucleotides of any one of SEQ ID NOs: 3, 7, 11, 15, 19, 22, 26, 30, 33, 37, 41, 45, 49, 53, 57, 61, 64, 68, 72, 76, 80, 84, 88, 92, 96, 100, 104, 108, 112, 116, 120, 123, 127, 131, 135, 138, 142, 146, 150, 154, 158, 162, 166, 170, 174, 178, 182, 186, 190, 194, 198, 202, 206, 210, 213, 217, 221, 225, 229, 233, 237, 241, 245, 249, 253, 257, 261, 265, 269, 273, 277, 281, 285, 289, 293, 297, 301, 305, 309, or 313. In further embodiments, the second homology arm comprises at least 100 contiguous nucleotides of any one of SEQ ID NOs: 4, 8, 12, 16, 20, 23, 27, 31, 34, 38, 42, 46, 50, 54, 58, 62, 65, 69, 73, 77, 81, 85, 89, 93, 97, 101, 105, 109, 113, 117, 121, 124, 128, 132, 136, 139, 143, 147, 151, 155, 159, 163, 167, 171, 175, 179, 183, 187, 191, 195, 199, 203, 207, 211, 214, 218, 222, 226, 230, 234, 238, 242, 246, 250, 254, 258, 262, 266, 270, 274, 278, 282, 286, 290, 294, 298, 302, 306, 310, or 314. In some embodiments, the first or second homology arm is between 0.1 and 2.0 kilobases in length.

Recombinant plastid transformation constructs of the invention may further comprise an insertion sequence positioned between the first homology arm and the second homology arm, which may comprise a transcribable nucleic acid sequence. Transcribable nucleic acid sequences may be genes of agronomic interest, for example a gene of agronomic interest that confers an agronomically beneficial trait selected from the group consisting of modified carbon fixation, modified nitrogen fixation, herbicide tolerance, insect resistance or control, modified or increased yield, fungal disease tolerance or resistance, virus tolerance or resistance, nematode tolerance or resistance, bacterial disease tolerance or resistance, modified starch production, modified oil production, modified fatty acid content, modified protein production, enhanced animal and human nutrition, environmental stress tolerance, drought tolerance, improved processing traits or fruit ripening, improved digestibility, improved taste and flavor characteristics, modified enzyme production, modified fiber production, synthesis of other biopolymers, peptides or proteins, and enhanced biofuel production. In further embodiments, recombinant plastid transformation constructs of the invention comprise a promoter active in plant plastids, for example a promoter selected from the group consisting of Prrn, psbA, and rbcL. In yet further embodiments, recombinant plastid transformation constructs of the invention comprise a selectable marker, for example a selectable marker is selected from the group consisting of aadA, nptII, aph IV, aac3, aacC4, CAT, EPSPS, bar, GOX, GAT, and β-glucuronidase. Recombinant plastid transformation constructs of the invention may also comprise a screenable marker.

In another aspect, the invention provides a DNA molecule or vector comprising a recombinant plastid transformation construct as described herein, or a plastid comprising a recombinant plastid transformation construct as described herein. The invention further provides a plant, plant cell, plant part, or seed comprising a plastid comprising a recombinant plastid transformation construct as described herein, for example a crop plant. Plants provided by the invention may be monocotyledonous plants, including corn (maize), wheat, rice, millet, barley, sorghum, sugarcane, oat, rye, and other plants within the Poaceae or Gramineae family, or dicotyledonous plants, including cotton, canola, and sugar beets, soybean, alfalfa and other Fabaceae or leguminous plants.

In yet another aspect, the invention provides a method of producing a transformed plant cell plastid, comprising the step of transforming at least one plastid of a plant cell with a recombinant plastid transformation construct provided herein to produce plant cell comprising a transformed plastid; wherein the insertion sequence is incorporated into a genome of said plastid flanked by plastid sequences corresponding to the first and second homology arms of the recombinant plastid transformation construct. In certain embodiments, the insertion sequence is incorporated into the genome of said transformed plastid by homologous recombination. The invention further provides transformed plastids produced by the methods provided herein. In further embodiments, methods of the invention further comprise a step of selecting for development or regeneration of a plastid transformed plant cell by contacting said plant cell with a selection agent. Plastid transformed plant cells produced by the methods disclosed herein are further provided by the invention. In yet further embodiments, methods of the invention further comprise regenerating a plastid transformed plant from a plastid transformed plant cell disclosed herein, or obtaining a plastid transformed seed from such a plastid transformed plant. The plastid transformed plant may be developed or regenerated from said plant cell under selection pressure by contacting the developing plant with a selection agent. Plastid transformed plants and plastid transformed seeds produced by the methods disclosed herein are further provided by the invention.

In another aspect, the invention provides a method of selecting a target site for plastid transformation comprising identifying a plastid target sequence, wherein said target sequence is located: (1) between two neighboring plastid genes in the plastid genome of a plant cell, (2) at least 20 base pairs from the 5′ terminus of the coding sequence of a tRNA or small RNA encoding plastid gene, (3) at least 100 base pairs from the 5′ terminus of the coding sequence of a structural protein-encoding plastid gene, (4) at least 20 base pairs from the 3′ terminus of the coding sequence of a tRNA or small RNA encoding plastid gene, and (5) at least 150 base pairs from the 3′ terminus of the coding sequence of a structural protein-encoding plastid gene.

In yet a further aspect, the invention provides a recombinant plastid transformation construct, said construct comprising a homology arm comprising a sequence that is at least 95% identical to at least 100 contiguous nucleotides of any one of SEQ ID NO: 3, 7, 11, 15, 19, 22, 26, 30, 33, 37, 41, 45, 49, 53, 57, 61, 64, 68, 72, 76, 80, 84, 88, 92, 96, 100, 104, 108, 112, 116, 120, 123, 127, 131, 135, 138, 142, 146, 150, 154, 158, 162, 166, 170, 174, 178, 182, 186, 190, 194, 198, 202, 206, 210, 213, 217, 221, 225, 229, 233, 237, 241, 245, 249, 253, 257, 261, 265, 269, 273, 277, 281, 285, 289, 293, 297, 301, 305, 309, or 313; or at least 95% identical to at least 100 contiguous nucleotides of any one of SEQ ID NO: 4, 8, 12, 16, 20, 23, 27, 31, 34, 38, 42, 46, 50, 54, 58, 62, 65, 69, 73, 77, 81, 85, 89, 93, 97, 101, 105, 109, 113, 117, 121, 124, 128, 132, 136, 139, 143, 147, 151, 155, 159, 163, 167, 171, 175, 179, 183, 187, 191, 195, 199, 203, 207, 211, 214, 218, 222, 226, 230, 234, 238, 242, 246, 250, 254, 258, 262, 266, 270, 274, 278, 282, 286, 290, 294, 298, 302, 306, 310, or 314.

### BRIEF DESCRIPTION OF THE DRAWINGS

FIG. 1: Schematic illustration of selection of plastid target loci.

### DESCRIPTION OF THE SEQUENCES

SEQ ID NOs: 1, 5, 9, 13, 17, 24, 28, 35, 39, 43, 47, 51, 55, 59, 66, 70, 74, 78, 82, 86, 90, 94, 98, 102, 106, 110, 114, 118, 125, 129, 133, 140, 144, 148, 152, 156, 160, 164, 168, 172, 176, 180, 184, 188, 192, 196, 200, 204, 208, 215, 219, 223, 227, 231, 235, 239, 243, 247, 251, 255, 259, 263, 267, 271, 275, 279, 283, 287, 291, 295, 299, 303, 307, and 311 represent plastomic regions comprising target sites for plastid transformation in corn (Zea mays), soybean (Glycine max), and cotton (Gossypium hirsutum) as detailed in Table 1.

SEQ ID NOs: 2, 6, 10, 14, 18, 21, 25, 29, 32, 36, 40, 44, 48, 52, 56, 60, 63, 67, 71, 75, 79, 83, 87, 91, 95, 99, 103, 107, 111, 115, 119, 122, 126, 130, 134, 137, 141, 145, 149, 153, 157, 161, 165, 169, 173, 177, 181, 185, 189, 193, 197, 201, 205, 209, 212, 216, 220, 224, 228, 232, 236, 240, 244, 248, 252, 256, 260, 264, 268, 272, 276, 280, 284, 288, 292, 296, 300, 304, 308, and 312 represent target sites for plastid transformation as detailed in Table 1.

SEQ ID NOs: 3, 7, 11, 15, 19, 22, 26, 30, 33, 37, 41, 45, 49, 53, 57, 61, 64, 68, 72, 76, 80, 84, 88, 92, 96, 100, 104, 108, 112, 116, 120, 123, 127, 131, 135, 138, 142, 146, 150, 154, 158, 162, 166, 170, 174, 178, 182, 186, 190, 194, 198, 202, 206, 210, 213, 217, 221, 225, 229, 233, 237, 241, 245, 249, 253, 257, 261, 265, 269, 273, 277, 281, 285, 289, 293, 297, 301, 305, 309, and 313 comprise sequences useful in designing a first homology arm of plastid transformation constructs.

SEQ ID NOs: 4, 8, 12, 16, 20, 23, 27, 31, 34, 38, 42, 46, 50, 54, 58, 62, 65, 69, 73, 77, 81, 85, 89, 93, 97, 101, 105, 109, 113, 117, 121, 124, 128, 132, 136, 139, 143, 147, 151, 155, 159, 163, 167, 171, 175, 179, 183, 187, 191, 195, 199, 203, 207, 211, 214, 218, 222, 226, 230, 234, 238, 242, 246, 250, 254, 258, 262, 266, 270, 274, 278, 282, 286, 290, 294, 298, 302, 306, 310, and 314 comprise sequences useful in designing a second homology arm of plastid transformation constructs.

TABLE 1
Sequences associated with target sites for plastid transformation.

| | | Plastomic | | | |
|---|---|---|---|---|---|
| | | Junction | Target | | Second |
| | Plastomic Junction | Sequence | Sequence | First Arm | Arm |
| Organism | Interval | SEQ ID NO | SEQ ID NO | SEQ ID NO | SEQ ID NO |
| Glycine max | rbcL/atpB junction | 1 | 2 | 3 | 4 |
| Glycine max | trnL_UAA and trnT-UGU | 5 | 6 | 7 | 8 |
| | junction | | | | |
| Glycine max | psbD and trnT-GGU | 9 | 10 | 11 | 12 |
| | junction | | | | |
| Glycine max | trnT-GGU and trnE-UUC | 13 | 14 | 15 | 16 |
| | junction | | | | |
| Glycine max | petN and rpoB junction | 17 | 18 | 19 | 20 |
| Glycine max | petN and rpoB junction | 17 | 21 | 22 | 23 |
| Glycine max | trnG-UCC and trnS_GCU | 24 | 25 | 26 | 27 |
| | junction | | | | |
| Glycine max | psbK and rps16 junction | 28 | 29 | 30 | 31 |
| Glycine max | psbK and rps16 junction | 28 | 32 | 33 | 34 |
| Glycine max | psbE and petL junction | 35 | 36 | 37 | 38 |
| Glycine max | rp120 and rps12 junction | 39 | 40 | 41 | 42 |
| Glycine max | Rp123 and ycf2 junction | 43 | 44 | 45 | 46 |
| | (repeat region) | | | | |
| Glycine max | Rpsrps12 and trnV-GAC | 47 | 48 | 49 | 50 |
| | junction (repeat region) | | | | |
| Glycine max | Rp132 and ndhF junction | 51 | 52 | 53 | 54 |
| Glycine max | rbcL downstream | 55 | 56 | 57 | 58 |
| Glycine max | ndhJ and trnL-UAA | 59 | 60 | 61 | 62 |
| | junction | | | | |
| Glycine max | ndhJ and trnL-UAA | 59 | 63 | 64 | 65 |
| | junction | | | | |
| Glycine max | Ycf3 downstream | 66 | 67 | 68 | 69 |
| Glycine max | psbZ downstream | 70 | 71 | 72 | 73 |
| Glycine max | psbM downstream | 74 | 75 | 76 | 77 |
| Glycine max | atpI downstream | 78 | 79 | 80 | 81 |
| Glycine max | Rps16 downstream | 82 | 83 | 84 | 85 |
| Glycine max | psaI downstream | 86 | 87 | 88 | 89 |
| Glycine max | petA downstream | 90 | 91 | 92 | 93 |
| Glycine max | psaJ downstream | 94 | 95 | 96 | 97 |
| Glycine max | ndhB downstream | 98 | 99 | 100 | 101 |
| Glycine max | trnR-ACG downstream | 102 | 103 | 104 | 105 |
| Glycine max | trnN-GUU downstream | 106 | 107 | 108 | 109 |
| Gossypium | Rps16/psbK junction | 110 | 111 | 112 | 113 |
| hirsutum | | | | | |
| Gossypium | trnS-GCU and trnG-GCC | 114 | 115 | 116 | 117 |
| hirsutum | junction | | | | |
| Gossypium | rpoB and petN junction | 118 | 119 | 120 | 121 |
| hirsutum | | | | | |
| Gossypium | rpoB and petN junction | 118 | 122 | 123 | 124 |
| hirsutum | | | | | |
| Gossypium | trnE-UUC and trnT-GGU | 125 | 126 | 127 | 128 |
| hirsutum | junction | | | | |
| Gossypium | trnT-GGU and psbD | 129 | 130 | 131 | 132 |
| hirsutum | upstream | | | | |
| Gossypium | Rps4 and trnL-UAA | 133 | 134 | 135 | 136 |
| hirsutum | junction | | | | |
| Gossypium | Rps4 and trnL-UAA | 133 | 137 | 138 | 139 |
| hirsutum | junction | | | | |
| Gossypium | atpB and rbcL junction | 140 | 141 | 142 | 143 |
| hirsutum | | | | | |
| Gossypium | psbE and petL junction | 144 | 145 | 146 | 147 |
| hirsutum | | | | | |
| Gossypium | Rps18 and rps12 junction | 148 | 149 | 150 | 151 |
| hirsutum | | | | | |
| Gossypium | Rps12 and trnV-GVC | 152 | 153 | 154 | 155 |
| hirsutum | junction | | | | |
| Gossypium | Rpl32 and ndhF junction | 156 | 157 | 158 | 159 |
| hirsutum | | | | | |
| Gossypium | trnK-UUU and rps16 | 160 | 161 | 162 | 163 |
| hirsutum | junction | | | | |
| Gossypium | Rps16 and trnQ-UUG | 164 | 165 | 166 | 167 |
| hirsutum | junction | | | | |
| Gossypium | atpF and atpH junction | 168 | 169 | 170 | 171 |
| hirsutum | | | | | |
| Gossypium | atpH and atpI junction | 172 | 173 | 174 | 175 |
| hirsutum | | | | | |
| Gossypium | petN and psbM junction | 176 | 177 | 178 | 179 |
| hirsutum | | | | | |
| Gossypium | psaA and ycf3 junction | 180 | 181 | 182 | 183 |
| hirsutum | | | | | |
| Gossypium | ndhC and trnV-UAC | 184 | 185 | 186 | 187 |
| hirsutum | junction | | | | |
| Gossypium | rbcL and accD junction | 188 | 189 | 190 | 191 |
| hirsutum | | | | | |
| Gossypium | accD and psaI junction | 192 | 193 | 194 | 195 |
| hirsutum | | | | | |
| Gossypium | ycf4 and cemA junction | 196 | 197 | 198 | 199 |
| hirsutum | | | | | |
| Gossypium | petA and psbJ junction | 200 | 201 | 202 | 203 |
| hirsutum | | | | | |
| Gossypium | Rpl20 and clpP junction | 204 | 205 | 206 | 207 |
| hirsutum | | | | | |
| Gossypium | ycf2 and ndhB junction | 208 | 209 | 210 | 211 |
| hirsutum | | | | | |
| Gossypium | ycf2 and ndhB junction | 208 | 212 | 213 | 214 |
| hirsutum | | | | | |
| Gossypium | trnR-ACG-trnN-GUU | 215 | 216 | 217 | 218 |
| hirsutum | junction | | | | |
| Gossypium | trnL-UAG and rpl32 | 219 | 220 | 221 | 222 |
| hirsutum | junction | | | | |
| Zea mays | Rps16-psbK junction | 223 | 224 | 225 | 226 |
| Zea mays | trnS-GCU and psbD | 227 | 228 | 229 | 230 |
| | junction | | | | |
| Zea mays | trnS-UGA and trnG-UCC | 231 | 232 | 233 | 234 |
| | junction | | | | |
| Zea mays | trnfM-CAU-trnG-GGU | 235 | 236 | 237 | 238 |
| | junction | | | | |
| Zea mays | trnD-GUC and psbM | 239 | 240 | 241 | 242 |
| | junction | | | | |
| Zea mays | psbM and petN junction | 243 | 244 | 245 | 246 |
| Zea mays | petN and trnC-GCA | 247 | 248 | 249 | 250 |
| | junction | | | | |
| Zea mays | trnC-GCA and rpoB | 251 | 252 | 253 | 254 |
| | junction | | | | |
| Zea mays | atpI and atpH junction | 255 | 256 | 257 | 258 |
| Zea mays | trnT-UGU and trnL-UAA | 259 | 260 | 261 | 262 |
| | junction | | | | |
| Zea mays | ndhC and trnV-UAC | 263 | 264 | 265 | 266 |
| | junction | | | | |
| Zea mays | atpB and rbcL junction | 267 | 268 | 269 | 270 |
| Zea mays | rbcL and psaI junction | 271 | 272 | 273 | 274 |
| Zea mays | petA and psbJ junction | 275 | 276 | 277 | 278 |
| Zea mays | psbE and petL junction | 279 | 280 | 281 | 282 |
| Zea mays | Rpl20 and rps12 junction | 283 | 284 | 285 | 286 |
| Zea mays | trnI-CAU and trnL-CAA | 287 | 288 | 289 | 290 |
| | junction | | | | |
| Zea mays | Rps7 and trnV-GAC | 291 | 292 | 293 | 294 |
| | junction (duplicate region) | | | | |
| Zea mays | trnN-GUU and rps15 | 295 | 296 | 297 | 298 |
| | junction (duplicate region) | | | | |
| Zea mays | ndhF and rpl32 | 299 | 300 | 301 | 302 |
| Zea mays | Rps15 and trnN-GUU | 303 | 304 | 305 | 306 |
| | junction (duplicate region) | | | | |
| Zea mays | trnV-GAC-rps7 junction | 307 | 308 | 309 | 310 |
| | (duplicate region) | | | | |
| Zea mays | trnL-CAA and trnI-CAU | 311 | 312 | 313 | 314 |
| | region | | | | |

### DETAILED DESCRIPTION

Plastid transformation provides a number of potential advantages over conventional nuclear transformation methods for generating transgenic plants, including generally higher levels of protein expression from transplastomic events due largely to multiple plastids being present in each cell and the presence of multiple copies of plastomic DNA molecules per plastid. Such a higher level of expression may be used to provide, for example, improved agronomic traits or increased biosynthesis of useful products. Plastids can also transcribe genes as operons, allowing for multiple transgenes or even entire pathways to be expressed together from a single expression cassette. In addition, integration of transgenes into the plastome is site-specific and generally less prone to silencing mechanisms, which may provide more consistent and reliable transgene expression levels among events for a given construct. Such consistency may thus reduce development costs in generating successful transplastomic events. Since plastids are generally maternally inherited, all R₁ seed from a plant homoplastomic for a given transgene would have the integrated transgene, unlike plants hemizygous for a nuclear transgenic event that require additional crosses to achieve stable transmission of the transgene due to chromosomal segregation. Transplastomic events may further target or sequester transgenic protein expression to plastids (or chloroplasts) which may direct or contain their function within these organelles without the need for additional target peptide sequences. As a result, plastid expression of a transgene may reduce cytotoxicity in some cases.

However, improved methods of plastid transformation are needed to efficiently and consistently produce transplastomic events within the plastid genome at sites that are less likely to interfere with endogenous structural and RNA encoding genes within the plastid genome. The present invention provides a list of plastid transformation target sites within plant plastid genomes of several crop plant species, useful in generating effective transformation events. Due to the conservation of plastid DNA sequences between related plant species, the polynucleotide sequences provided herein can be further used to identify and define similar plastid target sequences of additional plant species. Constructs for plastid transformation of the target sites provided herein are further disclosed, in addition to plants, plant parts, seeds, etc., transformed with DNA molecules or vectors comprising these constructs and having an insertion of a DNA sequence into their plastid genome. Methods of identifying and transforming useful target sequences within a plant plastid genome are further provided.

### I. Constructs for Plastid Transformation

A. Transformation Vectors and Molecules

An exogenous DNA molecule is provided for plastid transformation according to embodiments of the present invention, which may also be a recombinant DNA molecule. The exogenous DNA molecule may be a linear or circular DNA molecule, although circular DNA plasmids, vectors or constructs may be preferred. Vectors and constructs for plastid transformation according to methods of the present invention may comprise one or more genetic elements and/or transgenes to be introduced into a plant cell or tissue, which may include a selectable marker gene and/or a gene of agronomic interest. These genetic element(s) and/or transgene(s) may be incorporated into a recombinant, double-stranded plasmid or vector DNA molecule that may generally comprise at least the following components: (a) an insertion sequence comprising at least one transgene or expression cassette; and (b) two homology arms (derived from, and corresponding to, target plastid genome sequences of the plant species to be transformed) flanking the insertion sequence. Each of the at least one transgene(s) and/or expression cassette(s) of the insertion DNA sequence may further comprise (i) at least one promoter or regulatory element that functions in plant cells, and more particularly in plant plastids, to cause or drive expression of a transcribable nucleic acid sequence operably linked to the promoter, and (ii) a transcribable DNA sequence encoding a selectable marker or a gene product of agronomic interest (i.e., a selectable marker gene or gene of agronomic interest). Each transgene or expression cassette of the insertion sequence may further comprise a 5′ and a3′ untranslated sequences, intron sequences, additional regulatory or expression elements, etc., for transgene expression from a plant cell plastid transformation event.

According to embodiments of the present invention, the term “recombinant” in reference to a DNA molecule, construct, vector, etc., refers to a DNA molecule or sequence that is not found in nature and/or is present in a context in which it is not found in nature, including a DNA molecule, construct, etc., comprising a combination of DNA sequences that would not naturally occur contiguously or in close proximity together without human intervention, and/or a DNA molecule, construct, etc., comprising at least two DNA sequences that are heterologous with respect to each other. A recombinant DNA molecule, construct, etc., may comprise DNA sequence(s) that is/are separated from other polynucleotide sequence(s) that exist in proximity to such DNA sequence(s) in nature, and/or a DNA sequence that is adjacent to (or contiguous with) other polynucleotide sequence(s) that are not naturally in proximity with each other. A recombinant DNA molecule, construct, etc., may also refer to a DNA molecule or sequence that has been genetically engineered and constructed outside of a cell. For example, a recombinant DNA molecule may comprise any suitable plasmid, vector, etc., and may include a linear or circular DNA molecule. Such plasmids, vectors, etc., may contain various maintenance elements including a prokaryotic origin of replication and selectable marker, as well as a transgene or expression cassette perhaps in addition to a plant selectable marker gene, etc.

According to many embodiments, the insertion sequence between the homology arms may at least comprise a plant selectable marker transgene since selection pressure with a corresponding selection agent may be needed for successful generation of plastid transformants. However, additional transgene(s) and/or transcribable DNA sequence(s) may also be present within the insertion sequence and inserted into the target site of the plastid DNA molecule or genome along with the selectable marker gene, which may include one or more transgenes of agronomic interest conferring one or more agronomically or industrially desirable traits. For example, a transgene of agronomic interest may confer one or more of the following traits: modified carbon fixation, modified nitrogen fixation, herbicide tolerance, insect resistance, improved or increased yield, fungal disease tolerance, virus tolerance, nematode tolerance, bacterial disease tolerance, modified starch production, modified oil production, modified fatty acid content, modified protein production, enhanced animal and human nutrition, environmental stress or drought tolerance, improved processing traits, improved digestibility, modified enzyme production, modified fiber production, etc. An exogenous plasmid or DNA molecule may further comprise other sequence elements required for maintenance of the exogenous DNA molecule or vector, such as a bacterial replication origin, bacterial selection marker, etc., such as in the vector backbone (e.g., outside the homology arms and insertion sequence). Means for preparing DNA plasmids, constructs or vectors containing desired genetic components and sequences are well known in the art.

B. Homology Arms

An exogenous DNA molecule of the present invention may comprise at least two homology arms for homologous recombination at a particular target site or locus within a plastid or plastomic DNA molecule or genome of a target explant cell. The exogenous DNA molecule may comprise a first homology arm (or left homology arm) and a second homology arm (or right homology arm) flanking an insertion sequence between the left and right homology arms. Each of these homology arms may typically have a base pair (bp) length of up to about 5 kilobases (kb), such as in a range from about 0.1 kb to about 5 kb in length (i.e., about 100 to about 5000 nucleotides in length), or in a range from about 0.5 kb to about 2 kb in length, or in a range from about 1 kb to about 1.5 kb in length. The homology arms are positioned on either side of an insertion sequence comprising one or more transgene(s) for insertion into a plastid or plastomic DNA molecule or genome. Each of the homology arms may generally be highly homologous, nearly identical or identical to a corresponding target plastid DNA sequence present in a plastid genome. For example, each homology arm may be at least 80% identical, at least 90% identical, at least 91% identical, at least 92% identical, at least 93% identical, at least 94% identical, at least 95% identical, at least 95% identical, at least 96% identical, at least 97% identical, at least 98% identical, at least 99% identical or 100% identical to at least 50, at least 100, at least 150, at least 200, at least 250, at least 300, at least 400 or at least 500 contiguous nucleotides of a sequence selected from the group consisting of SEQ ID NOs: 3, 7, 11, 15, 19, 22, 26, 30, 33, 37, 41, 45, 49, 53, 57, 61, 64, 68, 72, 76, 80, 84, 88, 92, 96, 100, 104, 108, 112, 116, 120, 123, 127, 131, 135, 138, 142, 146, 150, 154, 158, 162, 166, 170, 174, 178, 182, 186, 190, 194, 198, 202, 206, 210, 213, 217, 221, 225, 229, 233, 237, 241, 245, 249, 253, 257, 261, 265, 269, 273, 277, 281, 285, 289, 293, 297, 301, 305, 309, and 313, or a sequence selected from the group consisting of SEQ ID NOs: 4, 8, 12, 16, 20, 23, 27, 31, 34, 38, 42, 46, 50, 54, 58, 62, 65, 69, 73, 77, 81, 85, 89, 93, 97, 101, 105, 109, 113, 117, 121, 124, 128, 132, 136, 139, 143, 147, 151, 155, 159, 163, 167, 171, 175, 179, 183, 187, 191, 195, 199, 203, 207, 211, 214, 218, 222, 226, 230, 234, 238, 242, 246, 250, 254, 258, 262, 266, 270, 274, 278, 282, 286, 290, 294, 298, 302, 306, 310, and 314, although lower percentages of identity are also possible.

According to some embodiments, each homology arm may comprise at least 50 contiguous nucleotides, or at least 100 contiguous nucleotides, or at least 150 contiguous nucleotides, or at least 200 contiguous nucleotides, or at least 250 contiguous nucleotides, or at least 300 contiguous nucleotides, or at least 400 contiguous nucleotides, or at least 500 contiguous nucleotides, or at least 1000 contiguous nucleotides of a sequence selected from the group consisting of SEQ ID NOs: 3, 7, 11, 15, 19, 22, 26, 30, 33, 37, 41, 45, 49, 53, 57, 61, 64, 68, 72, 76, 80, 84, 88, 92, 96, 100, 104, 108, 112, 116, 120, 123, 127, 131, 135, 138, 142, 146, 150, 154, 158, 162, 166, 170, 174, 178, 182, 186, 190, 194, 198, 202, 206, 210, 213, 217, 221, 225, 229, 233, 237, 241, 245, 249, 253, 257, 261, 265, 269, 273, 277, 281, 285, 289, 293, 297, 301, 305, 309, and 313, or a sequence selected from the group consisting of SEQ ID NOs: 4, 8, 12, 16, 20, 23, 27, 31, 34, 38, 42, 46, 50, 54, 58, 62, 65, 69, 73, 77, 81, 85, 89, 93, 97, 101, 105, 109, 113, 117, 121, 124, 128, 132, 136, 139, 143, 147, 151, 155, 159, 163, 167, 171, 175, 179, 183, 187, 191, 195, 199, 203, 207, 211, 214, 218, 222, 226, 230, 234, 238, 242, 246, 250, 254, 258, 262, 266, 270, 274, 278, 282, 286, 290, 294, 298, 302, 306, 310, and 314. However, except in cases where a targeted mutation or editing of the plastid genome sequence is desired, the homology arms will generally be perfectly or 100% identical to the corresponding target plastid DNA sequences to improve plastid transformation efficiency and avoid introduction of additional mutations.

In addition to the homology arms often being identical or highly homologous to corresponding target plastid DNA sequences, the corresponding target plastid DNA sequences may also be perfectly or almost perfectly continuous with each other prior to the transformation and insertion event (i.e., prior to the insertion sequence of the exogenous DNA molecule becoming inserted into the plastid genome) to avoid making any additional changes or mutations to the plastid DNA sequence as a result of the plastid transformation event—e.g., the deletion of one or more base pairs between the corresponding target plastid DNA sequences. The target site and junction of the corresponding target plastid DNA sequences will also generally or preferably be within an intergenic region or sequence of the plastid DNA to avoid insertion of a transgene into a plastid gene or coding sequence. However, each of the homology arms may comprise or encompass one or more plastid genes, or a portion(s) thereof, within their sequence. According to alternative embodiments, it is further contemplated that the target plastid DNA sequences corresponding to the homology arms may not be continuous with each other (prior to the transformation event), such that the intervening sequence will be deleted by the transformation event and replaced with the exogenous insertion sequence. This approach could thus be used to delete a portion(s) of the plastid genome and/or knockout gene(s) by the transformation event in addition to inserting the exogenous insertion sequence.

An exogenous DNA molecule used for plastid transformation according to present method embodiments may potentially comprise only one homology arm immediately adjacent or next to an insertion sequence comprising one or more transgene(s), such as a plant selectable marker gene and/or a transgene of agronomic interest. However, having only one homology arm may lead to further integration of the vector backbone and/or variable event quality. Even if a linear exogenous DNA molecule is used that lacks additional unwanted vector sequences, such as a bacterial replication origin, selectable marker, etc., such an exogenous DNA molecule may have a much lower transformation frequency and variable event quality. Accordingly, two homology arms flanking the insertion sequence comprising one or more transgene(s) and/or selectable marker gene(s) will generally be preferred for an exogenous DNA molecule or construct to provide a higher transformation efficiency and greater fidelity among transformation events.

According to some embodiments of the present invention, constructs and methods may be further used to engineer, create or introduce one or more mutations (e.g., point mutations or SNPs, deletions, additions, etc.) in the targeted plastid DNA molecule (with or without the additional insertion a gene of agronomic interest). In such a case, the one or more desired mutations relative to the target plastid DNA sequence may be incorporated into one or both of the homology arm(s) of the exogenous DNA molecule such that those mutation(s) may become introduced into the plastid DNA molecule via the homologous recombination event. Despite the possible absence of a gene of agronomic interest within exogenous DNA molecules used for sequence editing or creation of targeted mutations, a plant selectable marker gene may still be present between the two homology arms of the exogenous DNA sequence to allow for selection of transformed cells, tissues and plants with a selection agent. According to other embodiments, a targeted deletion or knockout of an endogenous plastid genome sequence, which may include one or more plastid gene(s), or one or more portion(s) thereof, may also be carried out by the two homologous arms having corresponding plastid target DNA sequences that are not continuous and are separated from each other in the non-transformed plastid genome.

In certain embodiments, the invention provides plastid transformation target sites within junction sequences or regions between transcribable sequences or genes within the plastid genome. Junction sequences may be between any transcribable sequences or genes, for example protein-coding (structural) sequences or genes and/or tRNA-encoding sequences or genes. For example, junction sequences provided by the present invention include SEQ ID NOs: 1, 5, 9, 13, 17, 24, 28, 35, 39, 43, 47, 51, 55, 59, 66, 70, 74, 78, 82, 86, 90, 94, 98, 102, 106, 110, 114, 118, 125, 129, 133, 140, 144, 148, 152, 156, 160, 164, 168, 172, 176, 180, 184, 188, 192, 196, 200, 204, 208, 215, 219, 223, 227, 231, 235, 239, 243, 247, 251, 255, 259, 263, 267, 271, 275, 279, 283, 287, 291, 295, 299, 303, 307, and 311. Target sites for plastid transformation are present within the intergenic regions of the junction sequences. Thus, the homology arms of an exogenous DNA molecule of the present invention are preferably designed such that the insertion sequence of the exogenous DNA molecule becomes inserted or transformed within the target site through recombination. Without being bound by theory, it is believed that target sties for plastid transformation may be closer to tRNA coding sequences than protein coding structural genes in the plastid genome. In some embodiments, a target site may be at least 100 base pairs (bp), at least 150 base pairs, at least 200 base pairs, at least 250 base pairs, at least 300 base pairs or at least 350 base pairs away from the 5′ terminus of a plastid structural (protein-coding) gene; at least 20 base pairs, at least 50 base pairs, at least 100 base pairs, at least 150 base pairs, at least 200 base pairs, at least 250 base pairs, at least 300 base pairs, or at least 350 base pairs away from the 5′ terminus of a transcribable tRNA encoding sequence; at least 100 base pairs, at least 150 base pairs, at least 200 base pairs, at least 250 base pairs, at least 300 base pairs, at least 350 base pairs or at least 400 base pairs away from the 3′ terminus of a plastid structural (protein-coding) gene; and/or at least 20 base pairs, at least 50 base pairs, at least 100 base pairs, at least 150 base pairs, at least 200 base pairs, at least 250 base pairs, at least 300 base pairs, or at least 350 base pairs away from the 3′ terminus of a transcribable tRNA encoding sequence. Target sites for plastid transformation in corn, soybean and cotton may include one of SEQ ID NOs: 2, 6, 10, 14, 18, 21, 25, 29, 32, 36, 40, 44, 48, 52, 56, 60, 63, 67, 71, 75, 79, 83, 87, 91, 95, 99, 103, 107, 111, 115, 119, 122, 126, 130, 134, 137, 141, 145, 149, 153, 157, 161, 165, 169, 173, 177, 181, 185, 189, 193, 197, 201, 205, 209, 212, 216, 220, 224, 228, 232, 236, 240, 244, 248, 252, 256, 260, 264, 268, 272, 276, 280, 284, 288, 292, 296, 300, 304, 308, and 312.

C. Transgene Expression Cassettes

According to embodiments of the present invention, recombinant DNA molecule for plastid transformation may generally comprise an insertion sequence comprising one or more transgene(s), transcribable nucleic acid sequence(s), and/or expression cassette(s) that is/are introduced into a plastid DNA molecule (i.e., a plastid genome or plastome) of a plant or plant cell. Each transgene, expression cassette, etc., will generally comprise a sequence encoding a gene product of agronomic interest and/or a plant selectable marker gene, which may each be operably linked to one or more regulatory element(s), such as promoters, enhancers, leaders, introns, linkers, untranslated regions, termination regions, etc., that are suitable for regulating plastid expression of the transgene or expression cassette. Known examples of plastid regulatory elements suitable for expression in plant plastids, such as from the ribosomal RNA operon plastid gene (Prrn; Staub and Maliga, Plant Cell 4:39-45, 1992), the psbA promoter (Staub and Maliga, EMBO Journal 12:601-606 1993) or the rbcL ribosome binding site region (Svab and Maliga, PNAS 90:913-917, 1993), may be operably linked to a transgene or plant selectable marker gene. See, e.g., Kung, S D, et al., “Chloroplast promoters from higher plants”, Nucleic Acids Res., 13(21): 7543-9 (1985); and Liere, K, et al., “The transcription machineries of plant mitochondria and chloroplasts: Composition, function, and regulation”, Journal of Plant Physiology, 168: 1345-1360 (2011), the entire contents and disclosures of which are hereby incorporated by reference. Plastid regulatory elements or promoters may include those naturally occurring in plastids of the plant species to be transformed, or possibly DNA sequences homologous to those plastid regulatory elements or promoters, or possibly even heterologous plastid regulatory elements or promoters from other closely, or even distantly, related species in plastids of transformed cells. Plastid regulatory elements and promoters may further include synthetic or engineered promoters, as well as promoters altered or derived from other regulatory elements or promoter sequences.

For purposes of the present invention, the term “heterologous” means that the plastid promoter, regulatory element, transgene, selectable marker gene, etc., is from a different species than the plant species to be transformed. Thus, a plastid promoter or regulatory element in exogenous DNA molecules of the present invention may include a homologous, heterologous, or even disparate or divergent plastid or regulatory element sequence(s), in addition to nucleotide sequence(s) identical to a plastid promoter or regulatory element sequence(s) from the plant species to be transformed. A plastid regulatory element or promoter may functionally include any nucleotide sequence element that drives, or at least affects, expression of a transgene operably linked to the regulatory element or promoter (at least transiently) when the plastid regulatory element or promoter and transgene are inserted or integrated into the plastid genome of the plant species to be transformed. Even if a plasmid promoter or regulatory element from the plant species to be transformed is used, the plasmid promoter or regulatory element may be operably linked to a transgene, transcribable nucleotide sequence, selectable marker gene, etc., in a manner, form or combination that (in terms of its exact nucleotide sequence) does not naturally exist in nature, or at least does not naturally exist in the plant species to be transformed.

D. Transcribable Nucleic Acid Sequences

The transcribable nucleic acid or DNA sequence of a transgene or expression cassette within the insertion sequence of an exogenous DNA molecule to be inserted into the plastid genome or plastomic DNA of target explant cells may include a gene of agronomic interest to be expressed in a transplastomic cell or plant. As used herein, the term “gene of agronomic interest” refers to any transgene or expression cassette comprising a transcribable nucleic acid or DNA sequence operably to one or more plastid regulatory element(s) that, when expressed in a plastid of a transgenic plant tissue or cell, provides or confers an agronomically beneficial trait or phenotype, such as a desirable product or characteristic associated with plant morphology, physiology, growth, development, yield, nutritional profile, disease or pest resistance, and/or environmental or chemical tolerance. In some embodiments, a trait of agronomic interest may be modified carbon fixation, modified nitrogen fixation, herbicide tolerance, insect resistance or control, modified or increased yield, fungal disease tolerance or resistance, virus tolerance or resistance, nematode tolerance or resistance, bacterial disease tolerance or resistance, modified starch production, modified oil production, modified fatty acid content, modified protein production, enhanced animal and human nutrition, environmental stress tolerance, drought tolerance, improved processing traits or fruit ripening, improved digestibility, improved taste and flavor characteristics, modified enzyme production, modified fiber production, synthesis of other biopolymers, peptides or proteins, biofuel production, etc.

A gene or transgene of agronomic interest may further include a gene or transcribable DNA sequence of interest that may have unknown characteristics but may be in testing or proposed or theorized for providing a desirable trait of agronomic interest to a plant. Indeed, a transgene of agronomic interest may include any known gene (or any putative or annotated gene sequence) believed, or tested or screened for its ability, to cause, confer, or create a trait or phenotype of agronomic or industrial interest in the transplastomic plant. A transgene of agronomic interest may further include any transcribable DNA sequence that produces a desirable effect in a plant, such as RNA molecule(s) used to confer insect resistance, etc.

Examples of genes of agronomic interest known in the art may include any known or later discovered genes, coding regions or transcribable DNA sequences providing herbicide resistance or tolerance, increased yield, insect resistance or control, fungal disease resistance, virus resistance, nematode resistance, bacterial disease resistance, plant growth and development, starch production, modified oils production, high oil production, modified fatty acid content, high protein production, fruit ripening, enhanced animal or human nutrition, biopolymers, environmental stress resistance, pharmaceutical peptides and secretable peptides, improved processing traits, improved digestibility, low raffinose, industrial enzyme production, improved flavor, nitrogen fixation, hybrid seed production, fiber production, biofuel production, etc.

Plastids can be transformed with polycistronic operons, and can effectively integrate and express large transgenic inserts, thereby enabling stacking of genes and/or simultaneous expression of genes from the same insertion sequence inserted into the plastid DNA by methods of the present invention. As mentioned above, transgenes integrated in plastomes are also generally not susceptiable to gene silencing, which often occurs with multi-copy nuclear events. Thus, plastid transformation according to methods of the present invention may be particularly useful in cases in which high levels of transgene expression are desirable and/or where multiple genes or possibly even entire pathways (or portions of a biochemical pathway) need to be expressed. Accordingly, the insertion sequence of an exogenous DNA molecule may comprise (i) multiple transgenes or cassettes under the control of separate regulatory element(s), and/or (ii) a single transgene or cassette that simultaneously encodes a polycistronic RNA molecule under the control of a common set of regulatory element(s). Such insertion sequences may thus be used to produce multiple gene products from a single plastid DNA insertion event.

E. Selectable markers

According to embodiments of the present invention, the insertion sequence of an exogenous DNA molecule for plastid transformation will generally comprise at least a plant selectable marker gene to allow for successful selection for, and production of, transplastomic R₀ plants. A plant selectable marker gene or transgene may include any gene conferring tolerance to a corresponding selection agent, such that plant cells transformed with the plant selectable marker transgene may tolerate and withstand the selection pressure imposed by the selection agent. As a result, transplastomic cells are favored to grow, proliferate, develop, etc., under selection. Although a plant selectable marker gene is generally used to confer tolerance to a selection agent, additional screenable marker gene(s) may also be used in addition to the selectable marker, perhaps also along with a gene of agronomic interest. Such screenable marker genes may include, for example, β-glucuronidase (GUS; e.g., as described in U.S. Pat. No. 5,599,670, which is hereby incorporated by reference) or green fluorescent protein and variants thereof (GFP described in U.S. Pat. Nos. 5,491,084 and 6,146,826, each of which is hereby incorporated by reference), or any other screenable marker gene known in the art. Additional examples of screenable markers may include secretable markers whose expression causes secretion of a molecule(s) that can be detected as a means for identifying transformed cells.

A plant selectable marker gene may comprise a gene encoding a protein that provides or confers tolerance or resistance to an herbicide, such as glyphosate and glufosinate. Useful plant selectable marker genes known in the art may include those encoding proteins that confer resistance or tolerance to streptomycin or spectinomycin (e.g., aadA, spec/strep), kanamycin (e.g., nptll), hygromycin B (e.g., aph IV), gentamycin (e.g., aac3 and aacC4), and chloramphenicol (e.g., CAT). Additional examples of known plant selectable marker genes encoding proteins that confer resistance or tolerance to an herbicide or other selection agent. For example, a transcribable DNA molecule encoding 5-enolpyruvylshikimate-3-phosphate synthase (EPSPS for glyphosate tolerance; e.g., as described in U.S. Pat. Nos. 5,627,061; 5,633,435; 6,040,497; and 5,094,945, all of which are hereby incorporated by reference); a transcribable DNA molecule encoding a glyphosate oxidoreductase and a glyphosate-N-acetyl transferase (GOX; e.g., as described in U.S. Pat. No. 5,463,175; GAT described in U.S. Patent Publication No. 20030083480); a transcribable DNA molecule encoding phytoene desaturase (crtI; e.g., as described in Misawa, et al., Plant Journal, 4:833-840 (1993) and Misawa, et al., Plant Journal, 6:481-489 (1994) for norflurazon tolerance, incorporated herein by reference); and the bar gene (e.g., as described in DeBlock, et al., EMBO Journal, 6:2513-2519 (1987) for glufosinate and bialaphos tolerance, incorporated herein by reference). See also, e.g., Bock, R., “Engineering Plastid Genomes: Methods, Tools, and Applications in Basic Research and Biotechnology,” Annu. Rev. Plant Biol., 66: 3.1-3.31 (2015), and Ziemienowicz, A., “Plant selectable markers and reporter genes,” Acta Physiologiae Plantarum 23(3): 363-374 (2001), the entire contents and disclosure of which are hereby incorporated by reference.

The insertion sequence of an exogenous DNA molecule may further comprise sequences for removal of one or more transgene(s) or expression cassette(s), such as a plant selectable marker transgene, or any portion or sequence thereof, after successful production and/or confirmation of a transplastomic plant(s), especially after the transgene or expression cassette is no longer needed. In some embodiments, this may be accomplished by flanking the transgene sequence to be removed, with known of later developed recombination sites (e.g., LoxP sites, FRT sites, etc.) that can be recognized and removed by an endogenous or exogenously provided recombinase enzyme (e.g., Cre, Flp, etc.). The recombinase enzyme may be introduced and expressed in trans, such as by crossing the transplastomic plant to another plant having the recombinase transgene, to accomplish excision of the transgene. Accordingly, the unwanted sequence element or transgene can be removed once its use or purpose has expired, thus preventing its further expression or transmission in the germ line.

F. 3′ Untranslated Regions

An insertion sequence according to the present invention may further comprise a 3′-untranslated region to facilitate mRNA stability in the plastid. Transgene transcripts may be stabilized, for example, by inclusion of the 3′-untranslated region of the plastid rps16 gene (Trps16; U.S. Pat. No. 5,877,402) or the 3′-untranslated region of the plastid petD gene (TpetD) downstream of a transcribable nucleic acid sequence.

### II. Methods of Plastid Transformation

Embodiments of the present invention provide methods of transforming plant plastids comprising introducing an exogenous DNA molecule into at least one cell of a plant tissue to produce a transplastomic event in at least one plastid of that cell. A “plastid” refers to a class of organelles in the cytoplasm of a plant cell which contain one or more small circular double-stranded DNA molecules (i.e., the plastome, plastomic DNA, or plastid DNA). Examples of plastids include, but are not limited to, proplastids, chloroplasts, chromoplasts, gerontoplasts, leucoplasts, elaioplasts, proteinoplasts, and tannosomes. As used herein, plant plastid refers to a plastid in higher plants (i.e., a dicot or a monocot).

Methods of the present invention employ homologous recombination to achieve site-specific insertion of a transgene from an exogenous DNA molecule into the plastid genome DNA (i.e., the “plastome”) of at least one cell of the explant target tissue. As described above, the exogenous DNA molecule may generally comprise two arm regions flanking an insertion sequence with each of the two arm regions being homologous to respective target plastid genome sequences to drive recombination and insertion of the transgene into the target site of the plastome. Site-directed integration of the transgene into the plastid genome of a plant cell via homologous recombination reduces event variability commonly associated with nuclear transformation events having transgene insertions at different locations throughout the nuclear genome. As a result, transgene expression levels in plastids should generally be consistent between transplastomic events of the same quality (unlike nuclear transformation events that exhibit variable and unpredictable levels of transgene expression depending on their insertion site). Such consistent and predictable transgene expression reduces development costs for producing transplastomic events.

Various methods have been developed for transferring genes into plant tissue cells including high velocity microprojection or particle bombardment, microinjection, electroporation, PEG-mediated transformation, direct DNA uptake, and bacterially-mediated transformation. According to embodiments of the present invention, an exogenous DNA molecule may preferably be introduced into at least one cell of a target plant tissue via particle-mediated bombardment of the explant using particles carrying one or more copies of the exogenous DNA molecule. Such particle-mediated bombardment may utilize any suitable particle gun device known in the art, such as a helium particle gun, electric particle gun, etc. Prior to bombardment, particles may be loaded or coated with copies of the exogenous DNA molecule. The particles themselves may include any suitable type of particle or bead known in the art, such as gold or tungsten beads, etc. According to embodiments of the present invention, a ratio in a range of approximately 0.5-2.0 μg of exogenous DNA molecules per mg of beads, such as about 1.2 μg of exogenous DNA per mg of beads, may be combined together for bead preparation and coating. Methods for coating beads with an exogenous DNA molecule are known in the art. Blasting conditions for the particle gun are also well known in the art, and various conventional screens, rupture disks, etc., may be used, such as for a helium particle gun. The electric gun may provide some advantages in reducing the amount of time required for transformation and by using fewer consumables in the process.

For particle bombardment, plant tissue may be plated onto a target medium or substrate that is able to hold the plant tissue in place and properly oriented for blasting. Such a target medium or substrate may contain, for example, a gelling agent, such as agar, and carboxymethylcellulose (CMC) to control the viscosity of the medium or substrate. Plant tissue may also be blasted with coated particles at various pressures, forces, and/or once or multiple times. Although particle mediated bombardment may be preferred for plastid transformation of explants according to embodiments of the present invention, other non-conventional methods are contemplated for use potentially in plastid transformation.

After transformation or bombardment, the plant tissue may be contacted with one or more selection media containing a selection agent to bias the survival, growth, proliferation and/or development of transplastomic cells having expression of a selectable marker gene integrated into the plastome from the exogenous DNA molecule used for transformation. The selectable marker gene will generally be paired to the selection agent used for selection such that the selectable marker gene confers tolerance to selection with the selection agent. For example, the selectable marker gene may be an adenylyltransferase gene (aadA) conferring tolerance to spectinomycin or streptomycin as the selection agent.

The methods of the present invention allow for identification and selection of sites for plastid transformation that will minimize or avoid interference with endogenous plastid genes. Candidate transplastomic plants from one or more transformed explant(s) may be identified and plastid transformed shoots and plants may be grown or developed to produce transplastomic plants. For example, after putative plastid transformants have been identified using selectable markers, plantlets may be subcultured and/or placed in soil or on a soil substitute such as on a rooting medium, in the presence or absence of the selection agent. Shoots elongating from explants may be assayed to determine whether they are transgenic. Transgenic R₁ seed may be collected from R₀ plants to produce progeny plants that are also transplastomic. Selection pressure with the appropriate selection agent may be maintained over one or more subsequent generations from the R₀ plant to produce a homoplastomic or nearly homoplastomic plant, which may be defined as being fixed with respect to inheritance of the plastome-integrated transgene (i.e., without segregation of the transgene among progeny and/or with stable maintenance of homoplastomy in progeny with self-crossing). Growth, survival, development, etc., of transplastomic cells in the R₀ plant may also be selectively achieved or favored by exerting a selection pressure with a selection agent during culturing, sub-culturing, shoot elongation and/or rooting step(s) of the explant to produce a homoplastomic or nearly homoplastomic R₀ plant, or at least a transplastomic R₀ plant having a uniform, ubiquitous or more widespread presence of transgenic plastids throughout the R₀ plant, although selection pressure may alternatively be continued (e.g., periodically, etc.) during the remaining life of the R₀ plant (e.g., as a topical spray, soil or seed application, etc.). Selection pressure may also be continued or maintained over subsequent generation(s) to produce a progeny plant that is homoplastomic or nearly homoplastomic, or at least has a more uniform, widespread and/or ubiquitous presence of transgenic plastids throughout the plant.

A variety of tissue culture media are known that, when supplemented appropriately, support plant tissue growth and development, including formation of mature plants from excised plant tissue. These tissue culture media can either be purchased as a commercial preparation or custom prepared and modified by those of skill in the art. Examples of such media include, but are not limited to those described by Murashige and Skoog, (1962); Chu et al., (1975); Linsmaier and Skoog, (1965); Uchimiya and Murashige, (1962); Gamborg et al., (1968); Duncan et al., (1985); McCown and Lloyd, (1981); Nitsch and Nitsch (1969); and Schenk and Hildebrandt, (1972), or derivations of these media supplemented accordingly. Those of skill in the art are aware that media and media supplements, such as nutrients and plant growth regulators for use in transformation and regeneration are usually optimized for the particular target crop or variety of interest. Tissue culture media may be supplemented with carbohydrates such as, but not limited to, glucose, sucrose, maltose, mannose, fructose, lactose, galactose, and/or dextrose, or ratios of carbohydrates. Reagents are commercially available and can be purchased from a number of suppliers (see, for example Sigma Chemical Co., St. Louis, Mo.; and PhytoTechnology Laboratories, Shawnee Mission, Kans.). These tissue culture media may be used as a resting media or as a selection media with the further addition of a selection agent.

A variety of assays may be performed to confirm the presence of an exogenous DNA and/or insertion sequence in transplastomic plants. Such assays include, for example, molecular biological assays, such as Southern and Northern blotting, sequencing, PCR, in situ hybridization, etc.; biochemical assays, such as detecting the presence of a protein product, e.g., by immunological means (ELISAs and Western blots) or by enzymatic function; visual determination with a screenable marker plant part assays, such as leaf or root assays; or by analyzing the phenotype of a whole regenerated plant or plant part.

Embodiments of the present invention also provide transplastomic plants and/or plant parts produced by the plastid transformation methods of the present invention as disclosed herein. Plant parts, without limitation, include fruit, seed, endosperm, ovule, pollen, leaf, stem, and roots. In certain embodiments of the present invention, the plant or plant part is a seed.

Plants for use with the method embodiments provided herein may include a wide variety of dicotyledonous (dicot) or monocotyledonous (monocot) plants. Examples of dicot plants may include various agricultural crop species, such as soybean, alfalfa and other Fabaceae or leguminous plants, and cotton, canola, and sugar beets. Other examples of dicot plants include a member of the Brassica sp. (e.g., B. napus, B. rapa, B. juncea), particularly those Brassica species useful as sources of seed oil, alfalfa (Medicago sativa), sunflower (Helianthus annuus), safflower (Carthamus tinctorius), oil palm (Elaeis spp.), sesame (Sesamum spp.), coconut (Cocos spp.), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), tea (Camellia spp.), fruit trees, such as apple (Malus spp.), Prunus spp., such as plum, apricot, peach, cherry, etc., pear (Pyrus spp.), fig (Ficus casica), banana (Musa spp.), etc., citrus trees (Citrus spp.), cocoa (Theobroma cacao), avocado (Persea americana), olive (Olea europaea), almond (Prunus amygdalus), walnut (Juglans spp.), strawberry (Fragaria spp.), watermelon (Citrullus lanatus), pepper (Capsicum spp.), sugar beet (Beta vulgaris), grape (Vitis, Muscadinia), tomato (Lycopersicon esculentum, Solanum lycopersicum), and cucumber (Cucumis sativis). Examples of monocotyledonous (monocot) plants various agricultural crop species, such as corn (maize), wheat, rice, millet, barley, sorghum, sugarcane, oat, rye, and other Poaceae or Gramineae family of plants that are typically harvested for their seed. Although examples of target and flanking sequences for plastid transformation are provided for a few plant species, one skilled in the art would be able to use the sequences provided herein to determine analogous plastid sequences in other plant species through sequence alignment and comparison.

Having described the present disclosure in detail, it will be apparent that modifications, variations, and equivalent embodiments are possible without departing from the spirit and scope of the present disclosure as further defined in the appended claims. Furthermore, it should be appreciated that all examples in the present disclosure are provided as non-limiting examples.

### EXAMPLES

### Example 1: Identification of Target Sequences for Plastid Transformation

Specific sequences from soybean, cotton, and corn plastid genomes useful as plastid transformation target sites are provided herein. Target sequences may include SEQ ID NOs: 2, 6, 10, 14, 18, 21, 25, 29, 32, 36, 40, 44, 48, 52, 56, 60, 63, 67, 71, 75, 79, 83, 87, 91, 95, 99, 103, 107, 111, 115, 119, 122, 126, 130, 134, 137, 141, 145, 149, 153, 157, 161, 165, 169, 173, 177, 181, 185, 189, 193, 197, 201, 205, 209, 212, 216, 220, 224, 228, 232, 236, 240, 244, 248, 252, 256, 260, 264, 268, 272, 276, 280, 284, 288, 292, 296, 300, 304, 308, and 312. Eighty target sites were evaluated for distance to the nearest 5′ end and distance to the nearest 3′ end of a structural gene, and/or distance to the nearest 5′ end and distance to the nearest 3′ end of a tRNA-encoding sequence, depending on the neighboring genes of the target sequence.

For corn, soybean and cotton, target sequences were determined to be approximately 100 bp to 450 bp away from the 5′ terminus of plastid structural (protein-coding) genes, and approximately 150 bp to 450 bp away from the 3′ terminus of plastid structural (protein-coding) genes. In certain examples, target sequences were determined to be approximately 140 bp to 400 bp from the 5′ terminus of structural gene sequences (e.g., 143-399 bp from the 5′ terminus), and approximately 170 bp to 420 bp from the 3′ terminus of structural gene sequences (e.g., 173-419 bp from the 3′ terminus).

For corn, soybean and cotton, target sequences were also determined to be approximately 20 bp to 400 bp away from the 5′ terminus and 3′ terminus of tRNA gene sequences. In certain examples, target sequences were determined to be approximately 25 bp to 350 bp from a 5′ terminus of tRNA gene sequences (e.g., 27-350 bp from the 5′ terminus), and approximately 20 bp to 400 bp from a 3′ terminus of tRNA gene sequences (e.g., 24-383 bp from the 3′ terminus).

Exogenous DNA insertion sequences may be specifically integrated into the identified target sites, or portions of the target sites, or completely or partially replace the identified target sites, as a result of a plastid transformation event.

An exemplary method of selecting a target site for plastid transformation includes identifying a sequence within a plastid genome which has the following characteristics:
The sequence is located between two neighboring plastid genes.
The sequence is at least 20 bp away from the 5′ terminus of a tRNA (trn) or other small RNA encoding gene within a plastid genome;
The sequence is at least 20 bp away from the 3′ terminus of a tRNA (trn) or other small RNA encoding gene within a plastid genome;
The sequence is at least 100 bp away from the 5′ terminus of a structural (protein encoding) gene within a plastid genome; and
The sequence is at least 150 bp away from the 3′ terminus of a structural (protein encoding) gene within a plastid genome.

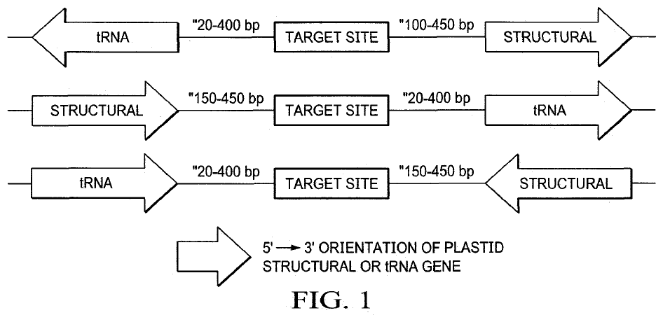

Three configurations of neighboring or flanking plastid genes next to target sites for targeted integration and/or recombination of exogenous DNA molecules in plant plastid genomes are shown schematically in FIG. 1 with distance ranges provided relative to neighboring structural or tRNA genes.

### Example 2: Target Sequences for Soybean Plastid Transformation

Target sites for plastid transformation were identified in the soybean chloroplast genome (NCBI, Accession No. NC_007942).

Junction regions of neighboring structural and/or tRNA gene sequences comprising target sites for soybean plastid transformation are provided as SEQ ID NOs: 1, 5, 9, 13, 17, 24, 28, 35, 39, 43, 47, 51, 55, 59, 66, 70, 74, 78, 82, 86, 90, 94, 98, 102, and 106. Target sites for soybean plastid transformation within the junction regions are provided as SEQ ID NOs: 2, 6, 10, 14, 18, 21, 25, 29, 32, 36, 40, 44, 48, 52, 56, 60, 63, 67, 71, 75, 79, 83, 87, 91, 95, 99, 103, and 107. Transgenic DNA inserted during transformation may be integrated between any two base pairs, and may partially or completely replace the target sequences by homologous recombination. The sequences identified and constructs provided are useful for introducing transgenic DNA into a specific site of a soybean chloroplast genome via transformation.

### Example 3: Target Sequences for Cotton Plastid Transformation

Target sites for plastid transformation were identified in the cotton chloroplast genome (NCBI, Accession No. NC_007944).

Junction regions of neighboring structural and/or tRNA gene sequences comprising target sites for cotton plastid transformation are provided as SEQ ID NOs: 110, 114, 118, 125, 129, 133, 140, 144, 148, 152, 156, 160, 164, 168, 172, 176, 180, 184, 188, 192, 196, 200, 204, 208, 215, and 219. Target sites for cotton plastid transformation within the junction regions are provided as SEQ ID NOs: 111, 115, 119, 122, 126, 130, 134, 137, 141, 145, 149, 153, 157, 161, 165, 169, 173, 177, 181, 185, 189, 193, 197, 201, 205, 209, 212, 216, and 220. Transgenic DNA inserted during transformation may be integrated between any two base pairs, and may partially or completely replace the target sequences by homologous recombination. The sequences identified and constructs provided are useful for introducing transgenic DNA into a specific site of a cotton chloroplast genome via transformation.

### Example 4: Target Sequences for Corn Plastid Transformation

Target sites for plastid transformation were identified in the corn chloroplast genome (NCBI, Accession No. KF241981).

Junction regions of neighboring structural and/or tRNA gene sequences comprising target sites for corn plastid transformation are provided as SEQ ID NOs: 223, 227, 231, 235, 239, 243, 247, 251, 255, 259, 263, 267, 271, 275, 279, 283, 287, 291, 295, 299, 303, 307, and 311. Target sites for corn plastid transformation within the junction regions are provided as SEQ ID NOs: 224, 228, 232, 236, 240, 244, 248, 252, 256, 260, 264, 268, 272, 276, 280, 284, 288, 292, 296, 300, 304, 308, and 312. Transgenic DNA inserted during transformation may be integrated between any two base pairs, and may partially or completely replace the target sequences by homologous recombination. The sequences identified and constructs provided are useful for introducing transgenic DNA into a specific site of a corn chloroplast genome via transformation.

### Example 5: Plastid Transformation Constructs

Plastid transformation constructs are designed to include a first homology arm region and a second homology arm region capable of directing insertion of an insertion sequence into a target site within a plastid DNA molecule or genome. Depending on the plant species, plastid transformation constructs are designed to insert heterologous sequences into plastid target sites including sequences selected from the group consisting of any one of SEQ ID NOs: 2, 6, 10, 14, 18, 21, 25, 29, 32, 36, 40, 44, 48, 52, 56, 60, 63, 67, 71, 75, 79, 83, 87, 91, 95, 99, 103, 107, 111, 115, 119, 122, 126, 130, 134, 137, 141, 145, 149, 153, 157, 161, 165, 169, 173, 177, 181, 185, 189, 193, 197, 201, 205, 209, 212, 216, 220, 224, 228, 232, 236, 240, 244, 248, 252, 256, 260, 264, 268, 272, 276, 280, 284, 288, 292, 296, 300, 304, 308, and 312.

Plastid transformation constructs are designed to include a first homology arm including a sequence corresponding (e.g., identical or similar) to a plastid target site or flanking region. Plastid transformation constructs may be designed to include, for example, a first homology arm sequence comprising at least a portion of a sequence selected from the group consisting of SEQ ID NOs: 3, 7, 11, 15, 19, 22, 26, 30, 33, 37, 41, 45, 49, 53, 57, 61, 64, 68, 72, 76, 80, 84, 88, 92, 96, 100, 104, 108, 112, 116, 120, 123, 127, 131, 135, 138, 142, 146, 150, 154, 158, 162, 166, 170, 174, 178, 182, 186, 190, 194, 198, 202, 206, 210, 213, 217, 221, 225, 229, 233, 237, 241, 245, 249, 253, 257, 261, 265, 269, 273, 277, 281, 285, 289, 293, 297, 301, 305, 309, and 313, or a variant or fragment thereof.

Plastid transformation constructs are designed to include a second homology arm including a sequence corresponding (e.g., identical or similar) to a plastid target site or flanking region. Plastid transformation constructs may be designed to include, for example, a second homology arm sequence comprising at least a portion of a sequence selected from the group consisting of SEQ ID NOs: 4, 8, 12, 16, 20, 23, 27, 31, 34, 38, 42, 46, 50, 54, 58, 62, 65, 69, 73, 77, 81, 85, 89, 93, 97, 101, 105, 109, 113, 117, 121, 124, 128, 132, 136, 139, 143, 147, 151, 155, 159, 163, 167, 171, 175, 179, 183, 187, 191, 195, 199, 203, 207, 211, 214, 218, 222, 226, 230, 234, 238, 242, 246, 250, 254, 258, 262, 266, 270, 274, 278, 282, 286, 290, 294, 298, 302, 306, 310, and 314, or a variant or fragment thereof.

Plastid transformation constructs comprising a first homology arm and a second homology arm as described are capable of inserting an insertion sequence into a particular target site or locus within a plastid genome via homologous recombination.

1. A recombinant plastid transformation construct, said construct comprising:
(i) a first homology arm comprising a sequence that is at least 95% identical to at least 100 contiguous nucleotides of any one of SEQ ID NOs: 3, 7, 11, 15, 19, 22, 26, 30, 33, 37, 41, 45, 49, 53, 57, 61, 64, 68, 72, 76, 80, 84, 88, 92, 96, 100, 104, 108, 112, 116, 120, 123, 127, 131, 135, 138, 142, 146, 150, 154, 158, 162, 166, 170, 174, 178, 182, 186, 190, 194, 198, 202, 206, 210, 213, 217, 221, 225, 229, 233, 237, 241, 245, 249, 253, 257, 261, 265, 269, 273, 277, 281, 285, 289, 293, 297, 301, 305, 309, or 313; and
(ii) a second homology arm comprising a sequence that is at least 95% identical to at least 100 contiguous nucleotides of any one of SEQ ID NOs: 4, 8, 12, 16, 20, 23, 27, 31, 34, 38, 42, 46, 50, 54, 58, 62, 65, 69, 73, 77, 81, 85, 89, 93, 97, 101, 105, 109, 113, 117, 121, 124, 128, 132, 136, 139, 143, 147, 151, 155, 159, 163, 167, 171, 175, 179, 183, 187, 191, 195, 199, 203, 207, 211, 214, 218, 222, 226, 230, 234, 238, 242, 246, 250, 254, 258, 262, 266, 270, 274, 278, 282, 286, 290, 294, 298, 302, 306, 310, or 314.

2. The recombinant plastid transformation construct of claim 1, wherein the first homology arm comprises at least 100 contiguous nucleotides of any one of SEQ ID NOs: 3, 7, 11, 15, 19, 22, 26, 30, 33, 37, 41, 45, 49, 53, 57, 61, 64, 68, 72, 76, 80, 84, 88, 92, 96, 100, 104, 108, 112, 116, 120, 123, 127, 131, 135, 138, 142, 146, 150, 154, 158, 162, 166, 170, 174, 178, 182, 186, 190, 194, 198, 202, 206, 210, 213, 217, 221, 225, 229, 233, 237, 241, 245, 249, 253, 257, 261, 265, 269, 273, 277, 281, 285, 289, 293, 297, 301, 305, 309, or 313.

3. The recombinant plastid transformation construct of claim 1, wherein the second homology arm comprises at least 100 contiguous nucleotides of any one of SEQ ID NOs: 4, 8, 12, 16, 20, 23, 27, 31, 34, 38, 42, 46, 50, 54, 58, 62, 65, 69, 73, 77, 81, 85, 89, 93, 97, 101, 105, 109, 113, 117, 121, 124, 128, 132, 136, 139, 143, 147, 151, 155, 159, 163, 167, 171, 175, 179, 183, 187, 191, 195, 199, 203, 207, 211, 214, 218, 222, 226, 230, 234, 238, 242, 246, 250, 254, 258, 262, 266, 270, 274, 278, 282, 286, 290, 294, 298, 302, 306, 310, or 314.

4. The recombinant plastid transformation construct of claim 1, wherein the first homology arm is between 0.1 and 2.0 kilobases in length.

5. The recombinant plastid transformation construct of claim 1, wherein the second homology arm is between 0.1 and 2.0 kilobases in length.

6. The recombinant plastid transformation construct of claim 1, further comprising an insertion sequence positioned between the first homology arm and the second homology arm.

7. The recombinant plastid transformation construct of claim 6, wherein said insertion sequence comprises a transcribable nucleic acid sequence.

8. The recombinant plastid transformation construct of claim 7, wherein said transcribable nucleic acid sequence is a gene of agronomic interest.

9. The recombinant plastid transformation construct of claim 8, wherein said gene of agronomic interest confers an agronomically beneficial trait selected from the group consisting of modified carbon fixation, modified nitrogen fixation, herbicide tolerance, insect resistance or control, modified or increased yield, fungal disease tolerance or resistance, virus tolerance or resistance, nematode tolerance or resistance, bacterial disease tolerance or resistance, modified starch production, modified oil production, modified fatty acid content, modified protein production, enhanced animal and human nutrition, environmental stress tolerance, drought tolerance, improved processing traits or fruit ripening, improved digestibility, improved taste and flavor characteristics, modified enzyme production, modified fiber production, synthesis of other biopolymers, peptides or proteins, and enhanced biofuel production.

10. The recombinant plastid transformation construct of claim 6, wherein said insertion sequence comprises a promoter active in plant plastids.

11. The recombinant plastid transformation construct of claim 10, wherein said promoter is selected from the group consisting of Prrn, psbA, and rbcL.

12. The recombinant plastid transformation construct of claim 6, wherein said insertion sequence comprises a selectable marker.

13. The recombinant plastid transformation construct of claim 12, wherein said selectable marker is selected from the group consisting of aadA, nptII, aph IV, aac3, aacC4, CAT, EPSPS, bar, GOX, GAT, and β-glucuronidase.

14. The recombinant plastid transformation construct of claim 6, wherein said insertion sequence comprises a screenable marker.

15. A DNA molecule or vector comprising the recombinant plastid transformation construct of claim 1.

16. A plastid comprising the recombinant plastid transformation construct of claim 1.

17. A plant, plant cell, plant part, or seed comprising the plastid of claim 16.

18. The plant, plant cell, plant part, or seed of claim 17, wherein said plant is a crop plant.

19. The plant, plant cell, plant part, or seed of claim 18, wherein said plant is a monocotyledonous plant.

20. The plant of claim 19, wherein said monocotyledonous plant is selected from the group consisting of corn (maize), wheat, rice, millet, barley, sorghum, sugarcane, oat, rye, and other plants within the Poaceae or Gramineae family.

21. The plant, plant cell, plant part, or seed of claim 18, wherein said plant is a dicotyledonous plant.

22. The plant of claim 21, wherein said dicotyledonous plant is selected from the group consisting of cotton, canola, and sugar beets, soybean, alfalfa and other Fabaceae or leguminous plants.

23. A method of producing a transformed plant cell plastid, comprising the step of:
transforming at least one plastid of a plant cell with the recombinant plastid transformation construct of claim 6 to produce plant cell comprising a transformed plastid; wherein the insertion sequence is incorporated into a genome of said plastid flanked by plastid sequences corresponding to the first and second homology arms of the recombinant plastid transformation construct.

24. The method of claim 23, wherein the insertion sequence is incorporated into the genome of said transformed plastid by homologous recombination.

25. A transformed plastid produced by the method of claim 23.

26. The method of claim 23, further comprising the step of:
selecting for development or regeneration of a plastid transformed plant cell by contacting said plant cell with a selection agent.

27. A plastid transformed plant cell produced by the method of claim 23.

28. The method of claim 23, further comprising the step of:
developing or regenerating a plastid transformed plant from said plant cell.

29. The method of claim 28, wherein said plastid transformed plant is developed or regenerated from said plant cell under selection pressure by contacting the developing plant with a selection agent.

30. A plastid transformed plant produced by the method of claim 23.

31. The method of claim 28, further comprising the step of:
obtaining a plastid transformed seed from said plastid transformed plant.

32. A plastid transformed seed produced by the method of claim 31.

33. A method of selecting a target site for plastid transformation, comprising identifying a plastid target sequence, wherein said target sequence is located: (1) between two neighboring plastid genes in the plastid genome of a plant cell, (2) at least 20 base pairs from the 5′ terminus of the coding sequence of a tRNA or small RNA encoding plastid gene, (3) at least 100 base pairs from the 5′ terminus of the coding sequence of a structural protein-encoding plastid gene, (4) at least 20 base pairs from the 3′ terminus of the coding sequence of a tRNA or small RNA encoding plastid gene, and (5) at least 150 base pairs from the 3′ terminus of the coding sequence of a structural protein-encoding plastid gene.

34. A recombinant plastid transformation construct, said construct comprising a homology arm comprising a sequence that is at least 95% identical to at least 100 contiguous nucleotides of any one of SEQ ID NO: 3, 7, 11, 15, 19, 22, 26, 30, 33, 37, 41, 45, 49, 53, 57, 61, 64, 68, 72, 76, 80, 84, 88, 92, 96, 100, 104, 108, 112, 116, 120, 123, 127, 131, 135, 138, 142, 146, 150, 154, 158, 162, 166, 170, 174, 178, 182, 186, 190, 194, 198, 202, 206, 210, 213, 217, 221, 225, 229, 233, 237, 241, 245, 249, 253, 257, 261, 265, 269, 273, 277, 281, 285, 289, 293, 297, 301, 305, 309, or 313; or at least 95% identical to at least 100 contiguous nucleotides of any one of SEQ ID NO: 4, 8, 12, 16, 20, 23, 27, 31, 34, 38, 42, 46, 50, 54, 58, 62, 65, 69, 73, 77, 81, 85, 89, 93, 97, 101, 105, 109, 113, 117, 121, 124, 128, 132, 136, 139, 143, 147, 151, 155, 159, 163, 167, 171, 175, 179, 183, 187, 191, 195, 199, 203, 207, 211, 214, 218, 222, 226, 230, 234, 238, 242, 246, 250, 254, 258, 262, 266, 270, 274, 278, 282, 286, 290, 294, 298, 302, 306, 310, or 314.
